SEQUENCE LISTING (119 sequences)

SEQ ID NO: 1 - protein, 1307 aa, synthetic construct
  REGION 1..1307: note = Synthetic construct
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 2 - protein, 492 aa, synthetic construct
  REGION 1..492: note = Synthetic construct
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 3 - protein, 529 aa, synthetic construct
  REGION 1..529: note = Synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 4 - protein, 1297 aa, synthetic construct
  REGION 1..1297: note = Synthetic construct
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 5 - protein, 1145 aa, synthetic construct
  REGION 1..1145: note = Synthetic construct
MCMKITKIDGISHKKYKEKGKLIKNNDTAKDIIEERFNDIEKKTKELFQKTLDFYVKNYEKCKEQNKERREKAKNYFSKVKILVDNKKITICNENTEKMEIEDFNEYDVRSGKYFNVLNKILNGENYTEEDLEVFENDLQKRTGRIKSIKNSLEENKAHFKKESINNNIIYDRVKGNNKKSLFYEYYRISSKHQEYVNNIFEAFDKLYSNSHEAMNNLFSEITKDSKDRNIRKIREAYHEILNKNKTEFGEELYKKIQDNRNNFDKLLEIEPEIKELTKSQIFYKYYIDKVNLDETSIKHCFCHLVEIEVNQLLKNYVYSKRNINKEKLENIFEYCKLKNLIKNKLVNKLNNYIRNCGKYNAYISNNDVVVNSEKISEIRTKEAFLRSIIGVSSSAYFSLRNILNTDNTQDITNKVDKEVDKLYQENKKIELEERLKLFFGNYFDINNQQEIKVFLMNIDKIISSIRHEIIHFKMETNAQNIFDFNNVNLGNTAKNIFSNEINEEKIKFKIFKQLNSANVFDYLSNKDITEYMDKVVFSFTNRNVSFVPSFTKIYNRVQDLANSLEIKKWKIPDKSEGKDAQIYLLKNIYYGKFLDEFLNEENGIFISIKDKIIELNRNQNKRTGFYKLEKFEKIEETNPKKYLEIIQSLYMINIEEIDSEGKNIFLDFIQKIFLKGFFEFIKNNYNYLLELKKIQDKKNIFDSEMSEYIAGEKTLEDIGEINEIIQDIKITEIDKILNQTDKINCFYLLLKLLNYKEITELKGNLEKYQILSKTNVYEKELMLLNIVNLDNNKVKIENFKILAEEIGKFIEKINIEEINKNKKIKTFEELRNFEKGENTGEYYNIYSDDKNIKNIRNLYNIKKYGMLDLLEKISEKTNYCIKKKDLEEYSELRKQLEDEKTNFYKIQEYLHSKYQQKPKKILLKNNKNDYEKYKKSIENIEKYVHLKNKIEFNELNLLQSLLLKILHRLVGFTSIWERDLRFRLIGEFPDELDVEDIFDHRKRYKGTGKGICKKYDRFINTHTEYKNNNKMENVKFADNNPVRNYIAHFNYLPNPKYSILKMMEKLRKLLDYDRKLKNAVMKSIKDILEEYGFKAEFIINSDKEIILNLVKSVEIIHLGKEDLKSRRNSEDLCKLVKAMLEYSK

SEQ ID NO: 6 - protein, 1271 aa, synthetic construct
  REGION 1..1271: note = Synthetic construct
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 7 - protein, 1229 aa, synthetic construct
  REGION 1..1229: note = Synthetic construct
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 8 - protein, 835 aa, synthetic construct
  REGION 1..835: note = Synthetic construct
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 9 - protein, 1264 aa, synthetic construct
  REGION 1..1264: note = Synthetic construct
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 10 - protein, 500 aa, synthetic construct
  REGION 1..500: note = Synthetic construct
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 11 - protein, 1261 aa, synthetic construct
  REGION 1..1261: note = Synthetic construct
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 12 - protein, 478 aa, synthetic construct
  REGION 1..478: note = Synthetic construct
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 13 - protein, 1229 aa, synthetic construct
  REGION 1..1229: note = Synthetic construct
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 14 - protein, 1242 aa, synthetic construct
  REGION 1..1242: note = Synthetic construct
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 15 - protein, 1168 aa, synthetic construct
  REGION 1..1168: note = Synthetic construct
MKITKIDGILHKKYIKEGKLVKSTSEENKTDERLSELLTIRLDTYIKNPDNASEEENRIRRETLKEFFSNKVLYLKDSILYLKDRREKNQLQNKNYSEEDISEYDLKNKNSFLVLKKILLNEDINSEELEIFRNDFEKKLDKINSLKYSLEENKANYQKINENNIKKVEGKSKRNIFYNYYKDSAKRNDYINNIQEAFDKLYKKEDIENLFFLIENSKKHEKYKIRECYHKIIGRKNDKENFATIIYEEIQNVNNMKELIEKVPNVSELKKSQVFYKYYLNKEKLNDENIKYVFCHFVEIEMSKLLKNYVYKKPSNISNDKVKRIFEYQSLKKLIENKLLNKLDTYVRNCGKYSFYLQDGEIATSDFIVGNRQNEAFLRNIIGVSSTAYFSLRNILETENENDITGRIKGKTVKNKKGEEKYISGEIDKLYDNNKQNEVKKNLKMFYSYDFNMNRKKEIEDFFSNIDEAISSIRHGIVHFNLELEGKDIFTFKNIVPSQISKKMFQNEINEKKLKLKIFRQLNSANVFRYLEKYKILNYLNRTRFEFVNKNIPFVPSFTKLYSRIDDLKNSLCIYWKIPKANDNNKTKEITDAQIYLLKNIYYGEFLNYFMSNNGNFFEIIKEIIELNKNDKRNLKTGFYKLQKFENLQEKTPKEYLANIQSFYMIDAGNKDEEEKDAYIDFIQKIFLKGFMTYLANNGRLSLMYIGNDEQINTSLAGKKQEFDKFLKKYEQNNNIEIPHEINEFVREIKLGKILKYTESLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGILNLLEKISDEAKYKISIEELKNYSNKKIEIEKNHTTQENLHRKYARPRKDEKFNDEDYKKYEKTIRNIQQYTHLKNKVEFNELNLLQSLLLRILHRLVGYTSIWERDLRFRLKGEFPENQYIEEIFNFDNSKNVKYKNGQIVEKYISFYKELYKDDMEKISIYSDKKVKELKKEKKDLYIRNYIAHFNYIPNAEVSLLEVLENLRKLLSYDRKLKNAIMKSIVDILKEYGFVVTFKIEKDKKIRIESLKSEEVVHLKKLKLKDNDKKKEPIKTYRNSKELCKLVKVMFEYKMKEKKSEN

SEQ ID NO: 16 - protein, 1168 aa, synthetic construct
  REGION 1..1168: note = Synthetic construct
MKITKIDGISHKKYIKEGKLVKSTSEENKTDERLSELLTIRLDTYIKNPDNASEEENRIRRENLKEFFSNKVLYLKDGILYLKDRREKNQLQNKNYSEEDISEYDLKNKNSFLVLKKILLNEDINSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIRECYHKIIGRKNDKENFAKIIYEEIQNVNNIKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIAGNRQNEAFLRNIIGVSSVAYFSLRNILETENKDDITGKMRGKTRIDSKTGEEKYIPGEVDQIYYENKQNEVKNKLKMFYGYDFDMDNKKEIEDFFANIDEAISSIRHGIVHFNLDLDGKDIFAFKNIVPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISREIIELNKNDKRNLKTGFYKLQKFEDIQEKTPKKYLANIQSLYMINAGNQDEEEKDTYIDFIQKIFLKGFMTYLANNGRLSLMYIGNDEQINTSLAGKKQEFDKFLKKYEQNNNIEIPHEINEFLREIKLGKILKYTESLNMFYLILKLLNHKELTNLKGSLEKYQSANKEETFSDELELINLLNLDNNRVTEDFELEANEIGKFLDFNGNKIKDRKELKKFDTKKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENQYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSVVDILKEYGFVATFKIGADKKIGIQTLESEKIVHLKNLKKKKLMTDRNSEELCKLVKVMFEYKMEEKNLKTKKCKVI

SEQ ID NO: 17 - protein, 1224 aa, synthetic construct
  REGION 1..1224: note = Synthetic construct
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 18 - protein, 1225 aa, synthetic construct
  REGION 1..1225: note = Synthetic construct
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 19 - protein, 1165 aa, synthetic construct
  REGION 1..1165: note = Synthetic construct
MKNQNTLPSNPTDILKDKPFWAAFFNLARHNVYLTVNHINKLLDLEKLYNKDKHKEIFEHEDIFNISDDVMNDVNSNGKKRKLDIKKIWANLDTDLTRKYQLRELILKHFPFIQPAIIGAQTKERTTIDKDKRSTSTSNDSLKPTGEGDINDPLSLSNVKSIFFRLLQMLEQLRNYYSHVKHSKSATMPNFDEGLLKSMYNIFIDSVNKVKEDYSSNSVIDPNTSFSHLISKDEQGEIKPCRYSFTSKDGSINASGLLFFVSLFLEKQDSIWMQKKIPGFKKTSENYMKMTNEVFCRNHILLPKMRLETVYDKDWMLLDMLNEVVRCPLSLYKRLAPADQNKFKVPEKSSDNANRQEDDNPFSRILVRHQNRFPYFALRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLERLNKQPEQKPNVAAKNTGTLLRNGQIADWLVKDMMRFQPVKRDKEGNPINCSKANSTEYQMLQRAFAFYTTDSYRLPRYFEQLHLINCDNSHLFLSRFEYDKQPNLIAFYAAYLEAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYTYNFNVGNVSKITEANYLSKEKRENLFKSYQNKFKNNIPAEKTKEYREYKNFSSWKKFERELRLIKNQDILTWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDINLEKYPLTKYQVDSELDLYQKYRIDIFKQTLDLEAQLLDKYSDLNTDNFNQMLSGWSEKEGIPRNIKQDVAFLIGVRNGFSHNQYPDSKRIAFSRIKKFNPKTSSLQESEGLNIAKQMYEEAQQVVNKIKNIESFD

SEQ ID NO: 20 - protein, 1194 aa, synthetic construct
  REGION 1..1194: note = Synthetic construct
MKVTKIDGISHKKFEDEGKLVKFTGHFNIKNEMKERLEKLKELKLSNYIKNPENVKNKDKNKEKETKSRRENLKKYFSEIILRKKEEKYLLKKTRKFKNITEEINYDDIKKRENQQKIFDVLKELLEQRINENDKEEILNFDSVKLKEAFEEDFIKKELKIKAIEESLEKNRADYRKDYVELENEKYEDVKGQNKRSLVFEYYKNPENREKFKENIKYAFENLYTEENIKNLYSEIKEIFEKVHLKSKVRYFYQNEIIGESEFSEKDEEGISILYKQIINSVEKKEKFIEFLQKVKIKDLTRSQIFYKYFLENEELNDENIKYVFSYFVEIEVNKLLKENVYKTKKFNEGNKYRVKNIFNYDKLKNLVVYKLENKLNNYVRNCGKYNYHMENGDIATSDINMKNRQTEAFLRSILGVSSFGYFSLRNILGVNDDDFYKIEKDERKNENFILKKAKEDFTSKNIFEKVVDKSFEKKGIYQIKENLKMFYGNSFDKVDKDELKKFFVNMLEAITSVRHRIVHYNINTNSENIFDFSNIEVSKLLKNIFEKEIDTRELKLKIFRQLNSAGVFDYWESWVIKKYLENVKFEFVNKNVPFVPSFKKLYDRIDNLKGWNALKLGNNINIPKRKEAKDSQIYLLKNIYYGEFVEKFVNDNKNFEKIVKEIIEINRGAGTNKKTGFYKLEKFETLKANTPTKYLEKLQSLHKISYDKEKIEEDKDVYVDFVQKIFLKGFVNYLKKLDSLKSLNLLNLRKDETITDKKSVHDEKLKLWENSGSNLSKMPEEIYEYVKKIKISNINYNDRMSIFYLLLKLIDYRELTNLRGNLEKYESMNKNKIYSEELTIINLVNLDNNKVRTNFSLEAEDIGKFLKSSITIKNIAQLNNFSKIFADGENVIKHRSFYNIKKYGILDLLEKIVAKADLKITKEEIKKYENLQNELKRNDFYKIQEQIHRNYNQKPFSIKKIENKKDFEKYKKVIEKIQDYTQLKNKIEFNDLNLLQSLIFRILHRLAGYTSLWERDLQFKLKGEFPEDKYIDEIFNSDGNNNQKYKHGGIADKYANFLIEKKEEKSGEILNKKQRKKKIKEDLEIRNYIAHFNYLPNAEKSILEILEELRELLKHDRKLKNAVMKSIKDIFREYGFIVEFTISHTKNGKKIKVCSVKSEKIKHLKNNELITTRNSEDLCELVKIMLEHKELQK

SEQ ID NO: 21 - protein, 1159 aa, synthetic construct
  REGION 1..1159: note = Synthetic construct
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 22 - protein, 1134 aa, synthetic construct
  REGION 1..1134: note = Synthetic construct
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 23 - protein, 1281 aa, synthetic construct
  REGION 1..1281: note = Synthetic construct
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRY

SEQ ID NO: 24 - protein, 1399 aa, synthetic construct
  REGION 1..1399: note = Synthetic construct
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 25 - protein, 1313 aa, synthetic construct
  REGION 1..1313: note = Synthetic construct
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 26 - protein, 1470 aa, synthetic construct
  REGION 1..1470: note = Synthetic construct
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 27 - protein, 1259 aa, synthetic construct
  REGION 1..1259: note = Synthetic construct
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 28 - protein, 477 aa, synthetic construct
  REGION 1..477: note = Synthetic construct
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 29 - protein, 1293 aa, synthetic construct
  REGION 1..1293: note = Synthetic construct
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 30 - protein, 1228 aa, synthetic construct
  REGION 1..1228: note = Synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 31 - protein, 1286 aa, synthetic construct
  REGION 1..1286: note = Synthetic construct
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 32 - protein, 1272 aa, synthetic construct
  REGION 1..1272: note = Synthetic construct
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 33 - protein, 477 aa, synthetic construct
  REGION 1..477: note = Synthetic construct
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 34 - protein, 992 aa, synthetic construct
  REGION 1..992: note = Synthetic construct
VKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 35 - protein, 1367 aa, synthetic construct
  REGION 1..1367: note = Synthetic construct
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 36 - protein, 809 aa, synthetic construct
  REGION 1..809: note = Synthetic construct
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 37 - protein, 839 aa, synthetic construct
  REGION 1..839: note = Synthetic construct
LNSIEKIKKPSNRNSIPSIIISDYDENKIKEIKVKYLKLARLDKITIQDMEIRDNIVEFKKILLNGIEHTIKDNQKIEFDNYEITAYVRASKQRRDGKITQAKYVVTITDKYLRDNEKEKRFKSTERELPNDTLLMRYKQISGFDTLTSKDIYKIKRYIDFKNEMLFYFQFIEEFFSPLLPKGTNFYSLNIEQNKDKVVKYIVYRLNDDFKNQSLNQFIKKTDTIKYDFLKIQKILSDFRHALAHFDFDFIQKFFDDELDKNRFDISTISLIKTMLQEKEEKYYQEKNNYIEDSDTLTLFDEKESNFSKIHNFYIKISQKKPAFNKLINSFLSKDGVPNEELKSYLATKKIDFFEDIHSNKEYKKIYIKHKNLVVEKQKEESQEKPNGQKLKNYNDELQKLKDEMNKITKQNSLNRLEVKLRLAFGFIANEYNYNFKNFNDKFTLDVKKEQKIKVFKNSSNEKLKEYFESTFIEKRFFHFCVKFFNKKTKKEETKQKNIFNLIENETLEELVKESPLLQIITLLYLFIPKELQGEFVGFILKIYHHTKNITNDTKEDEKSIEDTQNSFSLKLKILAKNLRGLQLFNYSLSHNTLYNTKEHFFYEKGNRWQSVYKSLEISHNQDEFDIHLVIPVIKYYINLNKLIGDFEIYALLTYADKNSITEKLSDITKRDDLKFRGYYNFSTLLFKTFMINTNYEQNQKSTQYIKQTRNDIAHQNIENMLKAFENNEIFAQREEIVNYLQKEHKMQEILHYNPINDFTMKTVQYLKSLNIHSQKESKIADIHKKESLVPNDYYLIYKLKVIELLKQKVIEAIGETKDEEKIKNAIAKEEQIKKGYNK

SEQ ID NO: 38 - protein, 840 aa, synthetic construct
  REGION 1..840: note = Synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 39 - protein, 840 aa, synthetic construct
  REGION 1..840: note = Synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 40 - protein, 849 aa, synthetic construct
  REGION 1..849: note = Synthetic construct
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 41 - protein, 822 aa, synthetic construct
  REGION 1..822: note = Synthetic construct
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 42 - protein, 806 aa, synthetic construct
  REGION 1..806: note = Synthetic construct
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 43 - protein, 832 aa, synthetic construct
  REGION 1..832: note = Synthetic construct
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 44 - protein, 1281 aa, synthetic construct
  REGION 1..1281: note = Synthetic construct
MKKSIFDQFVNQYALSKTLRFELKPVGETGRMLEEAKVFAKDETIKKKYEATKPFFNKLHREFVEEALNEVELAGLPEYFEIFKYWKRYKKKFEKDLQKKEKELRKSVVGFFNAQAKEWAKKYETLGVKKKDVGLLFEENVFAILKERYGNEEGSQIVDESTGKDVSIFDSWKGFTGYFIKFQETRKNFYKDDGTATALATRIIDQNLKRFCDNLLIFESIRDKIDFSEVEQTMGNSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 45 - protein, 1280 aa, synthetic construct
  REGION 1..1280: note = Synthetic construct
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 46 - protein, 832 aa, synthetic construct; identical to SEQ ID NO: 43
  REGION 1..832: note = Synthetic construct

SEQ ID NO: 47 - protein, 809 aa, synthetic construct; identical to SEQ ID NO: 36
  REGION 1..809: note = Synthetic construct

SEQ ID NO: 48 - protein, 1285 aa, synthetic construct
  REGION 1..1285: note = Synthetic construct
MEEKMLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 49 - protein, 1367 aa, synthetic construct; identical to SEQ ID NO: 35
  REGION 1..1367: note = Synthetic construct

SEQ ID NO: 50 - protein, 1366 aa, synthetic construct
  REGION 1..1366: note = Synthetic construct
MNTQKKEFNPKSFKDFTNLYSLNKTLRFSLTPNKKTAEILEFNKQKEVKCFSNDRKIAGAYQEIKKYLNKLHQEFIQEAMKFFAFSEEELKGFEKEYLNLLNFTDKDNFKKKNKIRNEYEQERKILTIKIATYFSKFKSEKYQSFNLANITGKKVFSILEQKYKEDKKTLKIIHIFKYKPTKDEKKEGEAVNFSTYLTGFNENRKNFYKSEDKAGQFATRTIDNLAQFIKNKKLFEDKYQKNYSKIGILDEQIKIFNLDYFNNLFLQEGLDEYNGILGNNKGEENKSNEGINQKINIFKQKEKARLKKEKENFNKSDFPLFKELYKQIGSIRKENDVYVEIKTDKELVEELNNFPKNVENYLKDIQSFYKTFFEKLQNEEYELDKIYLPKSVGTYFSYIAFSDWNKLAFIYNKRYKNEKIKIVEGGDVNVQYRSLEVLKNRIDELKDEDNLNFNKFFIDKLKFNEAKKENNWQNFWFCIEYYINSQFIGGEKNILNKEKNEYEILPFGSLKELKEKYFEAVKKYKEKMVDTESGLTDDEEKEIKETLKNYLDRIKEIERIAKYFDLKKSFEEIKQEDLDSNFYGEYQKVVDKTNELKIYQYYSEFRNYLTQNNSVEEKIKLNFNSGLLLDGWDLNKEKVKFSIIFQENGKYYLGIINKEKDKTILDKDKHPEIFTKNSDFRKMEYKLFPSPSKMLPKISFSETAKKGDEDVGWSEEIQKIKDEFAEFQEYKKKSKDNWKDEFNRGKLNKLIDYYKQVLEKHSEGYMNTYNFELKDSSKYKNLGEFNDDIARQNYKVKFVGIDKNYIDEKVANGELFLFQIYNKDFSEDKKEGSTNNLETIYFKELFSKENLENPVFKLSGGAEMFFRNKIEKKKEKKKLDKDGKPMISKKGEKVVDKRRFSENKILFHLPIEINYGKGKMPNFNKKINEYISKNPENIKIIGIDRGEKHLLYYSIIDQNGNNIESMSLNAVDEFGNFVNPEKLEEYEIDNNGKKERRWKYIVNDKEIKVTNYQRKLDELEKERQKSRQSWQNINKIKNLKKGYISFVVKKIVDLAIENNAIIILEDLNFGFKSFRQKIEKNVYQQFEKALIDKLGFVVDKQKQNQRFAPQLSAPFESFQKIGKQTGIVYYVLANNTSKVCPSCQWIKNFYLKYEKKNTIFNLQKNQKLKVFFEQEKNRFRFEYQMSKEYISVYSDVDRQRYDKTKNQNKGGYLEYKNSNQKEIIDKDGVIQKQSITLQLKELFKENHIDLEKEILKQLDNKKEKNSGYTGVYNKFIYLFNLILQIRNAISFREKDYIQCPSCHFDTRKENYLKINDGDGNGAYNIALRGLYLLKGKNGIINNLEKIKLIFSNNDYFQWAKKLKNKK

SEQ ID NO: 51 - protein, 1275 aa, synthetic construct
  REGION 1..1275: note = Synthetic construct
MQNKQSFADFTNLYSLSKTLRFELKPIGQTQAMLDENKIFEVDENRKKAYDKTKPYFDRLHREFINESLSNAQLKGISEYFETFKQFRSNQNNKDLKELINKQQKFLRHQIVTLFDENGKHWATTKYAHLKIKKKNLDILFDEQVFYILKERYGSEKETQLVDKETGAVTSIFDNWKGFTGYFTKFFETRKNFYKSDGTSTALATRIIDQNLNRFFDNLETFHKIKDKIDVKEVEIFFKLKADNVFSIDFYNQCLLQNGIDKYNDFLGGQTLENGEKQKGINEIINKYRQDNKDQKLPFLKKLDKQILSEKDRFINEIESKEEFFQVLTEFYQSATVKVTIIKTLLNDFVHNTDKYKLEKIYLTKEAFNTIANKWTDETQIFEDNLDLVLKNKKITAKQDFIPLAYIKEALEVIEKDRKFFKDRYYNDPQIGFFPDQSYWEQFLAILNFEFMTHFQRVAKDKITGKKIELGYFVFEKRIKELLDSDPSLNSQSKIIIKEFADEVLHIFQMAKYFALEKKREWKGDYYQLDDQFYNHIDYGFKDQFYENAYEKIVQPYNKIRNYLTKKPYSDVKWKLNFGNPTLANGWDKNKEADNTAVILKKDGNYYLGVMKKGKNKIFSDQNKEKYKAYNSAYYEKLVYKLFPDPSKMFPKVCFSKKGLNFFQPSEEILRIYKNNEFKKGNTFSISSMQKLIAFYIDCLGLYEGWKHYEFKNIKDVRQYKENIGEFYADVAESGYKLWFEKISEEYITQKNQLGELFLFQIYNKDFAKKTTGRKNLHTIYFEELFSQTNIDNNFPFKLNGQAELFYRPKSLEKIEEKRNFKRSIVNKKRYTQNKIFFHVPITLNRTSENIGRFNVRVNNFLANNSNVNIVGVDRGEKNLAYYSIIKQNGEVLKSGSLNIINGVDYHALLTDRAQRREQERRNWQDVESIKDLKRGYISQVVHELVSLAIKYNAIIVMEDLNMRFKQIRGGIEKSTYQQLEKALIEKLNFLVNKEETDSNQAGNLLNAYQLTAPFKTFKDMGKQTGIIFYTQASYTSKIDPLTGWRPNIYLRYSNAKQAKADILMFTNIYFSEKKDRFEFTYDLEKIDDKRKDLPIKTEWTVCSNVERFSWEKSLNNNKGGYVHYPIQDSNGEESITSKLKKLFMDFGIDLTDIKTQIESLDTNKKDNANFFRKFIFYFQLICQIRNTQVNKSDDGNDFIFSPVEPFFDSRFADKFRKNLPKNGDENGAYNIARKGLIILHKISDYFVKEGSTDKISWKDLSISQTEWDNFTTDK

SEQ ID NO: 52 - protein, 1368 aa, synthetic construct
  REGION 1..1368: note = Synthetic construct
MKKEKEFKSFGDFTNLYEISKTLRFELKPVENTQTMLDEADVFGKDKVIKDKYTKTKPFIDKLHREFVDESLKDVSLSGLKKYSEVLENWKKNKKDKDIVKELKKEEERLRKEVVEFFDNTAKKWANEKYKELGLKKKDIGILFEESVFDLLKEKYGEEQDSFLKEEKGDFLKNEKGEKVSIFDEWKGFVGYFTKFQETRKNFYKNDGTETALATRIIDQNLKRFCDNIDDFKKIKNKIDFSEVEKNFNKTADVFSLDFYNQCLLQKGIDSYNEFIGGKTLENGKKLKGVNELVNEYRQKNKNEKVSFLKLLDKQILSEKEKLSFGIENDEQLLVVLNSFYETAEEKTKILRTLFGDFVEHNENYDLDKTYISKVAFNTISHKWTNETHKFEELLYGAMKEDKPIGLNYDKKEDSYKFPDFIALGYLKKCLNNLDCDTKFWKEKYYENNADKKDKDKGFLTGGQNAWDQFLQIFIFEFNQLFNSEAFDNKGKEIKIGYDNFRKDFEEIINQKDFKNDENLKIAIKNFADSVLWIYQMAKYFAIEKKRGWDDDFELSEFYTNPSNGYSLFYDRAYEEIVQKYNDLRNYLTKKPYKEDKWKLNFENPTLANGFDKNKESDNSTVILRKKRKYYLGLMKKGNNKIFEDRNKAEFIRNIESGAYEKMAYKYLPDVAKMIPKCSTQLNEAKNHFRNSADDLEIKKSFSNPLKITKRIFDLNNIQYDKTNVSKKISGDNKGIKIFQKEYYKISGDFDVYKSALNDWIDFCKDFLSKYDSTKDFDFSILRKTKDYKSLDEFYVDVAKITYKISFTPVSESYIDQKNKNGELYLFEIYNQDFAKGKMGAKNLHTLYFENVFSPENISKNFPIKLNGNAELFFRPKSIESKKEKRNFVREIVNKKRYSEDKIFFHCPITLNRETGSIYRFNNYVNNFLSENNINIIGVDRGEKHLAYYSVIDKNGVKIGGGSFNEINKVDYAKKLEERAGEREQSRKDWQVVEGIKDLKKGYISQVVRELADLAIKHNAIIVLEDLNMRFKQIRGGIEKSIYQQLEKALIDKLSFLVEKGEKDPNQAGHILKAYQLAAPFTSFKDMGKQTGIVFYTQASYTSKTCPNCGFRKNNNKFYFENNIGKAQDALKKLKTFEYDSENKCFGLSYCLSDFANKEEVEKNKNKKRNNAPYSDIEKKDCFELSTKDAVRYRWHDKNTERGKTFFEGESVYEEKEEKEIGQTKRGLVKEYDISKCLIGLFEKTGLDYKQNLLDKINSGKFDGTFYKNLFNYLNLLFEIRNSISGTEIDYISCPECQFHTDKSKTIKNGDDNGSYNIARKGMIILDKIKQFKKENGSLDKMGWGELFIDLEEWDKFAQKKNNNIIDK

SEQ ID NO: 53 - protein, 1257 aa, synthetic construct
  REGION 1..1257: note = Synthetic construct
MKYTDFTGIYSVSKTLRFELIPQGSTVENMKREGILNNDMHRADSYKEMKKLIDEYHKAFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFDIVAKVPEMQEHFKTIADELRSHLQVGNDIDKMFNLQFFNKVLTQSQLDVYNAVIGGKSEGNKKIQGINEYINLYNQQHKKARLPMLKLLYKQILSDRVAISWLQDEFDNDQDMLDTIEAFYNKLNSNETGVLGEGKLKQILMGLDGYNLDGVFMRNDLQLSEVSQRLCGGWNIIKDAMTSDLKRSVQKKKKETDADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDNFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSRVQEFSPSKKILEIWESKSFKKGDNFNLDDCHALIDFYKDSIAKHPDWNKFNFKFSDTQSYTNISDFYRDVNQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHSLLEEKEAGNKKARQDWQTIESIKELKEGYLSQVIHKISMLMQRYHAIVVLEDLNGSFMRSRQKVEKQVYQKFEHMLINKLNYLVNKQYDATEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 54 - protein, 766 aa, synthetic construct
  REGION 1..766: note = Synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 55 - protein, 717 aa, synthetic construct
  REGION 1..717: note = Synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 56 - protein, 766 aa, synthetic construct
  REGION 1..766: note = Synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 57 - protein, 718 aa, synthetic construct
  REGION 1..718: note = Synthetic construct
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 58 - protein, 757 aa, synthetic construct
  REGION 1..757: note = Synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 59 - protein, 761 aa, synthetic construct
  REGION 1..761: note = Synthetic construct
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 60 - protein, 708 aa, synthetic construct
  REGION 1..708: note = Synthetic construct
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 61 - protein, 661 aa, synthetic construct
  REGION 1..661: note = Synthetic construct
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 62 - protein, 18 aa, synthetic construct
  REGION 1..18: note = Synthetic construct
KRPAATKKAGQAKKKKEF

SEQ ID NO: 63 - RNA, 51 nt, synthetic construct
  misc_feature 1..51: note = Synthetic construct
  source 1..51: mol_type = other RNA
ggccaccccaaaaatgaaggggactaaaacagtgataagtggaatgccatg

SEQ ID NO: 64 - RNA, 51 nt, synthetic construct
  misc_feature 1..51: note = Synthetic construct
  source 1..51: mol_type = other RNA
ggccaccccaaaaatgaaggggactaaaacacgacctactctcccatactc

SEQ ID NO: 65 - RNA, 41 nt, synthetic construct
  misc_feature 1..41: note = Synthetic construct
  source 1..41: mol_type = other RNA
taatttctactaagtgtagatcccccagcgcttcagcgttc

SEQ ID NO: 66 - RNA, 41 nt, synthetic construct; identical to SEQ ID NO: 65
  misc_feature 1..41: note = Synthetic construct
  source 1..41: mol_type = other RNA

SEQ ID NO: 67 - RNA, 46 nt, synthetic construct
  misc_feature 1..46: note = Synthetic construct
  modified_base 1..5: mod_base = OTHER; note = misc_feature - Where n is phosphorothioated thymine
  source 1..46: mol_type = other RNA
nnnnntaatttctactaagtgtagatcccccagcgcttcagcgttc

SEQ ID NO: 68 - RNA, 51 nt, synthetic construct
  misc_feature 1..51: note = Synthetic construct
  modified_base 1..10: mod_base = OTHER; note = misc_feature - Where n is phosphorothioated thymine
  source 1..51: mol_type = other RNA
nnnnnnnnnntaatttctactaagtgtagatcccccagcgcttcagcgttc

SEQ ID NO: 69 - RNA, 41 nt, synthetic construct; identical to SEQ ID NO: 65
  misc_feature 1..41: note = Synthetic construct
  source 1..41: mol_type = other RNA

SEQ ID NO: 70 - RNA, 41 nt, synthetic construct; identical to SEQ ID NO: 65
  misc_feature 1..41: note = Synthetic construct
  source 1..41: mol_type = other RNA

SEQ ID NO: 71 - RNA, 40 nt, synthetic construct
  misc_feature 1..40: note = Synthetic construct
  source 1..40: mol_type = other RNA
taatttcactaagtgtagatcccccagcgcttcagcgttc

SEQ ID NO: 72 - RNA, 40 nt, synthetic construct
  misc_feature 1..40: note = Synthetic construct
  source 1..40: mol_type = other RNA
taatttctactaagtgtagacccccagcgcttcagcgttc

SEQ ID NO: 73 - RNA, 41 nt, synthetic construct; identical to SEQ ID NO: 65
  misc_feature 1..41: note = Synthetic construct
  source 1..41: mol_type = other RNA

SEQ ID NO: 74 - RNA, 41 nt, synthetic construct; identical to SEQ ID NO: 65
  misc_feature 1..41: note = Synthetic construct
  source 1..41: mol_type = other RNA

SEQ ID NO: 75 - RNA, 41 nt, synthetic construct; identical to SEQ ID NO: 65
  misc_feature 1..41: note = Synthetic construct
  source 1..41: mol_type = other RNA

SEQ ID NO: 76 - DNA, 13 bp, synthetic construct
  misc_feature 1..13: note = Synthetic construct
  source 1..13: mol_type = other DNA
ttattattatatt

SEQ ID NO: 77 - DNA, 18 bp, synthetic construct
  misc_feature 1..18: note = Synthetic construct
  modified_base 1..3: mod_base = OTHER; note = misc_feature - Where T is phosphorothioated thymine
  source 1..18: mol_type = other DNA
tttattattattattatt

SEQ ID NO: 78 - DNA, 12 bp, synthetic construct
  misc_feature 1..12: note = Synthetic construct
  source 1..12: mol_type = other DNA
ttattattatta

SEQ ID NO: 79 - DNA, 30 bp, synthetic construct
  misc_feature 1..30: note = Synthetic construct
  source 1..30: mol_type = other DNA
tttttttttttttttttttttttttttttt

SEQ ID NO: 80 - DNA, 20 bp, synthetic construct
  misc_feature 1..20: note = Synthetic construct
  source 1..20: mol_type = other DNA
tttttttttttttttttttt

SEQ ID NO: 81 - DNA, 22 bp, synthetic construct
  misc_feature 1..22: note = Synthetic construct
  modified_base 1..3: mod_base = OTHER; note = misc_feature - Where T is phosphorothioated thymine
  source 1..22: mol_type = other DNA
tttttttttttttttttttttt

SEQ ID NO: 82 - DNA, 21 bp, synthetic construct
  misc_feature 1..21: note = Synthetic construct
  modified_base 20..21: mod_base = OTHER; note = misc_feature - Where T is phosphorothioated thymine
  source 1..21: mol_type = other DNA
ttttttttttttttttttttt

SEQ ID NO: 83 - DNA, 22 bp, synthetic construct
  misc_feature 1..22: note = Synthetic construct
  modified_base 1..3: mod_base = OTHER; note = misc_feature - Where T is phosphorothioated thymine
  source 1..22: mol_type = other DNA
tttttttttttttttttttttt

SEQ ID NO: 84 - DNA, 20 bp, synthetic construct
  misc_feature 1..20: note = Synthetic construct
  source 1..20: mol_type = other DNA
tttttttttttttttttttt

SEQ ID NO: 85 - DNA, 21 bp, synthetic construct
  misc_feature 1..21: note = Synthetic construct
  modified_base 20..21: mod_base = OTHER; note = misc_feature - Where T is phosphorothioated thymine
  source 1..21: mol_type = other DNA
ttttttttttttttttttttt

SEQ ID NO: 86 - DNA, 20 bp, synthetic construct
  misc_feature 1..20: note = Synthetic construct
  source 1..20: mol_type = other DNA
tttttttttttttttttttt

SEQ ID NO: 87 - DNA, 20 bp, synthetic construct
  misc_feature 1..20: note = Synthetic construct
  source 1..20: mol_type = other DNA
  misc_feature 1..9: note = DNA
  misc_feature 10..14: note = RNA
  misc_feature 15..20: note = DNA
tttttttttttttttttttt

SEQ ID NO: 88 - DNA, 12 bp, synthetic construct
  misc_feature 1..12: note = Synthetic construct
  source 1..12: mol_type = other DNA
tttttttttttt

SEQ ID NO: 89 - DNA, 12 bp, synthetic construct
  misc_feature 1..12: note = Synthetic construct
  source 1..12: mol_type = other DNA
tttttttttttt

SEQ ID NO: 90 - DNA, 12 bp, synthetic construct
  misc_feature 1..12: note = Synthetic construct
  source 1..12: mol_type = other DNA
tttttttttttt

SEQ ID NO: 91 - DNA, 20 bp, synthetic construct
  misc_feature 1..20: note = Synthetic construct
  source 1..20: mol_type = other DNA
tttttttttttttttttttt

SEQ ID NO: 92 - DNA, 20 bp, synthetic construct
  misc_feature 1..20: note = Synthetic construct
  source 1..20: mol_type = other DNA
tttttttttttttttttttt

SEQ ID NO: 93 - DNA, 19 bp, synthetic construct
  misc_feature 1..19: note = Synthetic construct
  source 1..19: mol_type = other DNA
ttttttttttttttttttt

SEQ ID NO: 94 - DNA, 19 bp, synthetic construct
  misc_feature 1..19: note = Synthetic construct
  source 1..19: mol_type = other DNA
ttttttttttttttttttt

SEQ ID NO: 95 - RNA, 41 nt, synthetic construct; identical to SEQ ID NO: 65
  misc_feature 1..41: note = Synthetic construct
  source 1..41: mol_type = other RNA

SEQ ID NO: 96 - RNA, 41 nt, synthetic construct; identical to SEQ ID NO: 65
  misc_feature 1..41: note = Synthetic construct
  source 1..41: mol_type = other RNA

SEQ ID NO: 97 - RNA, 41 nt, synthetic construct; identical to SEQ ID NO: 65
  misc_feature 1..41: note = Synthetic construct
  source 1..41: mol_type = other RNA

SEQ ID NO: 98 - RNA, 41 nt, synthetic construct; identical to SEQ ID NO: 65
  misc_feature 1..41: note = Synthetic construct
  source 1..41: mol_type = other RNA

SEQ ID NO: 99 - RNA, 41 nt, synthetic construct
  misc_feature 1..41: note = Synthetic construct
  source 1..41: mol_type = other RNA
taatttctactaagtgtagatttacatggctctggtccgag

SEQ ID NO: 100 - RNA, 41 nt, synthetic construct
  misc_feature 1..41: note = Synthetic construct
  source 1..41: mol_type = other RNA
taatttctactaagtgtagatgccgataatgatgtagggat

SEQ ID NO: 101 - RNA, 41 nt, synthetic construct; identical to SEQ ID NO: 65
  misc_feature 1..41: note = Synthetic construct
  source 1..41: mol_type = other RNA

SEQ ID NO: 102 - DNA, 22 bp, synthetic construct
  misc_feature 1..22: note = Synthetic construct
  modified_base 1..3: mod_base = OTHER; note = misc_feature - Where T is phosphorothioated thymine
  source 1..22: mol_type = other DNA
tttttttttttttttttttttt

SEQ ID NO: 103 - RNA, 41 nt, synthetic construct; identical to SEQ ID NO: 100
  misc_feature 1..41: note = Synthetic construct
  source 1..41: mol_type = other RNA

SEQ ID NO: 104 - RNA, 41 nt, synthetic construct; identical to SEQ ID NO: 99
  misc_feature 1..41: note = Synthetic construct
  source 1..41: mol_type = other RNA

SEQ ID NO: 105 - RNA, 41 nt, synthetic construct
  misc_feature 1..41: note = Synthetic construct
  source 1..41: mol_type = other RNA
taatttctactaagtgtagatctgccaattgcaggaatgat

SEQ ID NO: 106 - DNA, 20 bp, synthetic construct
  misc_feature 1..20: note = Synthetic construct
  source 1..20: mol_type = other DNA
ttacaaacattggccgcaaa

SEQ ID NO: 107 - DNA, 18 bp, synthetic construct
  misc_feature 1..18: note = Synthetic construct
  source 1..18: mol_type = other DNA
gcgcgacattccgaagaa

SEQ ID NO: 108 - RNA, 41 nt, synthetic construct; identical to SEQ ID NO: 65
  misc_feature 1..41: note = Synthetic construct
  source 1..41: mol_type = other RNA

SEQ ID NO: 109 - DNA, 20 bp, synthetic construct
  misc_feature 1..20: note = Synthetic construct
  source 1..20: mol_type = other DNA
cctccgtgatatggctcttc

SEQ ID NO: 110 - DNA, 18 bp, synthetic construct
  misc_feature 1..18: note = Synthetic construct
  source 1..18: mol_type = other DNA
agagtcctttgggcttcc

SEQ ID NO: 111 - RNA, 41 nt, synthetic construct; identical to SEQ ID NO: 99
  misc_feature 1..41: note = Synthetic construct
  source 1..41: mol_type = other RNA

SEQ ID NO: 112 - DNA, 25 bp, synthetic construct
  misc_feature 1..25: note = Synthetic construct
  source 1..25: mol_type = other DNA
aggtgcctggaatattggtgaacag

SEQ ID NO: 113 - DNA, 26 bp, synthetic construct
  misc_feature 1..26: note = Synthetic construct
  source 1..26: mol_type = other DNA
tcaagagtgcgggagaaaattgatcg

SEQ ID NO: 114 - RNA, 41 nt, synthetic construct
  misc_feature 1..41: note = Synthetic construct
  source 1..41: mol_type = other RNA
taatttctactaagtgtagatcatcagaggctgctcgtgtt

SEQ ID NO: 115 - DNA, 12 bp, synthetic construct
  misc_feature 1..12: note = Synthetic construct
  source 1..12: mol_type = other DNA
tttttttttttt

SEQ ID NO: 116 - DNA, 19 bp, synthetic construct
  misc_feature 1..19: note = Synthetic construct
  source 1..19: mol_type = other DNA
ttttttttttttttttttt

SEQ ID NO: 117 - DNA, 20 bp, synthetic construct
  misc_feature 1..20: note = Synthetic construct
  source 1..20: mol_type = other DNA
tttttttttttttttttttt

SEQ ID NO: 118 - RNA, 41 nt, synthetic construct; identical to SEQ ID NO: 65
  misc_feature 1..41: note = Synthetic construct
  source 1..41: mol_type = other RNA

SEQ ID NO: 119 - RNA, 41 nt, synthetic construct; identical to SEQ ID NO: 99
  misc_feature 1..41: note = Synthetic construct
  source 1..41: mol_type = other RNA

(19) United States
(12) Patent Application Publication
(10) Pub. No.: US 2024/0294970 A1
(43) Pub. Date: Sep. 5, 2024

(54) METHODS AND COMPOSITIONS FOR PERFORMING A DETECTION ASSAY

(71) Applicant: Mammoth Biosciences, Inc., Brisbane, CA (US)

(72) Inventors: Jesus CHING, Saratoga, CA (US); James Paul BROUGHTON, South San Francisco, CA (US); Janice Sha CHEN, San Francisco, CA (US); Sarah Jane SHAPIRO, Pacifica, CA (US); Elizabeth Mae HAWKINS, Redwood City, CA (US); Sonal JAIN, San Mateo, CA (US); Lior KREINDLER, Foster City, CA (US); Sophia HUBBELL, San Francisco, CA (US)

(21) Appl. No.: 18/334,327

(22) Filed: Jun. 13, 2023

Related U.S. Application Data
(63) Continuation of application No. PCT/US2021/063844, filed on Dec. 16, 2021.
(60) Provisional application No. 63/127,078, filed on Dec. 17, 2020.
(60) Provisional application No. 63/146,508, filed on Feb. 5, 2021.
(60) Provisional application No. 63/151,592, filed on Feb. 19, 2021.
(60) Provisional application No. 63/222,377, filed on Jul. 15, 2021.

(51) Int. Cl. C12Q 1/6823; C12N 15/10; C12Q 1/44; C12Q 1/6806; C12Q 1/6844; G01N 21/64

(52) U.S. Cl. CPC C12Q 1/6823; C12N 15/1096; C12Q 1/44; C12Q 1/6806; C12Q 1/6844; G01N 21/6428; G01N 2021/6432; G01N 2333/922

(57) ABSTRACT

Described herein are methods, devices, and compositions for immobilization of biomolecules in diagnostic assays onto a surface. The methods, devices, and compositions may utilize or comprise a plurality of different non-naturally occurring guide nucleic acids. Each of the different non-naturally occurring guide nucleic acids may be immobilized to a surface at a known location identified with the particular non-naturally occurring guide nucleic acid, Alternatively, or in combination, a plurality of programmable nucleases may be immobilized to the surface at each of the known locations. Optionally, the methods, devices, and compositions may utilize or comprise a plurality of reporters immobilized to the surface in proximity to each of the different non-naturally occurring guide nucleic acids and/or programmable nucleases at each of the known locations.

### CROSS-REFERENCE

This application claims the benefit of U.S. Provisional Application Ser. No. 63/127,078 filed on Dec. 17, 2020; U.S. Provisional Application Ser. No. 63/146,508 filed on Feb. 5, 2021; U.S. Provisional Application Ser. No. 63/151,592 filed on Feb. 19, 2021; and U.S. Provisional Application Ser. No. 63/222,377 filed on Jul. 15, 2021, each of which is incorporated herein by reference in its entirety for all purposes.

### STATEMENT AS TO FEDERALLY SPONSORED RESEARCH

This invention was made with government support under Contract No, N66001-21-C-4048 awarded by the Department of Defense, Defense Advanced Research Projects Agency (DARPA). The US government has certain rights in the invention.

### SEQUENCE LISTING

The instant application contains a Sequence Listing which has been submitted electronically in XML file format and is hereby incorporated by reference in its entirety. Said XML copy, created on Jul. 7, 2023, is named 203477-716301_US_SL.xml and is 219,592 bytes in size.

### BACKGROUND

The detection of ailments, especially at the early stages of disease or infection, may provide guidance on treatment or intervention to reduce the progression or transmission of the ailment. Diagnostic assays can detect ailments at the point of need through the use of diagnostic devices. Methods to immobilize diagnostic assay components on surfaces without compromising the performance of the assay are needed for manufacturing and using diagnostic assay devices.

### SUMMARY

In certain aspects, the present disclosure provides methods, devices and compositions for immobilizing a biomolecule onto a surface. In some embodiments, the present disclosure provides methods for immobilizing a biomolecule to a surface, as described herein. In some embodiments, the present disclosure provides an apparatus for use in a diagnostic assay comprises a biomolecule immobilized on a surface. In some embodiments, said biomolecule is a nucleic acid. In certain aspects, said nucleic acid can comprise a guide nucleic acid (e.g., a guide RNA). In various aspects, said biomolecule can comprise a reporter (e.g., a reporter molecule), In some embodiments, said diagnostic assay comprises a programmable nuclease and a guide nucleic acid as described herein.

In one aspect, the present disclosure provides a system for detecting any of a plurality of different target nucleic acids in a sample. The system can comprise: (a) a plurality of different non-naturally occurring guide nucleic acids, wherein each of the different non-naturally occurring guide nucleic acids is immobilized to a surface at a known location identified with the particular non-naturally occurring guide nucleic acid; and (b) a plurality of reporters immobilized to the surface in proximity to each of the different non-naturally occurring guide nucleic acids at each of the known locations. In some embodiments, each of the different non-naturally occurring guide nucleic acids comprises a sequence that hybridizes to a segment of one of the plurality of different target nucleic acids or an amplicon thereof. In some embodiments, each of the non-naturally occurring guide nucleic acids is effective to form a complex with a programmable nuclease that is activated upon binding the corresponding target nucleic acid or amplicon thereof at the known location. In some embodiments, formation of the activated complex is effective to induce detectable trans cleavage of the reporters at the respective known location.

In some embodiments, the plurality of different non-naturally occurring guide nucleic acids are each immobilized to the surface by a linkage. In some embodiments, the linkage comprises a covalent bond, a non-covalent bond, an electrostatic bond, a bond between members of a binding pair, an amide bond, or any combination thereof. In some embodiments, the linkage comprises a chain of at least 6 carbons, or at least 12 carbons. In some embodiments, the linkage comprises a linker polynucleotide. In some embodiments, the linker polynucleotide comprises a first member of a binding pair that binds to a second member of the binding pair on the surface. In some embodiments, the nucleic acid linker polynucleotide is double-stranded. In some embodiments, the linker polynucleotide comprises double-stranded DNA or single-stranded DNA. In some embodiments, the double-stranded DNA linker polynucleotide is about 60 to about 80 base pairs in length. In some embodiments, the linker polynucleotide is a cleavage substrate for the activated complex.

In some embodiments, the reporters and the non-naturally occurring guide nucleic acids are immobilized at separate discrete positions within each of the known locations. In some embodiments, each of the reporters comprises a fluorescent label and a quencher. In some embodiments, cleavage of the reporters is effective to produce a detectable loss of the quencher from the respective known location. In some embodiments, each of the reporters comprises a detection moiety. In some embodiments, cleavage of the reporters is effective to produce a detectable loss of the detection moiety from the respective known location. In some embodiments, the detection moiety comprises a fluorescent label.

In some embodiments, the system can further comprise programmable nucleases immobilized at the known locations by a linkage. In some embodiments, the plurality of different non-naturally occurring guide nucleic acids are immobilized to the surface by being releasably bound by the programmable nucleases. In some embodiments, the system can further comprise programmable nucleases bound to the non-naturally occurring guide nucleic acids. In some embodiments, the programmable nuclease comprises an RuvC catalytic domain. In some embodiments, the programmable nuclease is a type V CRISPR/Cas effector protein. In some embodiments, the type V CRISPR/Cas effector protein is a Cas12 protein. In some embodiments, the Cas12 protein comprises Cas12a, Cas12b, Cas12c, Cas12d, or Cas12e. In some embodiments, the type V CRISPR/Cas effector protein is a Cas14 protein. In some embodiments, the Cas14 protein comprises Cas14a, Cas14b, Cas14c, Cas14d, Cas14e, Cas14f, Cas14g, or Cas14h. In some embodiments, the type V CRISPR/Cas effector protein is a CasΦ protein. In some embodiments, the programmable nuclease comprises a HEPN catalytic domain. In some embodiments, the programmable nuclease is a type VI CRISPR/Cas effector protein. In some embodiments, the type VI CRISPR/Cas effector protein is a Cas13 protein. In some embodiments, the Cas13 protein comprises Cas13a, Cas13b, Cas13c, Cas13d, or Cas13e.

In some embodiments, the surface can comprise a surface of a fluidic chamber or a bead. In some embodiments, the surface comprises a polymer matrix. In some embodiments, the polymer matrix is formed from copolymerization of at least a first plurality of monomers with the reporters. In some embodiments, the polymer matrix comprises a hydrogel.

In some embodiments, the system can further comprise amplification reagents for an amplification reaction targeting the plurality of different target nucleic acids. In some embodiments, the amplification reagents comprise one or more oligonucleotide primers and a DNA polymerase. In any of the embodiments described herein, the known locations can form an array.

In another aspect, the present disclosure provides a method of assaying for a plurality of different target nucleic acids in a sample. The method can comprise: (a) contacting any of the systems disclosed herein with the sample; and (b) detecting at one or more of the known locations a change in signal resulting from cleavage of the reporters. In some embodiments, the known location at which the change in signal is detected identifies the target nucleic acid in the sample. In some embodiments, the polynucleotide sample comprises products of a nucleic acid amplification reaction. In some embodiments, the polynucleotide sample comprises products of a reverse transcription reaction.

In another aspect, the present disclosure provides a method of assaying for a plurality of different target nucleic acids in a sample. The method can comprise (a) contacting a surface with the sample, wherein the surface comprises: (i) a plurality of different non-naturally occurring guide nucleic acids, wherein each of the different non-naturally occurring guide nucleic acids is immobilized to the surface at a known location identified with the particular non-naturally occurring guide nucleic acid; and (ii) a plurality of reporters immobilized to the surface in proximity to each of the different non-naturally occurring guide nucleic acids at each of the known locations. In some embodiments, the method can further comprise (b) forming activated complexes at one or more of the known locations, wherein the activated complexes comprise (i) one of the different non-naturally occurring guide nucleic acids, (ii) a programmable nuclease, and (iii) one of the different target nucleic acids or an amplicon thereof. In some embodiments, the method can further comprise (c) cleaving the reporters with the activated complexes at the one or more known locations by trans cleavage. In some embodiments, the method can further comprise (d) detecting a change in a signal at the one or more known locations comprising the activated complexes, wherein the change in signal is a product of the trans cleavage, and wherein the known location at which the change in signal is detected identifies the target nucleic acid in the sample.

In some embodiments, the step of cleaving the reporters comprises incubation at a temperature of about 37° C. to about 70° C., about 50° C. to about 60° C. or about 55° C.

In some embodiments, the plurality of different non-naturally occurring guide nucleic acids are each immobilized to the surface by a linkage. In some embodiments, the linkage comprises a covalent bond, a non-covalent bond, an electrostatic bond, a bond between members of a binding pair, an amide bond, or any combination thereof. In some embodiments, the linkage comprises a chain of at least 6 carbons, or at least 12 carbons. In some embodiments, the linkage comprises a linker polynucleotide. In some embodiments, the linker polynucleotide comprises a first member of a binding pair that binds to a second member of the binding pair on the surface. In some embodiments, the nucleic acid linker polynucleotide is double-stranded. In some embodiments, the linker polynucleotide comprises double-stranded DNA or single-stranded DNA. In some embodiments, the double-stranded DNA linker polynucleotide is about 60 to about 80 base pairs in length. In some embodiments, the linker polynucleotide is a cleavage substrate for the activated complex.

In some embodiments, the reporters and the non-naturally occurring guide nucleic acids are immobilized at separate discrete positions within each of the known locations. In some embodiments, (i) each of the reporters comprises a fluorescent label and a quencher, (ii) the transcollateral cleavage of the reporters releases the quencher from the cleaved reporters, and (iii) the detecting comprises detecting fluorescence of the fluorescent label. In some embodiments, (i) each of the reporters comprises a detection moiety, (ii) the transcollateral cleavage of the reporters releases the detection moiety from the cleaved reporters, and (iii) the detecting comprises detecting a loss or reduction in signal from the reporter at the respective known location. In some embodiments, the detection moiety comprises a fluorescent label.

In some embodiments, the surface further comprises programmable nucleases immobilized at the known locations by a linkage. In some embodiments, the plurality of different non-naturally occurring guide nucleic acids are immobilized to the surface by being releasably bound by the programmable nucleases. In some embodiments, the surface further comprises programmable nucleases bound to the non-naturally occurring guide nucleic acids.

In some embodiments, the method may further comprise contacting the surface with programmable nucleases to form immobilized complexes at the known locations, wherein (i) the immobilized complexes comprise the programmable nucleases and the non-naturally occurring guide nucleic acids, and (ii) contacting the surface with the programmable nucleases is performed prior to or concurrently with contacting the surface with the sample. In some embodiments, the programmable nuclease comprises an RuvC catalytic domain. In some embodiments, the programmable nuclease is a type V CRISPR/Cas effector protein. In some embodiments, the type V CRISPR/Cas effector protein is a Cas12 protein. In some embodiments, the Cas12 protein comprises Cas12a, Cas12b, Cas12c, Cas12d, or Cas12e. In some embodiments, the type V CRISPR/Cas effector protein is a Cas14 protein. In some embodiments, the Cas14 protein comprises Cas14a, Cas14b, Cas14c, Cas14d, Cas14e, Cas14f, Cas14g, or Cas14h. In some embodiments, the type V CRISPR/Cas effector protein is a CasΦ protein. In some embodiments, the programmable nuclease comprises a HEPN catalytic domain. In some embodiments, the programmable nuclease is a type VI CRISPR/Cas effector protein. In some embodiments, the type VI CRISPR/Cas effector protein is a Cas13 protein. In some embodiments, the Cas13 protein comprises Cas13a, Cas13b, Cas13c, Cas13d, or Cas13e.

In some embodiments, the surface comprises a polymer matrix. In some embodiments, the polymer matrix is formed from copolymerization of at least a first plurality of monomers with the reporters. In some embodiments, the polymer matrix comprises a hydrogel.

In some embodiments, the polynucleotide sample comprises products of a nucleic acid amplification reaction.

In some embodiments, the method can further comprise performing a nucleic acid amplification reaction targeting the plurality of different target nucleic acids, wherein the nucleic acid amplification reaction is: (i) performed on an initial sample to prepare the sample prior to step (a); or (ii) performed after step (a) and before or concurrently with step (b). In some embodiments, the amplification reaction comprises amplification reagents comprising one or more oligonucleotide primers and a DNA polymerase. In some embodiments, the amplification reaction comprises loop mediated amplification (LAMP), wherein the LAMP comprises amplification with a first primer and a second primer targeted to the target nucleic acid and a strand-displacing polymerase, wherein the first primer comprises a 5′ region that is complementary to a sequence generated by extension of the first primer, and wherein the second primer comprises a 5′ region that is complementary to a sequence generated by extension of the second primer. In some embodiments, the amplification reaction comprises reverse transcription. In any of the embodiments described herein, the known locations can form an array.

In another aspect, the present disclosure provides a method of assaying for one or more target nucleic acids in a sample. The method can comprise: (a) amplifying the one or more target nucleic acids to produce DNA amplicons of the one or more target nucleic acids, wherein the amplifying comprises: (i) a plurality of cycles, wherein each cycle comprises denaturation at a first temperature and primer extension by a polymerase at a second temperature that is lower than the first temperature; (ii) each cycle is less than 15 seconds in duration; and (iii) the plurality of cycles comprises at least 20 cycles. In some embodiments, the method can further comprise (b) forming a complex comprising one of the DNA amplicons, a programmable nuclease, and a non-naturally occurring guide nucleic acid that hybridizes to a segment of the DNA amplicon, thereby activating the programmable nuclease. In some embodiments, the method can further comprise (c) cleaving reporters with the activated programmable nuclease. In some embodiments, the method can further comprise (d) detecting a change in a signal, wherein the change in the signal is produced by cleavage of the reporters. In some embodiments, the plurality of cycles comprises at least 25, 30, 35, or 40 cycles. In some embodiments, the plurality of cycles comprises about 45 cycles. In some embodiments, each of the cycles is less than 10 seconds in duration. In some embodiments, each cycle comprises denaturation at the first temperature for about 1 second and primer extension at the second temperature for about 3 seconds. In some embodiments, (i) the first temperature is about 94° C. to about 98° C. and (ii) the second temperature is about 50° C. to about 70° C.

In some embodiments, the amplifying further comprises an initial denaturation step at the denaturation temperature for a duration that is longer than the denaturation steps of the plurality of cycles. In some embodiments, the total duration of the amplifying step is less than 10 minutes, and optionally about 5 minutes.

In some embodiments, steps (b) and (c) are performed by incubation at a third temperature. In some embodiments, the third temperature is about 30° C. to about 70° C. about 37° C. to about 65° C., or about 37° C. In some embodiments, incubation at the third temperature is for a duration of about 10 minutes to about 2 hours, about 20 minutes to about 90 minutes, or about 30) minutes.

In some embodiments, the programmable nuclease is a type V CRISPR/Cas effector protein. In some embodiments, the programmable nuclease is a type V CRISPR/Cas effector protein is a Cas12 protein, a Cas14 protein, or a CasΦ protein. In some embodiments, steps (b) and (c) can be performed using any of the systems described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1A-1C illustrates immobilization strategies for CRISPR-based diagnostic assay components, as described herein.

FIG. 2 illustrates an embodiment where immobilization strategies are combined to enable CRISPR diagnostic readouts, as described herein.

FIG. 3 presents results for the evaluation of the compatibility of various chemical modifications to guide nucleic acids, as described herein.

FIG. 4 presents results for the immobilization of guide nucleic acids to a streptavidin coated surface, as described herein.

FIG. 5 presents results for immobilization of programmable nuclease-nucleic acid complexes, as described herein.

FIGS. 6A-6B presents results for immobilization of reporters, as described herein.

FIG. 7 presents results for functional testing of combined immobilized ribonucleoprotein (RNP) and reporter system, as described herein.

FIGS. 8A-8E presents results for evaluation of different reporters for immobilization in combination with programmable nuclease complex immobilization, as described herein.

FIGS. 9A-9C presents results for a Cy5 reporter that is functional for DETECTR, as described herein.

FIGS. 10A-10F presents results for immobilization optimization involving a complex formation step, as described herein.

FIGS. 11A-11B presents results for immobilization optimization involving a guide nucleic acid/reporter binding time and reporter concentration.

FIGS. 12A-12C presents results showing target discrimination of modified guide nucleic acids.

FIGS. 13A-13E presents results showing that a biotin-modified programmable nuclease-guide nucleic acid complex is functional.

FIG. 14 presents results of a streptavidin coated microscope slide with a biotinylated reporter.

FIGS. 15A-15B presents results of a DETECTR reaction on a glass slide.

FIG. 16 presents experimental conditions, as described herein.

FIG. 17 presents experimental conditions, as described herein. Sequences in the table from top to bottom correspond to SEQ ID NOs: 79-82, respectively.

FIGS. 18A-18B presents experimental conditions, as described herein.

FIGS. 19A-19B presents experimental conditions, as described herein.

FIGS. 20A-20B presents experimental conditions, as described herein.

FIGS. 21A-21B presents experimental conditions, as described herein.

FIGS. 22A-22B presents experimental conditions, as described herein.

FIG. 23 presents experimental conditions, as described herein.

FIG. 24 presents results for the FASTR assay, involving detection of SARS-COV-2 with rapid thermocycling and CRISPR diagnostics.

FIG. 25 presents results from a study to determine top performing polymerases and buffers for the FASTR assay.

FIG. 26 presents results for single copy detection of SARS-COV-2 using the FASTR assay.

FIG. 27 presents results for variations on rapid cycling times for denaturation and annealing/extension in the FASTR assay.

FIG. 28 presents results for minimizing reverse-transcription (RT) time for the FASTR assay.

FIG. 29 presents results for higher pH buffers that improve FASTR assay performance.

FIG. 30 presents results for FASTR assay compatibility with crude lysis buffers.

FIG. 31 presents results for non-optimized multiplexing of the FASTR assay.

FIG. 32 presents results for a multiplex FASTR assay.

FIG. 33 presents results for the limit of detection of a multiplex FASTR assay.

FIG. 34 presents a schematic of combined gRNA and reporter immobilization on the left and results for immobilization of DETECTR components using NHS-Amine chemistries on the right.

FIG. 35 presents results from optimizing the conjugation buffer to reduce non-specific binding.

FIG. 36 presents results from a study involving immobilizing different combinations of reporters, guides, and programmable nucleases.

FIG. 37 presents results from a study optimizing gRNA and target concentrations to improve signal-to-noise ratio for immobilized DETECTR.

FIGS. 38A-38B present modifications and results from evaluating various amino modifications for DETECTR immobilization, respectively.

FIG. 39 shows an exemplary hydrogel comprising immobilized reporters co-polymerized therein.

FIGS. 40A and 40B show exemplary multiplexing strategies for hydrogel immobilized DETECTR systems.

FIGS. 41A-41B show an exemplary positive feedback system for signal amplification.

FIG. 42 shows an exemplary workflow for DETECTR-based HotPot reactions.

FIG. 43 shows fluorescence results of HotPot reactions with reporters immobilized on glass beads.

FIG. 44 shows lateral flow strip results using samples from the same experiments conducted to yield results illustrated in FIG. 43.

FIG. 45 shows fluorescence results of HotPot reactions with reporters immobilized on magnetic beads.

FIGS. 46A-46B show lateral flow assay results of DETECTR-based OnePot and HotPot assays conducted with hydrogels comprising immobilized reporters.

### DETAILED DESCRIPTION

The present disclosure provides systems, devices, apparatuses, methods, and compositions for target detection (including multiplexed target detection), immobilization of biomolecules, and CRISPR immobilization. The systems and methods of the present disclosure can be implemented using devices that are configured for programmable nuclease-based detection of targets. In some embodiments, the systems, devices, and apparatuses described herein can be configured for single reaction detection of one or more targets. In other embodiments, the systems, devices, and apparatuses can be configured for multi-reaction detection of one or more targets. The systems, devices, apparatuses, methods, and compositions disclosed herein can be particularly well suited for carrying out highly efficient, rapid, and accurate reactions for detecting whether one or more targets are present in one or more samples (or any subsamples derived from the one or more samples).

The target can comprise, for example, a target sequence, a target molecule, or a target nucleic acid. As used herein, a target can be referred to interchangeably as a target sequence, target molecule, or target nucleic acid. Further, a target can be referred to as a target amplicon or a target nucleic acid amplicon if such target undergoes amplification (e.g., through a thermocycling process as described elsewhere herein). The target nucleic acid can be a portion of a nucleic acid of interest, e.g., a target nucleic acid from any human, plant, animal, virus, bacteria, or microbe of interest. The systems, devices, apparatuses, methods, and compositions provided herein can be used to perform rapid tests in a single integrated system. The single integrated system may be a reusable unit or a disposable unit.

The target can be, for example, a nucleic acid or a portion of a nucleic acid from a pathogen, virus, bacterium, fungi, protozoa, worm or other agents or organisms responsible for and/or related to a disease or condition in living organisms (e.g., humans, animals, plants, crops and the like). The target nucleic acid can be a nucleic acid, or a portion thereof. The target nucleic acid can be a portion of a nucleic acid from a gene expressed in a cancer or genetic disorder in the sample. The target nucleic acid can be a portion of an RNA or DNA from any organism in the sample.

The target may comprise, for example, a biological sequence. The biological sequence can comprise a nucleic acid sequence or an amino acid sequence. In some embodiments, the target may be associated with an organism of interest, a disease of interest, a disease state of interest, a phenotype of interest, a genotype of interest, or a gene of interest.

In some cases, the target can be associated with one or more pathogenic viruses, pathogenic bacteria, pathogenic worms, pathogenic fungi, or cancer cells. In some embodiments, the pathogenic viruses are selected from the group consisting of respiratory viruses, adenoviruses, parainfluenza viruses, severe acute respiratory syndrome (SARS), coronavirus, SARS-COV, SARS-COV-2, MERS, gastrointestinal viruses, noroviruses, rotaviruses, astroviruses, exanthematous viruses, hepatic viral diseases, cutaneous viral diseases, herpes, hemorrhagic viral diseases, Ebola, Lassa fever, dengue fever, yellow fever, Marburg hemorrhagic fever, Crimean-Congo hemorrhagic fever, neurologic viruses, polio, viral meningitis, viral encephalitis, rabies, sexually transmitted viruses, HIV, HPV, immunodeficiency viruses, influenza virus, dengue virus, West Nile virus, herpes virus, yellow fever virus, Hepatitis Virus C, Hepatitis Virus A, Hepatitis Virus B, and papillomavirus.

In some cases, the target can be indicative of a respiratory disorder or respiratory pathogen. In some embodiments, the respiratory disorder or respiratory pathogen is selected from the group consisting of SARS-COV-2 and corresponding variants, 29E), NL63, OC43, HKUI, MERS-COV, (MERS), SARS-COV (SARS, Flu A, Flu B, RSV, Rhinovirus, Strep A, and TB.

In some embodiments, the target is indicative of a sexually transmitted infection (STI) or infection related to a woman's health. In some embodiments, the STI or infection related to a woman's health is selected from the group consisting of CT, NG, MG, TV, HPV, Candida, B, Vaginosis Syphilis and UTI. In some embodiments, the target comprises a single nucleotide polymorphism (SNP). In some embodiments, the SNP is indicative of NASH disorder or Alpha-1 disorder. In some embodiments, the target is a blood borne pathogen selected from the group consisted of HIV, HBV, HCV and/or Zika. In some embodiments, the target is indicative of H. Pylori, C. Difficile, Norovirus, HSV and/or Meningitis.

### Programmable Nuclease

In some embodiments, one or more programmable nucleases as disclosed herein can be activated to initiate trans cleavage activity of a reporter molecule. A programmable nuclease as disclosed herein can, in some cases, bind to a target sequence or target nucleic acid to initiate trans cleavage of a reporter molecule. In general, the term “reporter” as used in this context refers to a reagent comprising a polynucleotide, wherein cleavage of the polynucleotide results in a change in a signal. For example, the reporter may comprise a fluorescent label joined to a quencher by a short polynucleotide sequence. Little to no fluorescence is detectable from the fluorescent label when joined to the quencher. However, upon cleavage of the polynucleotide, the fluorescent label is separated from the quencher, resulting in a significant and detectable increase in fluorescent signal upon excitation of the label. As a further example, the reporter may comprise a detection moiety (e.g., a fluorescent label) immobilized to a surface by a short polynucleotide sequence, cleavage of which releases the detection moiety and results in a decrease in signal from the detection moiety at the surface. Alternative detection moieties and arrangements for producing a change in signal upon cleavage of the polynucleotide portion of the reporter are possible, and illustrative examples are described herein. The polynucleotide of the reporter can comprise DNA, RNA, modified nucleotides, or a combination of two or more of these. The programmable nuclease can be, in some non-limiting embodiments, an RNA-activated programmable RNA nuclease. In some instances, the programmable nucleases disclosed herein can bind to a target DNA to initiate trans cleavage of a reporter. In some cases, the programmable nuclease can be a DNA-activated programmable RNA nuclease. In some cases, a programmable nuclease as described herein can be activated by a target RNA or a target DNA. For example, the programmable nuclease may comprise a Cas enzyme which can be activated by a target RNA nucleic acid or a target DNA nucleic acid to cleave one or more reporters (e.g., RNA reporter molecules). In some embodiments, the programmable nuclease can bind to a target ssDNA which initiates trans cleavage of RNA reporters. In some instances, a programmable nuclease as disclosed herein can bind to a target DNA to initiate trans cleavage of a DNA reporter. In some non-limiting embodiments, the programmable nuclease can comprise a DNA-activated programmable DNA nuclease.

In some embodiments, the programmable nuclease can comprise an enzyme. The enzyme may be, for example, a Cas enzyme. In some embodiments, the Cas enzyme is selected from the group consisting of a Cas12, Cas12a, Cas13, Cas14, Cas14a, Cas14a.1 (SEQ ID NO: 3), and CasPhi.

Several programmable nucleases are consistent with the systems and methods of the present disclosure. For example, CRISPR/Cas enzymes are programmable nucleases that can be used to implement the methods and systems disclosed herein, CRISPR/Cas enzymes can include any of the known Classes and Types of CRISPR/Cas enzymes. Programmable nucleases disclosed herein can include, for instance, Class 1 CRISPR/Cas enzymes, such as the Type I, Type IV, or Type III CRISPR/Cas enzymes. Programmable nucleases disclosed herein can also include, for example, the Class 2 CRISPR/Cas enzymes, such as the Type II, Type V, and Type VI CRISPR/Cas enzymes.

In some embodiments, the Type V CRISPR/Cas enzyme can comprise a programmable Cas12 nuclease. Type V CRISPR/Cas enzymes (e.g., Cas12 or Cas14) may lack an HNH domain, A Cas12 nuclease of the present disclosure can cleave a nucleic acid via a single catalytic RuvC domain. The RuvC domain is within a nuclease, or “NUC” lobe of the protein, and the Cas12 nuclease can further comprise a recognition, or “REC” lobe. The REC and NUC lobes are connected by a bridge helix and the Cas12 proteins additionally include two domains for PAM recognition termed the PAM interacting (PI) domain and the wedge (WED) domain. In some instances, a programmable Cas12 nuclease can be a Cas12a (also referred to as Cpf1) protein, a Cas12b protein, Cas12c protein, Cas12d protein, or a Cas12e protein.

In some embodiments, the programmable nuclease can be a Cas13 enzyme. Sometimes the Cas13 enzyme can be Cas13a, Cas13b, Cas13c, Cas13d, or Cas13e. In some cases, the programmable nuclease can be Mad7 or Mad2. In some cases, the programmable nuclease can be a Cas12 enzyme as described elsewhere herein. Sometimes the Cas12 can be Cas12a, Cas12b, Cas12c, Cas12d, or Cas12e. In some cases, the Cas12 can be a Cas12 variant (SEQ ID NO: 17), which is a specific protein variant within the Cas12 protein family/classification). In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. Sometimes the programmable nuclease can be an engineered nuclease that is not from a naturally occurring CRISPR-Cas system. In some cases, the programmable nuclease can be from at least one of Leptotrichia shahii (Ish), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp, (Psm), Capnocytophaga canimorsus (Cca, Lachnospiraceae bacterium (Lba), Bergeyella zoohelcum (Bzo), Prevotella intermedia (Pin), Prevotella buccae (Phu), Alistipes sp, (Asp), Riemerella anatipestifer (Ran), Prevotella aurantiaca (Pau), Prevotella saccharolytica (Psa), Prevotella intermedia (Pin2), Capnocytophaga canimorsus ((ca), Porphyromonas gulae (Pgu), Prevotella sp, (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a.

The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA

In some instances, programmable nucleases comprise a Type V CRISPR/Cas protein. In some instances, Type V CRISPR/Cas proteins comprise nucleic acid cleavage activity. In some instances, Type V CRISPR/Cas proteins cleave or nick single-stranded nucleic acids, double, stranded nucleic acids, or a combination thereof. In some cases, Type V CRISPR/Cas proteins cleave single-stranded nucleic acids. In some cases, Type V CRISPR/Cas proteins cleave double-stranded nucleic acids. In some cases, Type V CRISPR/Cas proteins nick double-stranded nucleic acids. Typically, guide RNAs of Type V CRISPR/Cas proteins hybridize to ssDNA or dsDNA. However, the trans cleavage activity of Type V CRISPR/Cas protein is typically directed towards ssDNA.

In some cases, the Type V CRISPR/Cas protein comprises a catalytically inactive nuclease domain. In some cases, the Type V CRISPR/Cas protein comprises a catalytically inactive nuclease domain. A catalytically inactive domain of a Type V CRISPR/Cas protein may comprise at least 1, at least 2, at least 3, at least 4, or at least 5 mutations relative to a wild type nuclease domain of the Type V CRISPR/Cas protein. Said mutations may be present within a cleaving or active site of the nuclease.

In some embodiments, the Type V CRISPR/Cas protein may be a Cas14 protein. The Cas14 protein may be a Cas14a.1 protein (SEQ ID NO: 3). In one example, the Cas14a.1 protein may comprise a sequence of: MAKNTITKTLKLRIVRPYNSAEVEKIVADEKNNREKIALEKNKDKVKEACSKHLKVAA YCTTQVERNACLFCKARKLDDKFYQKLRGQFPDAVFWQEISEIFRQLQKQAAEIYNQSL IELYYEIFIKGKGIANASSVEHYLSDVCYTRAAELFKNAAIASGLRSKIKSNFRLKELKN MKSGLPTTKSDNFPIPLVKQKGGQYTGFEISNHNSDFIIKIPFGRWQVKKEIDKYRPWEK FDFEQVQKSPKPISLLLSTQRRKRNKGWSKDEGTEAEIKKVMNGDYQTSYIEVKRGSKI GEKSAWMLNLSIDVPKIDKGVDPSIIGGIDVGVKSPLVCAINNAFSRYSISDNDLFHFNK KMFARRRILLKKNRHKRAGHGAKNKLKPITILTEKSERFRKKLIERWACEIADFFIKNKV GTVQMENLESMKRKEDSYFNIRLRGFWPYAEMQNKIEFKLKQYGIEIRKVAPNNTSKT CSKCGHLNNYFNFEYRKKNKFPHFKCEKCNFKENADYNAALNISNPKLKSTKEEP (SEQ ID NO: 3). In some cases, the Cas14 protein may comprise an amino acid sequence that is at least 70%, at least 75%, at least 80%, at least 85%, at least 90%, at least 95%, at least 98%, at least 99%, or 100% identical to any exemplary sequence described herein. In some cases, the Cas14 protein may comprise an amino acid sequence that is at least 70%, at least 75%, at least 80%, at least 85%, at least 90%, at least 95%, at least 98%, at least 99%, or 100% identical to any exemplary sequence described herein. In some cases, the Cas14 protein may comprise at least about 50, at least about 100, at least about 150, at least about 200, at least about 250, at least about 300, at least about 350, at least about 400, at least about 450), at least about 500) consecutive amino acids of any exemplary sequence described herein.

In some instances, the Type V CRISPR/Cas protein can be modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF (SEQ ID NO: 62). Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

In some instances, the Type V CRISPR/Cas protein has been modified (also referred to as an engineered protein). For example, a Type V CRISPR/Cas protein disclosed herein or a variant thereof may comprise a nuclear localization signal (NLS). In some cases, the NLS may comprise a sequence of KRPAATKKAGQAKKKKEF (SEQ ID NO: 62). Type V CRISPR/Cas proteins may be codon optimized for expression in a specific cell, for example, a bacterial cell, a plant cell, a eukaryotic cell, an animal cell, a mammalian cell, or a human cell. In some embodiments, the Type V CRISPR/Cas protein is codon optimized for a human cell.

In some instances, the Type V CRISPR/Cas protein comprises a Cas14 protein, Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand-barrel structure. A multi-strand ß-barrel structure may comprise an oligonucleotide/oligosaccharide-binding fold that is structurally comparable to those of some Cas12 proteins. The recognition domain and the zinc finger domain may each (individually or collectively) be inserted between β-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas14 proteins may comprise a RuvC domain or a partial RuvC domain. The RuvC domain may be defined by a single, contiguous sequence, or a set of partial RuvC domains that are not contiguous with respect to the primary amino acid sequence of the Cas14 protein. In some instances, a partial RuvC domain does not have any substrate binding activity or catalytic activity on its own, A Cas14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds, A Cas14 protein may comprise a linker loop connecting a carboxy terminal domain of the Cas14 protein with the amino terminal domain of the Cas14 protein, and wherein the carboxy terminal domain comprises one or more RuvC domains and the amino terminal domain comprises a recognition domain.

Cas14 proteins may comprise a zinc finger domain. In some instances, a carboxy terminal domain of a Cas14 protein comprises a zinc finger domain. In some instances, an amino terminal domain of a Cas14 protein comprises a zinc finger domain. In some instances, the amino terminal domain comprises a wedge domain (e.g., a multi-β-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

Cas14 proteins may be relatively small compared to many other Cas proteins, making them suitable for nucleic acid detection or gene editing. For instance, a Cas14 protein may be less likely to adsorb to a surface or another biological species due to its small size. The smaller nature of these proteins also allows for them to be more easily packaged as a reagent in a system or assay, and delivered with higher efficiency as compared to other larger Cas proteins. In some cases, a Cas14 protein is 400 to 800 amino acid residues long, 400 to 600 amino acid residues long, 440 to 580 amino acid residues long, 460 to 560 amino acid residues long, 460 to 540) amino acid residues long, 460 to 500 amino acid residues long, 400 to 500 amino acid residues long, or 500 to 600 amino acid residues long. In some cases, a Cas14 protein is less than about 550 amino acid residues long. In some cases, a Cas14 protein is less than about 500 amino acid residues long.

In some instances, a Cas14 protein may function as an endonuclease that catalyzes cleavage at a specific position within a target nucleic acid. In some instances, a Cas14 protein is capable of catalyzing non-sequence-specific cleavage of a single stranded nucleic acid. In some cases, a Cas14 protein is activated to perform trans cleavage activity after binding of a guide nucleic acid with a target nucleic acid. This trans cleavage activity is also referred to as “collateral” or “transcollateral” cleavage. Trans cleavage activity may be non-specific cleavage of nearby single-stranded nucleic acid by the activated programmable nuclease, such as trans cleavage of a reporter comprising a nucleic acid and a detection moiety.

In some embodiments, a programmable nuclease is used in a detection reaction. In some embodiments, the detection reaction comprises the steps of (a) forming an activated complex comprising a programmable nuclease, a guide nucleic acid, and a target nucleic acid (or an amplicon thereof), thereby activating the programmable nuclease, and (b) cleaving reporters with the activated programmable nuclease. In some embodiments, steps (a) and (b) are performed at an incubation temperature, such as a temperature of about 25° C. to about 80° C. about 30° C. to about 70° C. or about 37° C. to about 65° C. In some embodiments, the incubation temperature is about 37° C. In some embodiments, the incubation temperature is maintained for about 10) minutes to about 2 hours, about 20) minutes to about 90 minutes, or about 25 minutes to about 60) minutes. In some embodiments, the incubation temperature is maintained for a duration of about 30 minutes. In some embodiments, reporter signal is monitored during the incubation. In some embodiments, reporter signal is measured at the end of the incubation.

### Engineered Programmable Nuclease Probes

Disclosed herein are non-naturally occurring compositions and systems comprising at least one of an engineered programmable nuclease and an engineered guide nucleic acid, which may simply be referred to herein as a programmable nuclease and a guide nucleic acid, respectively. In general, an engineered programmable nuclease and an engineered guide nucleic acid can refer to programmable nucleases and guide nucleic acids, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a programmable nuclease that do not naturally occur together, Conversely, and for clarity, a programmable nuclease or guide nucleic acid that is “natural,” “naturally-occurring,” or “found in nature” includes programmable nucleases and guide nucleic acids from cells or organisms that have not been genetically modified by a human or machine.

In some instances, the guide nucleic acid may comprise a non-natural nucleobase sequence. In some instances, the non-natural sequence is a nucleobase sequence that is not found in nature. The non-natural sequence may comprise a portion of a naturally occurring sequence, wherein the portion of the naturally occurring sequence is not present in nature absent the remainder of the naturally-occurring sequence. In some instances, the guide nucleic acid may comprise two naturally occurring sequences arranged in an order or proximity that is not observed in nature. In some instances, compositions and systems comprise a ribonucleotide complex comprising a CRISPR/Cas effector protein and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

In some instances, compositions and systems provided herein can comprise a multi-vector system for encoding any programmable nuclease or guide nucleic acid described herein, wherein the guide nucleic acid and the programmable nuclease are encoded by the same or different vectors. In some embodiments, the engineered guide nucleic acid and the engineered programmable nuclease can be encoded by different vectors of the system.

### Thermostable Programmable Nuclease

Described herein are various embodiments of thermostable programmable nucleases. In some embodiments, a programmable nuclease is referred to as an effector protein. An effector protein may be thermostable. In some instances, known effector proteins (e.g., Cas12 nucleases) are relatively thermo-sensitive and only exhibit activity (e.g., cis and/or trans cleavage) sufficient to produce a detectable signal in a diagnostic assay at temperatures less than 40° C. and optimally at about 37° C. A thermostable protein may have enzymatic activity, stability, or folding comparable to those at 37° C. In some instances, the trans cleavage activity (e.g., the maximum trans cleavage rate as measured by fluorescent signal generation) of an effector protein in a trans cleavage assay at 40° C. may be at least 50% of that at 37° C. In some instances, the trans cleavage activity of an effector protein in a trans cleavage assay at 40° C. may be at least 55% of that at 37° C. In some instances, the trans cleavage activity of an effector protein in a trans cleavage assay at 40° C. may be at least 60% of that at 37° C. In some instances, the trans cleavage activity of an effector protein in a trans cleavage assay at 40° C. may be at least 65% of that at 37° C. In some instances, the trans cleavage activity of an effector protein in a trans cleavage assay at 40° C. may be at least 70% of that at 37° C. In some instances, the trans cleavage activity of an effector protein in a trans cleavage assay at 40° C. may be at least 75% of that at 37° C. In some instances, the trans cleavage activity of an effector protein in a trans cleavage assay at 40° C. may be at least 80% of that at 37° C. In some instances, the trans cleavage activity of an effector protein in a trans cleavage assay at 40° C. may be at least 85% of that at 37° C. In some instances, the trans cleavage activity of an effector protein in a trans cleavage assay at 40° C. may be at least 90% of that at 37° C. In some instances, the trans cleavage activity of an effector protein in a trans cleavage assay at 40° C. may be at least 95% of that at 37° C. In some instances, the trans cleavage activity of an effector protein in a trans cleavage assay at 40° C. may be at least 100% of that at 37° C. In some instances, the trans cleavage activity of an effector protein in a trans cleavage assay at 40° C. may be at least 1-fold of that at 37° C. In some instances, the trans cleavage activity of an effector protein in a trans cleavage assay at 40° C. may be at least 2-fold of that at 37° C. In some instances, the trans cleavage activity of an effector protein in a trans cleavage assay at 40° C. may be at least 3-fold of that at 37° C. In some instances, the trans cleavage activity of an effector protein in a trans cleavage assay at 40° C. may be at least 4-fold of that at 37° C. In some instances, the trans cleavage activity of an effector protein in a trans cleavage assay at 40° C. may be at least 5-fold of that at 37° C. In some instances, the trans cleavage activity of an effector protein in a trans cleavage assay at 40° C. may be at least 6-fold of that at 37° C. In some instances, the trans cleavage activity of an effector protein in a trans cleavage assay at 40° C. may be at least 7-fold of that at 37° C. In some instances, the trans cleavage activity of an effector protein in a trans cleavage assay at 40° C. may be at least 8-fold of that at 37° C. In some instances, the trans cleavage activity of an effector protein in a trans cleavage assay at 40° C. may be at least 9-fold of that at 37° C. In some instances, the trans cleavage activity of an effector protein in a trans cleavage assay at 40° C. may be at least 10-fold of that at 37° C. In some instances, the trans cleavage activity of an effector protein in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 45° C. may be at least 50% of that at 37° C. In some instances, the trans cleavage activity of an effector protein in a trans cleavage assay at 45° C. may be at least 55% of that at 37° C. In some instances, the trans cleavage activity of an effector protein in a trans cleavage assay at 45° C. may be at least 60% of that at 37° C. In some instances, the trans cleavage activity of an effector protein in a trans cleavage assay at 45° C. may be at least 65% of that at 37° C. In some instances, the trans cleavage activity of an effector protein in a trans cleavage assay at 45° C. may be at least 70% of that at 37° C. In some instances, the trans cleavage activity of an effector protein in a trans cleavage assay at 45° C. may be at least 75% of that at 37° C. In some instances, the trans cleavage activity of an effector protein in a trans cleavage assay at 45° C. may be at least 80% of that at 37° C. In some instances, the trans cleavage activity of an effector protein in a trans cleavage assay at 45° C. may be at least 85% of that at 37° C. In some instances, the trans cleavage activity of an effector protein in a trans cleavage assay at 45° C. may be at least 90% of that at 37° C. In some instances, the trans cleavage activity of an effector protein in a trans cleavage assay at 45° C. may be at least 95% of that at 37° C. In some instances, the trans cleavage activity of an effector protein in a trans cleavage assay at 45° C. may be at least 100% of that at 37° C. In some instances, the trans cleavage activity of an effector protein in a trans cleavage assay at 45° C. may be at least 1-fold of that at 37° C. In some instances, the trans cleavage activity of an effector protein in a trans cleavage assay at 45° C. may be at least 2-fold of that at 37° C. In some instances, the trans cleavage activity of an effector protein in a trans cleavage assay at 45° C. may be at least 3-fold of that at 37° C. In some instances, the trans cleavage activity of an effector protein in a trans cleavage assay at 45° C. may be at least 4-fold of that at 37° C. In some instances, the trans cleavage activity of an effector protein in a trans cleavage assay at 45° C. may be at least 5-fold of that at 37° C. In some instances, the trans cleavage activity of an effector protein in a trans cleavage assay at 45° C. may be at least 6-fold of that at 37° C. In some instances, the trans cleavage activity of an effector protein in a trans cleavage assay at 45° C. may be at least 7-fold of that at 37° C. In some instances, the trans cleavage activity of an effector protein in a trans cleavage assay at 45° C. may be at least 8-fold of that at 37° C. In some instances, the trans cleavage activity of an effector protein in a trans cleavage assay at 45° C. may be at least 9-fold of that at 37° C. In some instances, the trans cleavage activity of an effector protein in a trans cleavage assay at 45° C. may be at least 10-fold of that at 37° C. In some instances, the trans cleavage activity of an effector protein in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 50° C. may be at least 50% of that at 37° C. In some instances, the trans cleavage activity of an effector protein in a trans cleavage assay at 50° C. may be at least 55% of that at 37° C. In some instances, the trans cleavage activity of an effector protein in a trans cleavage assay at 50° C. may be at least 60% of that at 37° C. In some instances, the trans cleavage activity of an effector protein in a trans cleavage assay at 50° C. may be at least 65% of that at 37° C. In some instances, the trans cleavage activity of an effector protein in a trans cleavage assay at 50° C. may be at least 70% of that at 37° C. In some instances, the trans cleavage activity of an effector protein in a trans cleavage assay at 50° C. may be at least 75% of that at 37° C. In some instances, the trans cleavage activity of an effector protein in a trans cleavage assay at 50° C. may be at least 80% of that at 37° C. In some instances, the trans cleavage activity of an effector protein in a trans cleavage assay at 50° C. may be at least 85% of that at 37° C. In some instances, the trans cleavage activity of an effector protein in a trans cleavage assay at 50° C. may be at least 90% of that at 37° C. In some instances, the trans cleavage activity of an effector protein in a trans cleavage assay at 50° C. may be at least 95% of that at 37° C. In some instances, the trans cleavage activity of an effector protein in a trans cleavage assay at 50° C. may be at least 100% of that at 37° C. In some instances, the trans cleavage activity of an effector protein in a trans cleavage assay at 50° C. may be at least 1-fold of that at 37° C. In some instances, the trans cleavage activity of an effector protein in a trans cleavage assay at 50° C. may be at least 2-fold of that at 37° C. In some instances, the trans cleavage activity of an effector protein in a trans cleavage assay at 50° C. may be at least 3-fold of that at 37° C. In some instances, the trans cleavage activity of an effector protein in a trans cleavage assay at 50° C. may be at least 4-fold of that at 37° C. In some instances, the trans cleavage activity of an effector protein in a trans cleavage assay at 50° C. may be at least 5-fold of that at 37° C. In some instances, the trans cleavage activity of an effector protein in a trans cleavage assay at 50° C. may be at least 6-fold of that at 37° C. In some instances, the trans cleavage activity of an effector protein in a trans cleavage assay at 50° C. may be at least 7-fold of that at 37° C. In some instances, the trans cleavage activity of an effector protein in a trans cleavage assay at 50° C. may be at least 8-fold of that at 37° C. In some instances, the trans cleavage activity of an effector protein in a trans cleavage assay at 50° C. may be at least 9-fold of that at 37° C. In some instances, the trans cleavage activity of an effector protein in a trans cleavage assay at 50° C. may be at least 10-fold of that at 37° C. In some instances, the trans cleavage activity of an effector protein in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 55° C. may be at least 50% of that at 37° C. In some instances, the trans cleavage activity of an effector protein in a trans cleavage assay at 55° C. may be at least 55% of that at 37° C. In some instances, the trans cleavage activity of an effector protein in a trans cleavage assay at 55° C. may be at least 60% of that at 37° C. In some instances, the trans cleavage activity of an effector protein in a trans cleavage assay at 55° C. may be at least 65% of that at 37° C. In some instances, the trans cleavage activity of an effector protein in a trans cleavage assay at 55° C. may be at least 70% of that at 37° C. In some instances, the trans cleavage activity of an effector protein in a trans cleavage assay at 55° C. may be at least 75% of that at 37° C. In some instances, the trans cleavage activity of an effector protein in a trans cleavage assay at 55° C. may be at least 80% of that at 37° C. In some instances, the trans cleavage activity of an effector protein in a trans cleavage assay at 55° C. may be at least 85% of that at 37° C. In some instances, the trans cleavage activity of an effector protein in a trans cleavage assay at 55° C. may be at least 90% of that at 37° C. In some instances, the trans cleavage activity of an effector protein in a trans cleavage assay at 55° C. may be at least 95% of that at 37° C. In some instances, the trans cleavage activity of an effector protein in a trans cleavage assay at 55° C. may be about 100% of that at 37° C. In some instances, the trans cleavage activity of an effector protein in a trans cleavage assay at 55° C. may be at least 1-fold of that at 37° C. In some instances, the trans cleavage activity of an effector protein in a trans cleavage assay at 55° C. may be at least 2-fold of that at 37° C. In some instances, the trans cleavage activity of an effector protein in a trans cleavage assay at 55° C. may be at least 3-fold of that at 37° C. In some instances, the trans cleavage activity of an effector protein in a trans cleavage assay at 55° C. may be at least 4-fold of that at 37° C. In some instances, the trans cleavage activity of an effector protein in a trans cleavage assay at 55° C. may be at least 5-fold of that at 37° C. In some instances, the trans cleavage activity of an effector protein in a trans cleavage assay at 55° C. may be at least 6-fold of that at 37° C. In some instances, the trans cleavage activity of an effector protein in a trans cleavage assay at 55° C. may be at least 7-fold of that at 37° C. In some instances, the trans cleavage activity of an effector protein in a trans cleavage assay at 55° C. may be at least 8-fold of that at 37° C. In some instances, the trans cleavage activity of an effector protein in a trans cleavage assay at 55° C. may be at least 9-fold of that at 37° C. In some instances, the trans cleavage activity of an effector protein in a trans cleavage assay at 55° C. may be at least 10-fold of that at 37° C. In some instances, the trans cleavage activity of an effector protein in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 60° C. may be at least 50% of that at 37° C. In some instances, the trans cleavage activity of an effector protein in a trans cleavage assay at 60° C. may be at least 55% of that at 37° C. In some instances, the trans cleavage activity of an effector protein in a trans cleavage assay at 60° C. may be at least 60% of that at 37° C. In some instances, the trans cleavage activity of an effector protein in a trans cleavage assay at 60° C. may be at least 65% of that at 37° C. In some instances, the trans cleavage activity of an effector protein in a trans cleavage assay at 60° C. may be at least 70% of that at 37° C. In some instances, the trans cleavage activity of an effector protein in a trans cleavage assay at 60° C. may be at least 75% of that at 37° C. In some instances, the trans cleavage activity of an effector protein in a trans cleavage assay at 60° C. may be at least 80% of that at 37° C. In some instances, the trans cleavage activity of an effector protein in a trans cleavage assay at 60° C. may be at least 85% of that at 37° C. In some instances, the trans cleavage activity of an effector protein in a trans cleavage assay at 60° C. may be at least 90% of that at 37° C. In some instances, the trans cleavage activity of an effector protein in a trans cleavage assay at 60° C. may be at least 95% of that at 37° C. In some instances, the trans cleavage activity of an effector protein in a trans cleavage assay at 60° C. may be about 100% of that at 37° C. In some instances, the trans cleavage activity of an effector protein in a trans cleavage assay at 60° C. may be at least 1-fold of that at 37° C. In some instances, the trans cleavage activity of an effector protein in a trans cleavage assay at 60° C. may be at least 2-fold of that at 37° C. In some instances, the trans cleavage activity of an effector protein in a trans cleavage assay at 60° C. may be at least 3-fold of that at 37° C. In some instances, the trans cleavage activity of an effector protein in a trans cleavage assay at 60° C. may be at least 4-fold of that at 37° C. In some instances, the trans cleavage activity of an effector protein in a trans cleavage assay at 60° C. may be at least 5-fold of that at 37° C. In some instances, the trans cleavage activity of an effector protein in a trans cleavage assay at 60° C. may be at least 6-fold of that at 37° C. In some instances, the trans cleavage activity of an effector protein in a trans cleavage assay at 60° C. may be at least 7-fold of that at 37° C. In some instances, the trans cleavage activity of an effector protein in a trans cleavage assay at 60° C. may be at least 8-fold of that at 37° C. In some instances, the trans cleavage activity of an effector protein in a trans cleavage assay at 60° C. may be at least 9-fold of that at 37° C. In some instances, the trans cleavage activity of an effector protein in a trans cleavage assay at 60° C. may be at least 10-fold of that at 37° C. In some instances, the trans cleavage activity of an effector protein in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity of an effector protein in a trans cleavage assay at 65° C. may be at least 50% of that at 37° C. In some instances, the trans cleavage activity of an effector protein in a trans cleavage assay at 65° C. may be at least 55% of that at 37° C. In some instances, the trans cleavage activity of an effector protein in a trans cleavage assay at 65° C. may be at least 60% of that at 37° C. In some instances, the trans cleavage activity of an effector protein in a trans cleavage assay at 65° C. may be at least 65% of that at 37° C. In some instances, the trans cleavage activity of an effector protein in a trans cleavage assay at 65° C. may be at least 70% of that at 37° C. In some instances, the trans cleavage activity of an effector protein in a trans cleavage assay at 65° C. may be at least 75% of that at 37° C. In some instances, the trans cleavage activity of an effector protein in a trans cleavage assay at 65° C. may be at least 80% of that at 37° C. In some instances, the trans cleavage activity of an effector protein in a trans cleavage assay at 65° C. may be at least 85% of that at 37° C. In some instances, the trans cleavage activity of an effector protein in a trans cleavage assay at 65° C. may be at least 90% of that at 37° C. In some instances, the trans cleavage activity of an effector protein in a trans cleavage assay at 65° C. may be at least 95% of that at 37° C. In some instances, the trans cleavage activity of an effector protein in a trans cleavage assay at 65° C. may be about 100% of that at 37° C. In some instances, the trans cleavage activity of an effector protein in a trans cleavage assay at 65° C. may be at least 1-fold of that at 37° C. In some instances, the trans cleavage activity of an effector protein in a trans cleavage assay at 65° C. may be at least 2-fold of that at 37° C. In some instances, the trans cleavage activity of an effector protein in a trans cleavage assay at 65° C. may be at least 3-fold of that at 37° C. In some instances, the trans cleavage activity of an effector protein in a trans cleavage assay at 65° C. may be at least 4-fold of that at 37° C. In some instances, the trans cleavage activity of an effector protein in a trans cleavage assay at 65° C. may be at least 5-fold of that at 37° C. In some instances, the trans cleavage activity of an effector protein in a trans cleavage assay at 65° C. may be at least 6-fold of that at 37° C. In some instances, the trans cleavage activity of an effector protein in a trans cleavage assay at 65° C. may be at least 7-fold of that at 37° C. In some instances, the trans cleavage activity of an effector protein in a trans cleavage assay at 65° C. may be at least 8-fold of that at 37° C. In some instances, the trans cleavage activity of an effector protein in a trans cleavage assay at 65° C. may be at least 9-fold of that at 37° C. In some instances, the trans cleavage activity of an effector protein in a trans cleavage assay at 65° C. may be at least 10-fold of that at 37° C. In some instances, the trans cleavage activity of an effector protein in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C. In some instances, the trans cleavage activity of an effector protein in a trans cleavage assay at 70° C. 75° C. 80° C. or more may be at least 50, at least 60%, at least 65%, at least 70%, at least 75%, at least 80%, at least 85%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some instances, the trans cleavage activity may be measured against a negative control in a trans cleavage assay. In some instances, the trans cleavage activity of an effector protein against a nucleic acid in a trans cleavage assay at 37° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 37° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 40° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 45° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 50° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 55° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 60° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 65° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some instances, the trans cleavage activity of an effector protein against a nucleic acid in a trans cleavage assay at 70° C. 75° C. 80° C. or more may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some instances, the trans cleavage activity of an effector protein against a nucleic acid in a trans cleavage assay at 70° C. 75° C. 80° C. or more may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid.

### Additional Examples of Programmable Nucleases

As described elsewhere herein, one or more programmable nucleases may be used to detect one or more targets (e.g., one or more target nucleic acids). In some cases, a programmable nuclease is capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and can non-specifically degrade a non-target nucleic acid in its environment. The programmable nuclease has trans cleavage activity once activated. A programmable nuclease can be a Cas protein (also referred to, interchangeably, as a Cas nuclease or Cas effector protein). A guide nucleic acid (e.g., crRNA) and Cas protein can form a CRISPR enzyme.

In some embodiments, one or more programmable nucleases as disclosed herein can be activated to initiate trans cleavage activity of a reporter (also referred to herein as a reporter molecule). A programmable nuclease as disclosed herein can, in some cases, bind to a target sequence or target nucleic acid to initiate trans cleavage of a reporter. The programmable nuclease can be referred to as an RNA-activated programmable RNA nuclease. In some instances, the programmable nuclease as disclosed herein can bind to a target DNA to initiate trans cleavage of an RNA reporter. Such a programmable nuclease can be referred to herein as a DNA-activated programmable RNA nuclease. In some cases, a programmable nuclease as described herein can be activated by a target RNA or a target DNA. For example, a programmable nuclease, e.g., a Cas enzyme, can be activated by a target RNA nucleic acid or a target DNA nucleic acid to cleave RNA reporters. In some embodiments, the programmable nuclease can bind to a target ssDNA which initiates trans cleavage of RNA reporters. In some instances, a programmable nuclease as disclosed herein can bind to a target DNA to initiate trans cleavage of a DNA reporter, and this programmable nuclease can be referred to as a DNA-activated programmable DNA nuclease.

The programmable nuclease can become activated after binding of a guide nucleic acid that is complexed with the programmable nuclease with a target nucleic acid, and the activated programmable nuclease can cleave the target nucleic acid, which can result in a trans cleavage activity. Trans cleavage activity can be non-specific cleavage of nearby single-stranded nucleic acids by the activated programmable nuclease, such as trans cleavage of reporter nucleic acids comprising a detection moiety. Once the reporter is cleaved by the activated programmable nuclease, the detection moiety can be released or separated from the reporter and can directly or indirectly generate a detectable signal. The reporter and/or the detection moiety can be immobilized on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a SNP associated with a disease, cancer, or genetic disorder.

A programmable nuclease can be any enzyme that can be or has been designed, modified, or engineered by human contribution so that the enzyme targets or cleaves a nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases or Cpf1. Programmable nucleases can also include, for example, PfAgo and/or NgAgo.

Several programmable nucleases are consistent with the methods and devices of the present disclosure. For example, Cas proteins are programmable nucleases used in the methods and systems disclosed herein. Cas proteins can include any of the known Classes and Types of CRISPR/Cas enzymes. Programmable nucleases disclosed herein include Class 1 Cas proteins, such as the Type I, Type IV, or Type III Cas proteins. Programmable nucleases disclosed herein also include the Class 2 Cas proteins, such as the Type II, Type V, and Type VI Cas proteins. Programmable nucleases included in the methods disclosed herein and methods of use thereof include a Type V or Type VI Cas proteins.

In some instances, the programmable nuclease is a Type V Cas protein. In general, a Type V Cas effector protein comprises a RuvC domain, but lacks an HNH domain. In most instances, the RuvC domain of the Type V Cas effector protein comprises three partial RuvC domains (RuvC-I, RuvC-II, and RuvC-III, also referred to herein as subdomains). In some instances, the three RuvC subdomains are located within the C-terminal half of the Type V Cas effector protein. In some instances, none of the RuvC subdomains are located at the N terminus of the protein. In some instances, the RuvC subdomains are contiguous. In some instances, the RuvC subdomains are not contiguous with respect to the primary amino acid sequence of the Type V Cas protein, but form a ruvC domain once the protein is produced and folds. In some instances, there are zero to about 50 amino acids between the first and second RuvC subdomains. In some instances, there are zero to about 50 amino acids between the second and third RuvC subdomains. In some instances, the Cas effector is a Cas14 effector. In some instances, the Cas14 effector is a Cas14a, Cas14a.1 (SEQ ID NO: 3), Cas14b, Cas14c, Cas14d, Cas14e, Cas14f, Cas14g, Cas14h, or Cas14u effector. In some instances, the Cas effector is a CasPhi (also referred to herein as a Casϕ) effector. In some instances, the Cas effector is a Cas12 effector. In some instances, the Cas12 effector is a Cas12a, Cas12b, Cas12c, Cas12d, Cas12e, or Cas12j effector.

In some instances, the Type V Cas protein comprises a Cas14 protein. Cas14 proteins may comprise a bilobed structure with distinct amino-terminal and carboxy-terminal domains. The amino- and carboxy-terminal domains may be connected by a flexible linker. The flexible linker may affect the relative conformations of the amino- and carboxyl-terminal domains. The flexible linker may be short, for example less than 10 amino acids, less than 8 amino acids, less than 6 amino acids, less than 5 amino acids, or less than 4 amino acids in length. The flexible linker may be sufficiently long to enable different conformations of the amino- and carboxy-terminal domains among two Cas14 proteins of a Cas14 dimer complex (e.g., the relative orientations of the amino- and carboxy-terminal domains differ between two Cas14 proteins of a Cas14 homodimer complex). The linker domain may comprise a mutation which affects the relative conformations of the amino- and carboxyl-terminal domains. The linker may comprise a mutation which affects Cas14 dimerization. For example, a linker mutation may enhance the stability of a Cas14 dimer.

In some instances, the amino-terminal domain of a Cas14 protein comprises a wedge domain, a recognition domain, a zinc finger domain, or any combination thereof. The wedge domain may comprise a multi-strand β-barrel structure. A multi-strand β-barrel structure may comprise an oligonucleotide/oligosaccharide-binding fold that is structurally comparable to those of some Cas12 proteins. The recognition domain and the zinc finger domain may each (individually or collectively) be inserted between β-barrel strands of the wedge domain. The recognition domain may comprise a 4-α-helix structure, structurally comparable but shorter than those found in some Cas12 proteins. The recognition domain may comprise a binding affinity for a guide nucleic acid or for a guide nucleic acid-target nucleic acid heteroduplex. In some cases, a REC lobe may comprise a binding affinity for a PAM sequence in the target nucleic acid. The amino-terminal may comprise a wedge domain, a recognition domain, and a zinc finger domain. The carboxy-terminal may comprise a RuvC domain, a zinc finger domain, or any combination thereof. The carboxy-terminal may comprise one RuvC and one zinc finger domain.

Cas14 proteins may comprise a RuvC domain or a partial RuvC domain. The RuvC domain may be defined by a single, contiguous sequence, or a set of partial RuvC domains that are not contiguous with respect to the primary amino acid sequence of the Cas14 protein. In some instances, a partial RuvC domain does not have any substrate binding activity or catalytic activity on its own, A Cas14 protein of the present disclosure may include multiple partial RuvC domains, which may combine to generate a RuvC domain with substrate binding or catalytic activity. For example, a Cas14 may include 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds, A Cas14 protein may comprise a linker loop connecting a carboxy terminal domain of the Cas14 protein with the amino terminal domain of the Cas14 protein, and wherein the carboxy terminal domain comprises one or more RuvC domains and the amino terminal domain comprises a recognition domain.

Cas14 proteins may comprise a zinc finger domain. In some instances, a carboxy terminal domain of a Cas14 protein comprises a zinc finger domain. In some instances, an amino terminal domain of a Cas14 protein comprises a zinc finger domain. In some instances, the amino terminal domain comprises a wedge domain (e.g., a multi-β-barrel wedge structure), a zinc finger domain, or any combination thereof. In some cases, the carboxy terminal domain comprises the RuvC domains and a zinc finger domain, and the amino terminal domain comprises a recognition domain, a wedge domain, and a zinc finger domain.

In some instances, the Type V Cas protein is a Casϕ protein, A CasΦ protein can function as an endonuclease that catalyzes cleavage at a specific sequence in a target nucleic acid. A programmable CasΦ nuclease may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

In some instances, the programmable nuclease is a Type VI Cas protein. In some embodiments, the Type VI Cas protein is a programmable Cas13 nuclease. The general architecture of a Cas13 protein includes an N-terminal domain and two HEPN (higher eukaryotes and prokaryotes nucleotide-binding) domains separated by two helical domains. The HEPN domains each comprise aR-X₄-H motif. Shared features across Cas13 proteins include that upon binding of the crRNA of the guide nucleic acid to a target nucleic acid, the protein undergoes a conformational change to bring together the HEPN domains and form a catalytically active RNase, Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. Programmable Cas13 nucleases consistent with the present disclosure also Cas13 nucleases comprising catalytic components. In some instances, the Cas effector is a Cas13 effector. In some instances, the Cas13 effector is a Cas13a, a Cas13b, a Cas13c, a Cas13d, or a Cas13e effector protein.

In some cases, the programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the programmable nuclease can be Csm1, Cas9, C2c4, C2c8, C2c5, C2c10), C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum ((am), Prevotella sp, (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp, (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp, (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the programmable nuclease can be activated when the guide nucleic acid is complexed with the target nucleic acid. In some embodiments, the target nucleic acid can be RNA or DNA.

In some embodiments, the programmable nuclease comprises a Cas12 protein, wherein the Cas12 enzyme binds and cleaves double stranded DNA and single stranded DNA. In some embodiments, programmable nuclease comprises a Cas13 protein, wherein the Cas13 enzyme binds and cleaves single stranded RNA. In some embodiments, programmable nuclease comprises a Cas14 protein, wherein the Cas14 enzyme binds and cleaves both double stranded DNA and single stranded DNA.

Table 2A provides illustrative amino acid sequences of programmable nucleases having trans-cleavage activity. In some instances, programmable nucleases described herein comprise an amino acid sequence that is at least 65%, at least 70%, at least 75%, at least 80%, at least 85%, at least 90%, at least 95%, at least 97%, or at least 98%, at least 99%, or 100% identical to any one of SEQ ID Nos: 1-61. In some instances, programmable nucleases described herein may consist of an amino acid sequence that is at least 65%, at least 70%, at least 75%, at least 80%, at least 85%, at least 90%, at least 95%, at least 97%, or at least 98%, at least 99%, or 100% identical to any one of SEQ ID Nos: 1-61. In some instances, programmable nucleases described herein comprise at least about 50, at least about 100, at least about 150, at least about 200, at least about 250, at least about 300, at least about 350, at least about 400, at least about 450, at least about 500 consecutive amino acids of any one of SEQ ID Nos: 1-61,

TABLE 2A
Amino Acid Sequences of Exemplary
Programmable Nucleases

| SEQ ID | Programmable Nuclease |
|---|---|
| NO: | Amino Acid Sequence |
| SEQ ID: | MADLSQFTHKYQVPKTLRFELIPQGKTLEN |
| 1 | LSAYGMVADDKQRSENYKKLKPVIDRIYKY |
| | FIEESLKNTNLDWNPLYEAIREYRKEKTTA |
| | TITNLKEQQDICRRAIASRFEGKVPDKGDK |
| | SVKDFNKKQSKLFKELFGKELFTDSVLEQL |
| | PGVSLSDEDKALLKSFDKFTTYFVGFYDNR |
| | KNVFSSDDISTGIPHRLVQENFPKFIDNCD |
| | DYKRLVLVAPELKEKLEKAAEATKIFEDVS |
| | LDEIFSIKFYNRLLQQNQIDQFNQLLGGIA |
| | GAPGTPKIQGLNETLNLSMQQDKTLEQKLK |
| | SVPHRFSPLYKQILSDRSSLSFIPESFSCD |
| | AEVLLAVQEYLDNLKTEHVIEDLKEVFNRL |
| | TTLDLKHIYVNSTKVTAFSQALFGDWNLCR |
| | EQLRVYKMSNGNEKITKKALGELESWLKNS |
| | DIAFTELQEALADEALPAKVNLKVQEAISG |
| | LNEQMAKSLPKELKIPEEKEELKALLDAIQ |
| | EVYHTLEWFIVSDDVETDTDFYVPLKETLQ |
| | IIQPIIPLYNKVRNFATQKPYSVEKFKLNF |
| | ANPTLADGWDENKEQQNCAVLFQKGNNYYL |
| | GILNPKNKPDFDNVDTEKQGNCYQKMVYKQ |
| | FPDFSKMMPKCTTQLKEVKQHFEGKDSDYI |
| | LNNKNFIKPLTITREVYDLNNVLYDGKKKF |
| | QIDYLRKTKDEDGYYTALHTWIDFAKKFVA |
| | SYKSTSIYDTSTILPPEKYEKLNEFYGALD |
| | NLFYQIKFENIPEEIIDTYVEDGKLFLFQI |
| | YNKDFAAGATGAPNLHTIYWKAVFDPENVK |
| | DVVVKLNGQAELFYRPKSNMDVIRHKVGEK |
| | LVNRTLKDGSILTDELHKELYLYANGSLKK |
| | GLSEDAKIILDKNLAVIYDVHHEIVKDRRF |
| | TTDKFFFHVPLTLNYKCDKNPVKFNAEVQE |
| | YLKENPDTYVIGIDRGERNLIYAVVIDPKG |
| | RIVEQKSFNVINGFDYHGKLDQREKERVKA |
| | RQAWTAVGKIKELKQGYLSLVVHEISKMMV |
| | RYQAVVVLENLNVGFKRVRSGIAEKAVYQQ |
| | FEKMLINKLNYLMFKDAGGTEPGSVLNAYQ |
| | LTDRFESFAKMGLQTGFLFYIPAAFTSKID |
| | PATGFVDPFRWGAIKTLADKREFLSGFESL |
| | KFDSTTGNFILHFDVSKNKNFQKKLEGFVP |
| | DWDIIIEANKMKTGKGATYIAGKRIEFVRD |
| | NNSQGHYEDYLPCNALAETLRQCDIPYEEG |
| | KDILPLILEKNDSKLLHSVFKVVRLTLQMR |
| | NSNAETGEDYISSPVEDVSGSCFDSRMENE |
| | KLPKDADANGAYHIALKGMLALERLRKDEK |
| | MAISNNDWLNYIQEKRA* |
| SEQ ID: | MAGKKKDKDVINKTLSVRIIRPRYSDDIEK |
| 2 | EISDEKAKRKQDGKTGELDRAFFSELKSRN |
| | PDIITNDELFPLFTEIQKNLTEIYNKSISL |
| | LYMKLIVEEEGGSTASALSAGPYKECKARF |
| | NSYISLGLRQKIQSNFRRKELKGFQVSLPT |
| | AKSDRFPIPFCHQVENGKGGFKVYETGDDF |
| | IFEVPLIKYTATNKKSTSGKNYTKVQLNNP |
| | PVPMNVPLLLSTMRRRQTKKGMQWNKDEGT |
| | NAELRRVMSGEYKVSYAEIIRRTRFGKHDD |
| | WFVNFSIKFKNKTDELNQNVRGGIDIGVSN |
| | PLVCAVTNGLDRYIVANNDIMAFNERAMAR |
| | RRTLLRKNRFKRSGHGAKNKLEPITVLTEK |
| | NERFRKSILQRWAREVAEFFKRTSASVVNM |
| | EDLSGITEREDFFSTKLRTTWNYRLMQTTI |
| | ENKLKEYGIAVNYISPKYTSQTCHSCGKRN |
| | DYFTFSYRSENNYPPFECKECNKVKCNADF |
| | NAAKNIALKVVL |
| SEQ ID: | MAKNTITKTLKLRIVRPYNSAEVEKIVADE |
| 3 | KNNREKIALEKNKDKVKEACSKHLKVAAYC |
| | TTQVERNACLFCKARKLDDKFYQKLRGQFP |
| | DAVFWQEISEIFRQLQKQAAEIYNQSLIEL |
| | YYEIFIKGKGIANASSVEHYLSDVCYTRAA |
| | ELFKNAAIASGLRSKIKSNFRLKELKNMKS |
| | GLPTTKSDNFPIPLVKQKGGQYTGFEISNH |
| | NSDFIIKIPFGRWQVKKEIDKYRPWEKFDF |
| | EQVQKSPKPISLLLSTQRRKRNKGWSKDEG |
| | TEAEIKKVMNGDYQTSYIEVKRGSKIGEKS |
| | AWMLNLSIDVPKIDKGVDPSIIGGIDVGVK |
| | SPLVCAINNAFSRYSISDNDLFHFNKKMFA |
| | RRRILLKKNRHKRAGHGAKNKLKPITILTE |
| | KSERFRKKLIERWACEIADFFIKNKVGTVQ |
| | MENLESMKRKEDSYFNIRLRGFWPYAEMQN |
| | KIEFKLKQYGIEIRKVAPNNTSKTCSKCGH |
| | LNNYFNFEYRKKNKFPHFKCEKCNFKENAD |
| | YNAALNISNPKLKSTKEEP |
| SEQ ID: | MATLVSFTKQYQVQKTLRFELIPQGKTQAN |
| 4 | IDAKGFINDDLKRDENYMKVKGVIDELHKN |
| | FIEQTLVNVDYDWRSLATAIKNYRKDRSDT |
| | NKKNLEKTQEAARKEIIAWFEGKRGNSAFK |
| | NNQKSFYGKLFKKELFSEILRSDDLEYDEE |
| | TQDAIACFDKFTTYFVGFHENRKNMYSTEA |
| | KSTSVAYRVVNENFSKFLSNCEAFSVLEAV |
| | CPNVLVEAEQELHLHKAFSDLKLSDVFKVE |
| | AYNKYLSQTGIDYYNQIIGGISSAEGVRKI |
| | RGVNEVVNNAIQQNDELKVALRNKQFTMVQ |
| | LFKQILSDRSTLSFVSEQFTSDQEVITVVK |
| | QFNDDIVNNKVLAVVKTLFENFNSYDLEKI |
| | YINSKELASVSNALLKDWSKIRNAVLENKI |
| | IELGANPPKTKISAVEKEVKNKDFSIAELA |
| | SYNDKYLDKEGNDKEICSIANVVLEAVGAL |
| | EIMLAESLPADLKTLENKNKVKGILDAYEN |
| | LLHLLNYFKVSAVNDVDLAFYGAFEKVYVD |
| | ISGVMPLYNKVRNYATKKPYSVEKFKLNFA |
| | MPTLADGWDKNKERDNGSIILLKDGQYYLG |
| | VMNPQNKPVIDNAVCNDAKGYQKMVYKMFP |
| | EISKMVTKCSTQLNAVKAHFEDNTNDFVLD |
| | DTDKFISDLTITKEIYDLNNVLYDGKKKFQ |
| | IDYLRNTGDFAGYHKALETWIDFVKEFLSK |
| | YRSTAIYDLTTLLPTNYYEKLDVFYSDVNN |
| | LCYKIDYENISVEQVNEWVEEGNLYLFKIY |
| | NKDFATGSTGKPNLHTMYWNAVFAEENLHD |
| | VVVKLNGGAELFYRPKSNMPKVEHRVGEKL |
| | VNRKNVNGEPIADSVHKEIYAYANGKISKS |
| | ELSENAQEELPLAIIKDVKHNITKDKRYLS |
| | DKYFFHVPITLNYKANGNPSAFNTKVQAFL |
| | KNNPDVNIIGIDRGERNLLYVVVIDQQGNI |
| | IDKKQVSYNKVNGYDYYEKLNQREKERIEA |
| | RQSWGAVGKIKELKEGYLSLVVREIADMMV |
| | KYNAIVVMENLNAGFKRVRGGIAEKAVYQK |
| | FEKMLIDKLNYLVFKDVEAKEAGGVLNAYQ |
| | LTDKFDSFEKMGNQSGFLFYVPAAYTSKID |
| | PVTGFANVFSTKHITNTEAKKEFICSFNSL |
| | RYDEAKDKFVLECDLNKFKIVANSHIKNWK |
| | FIIGGKRIVYNSKNKTYMEKYPCEDLKATL |
| | NASGIDFSSSEIINLLKNVPANREYGKLFD |
| | ETYWAIMNTLQMRNSNALTGEDYIISAVAD |
| | DNEKVFDSRTCGAELPKDADANGAYHIALK |
| | GLYLLQRIDISEEGEKVDLSIKNEEWFKFV |
| | QQKEYAR* |
| SEQ ID: | MCMKITKIDGISHKKYKEKGKLIKNNDTAK |
| 5 | DIIEERFNDIEKKTKELFQKTLDFYVKNYE |
| | KCKEQNKERREKAKNYFSKVKILVDNKKIT |
| | ICNENTEKMEIEDFNEYDVRSGKYFNVLNK |
| | ILNGENYTEEDLEVFENDLQKRTGRIKSIK |
| | NSLEENKAHFKKESINNNIIYDRVKGNNKK |
| | SLFYEYYRISSKHQEYVNNIFEAFDKLYSN |
| | SHEAMNNLFSEITKDSKDRNIRKIREAYHE |
| | ILNKNKTEFGEELYKKIQDNRNNFDKLLEI |
| | EPEIKELTKSQIFYKYYIDKVNLDETSIKH |
| | CFCHLVEIEVNQLLKNYVYSKRNINKEKLE |
| | NIFEYCKLKNLIKNKLVNKLNNYIRNCGKY |
| | NAYISNNDVVVNSEKISEIRTKEAFLRSII |
| | GVSSSAYFSLRNILNTDNTQDITNKVDKEV |
| | DKLYQENKKIELEERLKLFFGNYFDINNQQ |
| | EIKVFLMNIDKIISSIRHEIIHFKMETNAQ |
| | NIFDENNVNLGNTAKNIFSNEINEEKIKFK |
| | IFKQLNSANVFDYLSNKDITEYMDKVVFSF |
| | TNRNVSFVPSFTKIYNRVQDLANSLEIKKW |
| | KIPDKSEGKDAQIYLLKNIYYGKFLDEFLN |
| | EENGIFISIKDKIIELNRNQNKRTGFYKLE |
| | KFEKIEETNPKKYLEIIQSLYMINIEEIDS |
| | EGKNIFLDFIQKIFLKGFFEFIKNNYNYLL |
| | ELKKIQDKKNIFDSEMSEYIAGEKTLEDIG |
| | EINEIIQDIKITEIDKILNQTDKINCFYLL |
| | LKLLNYKEITELKGNLEKYQILSKTNVYEK |
| | ELMLLNIVNLDNNKVKIENFKILAEEIGKF |
| | IEKINIEEINKNKKIKTFEELRNFEKGENT |
| | GEYYNIYSDDKNIKNIRNLYNIKKYGMLDL |
| | LEKISEKTNYCIKKKDLEEYSELRKQLEDE |
| | KTNFYKIQEYLHSKYQQKPKKILLKNNKND |
| | YEKYKKSIENIEKYVHLKNKIEFNELNLLQ |
| | SLLLKILHRLVGFTSIWERDLRFRLIGEFP |
| | DELDVEDIFDHRKRYKGTGKGICKKYDRFI |
| | NTHTEYKNNNKMENVKFADNNPVRNYIAHF |
| | NYLPNPKYSILKMMEKLRKLLDYDRKLKNA |
| | VMKSIKDILEEYGFKAEFIINSDKEIILNL |
| | VKSVEIIHLGKEDLKSRRNSEDLCKLVKAM |
| | LEYSK* |
| SEQ ID: | MEDKQFLERYKEFIGLNSLSKTLRNSLIPV |
| 6 | GSTLKHIQEYGILEEDSLRAQKREELKGIM |
| | DDYYRNYIEMHLRDVHDIDWNELFEALTEV |
| | KKNQTDDAKKRLEKIQEKKRKEIYQYLSDD |
| | AVFSEMFKEKMISGILPDFIRCNEGYSEEE |
| | KEEKLKTVALFHRFTSSFNDFFLNRKNVFT |
| | KEAIVTAIGYRVVHENAEIFLENMVAFQNI |
| | QKSAESQISIIERKNEHYFMEWKLSHIFTA |
| | DYYMMLMTQKAIEHYNEMCGVVNQQMREYC |
| | QKEKKNWNLYRMKRLHKQILSNASTSFKIP |
| | EKYENDAEVYESVNSFLQNVMEKTVMERIA |
| | VLKNSTDNFDLSKIYITAPYYEKISNYLCG |
| | SWNTITDCLTHYYEQQIAGKGARKDQKVKA |
| | AVKADKWKSLSEIEQLLKEYARAEEVKRKP |
| | EEYIAEIENIVSLKEAHLLEYHPEVNLIEN |
| | EKYATEIKDVLDNYMELFHWMKWFYIEEAV |
| | EKEVNFYGELDDLYEEIKDIVPLYNKVRNY |
| | VTQKPYSDTKIKLNFGTPTLANGWSKSKEY |
| | DYNAILLQKDGKYYMGIFNPIQKPEKEIIE |
| | GHSQPLEGNEYKKMVYYYLPSANKMLPKVL |
| | LSKKGMEIYQPSEYIINGYKERRHIKSEEK |
| | FDLQFCHDLIDYFKSGIERNSDWKVFGFDF |
| | SDTDTYQDISGFYREVEDQGYKIDWTYIKE |
| | ADIDRLNEEGKLYLFQIYNKDFSEKSTGRE |
| | NLHTMYLKNLFSEENVREQVLKLNGEAEIF |
| | FRKSSVKKPIIFKKGTMLVNRTYMEEVNGN |
| | SVRRNIPEKEYQEIYNYKNHRLKGELSTEA |
| | KKYLEKAVCHETKKDIVKDYRYSVDKFFIH |
| | LPITINYRASGKETLNSVAQRY+AHQNDMH |
| | VIGIDRGERNLIYVSVINMQGEIKEQKSFN |
| | IINEFNYKEKLKEREQSRGAARRNWKEIGQ |
| | IKDLKEGYLSGVIHEIAKMMIKYHAIIAME |
| | DLNYGFKRGRFKVERQVYQKFENMLIQKLN |
| | YLVFKDRPADEDGGVLRGYQLAYIPDSVKK |
| | MGRQCGMIFYVPAAFTSKIDPTTGFVDIFK |
| | HKVYTTEQAKREFILSFDEICYDVERQLFR |
| | FTFDYANFVTQNVTLARNNWTIYTNGTRAQ |
| | KEFGNGRMRDKEDYNPKDKMVELLESEGIE |
| | FKSGKNLLPALKKVSNAKVFEELQKIVRFT |
| | VQLRNSKSEENDVDYDHVISPVLNEEGNFF |
| | DSSKYKNKEEKKESLLPVDADANGAYCIAL |
| | KGLYIMQAIQKNWSEEKALSPDVLRLNNND |
| | WFDY+QNKRYR* |
| SEQ ID: | MEEKKMSKIEKFIGKYKISKTLRFRAVPVG |
| 7 | KTQDNIEKKGILEKDKKRSEDYEKVKAYLD |
| | SLHRDFIENTLKKVKLNELNEYACLFFSGT |
| | KDDGDKKKMEKLEEKMRKTISNEFCNDEMY |
| | KKIFSEKILSENNEEDVSDIVSSYKGFFTS |
| | LNGYVNNRKNLYVSDAKPTSIAYRCINENL |
| | PKFLRNVECYKKVVQVIPKEQIEYMSNNLN |
| | LSPYRIEDCFNIDFFEFCLSQGGIDLYNTF |
| | IGGYSKKDGTKVQGINEIVNLYNQKNKKDK |
| | EKYKLPQFTPLFKQILSDRDTKSFSIEKLE |
| | NIYEVVELVKKSYSDEMFDDIETVFSNLNY |
| | YDASGIYVKNGPAITHISMNLTKDWATIRN |
| | NWNYEYDEKHSTKKNKNIEKYEDTRNTMYK |
| | KIDSFTLEYISRLVGKDIDELVKYFENEVA |
| | NFVMDIKKTYSKLTPLFDRCQKENFDISED |
| | EVNDIKGYLDNVKLLESFMKSFTINGKENN |
| | IDYVFYGKFTDDYDKLHEFDHIYNKVRNYI |
| | TTSRKPYKLDKYKLYFDNPQLLGGWDINKE |
| | KDYRTVMLTKDGKYYFAIIDKGEHPFDNIP |
| | KDYFDNNGYYKKIIYRQIPNAAKYLSSKQI |
| | VPQNPPEEVKRILDKKKADSKSLTEEEKNI |
| | FIDYIKSDFLKNYKLLFDKNNNPYFNFAFR |
| | ESSTYESLNEFFEDVERQAYSVRYENLPAD |
| | YIDNLVNEGKIYLFEIYSKDFSEYSKGTNN |
| | LHTMYFKALFDNDNLKNTVFKLSGNAELFI |
| | RPASIKKDELVIHPKNQLLQNKNPLNPKKQ |
| | SIFDYDLVKDKRFFENQYMLHISIEINKNE |
| | RDAKKIKNINEMVRKELKDSDDNYIIGIDR |
| | GERNLLYVCVINSAGKIVEQMSLNEIINEY |
| | NGIKHTVDYQGLLDKCEKERNAQRQSWKSI |
| | ENIKELKDGYISQVVHKLCQLVEKYDAIIA |
| | MENLNGGFKRGRTKFEKQVYQKFENKLINK |
| | MEYMADKKRKTTENGGILRGYQLTNGCINN |
| | SYQNGFIFYVPAWLTSKIDPTTGFVDLLKP |
| | KYTNVEEAHLWINKFNSITYDKKLDMFAFN |
| | INYSQFPRADIDYRKIWTFYTNGYRIETFR |
| | NSEKNNEFDWKEVHLTSVIKKLLEEYQINY |
| | ISGKNIIDDLIQIKDKPFWNSFIKYIRLTL |
| | QMRNSITGRTDVDYIISPVINNEGTFYDSR |
| | KDLDEITLPQDADANGAYNIARKALWIIEK |
| | LKESPDEELNKVKLAITQREWLEYAQINI |
| SEQ ID: | MEKIKKPSNRNSIPSIIISDYDANKIKEIK |
| 8 | VKYLKLARLDKITIQDMEIVDNIVEFKKIL |
| | LNGVEHTIIDNQKIEFDNYEITGCIKPSNK |
| | RRDGRISQAKYVVTITDKYLRENEKEKRFK |
| | STERELPNNTLLSRYKQISGFDTLTSKDIY |
| | KIKRYIDFKNEMLFYFQFIEEFFNPLLPKG |
| | KNFYDLNIEQNKDKVAKFIVYRLNDDFKNK |
| | SLNSYITDTCMIINDFKKIQKILSDFRHAL |
| | AHFDFDFIQKFFDDQLDKNKFDINTISLIE |
| | TLLDQKEEKNYQEKNNYIDDNDILTIFDEK |
| | GSKFSKLHNFYTKISQKKPAFNKLINSFLS |
| | QDGVPNEEFKSYLVTKKLDFFEDIHSNKEY |
| | KKIYIQHKNLVIKKQKEESQEKPDGQKLKN |
| | YNDELQKLKDEMNTITKQNSLNRLEVKLRL |
| | AFGFIANEYNYNFKNFNDEFTNDVKNEQKI |
| | KAFKNSSNEKLKEYFESTFIEKRFFHFSVN |
| | FFNKKTKKEETKQKNIFNSIENETLEELVK |
| | ESPLLQIITLLYLFIPRELQGEFVGFILKI |
| | YHHTKNITSDTKEDEISIEDAQNSFSLKFK |
| | ILAKNLRGLQLFHYSLSHNTLYNNKQCFFY |
| | EKGNRWQSVYKSFQISHNQDEFDIHLVIPV |
| | IKYYINLNKLMGDFEIYALLKYADKNSITV |
| | KLSDITSRDDLKYNGHYNFATLLFKTFGID |
| | TNYKQNKVSIQNIKKTRNNLAHQNIENMLK |
| | AFENSEIFAQREEIVNYLQTEHRMQEVLHY |
| | NPINDFTMKTVQYLKSLSVHSQKEGKIADI |
| | HKKESLVPNDYYLIYKLKAIELLKQKVIEV |
| | IGESEDEKKIKNAIAKEEQIKKGNN |
| SEQ ID: | MENYGGFTGLYPLQKTLKFELRPQGRTMEH |
| 9 | LVSSNFFEEDRDRAEKYKIVKKVIDNYHKD |
| | FINECLSKRSFDWTPLMKTSEKYYASKEKN |
| | GKKKQDLDQKIIPTIENLSEKDRKELELEQ |
| | KRMRKEIVSVFKEDKRFKYLFSEKLFSILL |
| | KDEDYSKEKLTEKEILALKSFNKFSGYFIG |
| | LHKNRANFYSEGDESTAIAYRIVNENFPKF |
| | LSNLKKYREVCEKYPEIIQDAEQSLAGLNI |
| | KMDDIFPMENFNKVMTQDGIDLYNLAIGGK |
| | AQALGEKQKGLNEFLNEVNQSYKKGNDRIR |
| | MTPLFKQILSERTSYSYILDAFDDNSQLIT |
| | SINGFFTEVEKDKEGNTFDRAVGLIASYMK |
| | YDLSRVYIRKADLNKVSMEIFGSWERLGGL |
| | LRIFKSELYGDVNAEKTSKKVDKWLNSGEF |
| | SLSDVINAIAGSKSAETFDEYILKMRVARG |
| | EIDNALEKIKCINGNFSEDENSKMIIKAIL |
| | DSVQRLFHLFSSFQVRADFSQDGDFYAEYN |
| | EIYEKLFAIVPLYNRVRNYLTKNNLSMKKI |
| | KLNFKNPALANGWDLNKEYDNTAVIFLREG |
| | KYYLGIMNPSKKKNIKFEEGSGTGPFYKKM |
| | AYKLLPDPNKMLPKVFFAKKNINYYNPSDE |
| | IVKGYKAGKYKKGENFDIDFCHKLIDFFKE |
| | SIQKNEDWRAFNYLFSATESYKDISDFYSE |
| | VEDQGYRMYFLNVPVANIDEYVEKGDLFLF |
| | QIYNKDFASGAKGNKDMHTIYWNAAFSDEN |
| | LRNVVVKLNGEAELFYRDKSIIEPICHKKG |
| | EMLVNRTCFDKTPVPDKIHKELFDYHNGRA |
| | KTLSIEAKGYLDRVGVFQASYEIIKDRRYS |
| | ENKMYFHVPLKLNFKADGKKNLNKMVIEKF |
| | LSDKDVHIIGIDRGERNLLYYSVIDRRGNI |
| | IDQDSLNIIDGFDYQKKLGQREIERREARQ |
| | SWNSIGKIKDLKEGYLSKAVHKVSKMVLEY |
| | NAIVVLEDLNFGFKRGRFKVEKQVYQKFEK |
| | MLIDKLNYLVFKEVLDSRDAGGVLNAYQLT |
| | TQLESFNKLGKQSGILFYVPAAYTSKIDPT |
| | TGFVSLFNTSRIESDSEKKDFLSGFDSIVY |
| | SAKDGGIFAFKFDYRNRNFQREKTDHKNIW |
| | TVYTNGDRIKYKGRMKGYEITSPTKRIKDV |
| | LSSSGIRYDDGQELRDSIIQSGNKVLINEV |
| | YNSFIDTLQMRNSDGEQDYIISPVKNRNGE |
| | FFRTDPDRRELPVDADANGAYHIALRGELL |
| | MQKIAEDFDPKSDKFTMPKMEHKDWFEFMQ |
| | TRGD* |
| SEQ ID: | MEVQKTVMKTLSLRILRPLYSQEIEKEIKE |
| 10 | EKERRKQAGGTGELDGGFYKKLEKKHSEMF |
| | SFDRLNLLLNQLQREIAKVYNHAISELYIA |
| | TIAQGNKSNKHYISSIVYNRAYGYFYNAYI |
| | ALGICSKVEANFRSNELLTQQSALPTAKSD |
| | NFPIVLHKQKGAEGEDGGFRISTEGSDLIF |
| | EIPIPFYEYNGENRKEPYKWVKKGGQKPVL |
| | KLILSTFRRQRNKGWAKDEGTDAEIRKVTE |
| | GKYQVSQIEINRGKKLGEHQKWFANFSIEQ |
| | PIYERKPNRSIVGGLDVGIRSPLVCAINNS |
| | FSRYSVDSNDVFKFSKQVFAFRRRLLSKNS |
| | LKRKGHGAAHKLEPITEMTEKNDKFRKKII |
| | ERWAKEVTNFFVKNQVGIVQIEDLSTMKDR |
| | EDHFFNQYLRGFWPYYQMQTLIENKLKEYG |
| | IEVKRVQAKYTSQLCSNPNCRYWNNYFNFE |
| | YRKVNKFPKFKCEKCNLEISADYNAARNLS |
| | TPDIEKFVAKATKGINLPEK* |
| SEQ ID: | MIIHNCYIGGSFMKKIDSFTNCYSLSKTLR |
| 11 | FKLIPIGATQSNFDLNKMLDEDKKRAENYS |
| | KAKSIIDKYHRFFIDKVLSSVTENKAFDSF |
| | LEDVRAYAELYYRSNKDDSDKASMKTLESK |
| | MRKFIALALQSDEGFKDLFGQNLIKKTLPE |
| | FLESDTDKEIIAEFDGFSTYFTGFFNNRKN |
| | MYSADDQPTAISYRCINDNLPKFLDNVRTF |
| | KNSDVASILNDNLKILNEDFDGIYGTSAED |
| | VFNVDYFPFVLSQKGIEAYNSILGGYTNSD |
| | GSKIKGLNEYINLYNQKNENIHRIPKMKQL |
| | FKQILSERESVSFIPEKFDSDDDVLSSIND |
| | YYLERDGGKVLSIEKTVEKIEKLFSAVTDY |
| | STDGIFVKNAAELTAVCSGAFGYWGTVQNA |
| | WNNEYDALNGYKETEKYIDKRKKAYKSIES |
| | FSLADIQKYADVSESSETNAEVTEWLRNEI |
| | KEKCNLAVQGYESSKDLISKPYTESKKLFN |
| | NDNAVELIKNALDSVKELENVLRLLLGTGK |
| | EESKDENFYGEFLPCYERICEVDSLYDKVR |
| | NYMTQKLYKTDKIKLNFQNPQFLGGWDRNK |
| | EADYSAVLLRRNSLYYIAIMPSGYKRVFEK |
| | IPAPKADETVYEKVIYKLLPGPNKMLPKVF |
| | FSKKGIETFNPPKEILEKYELGTHKTGDGF |
| | NLDDCHALIDYFKSALDVHSDWSNFGFRFS |
| | DTSTYKNIADFYNEVKNQGYKITFCDVPQS |
| | YINELVDEGKLYLFQLYNKDFSEHSKGTPN |
| | LHTLYFKMLFDERNLENVVFKLNGEAEMFY |
| | REASISKDDMIVHPKNQPIKNKNEQNSRKQ |
| | STFEYDIVKDRRYTVDQFMLHIPITLNFTA |
| | NGGTNINNEVRKALKDCDKNYVIGIDRGER |
| | NLLYICVVDSEGRIIEQYSLNEIINEYNGN |
| | TYSTDYHALLDKKEKERLESRKAWKTVENI |
| | KELKEGYISQVVHKICELVEKYDAVIVMED |
| | LNLGFKQGRSGKFEKSVYQKFEKMLIDKLN |
| | YFADKKKSPEEIGSVLNAYQLTNAFESFEK |
| | MGKQNGFIFYVPAYLTSKIDPTTGFADLLH |
| | PSSKQSKESMRDFVGRFDSITFNKTENYFE |
| | FELDYNKFPRCNTDYRKKWTVCTYGSRIKT |
| | FRNPEKNSEWDNKTVELTPAFMALFEKYSI |
| | DVNGDIKAQIMSVDKKDFFVELIGLLRLTL |
| | QMRNSETGKVDRDYLISPVKNSEGVFYNSD |
| | DYKGIENASLPKDADANGAYNIARKGLWII |
| | EQIKACENDAELNKIRLAISNAEWLEYAQK |
| | K* |
| SEQ ID: | MKDYIRKTLSLRILRPYYGEEIEKEIAAAK |
| 12 | KKSQAEGGDGALDNKFWDRLKAEHPEIISS |
| | REFYDLLDAIQRETTLYYNRAISKLYHSLI |
| | VEREQVSTAKALSAGPYHEFREKFNAYISL |
| | GLREKIQSNFRRKELARYQVALPTAKSDTF |
| | PIPIYKGFDKNGKGGFKVREIENGDFVIDL |
| | PLMAYHRVGGKAGREYIELDRPPAVLNVPV |
| | ILSTSRRRANKTWFRDEGTDAEIRRVMAGE |
| | YKVSWVEILQRKRFGKPYGGWYVNFTIKYQ |
| | PRDYGLDPKVKGGIDIGLSSPLVCAVTNSL |
| | ARLTIRDNDLVAFNRKAMARRRTLLRQNRY |
| | KRSGHGSANKLKPIEALTEKNELYRKAIMR |
| | RWAREAADFFRQHRAATVNMEDLTGIKDRE |
| | DYFSQMLRCYWNYSQLQTMLENKLKEYGIA |
| | VKYIEPKDTSKTCHSCGHVNEYFDFNYRSA |
| | HKFPMFKCEKCGVECGADYNAARNIAQA |
| SEQ ID: | MKEQFINRYPLSKTLRFSLIPVGETENNFN |
| 13 | KNLLLKKDKQRAENYEKVKCYIDRFHKEYI |
| | ESVLSKARIEKVNEYANLYWKSNKDDSDIK |
| | AMESLENDMRKQISKQLTSTEIYKKRLFGK |
| | ELICEDLPSFLTDKDERETVECFRSFTTYF |
| | KGFNTNRENMYSSDGKSTAIAYRCINDNLP |
| | RFLDNVKSFQKVFDNLSDETITKLNTDLYN |
| | IFGRNIEDIFSVDYFEFVLTQSGIEIYNSM |
| | IGGYTCSDKTKIQGLNECINLYNQQVAKNE |
| | KSKKLPLMKPLYKQILSEKDSVSFIPEKFN |
| | SDNEVLHAIDDYYTGHIGDFDLLTELLQSL |
| | NTYNANGIFVKSGVAITDISNGAFNSWNVL |
| | RSAWNEKYEALHPVTSKTKIDKYIEKQDKI |
| | YKAIKSFSLFELQSLGNENGNEITDWYISS |
| | INESNSKIKEAYLQAQKLLNSDYEKSYNKR |
| | LYKNEKATELVKNLLDAIKEFQKLIKPLNG |
| | TGKEENKDELFYGKFTSYYDSIADIDRLYD |
| | KVRNYITQKPYSKDKIKLNFDNPQLLGGWD |
| | KNKESDYRTVLLHKDGLYYLAVMDKSHSKA |
| | FVDAPEITSDDKDYYEKMEYKLLPGPNKML |
| | PKVFFASKNIDTFQPSDRILDIRKRESFKK |
| | GATFNKAECHEFIDYFKDSIKKHDDWSQFG |
| | FKFSPTESYNDISEFYREISDQGYSVRFNK |
| | ISKNYIDGLVNNGYIYLFQIYNKDFSKYSK |
| | GTPNLHTLYFKMLFDERNLSNVVYKLNGEA |
| | EMFYREASIGDKEKITHYANQPIKNKNPDN |
| | EKKESVFEYDIVKDKRFTKRQFSLHLPITI |
| | NFKAHGQEFLNYDVRKAVKYKDDNYVIGID |
| | RGERNLIYISVINSNGEIVEQMSLNEIISD |
| | NGHKVDYQKLLDTKEKERDKARKNWTSVEN |
| | IKELKEGYISQVVHKICELVIKYDAVIAME |
| | DLNFGFKRGRFPVEKQVYQKFENMLISKLN |
| | LLIDKKAEPTEDGGLLRAYQLTNKFDGVNK |
| | AKQNGIIFYVPAWDTSKIDPATGFVNLLKP |
| | KCNTSVPEAKKLFETIDDIKYNANTDMFEF |
| | YIDYSKFPRCNSDFKKSWTVCTNSSRILTF |
| | RNKEKNNKWDNKQIVLTDEFKSLFNEFGID |
| | YKGNLKDSILSISNADFYRRLIKLLSLTLQ |
| | MRNSITGSTLPEDDYLISPVANKSGEFYDS |
| | RNYKGTNAALPCDADANGAYNIARKALWAI |
| | NVLKDTPDDMLNKAKLSITNAEWLEYTQK* |
| SEQ ID: | MKEQFVNQYPISKTLRFSLIPIGKTEENFN |
| 14 | KNLLLKEDEKKAEEYQKVKGYIDRYHKFFI |
| | ETALCNINFEGFEEYSLLYYKCSKDDNDLK |
| | TMEDIEIKLRKQISKTMTSHKLYKDLFGEN |
| | MIKTILPNFLDSDEEKNSLEMFRGFYTYFS |
| | GFNTNRKNMYTEEAKSTSIAYRCINDNLPK |
| | FLDNSKSFEKIKCALNKEELKAKNEEFYEI |
| | FQIYATDIFNIDFFNFVLTQPGIDKYNGII |
| | GGYTCSDGTKVQGLNEIINLYNQQIAKDDK |
| | SKRLPLLKMLYKQILSDRETVSFIPEKFSS |
| | DNEVLESINNYFSKNVSNAIKSLKELFQGF |
| | EAYNMNGIFISSGVAITDLSNAVFGDWNAI |
| | STAWEKAYFETNPPKKNKSQEKYEEELKAN |
| | YKKIKSFSLDEIQRLGSIAKSPDSIGSVAE |
| | YYKITVTEKIDNITELYDGSKELLNCNYSE |
| | SYDKKLIKNDTVIEKVKTLLDAVKSLEKLI |
| | KPLVGTGKEDKDELFYGTFLPLYTSLSAVD |
| | RLYDKVRNYATQKPYSKDKIKLNFNCSSFL |
| | SGWATDYSSNGGLIFEKDGLYYLGIVNKKF |
| | TTEEIDYLQQNADENPAQRIVYDFQKPDNK |
| | NTPRLFIRSKGTNYSPSVKEYNLPVEEIVE |
| | LYDKRYFTTEYRNKNPELYKASLVKLIDYF |
| | KLGFTRHESYRHYDFKWKKSEEYNDISEFY |
| | KDVEISCYSLKQEKINYNTLLNFVAENRIY |
| | LFQIYNKDFSKYSKGTPNLHTRYFKALFDE |
| | NNLSDVVFKLNGGSEMFFRKASIKDNEKVV |
| | HPANQPIDNKNPDNSKKQSTFDYELIKDKR |
| | FTKHQFSIHIPITMNFKARGRDFINNDIRK |
| | AIKSEYKPYVIGIDRGERNLIYISVINNNG |
| | EIVEQMSLNDIISDNGYKVDYQRLLDRKEK |
| | ERDNARKSWGTIENIKELKEGYISQVIHKI |
| | CELVIKYDAVIAMEDLNFGFKRGRFNVEKQ |
| | VYQKFENMLISKLNYLCDKKSEANSEGGLL |
| | KAYQLTNKFDGVNKGKQNGIIFYVPAWLTS |
| | KIDPVTGFVDLLHPKYISVEETHSLFEKLD |
| | DIRYNFEKDMFEFDIDYSKLPKCNADFKQK |
| | WTVCTNADRIMTFRNSEKNNEWDNKRILLS |
| | DEFKRLFEEFGIDYCHNLKNKILSISNKDF |
| | CYRFIKLFALTMQMRNSITGSTNPEDDYLI |
| | SPVRDENGVFYDSRNFIGSKAGLPIDADAN |
| | GAYNIARKGLWAINAIKSTADDMLDKVDLS |
| | ISNAKWLEYVQK* |
| SEQ ID: | MKITKIDGILHKKYIKEGKLVKSTSEENKT |
| 15 | DERLSELLTIRLDTYIKNPDNASEEENRIR |
| | RETLKEFFSNKVLYLKDSILYLKDRREKNQ |
| | LQNKNYSEEDISEYDLKNKNSFLVLKKILL |
| | NEDINSEELEIFRNDFEKKLDKINSLKYSL |
| | EENKANYQKINENNIKKVEGKSKRNIFYNY |
| | YKDSAKRNDYINNIQEAFDKLYKKEDIENL |
| | FFLIENSKKHEKYKIRECYHKIIGRKNDKE |
| | NFATIIYEEIQNVNNMKELIEKVPNVSELK |
| | KSQVFYKYYLNKEKLNDENIKYVFCHFVEI |
| | EMSKLLKNYVYKKPSNISNDKVKRIFEYQS |
| | LKKLIENKLLNKLDTYVRNCGKYSFYLQDG |
| | EIATSDFIVGNRQNEAFLRNIIGVSSTAYF |
| | SLRNILETENENDITGRIKGKTVKNKKGEE |
| | KYISGEIDKLYDNNKQNEVKKNLKMFYSYD |
| | FNMNRKKEIEDFFSNIDEAISSIRHGIVHF |
| | NLELEGKDIFTFKNIVPSQISKKMFQNEIN |
| | EKKLKLKIFRQLNSANVFRYLEKYKILNYL |
| | NRTRFEFVNKNIPFVPSFTKLYSRIDDLKN |
| | SLCIYWKIPKANDNNKTKEITDAQIYLLKN |
| | IYYGEFLNYFMSNNGNFFEIIKEIIELNKN |
| | DKRNLKTGFYKLQKFENLQEKTPKEYLANI |
| | QSFYMIDAGNKDEEEKDAYIDFIQKIFLKG |
| | FMTYLANNGRLSLMYIGNDEQINTSLAGKK |
| | QEFDKFLKKYEQNNNIEIPHEINEFVREIK |
| | LGKILKYTESLNMFYLILKLLNHKELTNLK |
| | GSLEKYQSANKEEAFSDQLELINLLNLDNN |
| | RVTEDFELEADEIGKFLDFNGNKVKDNKEL |
| | KKFDTNKIYFDGENIIKHRAFYNIKKYGIL |
| | NLLEKISDEAKYKISIEELKNYSNKKIEIE |
| | KNHTTQENLHRKYARPRKDEKFNDEDYKKY |
| | EKTIRNIQQYTHLKNKVEFNELNLLQSLLL |
| | RILHRLVGYTSIWERDLRFRLKGEFPENQY |
| | IEEIFNFDNSKNVKYKNGQIVEKYISFYKE |
| | LYKDDMEKISIYSDKKVKELKKEKKDLYIR |
| | NYIAHFNYIPNAEVSLLEVLENLRKLLSYD |
| | RKLKNAIMKSIVDILKEYGFVVTFKIEKDK |
| | KIRIESLKSEEVVHLKKLKLKDNDKKKEPI |
| | KTYRNSKELCKLVKVMFEYKMKEKKSEN* |
| SEQ ID: | MKITKIDGISHKKYIKEGKLVKSTSEENKT |
| 16 | DERLSELLTIRLDTYIKNPDNASEEENRIR |
| | RENLKEFFSNKVLYLKDGILYLKDRREKNQ |
| | LQNKNYSEEDISEYDLKNKNSFLVLKKILL |
| | NEDINSEELEIFRKDVEAKLNKINSLKYSF |
| | EENKANYQKINENNVEKVGGKSKRNIIYDY |
| | YRESAKRNDYINNVQEAFDKLYKKEDIEKL |
| | FFLIENSKKHEKYKIRECYHKIIGRKNDKE |
| | NFAKIIYEEIQNVNNIKELIEKVPDMSELK |
| | KSQVFYKYYLDKEELNDKNIKYAFCHFVEI |
| | EMSQLLKNYVYKRLSNISNDKIKRIFEYQN |
| | LKKLIENKLLNKLDTYVRNCGKYNYYLQDG |
| | EIATSDFIAGNRQNEAFLRNIIGVSSVAYF |
| | SLRNILETENKDDITGKMRGKTRIDSKTGE |
| | EKYIPGEVDQIYYENKQNEVKNKLKMFYGY |
| | DFDMDNKKEIEDFFANIDEAISSIRHGIVH |
| | FNLDLDGKDIFAFKNIVPSEISKKMFQNEI |
| | NEKKLKLKIFRQLNSANVFRYLEKYKILNY |
| | LKRTRFEFVNKNIPFVPSFTKLYSRIDDLK |
| | NSLGIYWKTPKTNDDNKTKEIIDAQIYLLK |
| | NIYYGEFLNYFMSNNGNFFEISREIIELNK |
| | NDKRNLKTGFYKLQKFEDIQEKTPKKYLAN |
| | IQSLYMINAGNQDEEEKDTYIDFIQKIFLK |
| | GFMTYLANNGRLSLMYIGNDEQINTSLAGK |
| | KQEFDKFLKKYEQNNNIEIPHEINEFLREI |
| | KLGKILKYTESLNMFYLILKLLNHKELTNL |
| | KGSLEKYQSANKEETFSDELELINLLNLDN |
| | NRVTEDFELEANEIGKFLDFNGNKIKDRKE |
| | LKKFDTKKIYFDGENIIKHRAFYNIKKYGM |
| | LNLLEKIADKAKYKISLKELKEYSNKKNEI |
| | EKNYTMQQNLHRKYARPKKDEKFNDEDYKE |
| | YEKAIGNIQKYTHLKNKVEFNELNLLQGLL |
| | LKILHRLVGYTSIWERDLRFRLKGEFPENQ |
| | YIEEIFNFDNSKNVKYKSGQIVEKYINFYK |
| | ELYKDNVEKRSIYSDKKVKKLKQEKKDLYI |
| | RNYIAHFNYIPHAEISLLEVLENLRKLLSY |
| | DRKLKNAIMKSVVDILKEYGFVATFKIGAD |
| | KKIGIQTLESEKIVHLKNLKKKKLMTDRNS |
| | EELCKLVKVMFEYKMEEKNLKTKKCKVI* |
| SEQ ID: | MKKIDNFVGCYPVSKTLRFKAIPIGKTQEN |
| 17 | IEKKRLVEEDEVRAKDYKAVKKLIDRYHRE |
| | FIEGVLDNVKLDGLEEYYMLFNKSDREESD |
| | NKKIEIMEERFRRVISKSFKNNEEYKKIFS |
| | KKIIEEILPNYIKDEEEKELVKGFKGFYTA |
| | FVGYAQNRENMYSDEKKSTAISYRIVNENM |
| | PRFITNIKVFEKAKSILDVDKINEINEYIL |
| | NNDYYVDDFFNIDFFNYVLNQKGIDIYNAI |
| | IGGIVTGDGRKIQGLNECINLYNQENKKIR |
| | LPQFKPLYKQILSESESMSFYIDEIESDDM |
| | LIDMLKESLQIDSTINNAIDDLKVLFNNIF |
| | DYDLSGIFINNGLPITTISNDVYGQWSTIS |
| | DGWNERYDVLSNAKDKESEKYFEKRRKEYK |
| | KVKSFSISDLQELGGKDLSICKKINEIISE |
| | MIDDYKSKIEEIQYLFDIKELEKPLVTDLN |
| | KIELIKNSLDGLKRIERYVIPFLGTGKEQN |
| | RDEVFYGYFIKCIDAIKEIDGVYNKTRNYL |
| | TKKPYSKDKFKLYFENPQLMGGWDRNKESD |
| | YRSTLLRKNGKYYVAIIDKSSSNCMMNIEE |
| | DENDNYEKINYKLLPGPNKMLPKVFFSKKN |
| | REYFAPSKEIERIYSTGTFKKDTNFVKKDC |
| | ENLITFYKDSLDRHEDWSKSFDFSFKESSA |
| | YRDISEFYRDVEKQGYRVSFDLLSSNAVNT |
| | LVEEGKLYLFQLYNKDFSEKSHGIPNLHTM |
| | YFRSLFDDNNKGNIRLNGGAEMFMRRASLN |
| | KQDVTVHKANQPIKNKNLLNPKKTTTLPYD |
| | VYKDKRFTEDQYEVHIPITMNKVPNNPYKI |
| | NHMVREQLVKDDNPYVIGIDRGERNLIYVV |
| | VVDGQGHIVEQLSLNEIINENNGISIRTDY |
| | HTLLDAKERERDESRKQWKQIENIKELKEG |
| | YISQVVHKICELVEKYDAVIAL |
| SEQ ID: | EDLNSGFKNSRVKVEKQVYQKFEKMLITKL |
| 18 | NYMVDKKKDYNKPGGVLNGYQLTTQFESFS |
| | KMGTQNGIMFYIPAWLTSKMDPTTGFVDLL |
| | KPKYKNKADAQKFFSQFDSIRYDNQEDAFV |
| | FKVNYTKFPRTDADYNKEWEIYTNGERIRV |
| | FRNPKKNNEYDYETVNVSERMKELFDSYDL |
| | LYDKGELKETICEMEESKFFEELIKLFRLT |
| | LQMRNSISGRTDVDYLISPVKNSNGYFYNS |
| | NDYKKEGAKYPKDADANGAYNIARKVLWAI |
| | EQFKMADEDKLDKTKISIKNQEWLEYAQTH |
| | CEMKKIDSFVNYYPLSKTLRFSLIPVGKTE |
| | DNFNAKLLLEEDEKRAIEYEKVKRYIDRYH |
| | KHFIETVLANFHLDDVNEYAELYYKAGKDD |
| | KDLKYMEKLEGKMRKSISAAFTKDKKYKEI |
| | FGQEIIKNILPEFLENEDEKESVKMFQGFF |
| | TYFTGFNDNRKNMYTHEAQTTAISYRCINE |
| | NLPKFLDNVQSFAKIKESISSDIMNKLDEV |
| | CMDLYGVYAQDMFCTDYFSFVLSQSGIDRY |
| | NNIIGGYVDDKGVKIQGINEYINLYNQQVD |
| | EKNKRLPLMKKLYKQILIEKESISFIPEKF |
| | ESDNIVINAISDYYHNNVENLFDDFNKLFN |
| | EFSEYDDNGIFVTSGLAVTDISNAVFGSWN |
| | IISDSWNEEYKDSHPMKKTTNAEKYYEDMK |
| | KEYKKNLSFTIAELQRLGEAGCNDECKGDI |
| | KEYYKTTVAEKIENIKNAYEISKDLLASDY |
| | EKSNDKKLCKNDSAISLLKNLLDSIKDLEK |
| | TIKPLLGTGKEENKDDVFYGKFTNLYEMIS |
| | EIDRLYDKVRNYVTQKPYSKDKIKLNFENP |
| | QHLGGWDKNKERDYRSVLLKKEDKYYLAIM |
| | DKSNNKAFIDFPDDGECYEKIEYKLLPGPN |
| | KMLPKVFFASSNIEYFAPSKKILEIRSRES |
| | FKKGDMFNLKDCHEFIDFFKESIKKHEDWS |
| | QFGFEFSPTEKYNDISEFYNEVKIQGYSLK |
| | YKNVSKKYIDELIECGQLYLFQIYNKDFSV |
| | YAKGNPNLHTMYFKMLFDERNLANVVYQLN |
| | GGAEMFYRKASIKDSEKIVHHANQPIKNKN |
| | ADNVKKESVFEYDIIKDKRFTKRQFSIHIP |
| | ITLNFKAKGQNFINNDVRMALKKADENYVI |
| | GIDRGERNLLYICVINSKGEIVEQKSLNEI |
| | IGDNGYRVDYHKLLDKKEAERDEARKSWGT |
| | IENIKELKEGYLSQIVHEISKLVIKYDAVI |
| | AIEDLNSGFKKGRFKVEKQVYQKFENMLCT |
| | KLNYLVDKNADANECGGLLKAYQLTNKEDG |
| | ANRGRQNGIIFSVPAWLTSKIDPVTGFADL |
| | LRPKYKSVSESVEFISKIDNIRYNSKEDYF |
| | EFDIDYSKFPNSTASYKKKWTVCTYGERII |
| | NVRNKEKNNMWDNKTIVLTDEFKKLFADFG |
| | VDVSKNIKESVLAIDSKDFYYRFINLLANT |
| | LQLRNSEVGNVDVDYLISPVKGVDGSFYDS |
| | RLVKEKTLPENADANGAYNIARKALWAIDV |
| | LKQTKDEELKNANLSIKNAEWLEYVQK* |
| SEQ ID: | MKNQNTLPSNPTDILKDKPFWAAFFNLARH |
| 19 | NVYLTVNHINKLLDLEKLYNKDKHKEIFEH |
| | EDIFNISDDVMNDVNSNGKKRKLDIKKIWA |
| | NLDTDLTRKYQLRELILKHFPFIQPAIIGA |
| | QTKERTTIDKDKRSTSTSNDSLKPTGEGDI |
| | NDPLSLSNVKSIFFRLLQMLEQLRNYYSHV |
| | KHSKSATMPNFDEGLLKSMYNIFIDSVNKV |
| | KEDYSSNSVIDPNTSFSHLISKDEQGEIKP |
| | CRYSFTSKDGSINASGLLFFVSLFLEKQDS |
| | IWMQKKIPGFKKTSENYMKMTNEVFCRNHI |
| | LLPKMRLETVYDKDWMLLDMLNEVVRCPLS |
| | LYKRLAPADQNKFKVPEKSSDNANRQEDDN |
| | PFSRILVRHQNRFPYFALRFFDLNEVFTTL |
| | RFQINLGCYHFAICKKQIGDKKEVHHLTRT |
| | LYGFSRLQNFTQNTRPEEWNTLVKTTEPSS |
| | GNDGKTVQGVPLPYISYTIPHYQIENEKIG |
| | IKIFDGDTAVDTDIWPSVSTEKQLNKPDKY |
| | TLTPGFKADVFLSVHELLPMMFYYQLLLCE |
| | GMLKTDAGNAVEKVLIDTRNAIFNLYDAFV |
| | QEKINTITDLENYLQDKPILIGHLPKQMID |
| | LLKGHQRDMLKAVEQKKAMLIKDTERRLER |
| | LNKQPEQKPNVAAKNTGTLLRNGQIADWLV |
| | KDMMRFQPVKRDKEGNPINCSKANSTEYQM |
| | LQRAFAFYTTDSYRLPRYFEQLHLINCDNS |
| | HLFLSRFEYDKQPNLIAFYAAYLEAKLEFL |
| | NELQPQNWASDNYFLLLRAPKNDRQKLAEG |
| | WKNGFNLPRGLFTEKIKTWFNEHKTIVDIS |
| | DCDIFKNRVGQVARLIPVFFDKKFKDHSQP |
| | FYTYNFNVGNVSKITEANYLSKEKRENLFK |
| | SYQNKFKNNIPAEKTKEYREYKNFSSWKKF |
| | ERELRLIKNQDILTWLMCKNLFDEKIKPKK |
| | DILEPRIAVSYIKLDSLQTNTSTAGSLNAL |
| | AKVVPMTLAIHIDSPKPKGKAGNNEKENKE |
| | FTVYIKEEGTKLLKWGNFKTLLADRRIKGL |
| | FSYIEHDDINLEKYPLTKYQVDSELDLYQK |
| | YRIDIFKQTLDLEAQLLDKYSDLNTDNFNQ |
| | MLSGWSEKEGIPRNIKQDVAFLIGVRNGFS |
| | HNQYPDSKRIAFSRIKKFNPKTSSLQESEG |
| | LNIAKQMYEEAQQVVNKIKNIESFD* |
| SEQ ID: | MKVTKIDGISHKKFEDEGKLVKFTGHFNIK |
| 20 | NEMKERLEKLKELKLSNYIKNPENVKNKDK |
| | NKEKETKSRRENLKKYFSEIILRKKEEKYL |
| | LKKTRKFKNITEEINYDDIKKRENQQKIFD |
| | VLKELLEQRINENDKEEILNFDSVKLKEAF |
| | EEDFIKKELKIKAIEESLEKNRADYRKDYV |
| | ELENEKYEDVKGQNKRSLVFEYYKNPENRE |
| | KFKENIKYAFENLYTEENIKNLYSEIKEIF |
| | EKVHLKSKVRYFYQNEIIGESEFSEKDEEG |
| | ISILYKQIINSVEKKEKFIEFLQKVKIKDL |
| | TRSQIFYKYFLENEELNDENIKYVFSYFVE |
| | IEVNKLLKENVYKTKKFNEGNKYRVKNIFN |
| | YDKLKNLVVYKLENKLNNYVRNCGKYNYHM |
| | ENGDIATSDINMKNRQTEAFLRSILGVSSF |
| | GYFSLRNILGVNDDDFYKIEKDERKNENFI |
| | LKKAKEDFTSKNIFEKVVDKSFEKKGIYQI |
| | KENLKMFYGNSFDKVDKDELKKFFVNMLEA |
| | ITSVRHRIVHYNINTNSENIFDFSNIEVSK |
| | LLKNIFEKEIDTRELKLKIFRQLNSAGVFD |
| | YWESWVIKKYLENVKFEFVNKNVPFVPSFK |
| | KLYDRIDNLKGWNALKLGNNINIPKRKEAK |
| | DSQIYLLKNIYYGEFVEKFVNDNKNFEKIV |
| | KEIIEINRGAGTNKKTGFYKLEKFETLKAN |
| | TPTKYLEKLQSLHKISYDKEKIEEDKDVYV |
| | DFVQKIFLKGFVNYLKKLDSLKSLNLLNLR |
| | KDETITDKKSVHDEKLKLWENSGSNLSKMP |
| | EEIYEYVKKIKISNINYNDRMSIFYLLLKL |
| | IDYRELTNLRGNLEKYESMNKNKIYSEELT |
| | IINLVNLDNNKVRTNFSLEAEDIGKFLKSS |
| | ITIKNIAQLNNFSKIFADGENVIKHRSFYN |
| | IKKYGILDLLEKIVAKADLKITKEEIKKYE |
| | NLQNELKRNDFYKIQEQIHRNYNQKPFSIK |
| | KIENKKDFEKYKKVIEKIQDYTQLKNKIEF |
| | NDLNLLQSLIFRILHRLAGYTSLWERDLQF |
| | KLKGEFPEDKYIDEIFNSDGNNNQKYKHGG |
| | IADKYANFLIEKKEEKSGEILNKKQRKKKI |
| | KEDLEIRNYIAHFNYLPNAEKSILEILEEL |
| | RELLKHDRKLKNAVMKSIKDIFREYGFIVE |
| | FTISHTKNGKKIKVCSVKSEKIKHLKNNEL |
| | ITTRNSEDLCELVKIMLEHKELQK* |
| SEQ ID: | MKVTKVGGISHKKYTSEGRLVKSESEENRT |
| 21 | DERLSALLNMRLDMYIKNPSSTETKENQKR |
| | IGKLKKFFSNKMVYLKDNTLSLKNGKKENI |
| | DREYSETDILESDVRDKKNFAVLKKIYLNE |
| | NVNSEELEVFRNDIKKKLNKINSLKYSFEK |
| | NKANYQKINENNIEKVEGKSKRNIIYDYYR |
| | ESAKRDAYVSNVKEAFDKLYKEEDIAKLVL |
| | EIENLTKLEKYKIREFYHEIIGRKNDKENF |
| | AKIIYEEIQNVNNMKELIEKVPDMSELKKS |
| | QVFYKYYLDKEELNDKNIKYAFCHFVEIEM |
| | SQLLKNYVYKRLSNISNDKIKRIFEYQNLK |
| | KLIENKLLNKLDTYVRNCGKYNYYLQDGEI |
| | ATSDFIARNRQNEAFLRNIIGVSSVAYFSL |
| | RNILETENENDITGRMRGKTVKNNKGEEKY |
| | VSGEVDKIYNENKKNEVKENLKMFYSYDFN |
| | MDNKNEIEDFFANIDEAISSIRHGIVHFNL |
| | ELEGKDIFAFKNIAPSEISKKMFQNEINEK |
| | KLKLKIFRQLNSANVFRYLEKYKILNYLKR |
| | TRFEFVNKNIPFVPSFTKLYSRIDDLKNSL |
| | GIYWKTPKTNDDNKTKEIIDAQIYLLKNIY |
| | YGEFLNYFMSNNGNFFEISKEIIELNKNDK |
| | RNLKTGFYKLQKFEDIQEKIPKEYLANIQS |
| | LYMINAGNQDEEEKDTYIDFIQKIFLKGFM |
| | TYLANNGRLSLIYIGSDEETNTSLAEKKQE |
| | FDKFLKKYEQNNNIKIPYEINEFLREIKLG |
| | NILKYTERLNMFYLILKLLNHKELTNLKGS |
| | LEKYQSANKEEAFSDQLELINLLNLDNNRV |
| | TEDFELEADEIGKFLDFNGNKVKDNKELKK |
| | FDTNKIYFDGENIIKHRAFYNIKKYGMLNL |
| | LEKIADKAGYKISIEELKKYSNKKNEIEKN |
| | HKMQENLHRKYARPRKDEKFTDEDYESYKQ |
| | AIENIEEYTHLKNKVEFNELNLLQGLLLRI |
| | LHRLVGYTSIWERDLRFRLKGEFPENQYIE |
| | EIFNFENKKNVKYKGGQIVEKYIKFYKELH |
| | QNDEVKINKYSSANIKVLKQEKKDLYIRNY |
| | IAHFNYIPHAEISLLEVLENLRKLLSYDRK |
| | LKNAVMKSVVDILKEYGFVATFKIGADKKI |
| | GIQTLESEKIVHLKNLKKKKLMTDRNSEEL |
| | CKLVKIMFEYKMEEKKSEN |
| SEQ ID: | MNELVKNRCKQTKTICQKLIPIGKTRETIE |
| 22 | KYNLMEIDRKIAANKELMNKLFSLIAGKHI |
| | NDTLSKCTDLDFEPLLTSLSSLNNAKENDR |
| | DNLREYYDSVFEEKKTLAEEISSRLTAVKF |
| | AGKDFFTKNIPDFLETYEGDDKNEMSELVS |
| | LVIENTVTAGYVKKLEKIDRSMEYRLVSGT |
| | VVKRVLTDNADIYEKNIEKAKDFDYGVLNI |
| | DEASQFTTLVAKDYANYLTADGIAIYNVGI |
| | GKINLALNEYCQKNKEYSYNKLALLPLQKM |
| | LYGEKLSLFEKLEDFTSDEELINSYNKFAK |
| | TVNESGLAEIIKKAVPSYDEIVIKPNKISN |
| | YSNSITGHWSLVNRIMKDYLENNGIKNADK |
| | YMEKGLTLSEIGDALENKNIKHSDFISNLI |
| | NDLGHTYTEIKENKESLKKDESVNALIIKK |
| | ELDMLLSILQNLKVFDIDNEMFDTGFGIEV |
| | SKAIEILGYGVPLYNKIRNYITKKPDPKKK |
| | FMTKFGSATIGTGITTSVEGSKKATFLKDG |
| | DAVFLLLYNTAGCKANNVSVSNLADLINSS |
| | LEIENSGKCYQKMIYQTPGDIKKQIPRVFV |
| | YKSEDDDLIKDFKAGLHKTDLSFLNGRLIP |
| | YLKEAFATHETYKNYTFSYRNSYESYDEFC |
| | EHMSEQAYILEWKWIDKKLIDDLVEDGSLL |
| | MFRVWNRFMKKKEGKISKHAKIVNELFSDE |
| | NASNAAIKLLSVFDIFYRDKQIDNPIVHKA |
| | GTTLYNKRTKDGEVIVDYTTMVKNKEKRPN |
| | VYTTTKKYDIIKDRRYTEEQFEIHLHVNIG |
| | KEENKEKLETSKVINEKKNTLVVTRSNEHL |
| | LYVVIFDENDNILLKKSLNTVKGMNFKSKL |
| | EVVEIQKKENMQSWKTVGSNQALMEGYLSF |
| | AIKEIADLVKEYDAILVLEQNSVGKNILNE |
| | RVYTRFKEMLITNLSLDVDYENKDFYSYTE |
| | LGGKVASWRDCVTNGICIQVPSAYKYKDPT |
| | TSFSTISMYAKTTAEKSKKLKQIKSFKYNR |
| | ERGLFELVIAKGVGLENNIVCDSFGSRSII |
| | ENDISKEVSCTLKIEKYLIDAGIEYNDEKE |
| | VLKDLDTAAKTDAVHKAVTLLLKCFNESPD |
| | GRYYISPCGEHFTLCDAPEVLSAINYYIRS |
| | RYIREQIVEGVKKMEYKKTILLAK* |
| SEQ ID: | MNGNRIIVYREFVGVTPVAKTLRNELRPIG |
| 23 | HTQEHIIHNGLIQEDELRQEKSTELKNIMD |
| | DYYREYIDKSLSGVTDLDFTLLFELMNLVQ |
| | SSPSKDNKKALEKEQSKMREQICTHMQSDS |
| | NYKNIFNAKFLKEILPDFIKNYNQYDAKDK |
| | AGKLETLALFNGFSTYFTDFFEKRKNVFTK |
| | EAVSTSIAYRIVHENSLTFLANMTSYKKIS |
| | EKALDEIEVIEKNNQDKMGDWELNQIFNPD |
| | FYNMVLIQSGIDFYNEICGVVNAHMNLYCQ |
| | QTKNNYNLFKMRKLHKQILAYTSTSFEVPK |
| | MFEDDMSVYNAVNAFIDETEKGNIIGKLKD |
| | IVNKYDELDEKRIYISKDFYETLSCFMSGN |
| | WNLITGCVENFYDENIHAKGKSKEEKVKKA |
| | VKEDKYKSINDVNDLVEKYIDEKERNEFKN |
| | SNAKQYIREISNIITDTETAHLEYDEHISL |
| | IESEEKADEMKKRLDMYMNMYHWAKAFIVD |
| | EVLDRDEMFYSDIDDIYNILENIVPLYNRV |
| | RNYVTQKPYNSKKIKLNFQSPTLANGWSQS |
| | KEFDNNAIILIRDNKYYLAIFNAKNKPDKK |
| | IIQGNSDKKNDNDYKKMVYNLLPGANKMLP |
| | KVFLSKKGIETFKPSDYIISGYNAHKHIKT |
| | SENFDISFCRDLIDYFKNSIEKHAEWRKYE |
| | FKFSATDSYNDISEFYREVEMQGYRIDWTY |
| | ISEADINKLDEEGKIYLFQIYNKDFAENST |
| | GKENLHTMYFKNIFSEENLKDIIIKLNGQA |
| | ELFYRRASVKNPVKHKKDSVLVNKTYKNQL |
| | DNGDVVRIPIPDDIYNEIYKMYNGYIKEND |
| | LSEAAKEYLDKVEVRTAQKDIVKDYRYTVD |
| | KYFIHTPITINYKVTARNNVNDMAVKYIAQ |
| | NDDIHVIGIDRGERNLIYISVIDSHGNIVK |
| | QKSYNILNNYDYKKKLVEKEKTREYARKNW |
| | KSIGNIKELKEGYISGVVHEIAMLMVEYNA |
| | IIAMEDLNYGFKRGRFKVERQVYQKFESML |
| | INKLNYFASKGKSVDEPGGLLKGYQLTYVP |
| | DNIKNLGKQCGVIFYVPAAFTSKIDPSTGF |
| | ISAFNFKSISTNASRKQFFMQFDEIRYCAE |
| | KDMFSFGFDYNNFDTYNITMSKTQWTVYTN |
| | GERLQSEFNNARRTGKTKSINLTETIKLLL |
| | EDNEINYADGHDVRIDMEKMDEDKNSEFFA |
| | QLLSLYKLTVQMRNSYTEAEEQEKGISYDK |
| | IISPVINDEGEFFDSDNYKESDDKECKMPK |
| | DADANGAYCIALKGLYEVLKIKSEWTEDGF |
| | DRNCLKLPHAEWLDFIQNKRYE* |
| SEQ ID: | MNKDIRKNFTDFVGISEIQKTLRFILIPIG |
| 24 | KTAQNIDKYNMFEDDEIRHEYYPILKEACD |
| | DFYRNHIDQQFENLELDWSKLDEALASEDR |
| | DLINETRATYRQVLFNRLKNSVDIKGDSKK |
| | NKTLSLESSDKNLGKKKTKNTFQYNFNDLF |
| | KAKLIKAILPLYIEYIYEGEKLENAKKALK |
| | MYNRFTSRLSNFWQARANIFTDDEISTGSP |
| | YRLVNDNFTIFRINNSIYTKNKPFIEEDIL |
| | EFEKKLKSKKIIKDFESVDDYFTVNAFNKL |
| | CTQNGIDKYNSILGGFTTKEREKVKGLNEL |
| | FNLAQQSINKGKKGEYRKNIRLGKLTKLKK |
| | QILAISDSTSFLIEQIEDDQDLYNKIKDFF |
| | ELLLKEEIENENIFTQYANLQKLIEQADLS |
| | KIYINAKHLNKISHQVTGKWDSLNKGIALL |
| | LENININEESKEKSEVISNGQTKDISSEAY |
| | KRYLQIQSEEKDIERLRTQIYFSLEDLEKA |
| | LDLVLIDENMDRSDKSILSYVQSPDLNVNF |
| | ERDLTDLYSRIMKLEENNEKLLANHSAIDL |
| | IKEFLDLIMLRYSRWQILFCDSNYELDQTF |
| | YPIYDAVMEILSNIIRLYNLARNYLSRKPD |
| | RMKKKKINFNNPTLADGWSESKIPDNSSML |
| | FIKDGMYYLGIIKNRAAYSELLEAESLQSS |
| | EKKKSENSSYERMNYHFLPDAFRSIPKSSI |
| | AMKAVKEHFEINQKTADLLLDTDKFSKPLR |
| | ITKEIFDMQYVDLHKNKKKYQVDYLRDTGD |
| | KKGYRKALNTWLNFCKDFISKYKGRNLFDY |
| | SKIKDADHYETVNEFYNDVDKYSYHIFFTS |
| | VAETTVEKFISEGKLYLFQLYNKDFSPHST |
| | GKPNLHTIYWRALFSEENLTSKNIKLNGQA |
| | EIFFRPKQIETPFTHKKGSILVNRFDVNGN |
| | PIPINVYQEIKGFKNNVIKWDDLNKTTQEG |
| | LENDQYLYFESEFEIIKDRRYTEDQLFFHV |
| | PISFNWDIGSNPKINDLATQYIVNSNDIHI |
| | IGIDRGENHLIYYSVIDLQGAIVEQGSLNT |
| | ITEYTENKFLNNKTNNLRKIPYKDILQQRE |
| | DERADARIKWHAIDKIKDLKDGYLGQIVHF |
| | LAKLIIKYNAIVILEDLNYGFKRGRFKVER |
| | QVYQKFEMALMKKLNVLVFKDYDIDEIGGP |
| | LKPWQLTRPIDSYERMGRQNGILFYVPAAY |
| | TSAVDPVTGFANLFYLNNVKNSEKFHFFSK |
| | FESIKYHSDQDMFSFAFDYNNFGTTTRIND |
| | LSKSKWQVFTNHERSVWNNKEKNYVTQNLT |
| | DLIKKLLRTYNIEFKNNQNVLDSILKIENN |
| | TDKENFARELFRLFRLTIQLRNTTVNENNT |
| | EITENELDYIISPVKDKNGNFFDSRDELKN |
| | LPDNGDANGAYNIARKGLLYIEQLQESIKT |
| | GKLPTLSISTLDWFNYIMK* |
| SEQ ID: | MNKGGYVIMEKMTEKNRWENQFRITKTIKE |
| 25 | EIIPTGYTKVNLQRVNMLKREMERNEDLKK |
| | MKEICDEYYRNMIDVSLRLEQVRTLGWESL |
| | IHKYRMLNKDEKEIKALEKEQEDLRKKISK |
| | GFGEKKAWTGEQFIKKILPQYLMDHYTGEE |
| | LEEKLRIVKKFKGCTMFLSTFFKNRENIFT |
| | DKPIHTAVGHRITSENAMLFAANINTYEKM |
| | ESNVTLEIERLQREFWRRGINISEIFTDAY |
| | YVNVLTQKQIEAYNKICGDINQHMNEYCQK |
| | QKLKFSEFRMRELKKQILAVVGEHFEIPEK |
| | IESTKEVYRELNEYYESLKELHGQFEELKS |
| | VQLKYSQIYVQKKGYDRISRYIGGQWDLIQ |
| | ECMKKDCASGMKGTKKNHDAKIEEEVAKVK |
| | YQSIEHIQKLVCTYEEDRGHKVTDYVDEFI |
| | VSVCDLLGADHIITRDGERIELPLQYEPGT |
| | DLLKNDTINQRRLSDIKTILDWHMDMLEWL |
| | KTFLVNDLVIKDEEFYMAIEELNERMQCVI |
| | SVYNRIRNYVTQKGYEPEKIRICFDKGTIL |
| | TGWTTGDNYQYSGFLLMRNDKYYLGIINTN |
| | EKSVRKILDGNEECKDENDYIRVGYHLIND |
| | ASKQLPRIFVMPKAGKKSEILMKDEQCDYI |
| | WDGYCHNKHNESKEFMRELIDYYKRSIMNY |
| | DKWEGYCFKFSSTESYDNMQDFYKEVREQS |
| | YNISFSYINENVLEQLDKDGKIYLFQVYNK |
| | DFAAGSTGTPNLHTMYLQNLFSSQNLELKR |
| | LRLGGNAELFYRPGTEKDVTHRKGSILVDR |
| | TYVREEKDGIEVRDTVPEKEYLEIYRYLNG |
| | KQKGDLSESAKQWLDKVHYREAPCDIIKDK |
| | RYAQEKYFLHFSVEINPNAEGQTALNDNVR |
| | RWLSEEEDIHVIGIDRGERNLIYVSLMDGK |
| | GRIKDQKSYNIVNSGNKEPVDYLAKLKVRE |
| | KERDEARRNWKAIGKIKDIKTGYLSYVVHE |
| | IVEMAVREKAIIVMEDLNYGFKRGRFKVER |
| | QVYQKFEEMLINKLNYVVDKQLSVDEPGGL |
| | LRGYQLAFIPKDKKSSMRQNGIVFYVPAGY |
| | TSKIDPTTGFVNIFKFPQFGKGDDDGNGKD |
| | YDKIRAFFGKFDEIRYECDEKVTADNTREV |
| | KERYRFDFDYSKFETHLVHMKKTKWTVYAE |
| | GERIKRKKVGNYWTSEVISDIALRMSNTLN |
| | IAGIEYKDGHNLVNEICALRGKQAGIILNE |
| | LLEIVRLTVQLRNSTTEGDVDERDEIISPV |
| | LNEKYGCFYHSTEYKQQNGDVLPKDADANG |
| | AYCIGLKGIYEIRQIKNKWKEDMTKGEGKA |
| | LNEGMRISHDQWFEFIQNMNKGE* |
| SEQ ID: | MNNPRGAFGGFTNLYSLSKTLRFELKPYLE |
| 26 | IPEGEKGKLFGDDKEYYKNCKTYTEYYLKK |
| | ANKEYYDNEKVKNTDLQLVNFLHDERIEDA |
| | YQVLKPVFDTLHEEFITDSLESAEAKKIDF |
| | GNYYGLYEKQKSEQNKDEKKKIDKPLETER |
| | GKLRKAFTPIYEAEGKNLKNKAGKEKKDKD |
| | ILKESGFKVLIEAGILKYIKNNIDEFADKK |
| | LKNNEGKEITKKDIETALGAENIEGIFDGF |
| | FTYFSGFNQNRENYYSTEEKATAVASRIVD |
| | ENLSKFCDNILLYRKNENDYLKIFNFLKNK |
| | GKDLKLKNSKFGKENEPEFIPAYDMKNDEK |
| | SFSVADFVNCLSQGEIEKYNAKIANANYLI |
| | NLYNQNKDGNSSKLSMFKILYKQIGCGEKK |
| | DFIKTIKDNAELKQILEKACEAGKKYFIRG |
| | KSEDGGVSNIFDFTDYIQSHENYKGVYWSD |
| | KAINTISGKYFANWDTLKNKLGDAKVFNKN |
| | TGEDKADVKYKVPQAVMLSELFAVLDDNAG |
| | EDWREKGIFFKASLFEGDQNKSEIIKNANR |
| | PSQALLKMICDDMESLAKNFIDSGDKILKI |
| | SDRDYQKDENKQKIKNWLDNALWINQILKY |
| | FKVKANKIKGDSIDARIDSGLDMLVFSSDN |
| | PAEDYDMIRNYLTQKPQDEINKLKLNFENS |
| | SLAGGWDENKEKDNSCIILKDEQDKQYLAV |
| | MKYENTKVFEQKNSQLYIADNAAWKKMIYK |
| | LVPGASKTLPKVFFSKKWTANRPTPSDIVE |
| | IYQKGSFKKENVDFNDKKEKDESRKEKNRE |
| | KIIAELQKTCWMDIRYNIDGKIESAKYVNK |
| | EKLAKLIDFYKENLKKYPSEEESWDRLFAF |
| | GFSDTKSYKSIDQFYIEVDKQGYKLEFVTI |
| | NKARLDEYVRDGKIYLFEIRSRDNNLVNGE |
| | EKTSAKNLQTIYWNAAFGGDDNKPKLNGEA |
| | EIFYRPAIAENKLNKKKDKNGKEIIDGYRF |
| | SKEKFIFHCPITLNFCLKETKINDKLNAAL |
| | AKPENGQGVYFLGIDRGEKHLAYYSLVNQK |
| | GEILEQGTLNLPFLDKNGKSRSIKVEKKSF |
| | EKDSNGGIIKDKDGNDKIKIEFVECWNYND |
| | LLDARAGDRDYARKNWTTIGTIKELKDGYI |
| | SQVVRKIVDLSIYKNTETKEFREMPAFIVL |
| | EDLNIGFKRGRQKIEKQVYQKLELALAKKL |
| | NFLVDKKADIGEIGSVTKAIQLTPPVNNFG |
| | DMENRKQFGNMLYIRADYTSQTDPATGWRK |
| | SIYLKSGSESNVKEQIEKSFFDIRYESGDY |
| | CFEYRDRHGKMWQLYSSKNGVSLDRFHGER |
| | NNSKNVWESEKQPLNEMLDILFDEKRFDKS |
| | KSLYEQMFKGGVALTRLPKEINKKDKPAWE |
| | SLRFVIILIQQIRNTGKNGDDRNGDFIQSP |
| | VRDEKTGEHFDSRIYLDKEQKGEKADLPTS |
| | GDANGAYNIARKGIVVAEHIKRGFDKLYIS |
| | DEEWDTWLAGDEIWDKWLKENRESLTKTRK |
| | * |
| SEQ ID: | MNYKTGLEDFIGKESLSKTLRNALIPTEST |
| 27 | KIHMEEMGVIRDDELRAEKQQELKEIMDDY |
| | YRTFIEEKLGQIQGIQWNSLFQKMEETMED |
| | ISVRKDLDKIQNEKRKEICCYFTSDKRFKD |
| | LFNAKLITDILPNFIKDNKEYTEEEKAEKE |
| | QTRVLFQRFATAFTNYFNQRRNNFSEDNIS |
| | TAISFRIVNENSEIHLQNMRAFQRIEQQYP |
| | EEVCGMEEEYKDMLQEWQMKHIYSVDFYDR |
| | ELTQPGIEYYNGICGKINEHMNQFCQKNRI |
| | NKNDFRMKKLHKQILCKKSSYYEIPFRFES |
| | DQEVYDALNEFIKTMKKKEIIRRCVHLGQE |
| | CDDYDLGKIYISSNKYEQISNALYGSWDTI |
| | RKCIKEEYMDALPGKGEKKEEKAEAAAKKE |
| | EYRSIADIDKIISLYGSEMDRTISAKKCIT |
| | EICDMAGQISIDPLVCNSDIKLLQNKEKTT |
| | EIKTILDSFLHVYQWGQTFIVSDIIEKDSY |
| | FYSELEDVLEDFEGITTLYNHVRSYVTQKP |
| | YSTVKFKLHFGSPTLANGWSQSKEYDNNAI |
| | LLMRDQKFYLGIFNVRNKPDKQIIKGHEKE |
| | EKGDYKKMIYNLLPGPSKMLPKVFITSRSG |
| | QETYKPSKHILDGYNEKRHIKSSPKFDLGY |
| | CWDLIDYYKECIHKHPDWKNYDFHFSDTKD |
| | YEDISGFYREVEMQGYQIKWTYISADEIQK |
| | LDEKGQIFLFQIYNKDFSVHSTGKDNLHTM |
| | YLKNLFSEENLKDIVLKLNGEAELFFRKAS |
| | IKTPIVHKKGSVLVNRSYTQTVGNKEIRVS |
| | IPEEYYTEIYNYLNHIGKGKLSSEAQRYLD |
| | EGKIKSFTATKDIVKNYRYCCDHYFLHLPI |
| | TINFKAKSDVAVNERTLAYIAKKEDIHIIG |
| | IDRGERNLLYISVVDVHGNIREQRSFNIVN |
| | GYDYQQKLKDREKSRDAARKNWEEIEKIKE |
| | LKEGYLSMVIHYIAQLVVKYNAVVAMEDLN |
| | YGFKTGRFKVERQVYQKFETMLIEKLHYLV |
| | FKDREVCEEGGVLRGYQLTYIPESLKKVGK |
| | QCGFIFYVPAGYTSKIDPTTGFVNLFSFKN |
| | LTNRESRQDFVGKFDEIRYDRDKKMFEFSF |
| | DYNNYIKKGTILASTKWKVYTNGTRLKKIV |
| | VNGKYTSQSMEVELTDAMEKMLQRAGIEYH |
| | DGKDLKGQIVEKGIEAEIIDIFRLTVQMRN |
| | SRSESEDREYDRLISPVLNDKGEFFDTATA |
| | DKTLPQDADANGAYCIALKGLYEVKQIKEN |
| | WKENEQFPRNKLVQDNKTWFDFMQKKRYL* |
| SEQ ID: | MRISKTLSLRIVRPFYTPEVEAGIKAEKDK |
| 28 | REAQGQTRSLDAKFFNELKKKHSEIILSSE |
| | FYSLLSEVQRQLTSIYNHAMSNLYHKIIVE |
| | GEKTSTSKALSNIGYDECKAIFPSYMALGL |
| | RQKIQSNFRRRDLKNFRMAVPTAKSDKFPI |
| | PIYRQVDGSKGGFKISENDGKDFIVELPLV |
| | DYVAEEVKTAKGRFTKINISKPPKIKNIPV |
| | ILSTLRRRQSGQWFSDDGTNAEIRRVISGE |
| | YKVSWIEIVRRTRFGKHDDWFVNMVIKYDK |
| | PEEGLDSKVVGGIDVGVSSPLVCALNNSLD |
| | RYFVKSSDIIAFNKRAMARRRTLLRQNKYK |
| | RSGHGSKNKLEPITVLTEKNERFKKSIMQR |
| | WAKEVAEFFRGKGASVVRMEELSGLKEKDN |
| | FFSSYLRMYWNYGQLQQIIENKLKEYGIKV |
| | NYVSPKDTSKKCHSCTHINEFFTFEYRQKN |
| | NFPLFKCEKCGVECSADYNAAKNMAIA |
| SEQ ID: | MRTMVTFEDFTKQYQVSKTLRFELIPQGKT |
| 29 | LENMKRDGIISVDRQRNEDYQKAKGILDKL |
| | YKYILDFTMETVVIDWEALATATEEFRKSK |
| | DKKTYEKVQSKIRTALLEHVKKQKVGTEDL |
| | FKGMFSSKIITGEVLAAFPEIRLSDEENLI |
| | LEKFKDFTTYFTGFFENRKNVFTDEALSTS |
| | FTYRLVNDNFIKFFDNCIVFKNVVNISPHM |
| | AKSLETCASDLGIFPGVSLEEVFSISFYNR |
| | LLTQTGIDQFNQLLGGISGKEGEHKQQGLN |
| | EIINLAMQQNLEVKEVLKNKAHRFTPLFKQ |
| | ILSDRSTMSFIPDAFADDDEVLSAVDAYRK |
| | YLSEKNIGDRAFQLISDMEAYSPELMRIGG |
| | KYVSVLSQLLFYSWSEIRDGVKAYKESLIT |
| | GKKTKKELENIDKEIKYGVTLQEIKEALPK |
| | KDIYEEVKKYAMSVVKDYHAGLAEPLPEKI |
| | ETDDERASIKHIMDSMLGLYRFLEYFSHDS |
| | IEDTDPVFGECLDTILDDMNETVPLYNKVR |
| | NFSTRKVYSTEKFKLNFNNSSLANGWDKNK |
| | EQANGAILLRKEGEYFLGIFNSKNKPKLVS |
| | DGGAGIGYEKMIYKQFPDFKKMLPKCTISL |
| | KDTKAHFQKSDEDFTLQTDKFEKSIVITKQ |
| | IYDLGTQTVNGKKKFQVDYPRLTGDMEGYR |
| | AALKEWIDFGKEFIQAYTSTAIYDTSLFRD |
| | SSDYPDLPSFYKDVDNICYKLTFEWIPDAV |
| | IDDCIDDGSLYLFKLHNKDFSSGSIGKPNL |
| | HTLYWKALFEEENLSDVVVKLNGQAELFYR |
| | PKSLTRPVVHEEGEVIINKTTSTGLPVPDD |
| | VYVELSKFVRNGKKGNLTDKAKNWLDKVTV |
| | RKMPHAITKDRRFTVDKFFFHVPITLNYKA |
| | DSSPYRFNDFVRQYIKDCSDVKIIGIDRGE |
| | RNLIYAVVIDGKGNIIEQRSFNTVGTYNYQ |
| | EKLEQKEKERQTARQDWATVTKIKDLKKGY |
| | LSAVVHELSKMIVKYKAIVALENLNVGFKR |
| | MRGGIAERSVYQQFEKALIDKLNYLVFKDE |
| | EQSGYGGVLNAYQLTDKFESFSKMGQQTGF |
| | LFYVPAAYTSKIDPLTGFINPFSWKHVKNR |
| | EDRRNFLNLFSKLYYDVNTHDFVLAYHHSN |
| | KDSKYTIKGNWEIADWDILIQENKEVFGKT |
| | GTPYCVGKRIVYMDDSTTGHNRMCAYYPHT |
| | ELKKLLSEYGIEYTSGQDLLKIIQEFDDDK |
| | LVKGLFYIIKAALQMRNSNSETGEDYISSP |
| | IEGRPGICFDSRAEADTLPYDADANGAFHI |
| | AMKGLLLTERIRNDDKLAISNEEWLNYIQE |
| | MRG* |
| SEQ ID: | MSKLEKFTNCYSLSKTLRFKAIPVGKTQEN |
| 30 | IDNKRLLVEDEKRAEDYKGVKKLLDRYYLS |
| | FINDVLHSIKLKNLNNYISLFRKKTRTEKE |
| | NKELENLEINLRKEIAKAFKGNEGYKSLFK |
| | KDIIETILPEFLDDKDEIALVNSFNGFTTA |
| | FTGFFDNRENMFSEEAKSTSIAFRCINENL |
| | TRYISNMDIFEKVDAIFDKHEVQEIKEKIL |
| | NSDYDVEDFFEGEFFNFVLTQEGIDVYNAI |
| | IGGFVTESGEKIKGLNEYINLYNQKTKQKL |
| | PKFKPLYKQVLSDRESLSFYGEGYTSDEEV |
| | LEVFRNTLNKNSEIFSSIKKLEKLFKNFDE |
| | YSSAGIFVKNGPAISTISKDIFGEWNVIRD |
| | KWNAEYDDIHLKKKAVVTEKYEDDRRKSFK |
| | KIGSFSLEQLQEYADADLSVVEKLKEIIIQ |
| | KVDEIYKVYGSSEKLFDADFVLEKSLKKND |
| | AVVAIMKDLLDSVKSFENYIKAFFGEGKET |
| | NRDESFYGDFVLAYDILLKVDHIYDAIRNY |
| | VTQKPYSKDKFKLYFQNPQFMGGWDKDKET |
| | DYRATILRYGSKYYLAIMDKKYAKCLQKID |
| | KDDVNGNYEKINYKLLPGPNKMLPKVFFSK |
| | KWMAYYNPSEDIQKIYKNGTFKKGDMFNLN |
| | DCHKLIDFFKDSISRYPKWSNAYDFNFSET |
| | EKYKDIAGFYREVEEQGYKVSFESASKKEV |
| | DKLVEEGKLYMFQIYNKDFSDKSHGTPNLH |
| | TMYFKLLFDENNHGQIRLSGGAELFMRRAS |
| | LKKEELVVHPANSPIANKNPDNPKKTTTLS |
| | YDVYKDKRFSEDQYELHIPIAINKCPKNIF |
| | KINTEVRVLLKHDDNPYVIGIDRGERNLLY |
| | IVVVDGKGNIVEQYSLNEIINNENGIRIKT |
| | DYHSLLDKKEKERFEARQNWTSIENIKELK |
| | AGYISQVVHKICELVEKYDAVIALEDLNSG |
| | FKNSRVKVEKQVYQKFEKMLIDKLNYMVDK |
| | KSNPCATGGALKGYQITNKFESFKSMSTQN |
| | GFIFYIPAWLTSKIDPSTGFVNLLKTKYTS |
| | IADSKKFISSFDRIMYVPEEDLFEFALDYK |
| | NFSRTDADYIKKWKLYSYGNRIRIFRNPKK |
| | NNVFDWEEVCLTSAYKELFNKYGINYQQGD |
| | IRALLCEQSDKAFYSSFMALMSLMLQMRNS |
| | ITGRTDVDFLISPVKNSDGIFYDSRNYEAQ |
| | ENAILPKNADANGAYNIARKVLWAIGQFKK |
| | AEDEKLDKVKIAISNKEWLEYAQTSVKH |
| SEQ ID: | MTNFDNFTKKYVNSKTIRLEAIPVGKTLKN |
| 31 | IEKMGFIAADRQRDEDYQKAKSVIDHIYKA |
| | FMDDCLKDLFLDWDPLYEAVVACWRERSPE |
| | GRQALQIMQADYRKKIADRFRNHELYGSLF |
| | TKKIFDGSVAQRLPDLEQSAEEKSLLSNFN |
| | KFTSYFRDFFDKRKRLFSDDEKHSAIAYRL |
| | INENFLKFVANCEAFRRMTERVPELREKLQ |
| | NTGSLQVYNGLALDEVFSADFYNQLIVQKQ |
| | IDLYNQLIGGIAGEPGTPNIQGLNATINLA |
| | LQGDSSLHEKLAGIPHRFNPLYKQILSDVS |
| | TLSFVPSAFQSDGEMLAAVRGFKVQLESGR |
| | VLQNVRRLFNGLETEADLSRVYVNNSKLAA |
| | FSSMFFGRWNLCSDALFAWKKGKQKKITNK |
| | KLTEIKKWLKNSDIAIAEIQEAFGEDFPRG |
| | KINEKIQAQADALHSQLALPIPENLKALCA |
| | KDGLKSMLDTVLGLYRMLQWFIVGDDNEKD |
| | SDFYFGLGKILGSLDPVLVLYNRVRNYITK |
| | KPYSLTKFRLNFDNSQLLNGWDENNLDTNC |
| | ASIFIKDGKYYLGISNKNNRPQFDTVATSG |
| | KSGYQRMVYKQFANWGRDLPHSTTQMKKVK |
| | KHFSASDADYVLDGDKFIRPLIITKEIFDL |
| | NNVKFNGKKKLQVDYLRNTGDREGYTHALH |
| | TWINFAKDFCACYKSTSIYDISCLRPTDQY |
| | DNLMDFYADLGNLSHRIVWQTIPEEAIDNY |
| | VEQGQLFLFQLYNKDFAPGADGKPNLHTLY |
| | WKAVFNPENLEDVVVKLNGKAELFYRPRSN |
| | MDVVRHKVGEKLVNRKLKNGLTLPSRLHEE |
| | IYRYVNGTLNKDLSADARSVLPLAVVRDVQ |
| | HEIIKDRRFTADKFFFHASLTFNFKSSDKP |
| | VGFNEDVREYLREHPDTYVVGVDRGERNLI |
| | YIVVIDPQGNIVEQRSFNMINGIDYWSLLD |
| | QKEKERVEAKQAWETVGKIKDLKCGYLSFL |
| | IHEITKIIIKYHAVVILENLSLGFKRVRTG |
| | IAEKAVYQQFERMLVTKLGYVVFKDRAGKA |
| | PGGVLNAYQLTDNTRTAENTGIQNGFLFYV |
| | PAAFTSRVDPATGFFDFYDWGKIKTATDKK |
| | NFIAGFNSVRYERSTGDFIVHVGAKNLAVR |
| | RVAEDVRTEWDIVIEANVRKMGIDGNSYIS |
| | GKRIRYRSGEQGHGQYENHLPCQELIRALQ |
| | QYGIQYETGKDILPAILQQDDAKLTDTVFD |
| | VFRLALQMRNTSAETGEDYFNSVVRDRSGR |
| | CFDTRRAEAAMPKEADANDAYHIALKGLFV |
| | LEKLRKGESIGIKNTEWLRYVQQRHS* |
| SEQ ID: | MTPIFCNFVVYQIMLFNNNININVKTMNKK |
| 32 | HLSDFTNLFPVSKTLRFRLEPQGKTMENIV |
| | KAQTIETDEERSHDYEKTKEYIDDYHRQFI |
| | DDTLDKFAFKVESTGNNDSLQDYLDAYLSA |
| | NDNRTKQTEEIQTNLRKAIVSAFKMQPQFN |
| | LLFKKEMVKHLLPQFVDTDDKKRIVAKFND |
| | FTTYFTGFFTNRENMYSDEAKSTSIAYRIV |
| | NQNLIKFVENMLTFKSHILPILPQEQLATL |
| | YDDFKEYLNVASIAEMFELDHFSIVLTQRQ |
| | IEVYNSVIGGRKDENNKQIKPGLNQYINQH |
| | NQAVKDKSARLPLLKPLFNQILSEKAGVSF |
| | LPKQFKSASEVVKSLNEAYAELSPVLAAIQ |
| | DVVTNITDYDCNGIFIKNDLGLTDIAQRFY |
| | GNYDAVKRGLRNQYELETPMHNGQKAEKYE |
| | EQVAKHLKSIESVSLAQINQVVTDGGDICD |
| | YFKAFGATDDGDIQRENLLASINNAHTAIS |
| | PVLNKENANDNELRKNTMLIKDLLDAIKRL |
| | QWFAKPLLGAGDETNKDQVFYGKFEPLYNQ |
| | LDETISPLYDKVRSYLTKKPYSLDKFKINF |
| | EKSNLLGGWDPGADRKYQYNAVILRKDNDF |
| | YLGIMRDEATSKRKCIQVLDCNDEGLDENF |
| | EKVEYKQIKPSQNMPRCAFAKKECEENADI |
| | MELKRKKNAKSYNTNKDDKNALIRHYQRYL |
| | DRTYPEFGFVYKDADEYDTVKAFTDSMDSQ |
| | DYKLSFLQVSETGLNKLVDEGDLYLFKITN |
| | KDFSSYAKGRPNLHTIYWRMLFDPKNLANV |
| | VYKLEGKAEVFFRRKSLASTTTHKAKQAIK |
| | NKSRYNEAVKPQSTFDYDIIKDRRFTADKF |
| | EFHVPIKMNFKAAGWNSTRLTNEVREFIKS |
| | QGVRHIIGIDRGERHLLYLTMIDMDGNIVK |
| | QCSLNAPAQDNARASEVDYHQLLDSKEADR |
| | LAARRNWGTIENIKELKQGYLSQVVHLLAT |
| | MMVDNDAILVLENLNAGFMRGRQKVEKSVY |
| | QKFEKMLIDKLNYIVDKGQSPDKPTGALHA |
| | VQLTGLYSDFNKSNMKRANVRQCGFVFYIP |
| | AWNTSKIDPVTGFVNLFDTHLSSMGEIKAF |
| | FSKFDSIRYNQDKGWFEFKFDYSRFTTRAE |
| | GCRTQWTVCTYGERIWTHRSKNQNNQFVND |
| | TVNVTQQMLQLLQDCGIDPNGNLKEAIANI |
| | DSKKSLETLLHLFKLTVQMRNSVTGSEVDY |
| | MISPVADERGHFFDSRESDEHLPANADANG |
| | AFNIARKGLMVVRQIMATDDVSKIKFAVSN |
| | KDWLRFAQHIDD* |
| SEQ ID: | VKISKTLSLRIIRPYYTPEVESAIKAEKDK |
| 33 | REAQGQTRNLDAKFFNELKKKHPQIILSGE |
| | FYSLLFEMQRQLTSIYNRAMSSLYHKIIVE |
| | GEKTSTSKALSDIGYDECKSVFPSYIALGL |
| | RQKIQSNFRRKELKGFRMAVPTAKSDKFPI |
| | PIYKQVDDGKGGFKISENKEGDFIVELPLV |
| | EYTAEDVKTAKGKFTKINISKPPKIKNIPV |
| | ILSTLRRKQSGQWFSDEGTNAEIRRVISGE |
| | YKVSWIEVVRRTRFGKHDDWFLNIVIKYDK |
| | TEDGLDPEVVGGIDVGVSTPLVCAVNNSLD |
| | RYFVKSSDIIAFKKRAMARRRTLLRQNRFK |
| | RSGHGSKSKLEPITILTEKNERFKKSIMQR |
| | WAKEVAEFFKGERASVVQMEELSGLKEKDN |
| | FFGSYLRMYWNYGQLQQIIENKLKEYGIKV |
| | NYVSPKDTSKKCHSCGYINEFFTFEFRQKN |
| | NFPLFKCKKCGVECNADYNAAKNIAIA |
| SEQ ID: | VKLPILKPLHKQILSEEYSTSFKIKAFEND |
| 34 | NEVLKAIDTFWNEHIEKSIHPVTGNKFNIL |
| | SKIENLCDQLQKYKDKDLEKLFIERKNLST |
| | VSHQVYGQWNIIRDALRMHLEMNNKNIKEK |
| | DIDKYLDNDAFSWKEIKDSIKIYKEHVEDA |
| | KELNENGIIKYFSAMSINEEDDEKEYSISL |
| | IKNINEKYNNVKSILQEDRTGKSDLHQDKE |
| | KVGIIKEFLDSLKQLQWFLRLLYVTVPLDE |
| | KDYEFYNELEVYYEALLPLNSLYNKVRNYM |
| | TRKPYSVEKFKLNFNSPTLLDGWDKNKETA |
| | NLSIILRKNGKYYLGIMNKENNTIFEYYPG |
| | TKSNDYYEKMIYKLLPGPNKMLPKVFFSKK |
| | GLEYYNPPKEILNIYEKGEFKKDKSGNFKK |
| | ESLHTLIDFYKEAIAKNEDWEVFNFKFKNT |
| | KEYEDISQFYRDVEEQGYLITFEKVDANYV |
| | DKLVKEGKLYLFQIYNKDFSENKKSKGNPN |
| | LHTIYWKGLYDSENLKNVVYKLNGEAEVFY |
| | RKKSIDYPEEIYNHGHHKEELLGKFNYPII |
| | KDRRYTQDKFLFHVPITMNFISKEEKRVNQ |
| | LACEYLSATKEDVHIIGIDRGERHLLYLSL |
| | IDKEGNIKKQLSLNTIKNENYDKEIDYRVK |
| | LDEKEKKRDEARKNWDVIENIKELKEGYMS |
| | QVIHIIAKMMVEEKAILIMEDLNIGFKRGR |
| | FKVEKQVYQKFEKMLIDKLNYLVFKNKNPL |
| | EPGGSLNAYQLTSKFDSFKKLGKQSGFIFY |
| | VPSAYTSKIDPTTGFYNFIQVDVPNLEKGK |
| | EFFSKFEKIIYNTKEDYFEFHCKYGKFVSE |
| | PKNKDNDRKTKESLTYYNAIKDTVWVVCST |
| | NHERYKIVRNKAGYYESHPVDVTKNLKDIF |
| | SQANINYNEGKDIKPIIIESNNAKLLKSIA |
| | EQLKLILAMRYNNGKHGDDEKDYILSPVKN |
| | KQGKFFCTLDGNQTLPINADANGAYNIALK |
| | GLLLIEKIKKQQGKIKDLYISNLEWFMFMM |
| | SR |
| SEQ ID: | MEKSLNDFIGLYSVSKTLRFELKPVSETLE |
| 35 | NIKKFHFLEEDKKKANDYKDVKKIIDNYHK |
| | YFIDDVLKNASFNWKKLEEAIREYNKNKSD |
| | DSALVAEQKKLGDAILKLFTSDKRYKALTA |
| | ATPKELFESILPDWFGEQCNQDLNKAALKT |
| | FQKFTSYFTGFQENRKNVYSAEAIPTAVPY |
| | RIVNDNFPKFLQNVLIFKTIQEKCPQIIDE |
| | VEKELSSYLGKEKLAGIFTLESFNKYLGQG |
| | GKENQRGIDFYNQIIGGVVEKEGGINLRGV |
| | NQFLNLYWQQHPDFTKEDRRIKMVPLYKQI |
| | LSDRSSLSFKIESIENDEELKNALLECADK |
| | LELKNDEKKSIFEEVCDLFSSVKNLDLSGI |
| | YINRKDINSVSRILTGDWSWLQSRMNVYAE |
| | EKFTTKAEKARWQKSLDDEGENKSKGFYSL |
| | TDLNEVLEYSSENVAETDIRITDYFEHRCR |
| | YYVDKETEMFVQGSELVALSLQEMCDDILK |
| | KRKAMNTVLENLSSENKLREKTDDVAVIKE |
| | YLDAVQELLHRIKPLKVNGVGDSTFYSVYD |
| | SIYSALSEVISVYNKTRNYITKKAASPEKY |
| | KLNFDNPTLADGWDLNKEQANTSVILRKDG |
| | MFYLGIMNPKNKPKFAEKYDCGNESCYEKM |
| | IYKQFDATKQIPKCSTQKKEVQKYFLSGAT |
| | EPYILNDKKSFKSELIITKDIWFMNNHVWD |
| | GEKFVPKRDNETRPKKFQIGYFKQTGDFDG |
| | YKNALSNWISFCKNFLQSYLSATVYDYNFK |
| | NSEEYEGLDEFYNYLNATCYKLNFINIPET |
| | EINKMVSEGKLYLFQIYNKDFASGSTGMPN |
| | MHTLYWKNLFSDENLKNVCLKLNGEAELFY |
| | RPAGIKEPVIHKEGSYLVNRTTEDGESIPE |
| | KIYFEIYKNANGKLEKLSDEAQNYISNHEV |
| | VIKKAGHEIIKDRHYTEPKFLFHVPLTINF |
| | KASGNSYSINENVRKFLKNNPDVNIIGLDR |
| | GERHLIYLSLINQKGEIIKQFTFNEVERNK |
| | NGRTIKVNYHEKLDQREKERDAARKSWQAI |
| | GKIAELKEGYLSAVIHQLTKLMVEYNAVVV |
| | MEDLNFGFKRGRFHVEKQVYQKFEHILIDK |
| | SNYLVFKDRGLNEPGGVLNGYQIAGQFESF |
| | QKLGKQSGMLFYVPAGYTSKIDPKTGFVSM |
| | MNFKDLTNVHKKRDFFSKFDNIHYDEANGS |
| | FVFTFDYKKFDGKAKEEMKLTKWSVYSRDK |
| | RIVYFAKTKSYEDVLPTEKLQKIFESNGID |
| | YKSGNNIQDSVMAIGADLKEGAKPSKEISD |
| | FWDGLLSNFKLILQMRNSNARTGEDYIISP |
| | VMADDGTFFDSREEFKKGEDAKLPLDADAN |
| | GAYHIALKGLSLINKINLSKDEELKKFDMK |
| | ISNADWFKFAQEKNYAK* |
| SEQ ID: | MIKNPSNRHSLPKVIISEVDHEKILEFKIK |
| 36 | YEKLARLDRFEVKAMHYEGKEIVFDEVLVN |
| | GGLIEVEYQDDNKTLFVKVGEKSYSIRGKK |
| | VGGKQRLLEDRVSKTKVQLELSDGVVDNKG |
| | NLRKSRTERELIVADNIKLYSQIVGREVTT |
| | TKEIYLVKRFLAYRSDLLFYYSFVDNFFKV |
| | AGNEKELWKINFDDATSAQFMGYIPFMVND |
| | NLKNDNAYLKDYVRNDVQIKDDLKKVQTIF |
| | SALRHTLLHFNYEFFEKLFNGEDVGFDFDI |
| | GFLNLLIENIDKLNIDAKKEFIDNEKIRLF |
| | GENLSLAKVYRLYSDICVNRVGFNKFINSM |
| | LIKDGVENQVLKAEFNRKFGGNAYTIDIHS |
| | NQEYKRIYNEHKKLVIKVSTLKDGQAIRRG |
| | NKKISELKEQMKSMTKKNSLARLECKMRLA |
| | FGFLYGEYNNYKAFKNNFDTNIKNSQFDVN |
| | DVEKSKAYFLSTYERRKPRTREKLEKVAKD |
| | IESLELKTVIANDTLLKFILLMFVFMPQEL |
| | KGDFLGFVKKYYHDVHSIDDDTKEQEEDVV |
| | EAMSTSLKLKILGRNIRSLTLFKYALSSQV |
| | NYNSTDNIFYVEGNRYGKIYKKLGISHNQE |
| | EFDKTLVVPLLRYYSSLFKLMNDFEIYSLA |
| | KANPTAVSLQELVDDETSPYKQGNYFNFNK |
| | MLRDIYGLTSDEIKSGQVVFMRNKIAHFDT |
| | EVLLSKPLLGQTKMNLQRKDIVSFIEARGD |
| | IKELLGYDAINDFRMKVIHLRTKMRVYSDK |
| | LQTMMDLLRNAKTPNDFYNVYKVKGVESIN |
| | KHLLEVLAQTAEERTVEKQIRDGNEKYDL |
| SEQ ID: | LNSIEKIKKPSNRNSIPSIIISDYDENKIK |
| 37 | EIKVKYLKLARLDKITIQDMEIRDNIVEFK |
| | KILLNGIEHTIKDNQKIEFDNYEITAYVRA |
| | SKQRRDGKITQAKYVVTITDKYLRDNEKEK |
| | RFKSTERELPNDTLLMRYKQISGFDTLTSK |
| | DIYKIKRYIDFKNEMLFYFQFIEEFFSPLL |
| | PKGTNFYSLNIEQNKDKVVKYIVYRLNDDF |
| | KNQSLNQFIKKTDTIKYDFLKIQKILSDFR |
| | HALAHFDFDFIQKFFDDELDKNRFDISTIS |
| | LIKTMLQEKEEKYYQEKNNYIEDSDTLTLF |
| | DEKESNFSKIHNFYIKISQKKPAFNKLINS |
| | FLSKDGVPNEELKSYLATKKIDFFEDIHSN |
| | KEYKKIYIKHKNLVVEKQKEESQEKPNGQK |
| | LKNYNDELQKLKDEMNKITKQNSLNRLEVK |
| | LRLAFGFIANEYNYNFKNFNDKFTLDVKKE |
| | QKIKVFKNSSNEKLKEYFESTFIEKRFFHF |
| | CVKFFNKKTKKEETKQKNIFNLIENETLEE |
| | LVKESPLLQIITLLYLFIPKELQGEFVGFI |
| | LKIYHHTKNITNDTKEDEKSIEDTQNSFSL |
| | KLKILAKNLRGLQLFNYSLSHNTLYNTKEH |
| | FFYEKGNRWQSVYKSLEISHNQDEFDIHLV |
| | IPVIKYYINLNKLIGDFEIYALLTYADKNS |
| | ITEKLSDITKRDDLKFRGYYNFSTLLFKTF |
| | MINTNYEQNQKSTQYIKQTRNDIAHQNIEN |
| | MLKAFENNEIFAQREEIVNYLQKEHKMQEI |
| | LHYNPINDFTMKTVQYLKSLNIHSQKESKI |
| | ADIHKKESLVPNDYYLIYKLKVIELLKQKV |
| | IEAIGETKDEEKIKNAIAKEEQIKKGYNK |
| SEQ ID: | MLKHKRKNKNSLARVVLSNYDSNNIYEIKI |
| 38 | KYEKLAKLDKINIIEMDYDADNNVMFKKVL |
| | FNNKEIDLSHKDKTKINIELDNKKYNISAK |
| | KQIGKTHLVVRNKQTSKISRIKKIQDTYYR |
| | GKDVFILDNNIEILDKKQTKDKFIVTLNDI |
| | TNNKTTSTEAELIDDTKDIFKKISAKKDLK |
| | SSDIYKIKRFISIRSNFSFYYTFVDNYFKI |
| | FHAKKDKNKEELYKIKFKDEINIKPYLENI |
| | LDNMKNKNGILYNYANDRKKVLNDLRNIQY |
| | VFKEFRHKLAHFDYNFLDNFFSNSVEEKYK |
| | QKVNEIKLLDILLDNIDSLNVVPKQNYIED |
| | ETISVFDAKDIKLKRLYTYYIKLTINYPGF |
| | KKLINSFFIQDGIENQELKEYINNKEKDTQ |
| | VLKELDNKAYYMDISQYRKYKNIYNKHKEL |
| | VSEKELSSDGKKINSLNQKINKLKIDMKNI |
| | TKPNALNRLIYRLRVAFGFIYKEYATINNF |
| | NKSFLQDTKTKRFENISQQDIKSYLDISYQ |
| | DKGKFFVKSKKTFKNKTTVKYTFEDLDLTL |
| | NEIITQDDIFVKVIFLFSIFMPKELNGDFF |
| | GFINMYYHKMKNISYDTKDIDMLDTISQNM |
| | KLKILEQNIKKTYVFKYYLDLDSSIYSKLV |
| | QNIKITEDIDSKKYLYAKIFKYYQHLYKLI |
| | SDVEIYLLYKYNSKENLSITIDKDELKHRG |
| | YYNFQSLLIKNNINKDDAYWSIVNMRNNLS |
| | HQNIDELVGHFCKGCLRKSTTDIAELWLRK |
| | DILTITNEIINKIESFKDIKITLGYDCVND |
| | FTQKVKQYKQKLKASNERLAKKIEEKQNQV |
| | VDEKNKEELEKNILNMKNIQKINRYILDIL |
| SEQ ID: | MLKHKRKNKNSLARVVLSNYDSNNIYEIKI |
| 39 | KYEKLAKLDKINIIEMDYDADNNVMFKKVL |
| | FNNKEIDLSHKDKTKINIELDNKKYNISAK |
| | KQIGKTHLVVRDKQTSKISRIKKIQDTYYR |
| | GKDVFILDNNIEILDKKQTKDKFIVTLNDI |
| | TNDKTTSTEAELIDDTKDIFKKISAKKDLK |
| | SSDIYKIKRFISIRSNFSFYYTFVDNYFKI |
| | FHAKKDKNKEELYKIKFKDEINIKPYLENI |
| | LDNMKNKNGILYDYADDREKVLNDLKNIQY |
| | VFTEFRHKLAHFDYNFLDNFFSNSVTDQYK |
| | QKVNEIKLLDILLDNIDSLNVVPKQNYIED |
| | ETISVFDAKDIKLKRLYTYYIKLTINYPGF |
| | KKLINSFFIQDGIENQELKEYINNKEKDTQ |
| | VLKELDNKAYYMDISQYRKYKNIYNKHKEL |
| | VSEKELSSDGQKINSLNQKINKLKIEMKNI |
| | TKPNALNRLIYRLRVAFGFIYKEYATINNF |
| | NKSFLQDTKIKRFENISQQDIKNYLDISYQ |
| | DKGKFFVKSKKTFKNKTTIKYTFEDLDLTL |
| | NEIITQDDIFVKVIFLFSIFMPKELNGDFF |
| | GFINMYYHKMKNISYDTKDIDMLDTISQNM |
| | KLKILEQNIKKTYVFKYYLDLDSSIYSKLV |
| | QNIKITEDIDSKKYLYAKIFKYYQHLYKLI |
| | SDVEIYLLYKYNSKENLSITIDKDELKHRG |
| | YYNFQSLLIKNNINKDDAYWSIVNMRNNLS |
| | HQNIDELVGHFCKGCLRKSTTDIAELWLRK |
| | DILTITNEIINKIESFKDIKITLGYDCVND |
| | FTQKVKQYKQKLKASNERLAKKIEEKQNQV |
| | VDEKNKEELEKKILNMKNIQKINRYILDIL |
| SEQ ID: | MSQLKNPSNKNSLPRIIISDFNEIKINEIK |
| 40 | IKYHKLDRLDKIIVKEMEIINNKIFFKKIL |
| | FNNQIKDINSENIELENYILAGEVKPSNTK |
| | IILNRDGKEKSFIVYDGFTFKYKPNDKRIS |
| | ETKTNAKYILTIKDKTRHRESSTQRDILKS |
| | SIIETYKQISGFENITSKDIYTIKRYIDFK |
| | NEMMFYYTFIDDFFFPITGKNKQDKKNNFY |
| | NYKIKENAKKFISLINYRINDDFKNKNGIL |
| | YDYLSNKEEIIINDFIHIQTILKDVRHAIA |
| | HFNFDFIQKLFDNEQAFNSKFDGIEILNIL |
| | FNQKQEKYFEAQTNYIEEETIKILDEKELS |
| | FKKLHSFYSQICQKKPAFNKLINSFIIQDG |
| | IENKELKDYISQKYNSKFDYYLDIHTCKIY |
| | KDIYNQHKKFVADKQFLENQKTDGQKIKKL |
| | NDQINQLKTKMNNLTKKNSLKRLEIKFRLA |
| | FGFIFTEYQTFKNFNERFIEDIKANKYSTK |
| | IELLDYGKIKEYISITHEEKRFFNYKTFNK |
| | KTNKNINKTIFQSLEKETFENLVKNDNLIK |
| | MMFLFQLLLPRELKGEFLGFILKIYHDLKN |
| | IDNDTKPDEKSLSELNISTALKLKILVKNI |
| | RQINLFNYTISNNTKYEEKEKRFYEEGNQW |
| | KDIYKKLYISHDFDIFDIHLIIPIIKYNIN |
| | LYKLIGDFEVYLLLKYLERNTNYKTLDKLI |
| | EAEELKYKGYYNFTTLLSKAINIALNDKEY |
| | HNITHLRNNTSHQDIQNIISSFKNNKLLEQ |
| | RENIIELISKESLKKKLHFDPINDFTMKTL |
| | QLLKSLEVHSDKSEKIENLLKKEPLLPNDV |
| | YLLYKLKGIEFIKKELISNIGITKYEEKIQ |
| | EKIAKGVEK |
| SEQ ID: | MVKNPANRHALPKVIISEVDNNNILEFKIK |
| 41 | YEKLARLDKVEVKSMHFDNNKQVVFDEVVI |
| | NGGLIEPTYEDKHKKLVVTAGEKSYSIVGQ |
| | KVGGKPRLLEDRVSKTKVQLELTNYVEDKE |
| | GKKRVSKTERELIVADNIELYSQIVGREVK |
| | TTKEIYLIKRFLEYRSDLLFYYGFVDNFFK |
| | VAGNGKELWKIDFTNSDSLHLIEYFKFSIN |
| | DNLKNDENYLKNYVSDNTKIENDLVKCQNN |
| | FNSLRHALMHFDYDFFEKLFNGEDVGFDFD |
| | IEFLNIMIDKVDKLNIDTKKEFIDDEEVTL |
| | FGEALSLKKLYGLFSHIAINRVAFNKLINS |
| | FIIEDGIENKELKDFFNNKKESQAYEIDIH |
| | SNAEYKALYVQHKKLVMATSAMTDGDEIAK |
| | KNQEISDLKEKMKVITKENSLARLEHKLRL |
| | AFGFIYTEYKDYKTFKKHFDQDIKGAKYKG |
| | LNVEKLKEYYETTLKNSKPKTDEKLEDVAK |
| | KIDKLSLKELIDDDTLLKFVLLLFIFMPQE |
| | LKGDFLGFIKKYYHDKKHIDQDTKDKDTEI |
| | EELSTGLKLKVLDKNIRSLSILKHSFSFQV |
| | KYNRKDKNFYEDGNLHGKFYKKLSISHNQE |
| | EFNKSVYAPLFRYYSALYKLINDFEIYALA |
| | QHVENHETLADQVNKSQFIQKSYFNFRKLL |
| | DNTDSISQSSSYNTLIVMRNDISHLSYEPL |
| | FNYPLDERKSYKKKTQKGVKTFHVELLYIS |
| | RAKIIELISLQTDMKKLLGYDAVNDFNMKV |
| | VHLRKRLSVYANKEESIRKMQADAKTPNDF |
| | YNIYKVKGVESINQHLLKVIGVTEAEKSIE |
| | KQINEGNKKHNT |
| SEQ ID: | MTKKPSNRNSLPKVIINKVDESSILEFKIK |
| 42 | YEKLARLDRFEVRSMRYDGDGRIIFDEVVA |
| | NAGLLDVDYEDDNRTIVVKIENKAYNIYGK |
| | KVGGEKRLNGKISKAKVQLILTDSIRKNAN |
| | DTHRHSLTERELINKNEVDLYSKIAEREIS |
| | TTKDIYLVKRFLAYRSDLLLYYAFINHYVR |
| | VNGNKKEFWKTEIDDKIIDYFIYTINDTLK |
| | NKEGYLEKYIVDRDQIKKDLEKIKQIFSHL |
| | RHKLMHYDFRFFTDLFDGKDVDIKVDNSIQ |
| | KISELLDIEFLNIVIDKLEKLNIDAKKEFI |
| | DDEKITLFGQEIELKKLYSLYAHTSINRVA |
| | FNKLINSFLIKDGVENKELKEYFNAHNQGK |
| | ESYYIDIHQNQEYKKLYIEHKNLVAKLSAT |
| | TDGKEIAKINRELADKKEQMKQITKANSLK |
| | RLEYKLRLAFGFIYTEYKDYERFKNSFDTD |
| | TKKKKFDAIDNAKIIEYFEATNKAKKIEKL |
| | EEILKGIDKLSLKTLIQDDILLKFLLLFFT |
| | FLPQEIKGEFLGFIKKYYHDITSLDEDTKD |
| | KDDEITELPRSLKLKIFSKNIRKLSILKHS |
| | LSYQIKYNKKESSYYEAGNVFNKMFKKQAI |
| | SHNLEEFGKSIYLPMLKYYSALYKLINDFE |
| | IYALYKDMDTSETLSQQVDKQEYKRNEYFN |
| | FETLLRKKFGNDIEKVLVTYRNKIAHLDFN |
| | FLYDKPINKFISLYKSREKIVNYIKNHDIQ |
| | AVLKYDAVNDFVMKVIQLRTKLKVYADKEQ |
| | TIESMIQNTQNPNGFYNIYKVKAVENINRH |
| | LLKVIGYTESEKAVEEKIRAGNTSKS |
| SEQ ID: | MIKNPSNRYALPKVIISKIDNQNILEFKIK |
| 43 | YKKLSKLDIVKVKSMHYDDRAIIFDEVIVN |
| | DGLIDVEYRDNHKTIFVKVGNKSYSISGQK |
| | VGGKERLLENRVSKTKVQLELKDKATNRVS |
| | KTERELIVDDNIKIYSQIVGRDVKTTKDIY |
| | LIKRFLAYRSDLLFYYGFVNNFFHVANNRS |
| | EFWKIDFNDSNNSKLIEYFKFTINDHLKND |
| | ENYLKDYISDNEKLKNDLIKVKNSFEKIRH |
| | ALMHFDYDFFVKLFNGEDVGLELDIEFLDI |
| | MIDKLDKLNIDTKKEFIDDEKITIFGEELS |
| | LAKLYRFYAHTAINRVAFNKLINSFIIENG |
| | VENQSLKEYFNQQAGGIAYEIDIHQNREYK |
| | NLYNEHKKLVSRVLSISDGQEIAILNQKIA |
| | KLKDQMKQITKANSIKRLEYKLRLALGFIY |
| | TEYENYEEFKNNFDTDIKNGRFTPKDNDGN |
| | KRAFDSRELEQLKGYYEATIQTQKPKTDEK |
| | IEEVSKKIDRLSLKSLIADDILLKFILLMF |
| | TFMPQELKGEFLGFIKKYYHDTKHIDQDTI |
| | SDSDDTIETLSIGLKLKILDKNIRSLSILK |
| | HSLSFQTKYNKKDRNYYEDGNIHGKFFKKL |
| | GISHNQEEFNKSVYAPLFRYYSALYKLIND |
| | FEIYTLSLHIVGSETLTDQVNKSQFLSGRY |
| | FNFRKLLTQSYHINNNSTHSTIFNAVINMR |
| | NDISHLSYEPLFDCPLNGKKSYKRKIRNQF |
| | KTINIKPLVESRKIIIDFITLQTDMQKVLG |
| | YDAVNDFTMKIVQLRTRLKAYANKEQTIQK |
| | MITEAKTPNDFYNIYKVQGVEEINKYLLEV |
| | IGETQAEKEIREKIERGNIANF |
| SEQ ID: | MKKSIFDQFVNQYALSKTLRFELKPVGETG |
| 44 | RMLEEAKVFAKDETIKKKYEATKPFFNKLH |
| | REFVEEALNEVELAGLPEYFEIFKYWKRYK |
| | KKFEKDLQKKEKELRKSVVGFFNAQAKEWA |
| | KKYETLGVKKKDVGLLFEENVFAILKERYG |
| | NEEGSQIVDESTGKDVSIFDSWKGFTGYFI |
| | KFQETRKNFYKDDGTATALATRIIDQNLKR |
| | FCDNLLIFESIRDKIDFSEVEQTMGNSIDK |
| | VFSVIFYSSCLLQEGIDFYNCVLGGETLPN |
| | GEKRQGINELINLYRQKTSEKVPFLKLLDK |
| | QILSEKEKFMDEIENDEALLDTLKIFRKSA |
| | EEKTTLLKNIFGDFVMNQGKYDLAQIYISR |
| | ESLNTISRKWTSETDIFEDSLYEVLKKSKI |
| | VSASVKKKDGGYAFPEFIALIYVKSALEQI |
| | PTEKFWKERYYKNIGDVLNKGFLNGKEGVW |
| | LQFLLIFDFEFNSLFEREIIDENGDKKVAG |
| | YNLFAKGFDDLLNNFKYDQKAKVVIKDFAD |
| | EVLHIYQMGKYFAIEKKRSWLADYDIDSFY |
| | TDPEKGYLKFYENAYEEIIQVYNKLRNYLT |
| | KKPYSEDKWKLNFENPTLADGWDKNKEADN |
| | STVILKKDGRYYLGLMARGRNKLFDDRNLP |
| | KILEGVENGKYEKVVYKYFPDQAKMFPKVC |
| | FSTKGLEFFQPSEEVITIYKNSEFKKGYTF |
| | NVRSMQRLIDFYKDCLVRYEGWQCYDFRNL |
| | RKTEDYRKNIEEFFSDVAMDGYKISFQDVS |
| | ESYIKEKNQNGDLYLFEIKNKDWNEGANGK |
| | KNLHTIYFESLFSADNIAMNFPVKLNGQAE |
| | IFYRPRTEGLEKERIITKKGNVLEKGDKAF |
| | HKRRYTENKVFFHVPITLNRTKKNPFQFNA |
| | KINDFLAKNSDINVIGVDRGEKQLAYFSVI |
| | SQRGKILDRGSLNVINGVNYAEKLEEKARG |
| | REQARKDWQQIEGIKDLKKGYISQVVRKLA |
| | DLAIQYNAIIVFEDLNMRFKQIRGGIEKSV |
| | YQQLEKALIDKLTFLVEKEEKDVEKAGHLL |
| | KAYQLAAPFETFQKMGKQTGIVFYTQAAYT |
| | SRIDPVTGWRPHLYLKYSSAEKAKADLLKF |
| | KKIKFVDGRFEFTYDIKSFREQKEHPKATV |
| | WTVCSCVERFRWNRYLNSNKGGYDHYSDVT |
| | KFLVELFQEYGIDFERGDIVGQIEVLETKG |
| | NEKFFKNFVFFFNLICQIRNTNASELAKKD |
| | GKDDFILSPVEPFFDSRNSEKFGEDLPKNG |
| | DDNGAFNIARKGLVIMDKITKFADENGGCE |
| | KMKWGDLYVSNVEWDNFVANK |
| SEQ ID: | MFNNFIKKYSLQKTLRFELKPVGETADYIE |
| 45 | DFKSEYLKDTVLKDEQRAKDYQEIKTLIDD |
| | YHREYIEECLREPVDKKTGEILDFTQDLED |
| | AFSYYQKLKENPTENRVGWEKEQESLRKKL |
| | VTSFVGNDGLFKKEFITRDLPEWLQKKGLW |
| | GEYKDTVENFKKFTTYFSGFHENRKNMYTA |
| | EAQSTAIANRLMNDNLPKFFNNYLAYQTIK |
| | EKHPDLVFRLDDALLQAAGVEHLDEAFQPR |
| | YFSRLFAQSGITAFNELIGGRTTENGEKIQ |
| | GLNEQINLYRQQNPEKAKGFPRFMPLFKQI |
| | LSDRETHSFLPDAFENDKELLQALRDYVDA |
| | ATSEEGMISQLNKAMNQFVTADLKRVYIKS |
| | AALTSLSQELFHFFGVISDAIAWYAEKRLS |
| | PKKAQESFLKQEVYAIEELNQAVVGYIDQL |
| | EDQSELQQLLVDLPDPQKPVSSFILTHWQK |
| | SQEPLQAVIAKVEPLFELEELSKNKRAPKH |
| | DKDQGGEGFQQVDAIKNMLDAFMEVSHAIK |
| | PLYLVKGRKAIDMPDVDTGFYADFAEAYSA |
| | YEQVTVSLYNKTRNHLSKKPFSKDKIKINF |
| | DAPTLLNGWDLNKESDNKSIILRKDGNFYL |
| | AIMHPKHTKVFDCYSASEAAGKCYEKMNYK |
| | LLSGANKMLPKVFFSKKGIETFSPPQEILD |
| | LYKNNEHKKGATFKLESCHKLIDFFKRNIP |
| | KYKVHPTDNFGWDVFGFHFSPTSSYGDLSG |
| | FYREVEAQGYKLWFSDVSEAYINKCVEEGK |
| | LFLFQIYNKDFSPNSTGKPNLHTLYWKGLF |
| | EPENLKDVVLKLNGEAEIFYRKHSIKHEDK |
| | TIHRAKDPIANKNADNPKKQSVFDYDIIKD |
| | KRYTQDKFFFHVPISLNFKSQGVVRFNDKI |
| | NGLLAAQDDVHVIGIDRGERHLLYYTVVNG |
| | KGEVVEQGSLNQVATDQGYVVDYQQKLHAK |
| | EKERDQARKNWSTIENIKELKAGYLSQVVH |
| | KLAQLIVKHNAIVCLEDLNFGFKRGRFKVE |
| | KQVYQKFEKALIDKLNYLVFKERGATQAGG |
| | YLNAYQLAAPFESFEKLGKQTGILYYVRSD |
| | YTSKIDPATGFVDFLKPKYESMAKSKVFFE |
| | SFERIQWNQAKGYFEFEFDYKKMCPSRKFG |
| | DYRTRWVVCTFGDTRYQNRRNKSSGQWETE |
| | TIDVTAQLKALFAAYGITYNQEDNIKDAIA |
| | AVKYTKFYKQLYWLLRLTLSLRHSVTGTDE |
| | DFILSPVADENGVFFDSRKATDKQPKDADA |
| | NGAYHIALKGLWNLQQIRQHDWNVEKPKKL |
| | NLAMKNEEWFGFAQKKKFRA |
| SEQ ID: | MIKNPSNRYALPKVIISKIDNQNILEFKIK |
| 46 | YKKLSKLDIVKVKSMHYDDRAIIFDEVIVN |
| | DGLIDVEYRDNHKTIFVKVGNKSYSISGQK |
| | VGGKERLLENRVSKTKVQLELKDKATNRVS |
| | KTERELIVDDNIKIYSQIVGRDVKTTKDIY |
| | LIKRFLAYRSDLLFYYGFVNNFFHVANNRS |
| | EFWKIDFNDSNNSKLIEYFKFTINDHLKND |
| | ENYLKDYISDNEKLKNDLIKVKNSFEKIRH |
| | ALMHFDYDFFVKLFNGEDVGLELDIEFLDI |
| | MIDKLDKLNIDTKKEFIDDEKITIFGEELS |
| | LAKLYRFYAHTAINRVAFNKLINSFIIENG |
| | VENQSLKEYFNQQAGGIAYEIDIHQNREYK |
| | NLYNEHKKLVSRVLSISDGQEIAILNQKIA |
| | KLKDQMKQITKANSIKRLEYKLRLALGFIY |
| | TEYENYEEFKNNFDTDIKNGRFTPKDNDGN |
| | KRAFDSRELEQLKGYYEATIQTQKPKTDEK |
| | IEEVSKKIDRLSLKSLIADDILLKFILLMF |
| | TFMPQELKGEFLGFIKKYYHDTKHIDQDTI |
| | SDSDDTIETLSIGLKLKILDKNIRSLSILK |
| | HSLSFQTKYNKKDRNYYEDGNIHGKFFKKL |
| | GISHNQEEFNKSVYAPLFRYYSALYKLIND |
| | FEIYTLSLHIVGSETLTDQVNKSQFLSGRY |
| | FNFRKLLTQSYHINNNSTHSTIFNAVINMR |
| | NDISHLSYEPLFDCPLNGKKSYKRKIRNQF |
| | KTINIKPLVESRKIIIDFITLQTDMQKVLG |
| | YDAVNDFTMKIVQLRTRLKAYANKEQTIQK |
| | MITEAKTPNDFYNIYKVQGVEEINKYLLEV |
| | IGETQAEKEIREKIERGNIANF |
| SEQ ID: | MIKNPSNRHSLPKVIISEVDHEKILEFKIK |
| 47 | YEKLARLDRFEVKAMHYEGKEIVFDEVLVN |
| | GGLIEVEYQDDNKTLFVKVGEKSYSIRGKK |
| | VGGKQRLLEDRVSKTKVQLELSDGVVDNKG |
| | NLRKSRTERELIVADNIKLYSQIVGREVTT |
| | TKEIYLVKRFLAYRSDLLFYYSFVDNFFKV |
| | AGNEKELWKINFDDATSAQFMGYIPFMVND |
| | NLKNDNAYLKDYVRNDVQIKDDLKKVQTIF |
| | SALRHTLLHFNYEFFEKLFNGEDVGFDFDI |
| | GFLNLLIENIDKLNIDAKKEFIDNEKIRLF |
| | GENLSLAKVYRLYSDICVNRVGFNKFINSM |
| | LIKDGVENQVLKAEFNRKFGGNAYTIDIHS |
| | NQEYKRIYNEHKKLVIKVSTLKDGQAIRRG |
| | NKKISELKEQMKSMTKKNSLARLECKMRLA |
| | FGFLYGEYNNYKAFKNNFDTNIKNSQFDVN |
| | DVEKSKAYFLSTYERRKPRTREKLEKVAKD |
| | IESLELKTVIANDTLLKFILLMFVFMPQEL |
| | KGDFLGFVKKYYHDVHSIDDDTKEQEEDVV |
| | EAMSTSLKLKILGRNIRSLTLFKYALSSQV |
| | NYNSTDNIFYVEGNRYGKIYKKLGISHNQE |
| | EFDKTLVVPLLRYYSSLFKLMNDFEIYSLA |
| | KANPTAVSLQELVDDETSPYKQGNYFNFNK |
| | MLRDIYGLTSDEIKSGQVVFMRNKIAHFDT |
| | EVLLSKPLLGQTKMNLQRKDIVSFIEARGD |
| | IKELLGYDAINDFRMKVIHLRTKMRVYSDK |
| | LQTMMDLLRNAKTPNDFYNVYKVKGVESIN |
| | KHLLEVLAQTAEERTVEKQIRDGNEKYDL |
| SEQ ID: | MEEKMLKSYDYFTKLYSLQKTLRFELKPIG |
| 48 | KTLEHIKNSGIIESDETLEEQYAIVKNIID |
| | KLHRKHIDEALSLVDFTKHLDTLKTFQELY |
| | LKRGKTDKEKEELEKLSADLRKLIVSYLKG |
| | NVKEKTQHNLNPIKERFEILFGKELFTNEE |
| | FFLLAENEKEKKAIQAFKGFTTYFKGFQEN |
| | RKNMYSEEGNSTSIAYRIINENLPLFIENI |
| | ARFQKVMSTIEKTTIKKLEQNLKTELKKHN |
| | LPGIFTIEYFNNVLTQEGISRYNTIIGGKT |
| | THEGVKIQGLNEIINLYNQQSKDVKLPILK |
| | PLHKQILSEEYSTSFKIKAFENDNEVLKAI |
| | DTFWNEHIEKSIHPVTGNKFNILSKIENLC |
| | DQLQKYKDKDLEKLFIERKNLSTVSHQVYG |
| | QWNIIRDALRMHLEMNNKNIKEKDIDKYLD |
| | NDAFSWKEIKDSIKIYKEHVEDAKELNENG |
| | IIKYFSAMSINEEDDEKEYSISLIKNINEK |
| | YNNVKSILQEDRTGKSDLHQDKEKVGIIKE |
| | FLDSLKQLQWFLRLLYVTVPLDEKDYEFYN |
| | ELEVYYEALLPLNSLYNKVRNYMTRKPYSV |
| | EKFKLNFNSPTLLDGWDKNKETANLSIILR |
| | KNGKYYLGIMNKENNTIFEYYPGTKSNDYY |
| | EKMIYKLLPGPNKMLPKVFFSKKGLEYYNP |
| | PKEILNIYEKGEFKKDKSGNFKKESLHTLI |
| | DFYKEAIAKNEDWEVFNFKFKNTKEYEDIS |
| | QFYRDVEEQGYLITFEKVDANYVDKLVKEG |
| | KLYLFQIYNKDFSENKKSKGNPNLHTIYWK |
| | GLYDSENLKNVVYKLNGEAEVFYRKKSIDY |
| | PEEIYNHGHHKEELLGKFNYPIIKDRRYTQ |
| | DKFLFHVPITMNFISKEEKRVNQLACEYLS |
| | ATKEDVHIIGIDRGERHLLYLSLIDKEGNI |
| | KKQLSLNTIKNENYDKEIDYRVKLDEKEKK |
| | RDEARKNWDVIENIKELKEGYMSQVIHIIA |
| | KMMVEEKAILIMEDLNIGFKRGRFKVEKQV |
| | YQKFEKMLIDKLNYLVFKNKNPLEPGGSLN |
| | AYQLTSKFDSFKKLGKQSGFIFYVPSAYTS |
| | KIDPTTGFYNFIQVDVPNLEKGKEFFSKFE |
| | KIIYNTKEDYFEFHCKYGKFVSEPKNKDND |
| | RKTKESLTYYNAIKDTVWVVCSTNHERYKI |
| | VRNKAGYYESHPVDVTKNLKDIFSQANINY |
| | NEGKDIKPIIIESNNAKLLKSIAEQLKLIL |
| | AMRYNNGKHGDDEKDYILSPVKNKQGKFFC |
| | TLDGNQTLPINADANGAYNIALKGLLLIEK |
| | IKKQQGKIKDLYISNLEWFMFMMSR |
| SEQ ID: | MEKSLNDFIGLYSVSKTLRFELKPVSETLE |
| 49 | NIKKFHFLEEDKKKANDYKDVKKIIDNYHK |
| | YFIDDVLKNASFNWKKLEEAIREYNKNKSD |
| | DSALVAEQKKLGDAILKLFTSDKRYKALTA |
| | ATPKELFESILPDWFGEQCNQDLNKAALKT |
| | FQKFTSYFTGFQENRKNVYSAEAIPTAVPY |
| | RIVNDNFPKFLQNVLIFKTIQEKCPQIIDE |
| | VEKELSSYLGKEKLAGIFTLESFNKYLGQG |
| | GKENQRGIDFYNQIIGGVVEKEGGINLRGV |
| | NQFLNLYWQQHPDFTKEDRRIKMVPLYKQI |
| | LSDRSSLSFKIESIENDEELKNALLECADK |
| | LELKNDEKKSIFEEVCDLFSSVKNLDLSGI |
| | YINRKDINSVSRILTGDWSWLQSRMNVYAE |
| | EKFTTKAEKARWQKSLDDEGENKSKGFYSL |
| | TDLNEVLEYSSENVAETDIRITDYFEHRCR |
| | YYVDKETEMFVQGSELVALSLQEMCDDILK |
| | KRKAMNTVLENLSSENKLREKTDDVAVIKE |
| | YLDAVQELLHRIKPLKVNGVGDSTFYSVYD |
| | SIYSALSEVISVYNKTRNYITKKAASPEKY |
| | KLNFDNPTLADGWDLNKEQANTSVILRKDG |
| | MFYLGIMNPKNKPKFAEKYDCGNESCYEKM |
| | IYKQFDATKQIPKCSTQKKEVQKYFLSGAT |
| | EPYILNDKKSFKSELIITKDIWFMNNHVWD |
| | GEKFVPKRDNETRPKKFQIGYFKQTGDFDG |
| | YKNALSNWISFCKNFLQSYLSATVYDYNFK |
| | NSEEYEGLDEFYNYLNATCYKLNFINIPET |
| | EINKMVSEGKLYLFQIYNKDFASGSTGMPN |
| | MHTLYWKNLFSDENLKNVCLKLNGEAELFY |
| | RPAGIKEPVIHKEGSYLVNRTTEDGESIPE |
| | KIYFEIYKNANGKLEKLSDEAQNYISNHEV |
| | VIKKAGHEIIKDRHYTEPKFLFHVPLTINF |
| | KASGNSYSINENVRKFLKNNPDVNIIGLDR |
| | GERHLIYLSLINQKGEIIKQFTFNEVERNK |
| | NGRTIKVNYHEKLDQREKERDAARKSWQAI |
| | GKIAELKEGYLSAVIHQLTKLMVEYNAVVV |
| | MEDLNFGFKRGRFHVEKQVYQKFEHILIDK |
| | SNYLVFKDRGLNEPGGVLNGYQIAGQFESF |
| | QKLGKQSGMLFYVPAGYTSKIDPKTGFVSM |
| | MNFKDLTNVHKKRDFFSKFDNIHYDEANGS |
| | FVFTFDYKKFDGKAKEEMKLTKWSVYSRDK |
| | RIVYFAKTKSYEDVLPTEKLQKIFESNGID |
| | YKSGNNIQDSVMAIGADLKEGAKPSKEISD |
| | FWDGLLSNFKLILQMRNSNARTGEDYIISP |
| | VMADDGTFFDSREEFKKGEDAKLPLDADAN |
| | GAYHIALKGLSLINKINLSKDEELKKFDMK |
| | ISNADWFKFAQEKNYAK |
| SEQ ID: | MNTQKKEFNPKSFKDFTNLYSLNKTLRFSL |
| 50 | TPNKKTAEILEFNKQKEVKCFSNDRKIAGA |
| | YQEIKKYLNKLHQEFIQEAMKFFAFSEEEL |
| | KGFEKEYLNLLNFTDKDNFKKKNKIRNEYE |
| | QERKILTIKIATYFSKFKSEKYQSFNLANI |
| | TGKKVFSILEQKYKEDKKTLKIIHIFKYKP |
| | TKDEKKEGEAVNFSTYLTGFNENRKNFYKS |
| | EDKAGQFATRTIDNLAQFIKNKKLFEDKYQ |
| | KNYSKIGILDEQIKIFNLDYFNNLFLQEGL |
| | DEYNGILGNNKGEENKSNEGINQKINIFKQ |
| | KEKARLKKEKENFNKSDFPLFKELYKQIGS |
| | IRKENDVYVEIKTDKELVEELNNFPKNVEN |
| | YLKDIQSFYKTFFEKLQNEEYELDKIYLPK |
| | SVGTYFSYIAFSDWNKLAFIYNKRYKNEKI |
| | KIVEGGDVNVQYRSLEVLKNRIDELKDEDN |
| | LNFNKFFIDKLKFNEAKKENNWQNFWFCIE |
| | YYINSQFIGGEKNILNKEKNEYEILPFGSL |
| | KELKEKYFEAVKKYKEKMVDTESGLTDDEE |
| | KEIKETLKNYLDRIKEIERIAKYFDLKKSF |
| | EEIKQEDLDSNFYGEYQKVVDKTNELKIYQ |
| | YYSEFRNYLTQNNSVEEKIKLNFNSGLLLD |
| | GWDLNKEKVKFSIIFQENGKYYLGIINKEK |
| | DKTILDKDKHPEIFTKNSDFRKMEYKLFPS |
| | PSKMLPKISFSETAKKGDEDVGWSEEIQKI |
| | KDEFAEFQEYKKKSKDNWKDEFNRGKLNKL |
| | IDYYKQVLEKHSEGYMNTYNFELKDSSKYK |
| | NLGEFNDDIARQNYKVKFVGIDKNYIDEKV |
| | ANGELFLFQIYNKDFSEDKKEGSTNNLETI |
| | YFKELFSKENLENPVFKLSGGAEMFFRNKI |
| | EKKKEKKKLDKDGKPMISKKGEKVVDKRRF |
| | SENKILFHLPIEINYGKGKMPNFNKKINEY |
| | ISKNPENIKIIGIDRGEKHLLYYSIIDQNG |
| | NNIESMSLNAVDEFGNFVNPEKLEEYEIDN |
| | NGKKERRWKYIVNDKEIKVTNYQRKLDELE |
| | KERQKSRQSWQNINKIKNLKKGYISFVVKK |
| | IVDLAIENNAIIILEDLNFGFKSFRQKIEK |
| | NVYQQFEKALIDKLGFVVDKQKQNQRFAPQ |
| | LSAPFESFQKIGKQTGIVYYVLANNTSKVC |
| | PSCQWIKNFYLKYEKKNTIFNLQKNQKLKV |
| | FFEQEKNRFRFEYQMSKEYISVYSDVDRQR |
| | YDKTKNQNKGGYLEYKNSNQKEIIDKDGVI |
| | QKQSITLQLKELFKENHIDLEKEILKQLDN |
| | KKEKNSGYTGVYNKFIYLFNLILQIRNAIS |
| | FREKDYIQCPSCHFDTRKENYLKINDGDGN |
| | GAYNIALRGLYLLKGKNGIINNLEKIKLIF |
| | SNNDYFQWAKKLKNKK |
| SEQ ID: | MQNKQSFADFTNLYSLSKTLRFELKPIGQT |
| 51 | QAMLDENKIFEVDENRKKAYDKTKPYFDRL |
| | HREFINESLSNAQLKGISEYFETFKQFRSN |
| | QNNKDLKELINKQQKFLRHQIVTLFDENGK |
| | HWATTKYAHLKIKKKNLDILFDEQVFYILK |
| | ERYGSEKETQLVDKETGAVTSIFDNWKGFT |
| | GYFTKFFETRKNFYKSDGTSTALATRIIDQ |
| | NLNRFFDNLETFHKIKDKIDVKEVEIFFKL |
| | KADNVFSIDFYNQCLLQNGIDKYNDFLGGQ |
| | TLENGEKQKGINEIINKYRQDNKDQKLPFL |
| | KKLDKQILSEKDRFINEIESKEEFFQVLTE |
| | FYQSATVKVTIIKTLLNDFVHNTDKYKLEK |
| | IYLTKEAFNTIANKWTDETQIFEDNLDLVL |
| | KNKKITAKQDFIPLAYIKEALEVIEKDRKF |
| | FKDRYYNDPQIGFFPDQSYWEQFLAILNFE |
| | FMTHFQRVAKDKITGKKIELGYFVFEKRIK |
| | ELLDSDPSLNSQSKIIIKEFADEVLHIFQM |
| | AKYFALEKKREWKGDYYQLDDQFYNHIDYG |
| | FKDQFYENAYEKIVQPYNKIRNYLTKKPYS |
| | DVKWKLNFGNPTLANGWDKNKEADNTAVIL |
| | KKDGNYYLGVMKKGKNKIFSDQNKEKYKAY |
| | NSAYYEKLVYKLFPDPSKMFPKVCFSKKGL |
| | NFFQPSEEILRIYKNNEFKKGNTFSISSMQ |
| | KLIAFYIDCLGLYEGWKHYEFKNIKDVRQY |
| | KENIGEFYADVAESGYKLWFEKISEEYITQ |
| | KNQLGELFLFQIYNKDFAKKTTGRKNLHTI |
| | YFEELFSQTNIDNNFPFKLNGQAELFYRPK |
| | SLEKIEEKRNFKRSIVNKKRYTQNKIFFHV |
| | PITLNRTSENIGRFNVRVNNFLANNSNVNI |
| | VGVDRGEKNLAYYSIIKQNGEVLKSGSLNI |
| | INGVDYHALLTDRAQRREQERRNWQDVESI |
| | KDLKRGYISQVVHELVSLAIKYNAIIVMED |
| | LNMRFKQIRGGIEKSTYQQLEKALIEKLNF |
| | LVNKEETDSNQAGNLLNAYQLTAPFKTFKD |
| | MGKQTGIIFYTQASYTSKIDPLTGWRPNIY |
| | LRYSNAKQAKADILMFTNIYFSEKKDRFEF |
| | TYDLEKIDDKRKDLPIKTEWTVCSNVERFS |
| | WEKSLNNNKGGYVHYPIQDSNGEESITSKL |
| | KKLFMDFGIDLTDIKTQIESLDTNKKDNAN |
| | FFRKFIFYFQLICQIRNTQVNKSDDGNDFI |
| | FSPVEPFFDSRFADKFRKNLPKNGDENGAY |
| | NIARKGLIILHKISDYFVKEGSTDKISWKD |
| | LSISQTEWDNFTTDK |
| SEQ ID: | MKKEKEFKSFGDFTNLYEISKTLRFELKPV |
| 52 | ENTQTMLDEADVFGKDKVIKDKYTKTKPFI |
| | DKLHREFVDESLKDVSLSGLKKYSEVLENW |
| | KKNKKDKDIVKELKKEEERLRKEVVEFFDN |
| | TAKKWANEKYKELGLKKKDIGILFEESVFD |
| | LLKEKYGEEQDSFLKEEKGDFLKNEKGEKV |
| | SIFDEWKGFVGYFTKFQETRKNFYKNDGTE |
| | TALATRIIDQNLKRFCDNIDDFKKIKNKID |
| | FSEVEKNFNKTADVFSLDFYNQCLLQKGID |
| | SYNEFIGGKTLENGKKLKGVNELVNEYRQK |
| | NKNEKVSFLKLLDKQILSEKEKLSFGIEND |
| | EQLLVVLNSFYETAEEKTKILRTLFGDFVE |
| | HNENYDLDKTYISKVAFNTISHKWTNETHK |
| | FEELLYGAMKEDKPIGLNYDKKEDSYKFPD |
| | FIALGYLKKCLNNLDCDTKFWKEKYYENNA |
| | DKKDKDKGFLTGGQNAWDQFLQIFIFEFNQ |
| | LFNSEAFDNKGKEIKIGYDNFRKDFEEIIN |
| | QKDFKNDENLKIAIKNFADSVLWIYQMAKY |
| | FAIEKKRGWDDDFELSEFYTNPSNGYSLFY |
| | DRAYEEIVQKYNDLRNYLTKKPYKEDKWKL |
| | NFENPTLANGFDKNKESDNSTVILRKKRKY |
| | YLGLMKKGNNKIFEDRNKAEFIRNIESGAY |
| | EKMAYKYLPDVAKMIPKCSTQLNEAKNHFR |
| | NSADDLEIKKSFSNPLKITKRIFDLNNIQY |
| | DKTNVSKKISGDNKGIKIFQKEYYKISGDF |
| | DVYKSALNDWIDFCKDFLSKYDSTKDFDFS |
| | ILRKTKDYKSLDEFYVDVAKITYKISFTPV |
| | SESYIDQKNKNGELYLFEIYNQDFAKGKMG |
| | AKNLHTLYFENVFSPENISKNFPIKLNGNA |
| | ELFFRPKSIESKKEKRNFVREIVNKKRYSE |
| | DKIFFHCPITLNRETGSIYRFNNYVNNFLS |
| | ENNINIIGVDRGEKHLAYYSVIDKNGVKIG |
| | GGSFNEINKVDYAKKLEERAGEREQSRKDW |
| | QVVEGIKDLKKGYISQVVRELADLAIKHNA |
| | IIVLEDLNMRFKQIRGGIEKSIYQQLEKAL |
| | IDKLSFLVEKGEKDPNQAGHILKAYQLAAP |
| | FTSFKDMGKQTGIVFYTQASYTSKTCPNCG |
| | FRKNNNKFYFENNIGKAQDALKKLKTFEYD |
| | SENKCFGLSYCLSDFANKEEVEKNKNKKRN |
| | NAPYSDIEKKDCFELSTKDAVRYRWHDKNT |
| | ERGKTFFEGESVYEEKEEKEIGQTKRGLVK |
| | EYDISKCLIGLFEKTGLDYKQNLLDKINSG |
| | KFDGTFYKNLFNYLNLLFEIRNSISGTEID |
| | YISCPECQFHTDKSKTIKNGDDNGSYNIAR |
| | KGMIILDKIKQFKKENGSLDKMGWGELFID |
| | LEEWDKFAQKKNNNIIDK |
| SEQ ID: | MKYTDFTGIYSVSKTLRFELIPQGSTVENM |
| 53 | KREGILNNDMHRADSYKEMKKLIDEYHKAF |
| | IERCLSDFSLKYDDTGKHDSLEEYFFYYEQ |
| | KRNDKTKKIFEDIQVALRKQISKRFTGDTA |
| | FKRLFKKELIKEDLPSFVKNDPVKTELIKE |
| | FSDFTTYFQEFHKNRKNMYTSDAKSTAIAY |
| | RIINENLPKFIDNINAFDIVAKVPEMQEHF |
| | KTIADELRSHLQVGNDIDKMFNLQFFNKVL |
| | TQSQLDVYNAVIGGKSEGNKKIQGINEYIN |
| | LYNQQHKKARLPMLKLLYKQILSDRVAISW |
| | LQDEFDNDQDMLDTIEAFYNKLNSNETGVL |
| | GEGKLKQILMGLDGYNLDGVFMRNDLQLSE |
| | VSQRLCGGWNIIKDAMTSDLKRSVQKKKKE |
| | TDADFEERVSKLFSAQNSFSIAYINQCLGQ |
| | AGIRCKIQDYFACLGAKEGENEAETTPDIF |
| | DQIAEAYHGAAPILNARPSSHNLAQDIEKV |
| | KAIKALLDALKRLQRFVKPLLGRGDEGDKD |
| | NFFYGDFMPIWEVLDQLTPLYNKVRNRMTR |
| | KPYSQEKIKLNFENSTLLNGWDLNKEHDNT |
| | SVILRREGLYYLGIMNKNYNKIFDANNVET |
| | IGDCYEKMIYKLLPGPNKMLPKVFFSKSRV |
| | QEFSPSKKILEIWESKSFKKGDNFNLDDCH |
| | ALIDFYKDSIAKHPDWNKFNFKFSDTQSYT |
| | NISDFYRDVNQQGYSLSFTKVSVDYVNRMV |
| | DEGKLYLFQIYNKDFSPQSKGTPNMHTLYW |
| | RMLFDERNLHNVIYKLNGEAEVFYRKASLR |
| | CDRPTHPAHQPITCKNENDSKRVCVFDYDI |
| | IKNRRYTVDKFMFHVPITINYKCTGSDNIN |
| | QQVCDYLRSAGDDTHIIGIDRGERNLLYLV |
| | IIDQHGTIKEQFSLNEIVNEYKGNTYCTNY |
| | HSLLEEKEAGNKKARQDWQTIESIKELKEG |
| | YLSQVIHKISMLMQRYHAIVVLEDLNGSFM |
| | RSRQKVEKQVYQKFEHMLINKLNYLVNKQY |
| | DATEPGGLLHALQLTSRMDSFKKLGKQSGF |
| | LFYIPAWNTSKIDPVTGFVNLFDTRYCNEA |
| | KAKEFFEKFDDISYNDERDWFEFSFDYRHF |
| | TNKPTGTRTQWTLCTQGTRVRTFRNPEKSN |
| | HWDNEEFDLTQAFKDLFNKYGIDIASGLKA |
| | RIVNGQLTKETSAVKDFYESLLKLLKLTLQ |
| | MRNSVTGTDIDYLVSPVADKDGIFFDSRTC |
| | GSLLPANADANGAFNIARKGLMLLRQIQQS |
| | SIDAEKIQLAPIKNEDWLEFAQEKPYL |
| SED ID: | MEKEITELTKIRREFPNKKFSSTDMKKAGK |
| 54 | LLKAEGPDAVRDFLNSCQEIIGDFKPPVKT |
| | NIVSISRPFEEWPVSMVGRAIQEYYFSLTK |
| | EELESVHPGTSSEDHKSFFNITGLSNYNYT |
| | SVQGLNLIFKNAKAIYDGTLVKANNKNKKL |
| | EKKFNEINHKRSLEGLPIITPDFEEPFDEN |
| | GHLNNPPGINRNIYGYQGCAAKVFVPSKHK |
| | MVSLPKEYEGYNRDPNLSLAGFRNRLEIPE |
| | GEPGHVPWFQRMDIPEGQIGHVNKIQRFNF |
| | VHGKNSGKVKFSDKTGRVKRYHHSKYKDAT |
| | KPYKFLEESKKVSALDSILAIITIGDDWVV |
| | FDIRGLYRNVFYRELAQKGLTAVQLLDLFT |
| | GDPVIDPKKGVVTFSYKEGVVPVFSQKIVP |
| | RFKSRDTLEKLTSQGPVALLSVDLGQNEPV |
| | AARVCSLKNINDKITLDNSCRISFLDDYKK |
| | QIKDYRDSLDELEIKIRLEAINSLETNQQV |
| | EIRDLDVFSADRAKANTVDMFDIDPNLISW |
| | DSMSDARVSTQISDLYLKNGGDESRVYFEI |
| | NNKRIKRSDYNISQLVRPKLSDSTRKNLND |
| | SIWKLKRTSEEYLKLSKRKLELSRAVVNYT |
| | IRQSKLLSGINDIVIILEDLDVKKKFNGRG |
| | IRDIGWDNFFSSRKENRWFIPAFHKAFSEL |
| | SSNRGLCVIEVNPAWTSATCPDCGFCSKEN |
| | RDGINFTCRKCGVSYHADIDVATLNIARVA |
| | VLGKPMSGPADRERLGDTKKPRVARSRKTM |
| | KRKDISNSTVEAMVTA |
| SED ID: | MIKPTVSQFLTPGFKLIRNHSRTAGLKLKN |
| 55 | EGEEACKKFVRENEIPKDECPNFQGGPAIA |
| | NIIAKSREFTEWEIYQSSLAIQEVIFTLPK |
| | DKLPEPILKEEWRAQWLSEHGLDTVPYKEA |
| | AGLNLIIKNAVNTYKGVQVKVDNKNKNNLA |
| | KINRKNEIAKLNGEQEISFEEIKAFDDKGY |
| | LLQKPSPNKSIYCYQSVSPKPFITSKYHNV |
| | NLPEEYIGYYRKSNEPIVSPYQFDRLRIPI |
| | GEPGYVPKWQYTFLSKKENKRRKLSKRIKN |
| | VSPILGIICIKKDWCVFDMRGLLRTNHWKK |
| | YHKPTDSINDLFDYFTGDPVIDTKANVVRF |
| | RYKMENGIVNYKPVREKKGKELLENICDQN |
| | GSCKLATVDVGQNNPVAIGLFELKKVNGEL |
| | TKTLISRHPTPIDFCNKITAYRERYDKLES |
| | SIKLDAIKQLTSEQKIEVDNYNNNFTPQNT |
| | KQIVCSKLNINPNDLPWDKMISGTHFISEK |
| | AQVSNKSEIYFTSTDKGKTKDVMKSDYKWF |
| | QDYKPKLSKEVRDALSDIEWRLRRESLEFN |
| | KLSKSREQDARQLANWISSMCDVIGIENLV |
| | KKNNFFGGSGKREPGWDNFYKPKKENRWWI |
| | NAIHKALTELSQNKGKRVILLPAMRTSITC |
| | PKCKYCDSKNRNGEKFNCLKCGIELNADID |
| | VATENLATVAITAQSMPKPTCERSGDAKKP |
| | VRARKAKAPEFHDKLAPSYTVVLREAV |
| SED ID: | MEKEITELTKIRREFPNKKFSSTDMKKAGK |
| 56 | LLKAEGPDAVRDFLNSCQEIIGDFKPPVKT |
| | NIVSISRPFEEWPVSMVGRAIQEYYFSLTK |
| | EELESVHPGTSSEDHKSFFNITGLSNYNYT |
| | SVQGLNLIFKNAKAIYDGTLVKANNKNKKL |
| | EKKFNEINHKRSLEGLPIITPDFEEPFDEN |
| | GHLNNPPGINRNIYGYQGCAAKVFVPSKHK |
| | MVSLPKEYEGYNRDPNLSLAGFRNRLEIPE |
| | GEPGHVPWFQRMDIPEGQIGHVNKIQRFNF |
| | VHGKNSGKVKFSDKTGRVKRYHHSKYKDAT |
| | KPYKFLEESKKVSALDSILAIITIGDDWVV |
| | FDIRGLYRNVFYRELAQKGLTAVQLLDLFT |
| | GDPVIDPKKGVVTFSYKEGVVPVFSQKIVP |
| | RFKSRDTLEKLTSQGPVALLSVDLGQNEPV |
| | AARVCSLKNINDKITLDNSCRISFLDDYKK |
| | QIKDYRDSLDELEIKIRLEAINSLETNQQV |
| | EIRDLDVFSADRAKANTVDMFDIDPNLISW |
| | DSMSDARVSTQISDLYLKNGGDESRVYFEI |
| | NNKRIKRSDYNISQLVRPKLSDSTRKNLND |
| | SIWKLKRTSEEYLKLSKRKLELSRAVVNYT |
| | IRQSKLLSGINDIVIILEDLDVKKKFNGRG |
| | IRDIGWDNFFSSRKENRWFIPAFHKTFSEL |
| | SSNRGLCVIEVNPAWTSATCPDCGFCSKEN |
| | RDGINFTCRKCGVSYHADIDVATLNIARVA |
| | VLGKPMSGPADRERLGDTKKPRVARSRKTM |
| | KRKDISNSTVEAMVTA |
| SED ID: | VPDKKETPLVALCKKSFPGLRFKKHDSRQA |
| 57 | GRILKSKGEGAAVAFLEGKGGTTQPNFKPP |
| | VKCNIVAMSRPLEEWPIYKASVVIQKYVYA |
| | QSYEEFKATDPGKSEAGLRAWLKATRVDTD |
| | GYFNVQGLNLIFQNARATYEGVLKKVENRN |
| | SKKVAKIEQRNEHRAERGLPLLTLDEPETA |
| | LDETGHLRHRPGINCSVFGYQHMKLKPYVP |
| | GSIPGVTGYSRDPSTPIAACGVDRLEIPEG |
| | QPGYVPPWDRENLSVKKHRRKRASWARSRG |
| | GAIDDNMLLAVVRVADDWALLDLRGLLRNT |
| | QYRKLLDRSVPVTIESLLNLVTNDPTLSVV |
| | KKPGKPVRYTATLIYKQGVVPVVKAKVVKG |
| | SYVSKMLDDTTETFSLVGVDLGVNNLIAAN |
| | ALRIRPGKCVERLQAFTLPEQTVEDFFRFR |
| | KAYDKHQENLRLAAVRSLTAEQQAEVLALD |
| | TFGPEQAKMQVCGHLGLSVDEVPWDKVNSR |
| | SSILSDLAKERGVDDTLYMFPFFKGKGKKR |
| | KTEIRKRWDVNWAQHFRPQLTSETRKALNE |
| | AKWEAERNSSKYHQLSIRKKELSRHCVNYV |
| | IRTAEKRAQCGKVIVAVEDLHHSFRRGGKG |
| | SRKSGWGGFFAAKQEGRWLMDALFGAFCDL |
| | AVHRGYRVIKVDPYNTSRTCPECGHCDKAN |
| | RDRVNREAFICVCCGYRGNADIDVAAYNIA |
| | MVAITGVSLRKAARASVASTPLESLAAE |
| SEQ ID: | MPKPAVESEFSKVLKKHFPGERFRSSYMKR |
| 58 | GGKILAAQGEEAVVAYLQGKSEEEPPNFQP |
| | PAKCHVVTKSRDFAEWPIMKASEAIQRYIY |
| | ALSTTERAACKPGKSSESHAAWFAATGVSN |
| | HGYSHVQGLNLIFDHTLGRYDGVLKKVQLR |
| | NEKARARLESINASRADEGLPEIKAEEEEV |
| | ATNETGHLLQPPGINPSFYVYQTISPQAYR |
| | PRDEIVLPPEYAGYVRDPNAPIPLGVVRNR |
| | CDIQKGCPGYIPEWQREAGTAISPKTGKAV |
| | TVPGLSPKKNKRMRRYWRSEKEKAQDALLV |
| | TVRIGTDWVVIDVRGLLRNARWRTIAPKDI |
| | SLNALLDLFTGDPVIDVRRNIVTFTYTLDA |
| | CGTYARKWTLKGKQTKATLDKLTATQTVAL |
| | VAIDLGQTNPISAGISRVTQENGALQCEPL |
| | DRFTLPDDLLKDISAYRIAWDRNEEELRAR |
| | SVEALPEAQQAEVRALDGVSKETARTQLCA |
| | DFGLDPKRLPWDKMSSNTTFISEALLSNSV |
| | SRDQVFFTPAPKKGAKKKAPVEVMRKDRTW |
| | ARAYKPRLSVEAQKLKNEALWALKRTSPEY |
| | LKLSRRKEELCRRSINYVIEKTRRRTQCQI |
| | VIPVIEDLNVRFFHGSGKRLPGWDNFFTAK |
| | KENRWFIQGLHKAFSDLRTHRSFYVFEVRP |
| | ERTSITCPKCGHCEVGNRDGEAFQCLSCGK |
| | TCNADLDVATHNLTQVALTGKTMPKREEPR |
| | DAQGTAPARKTKKASKSKAPPAEREDQTPA |
| | QEPSQTS |
| SEQ ID: | MSNKTTPPSPLSLLLRAHFPGLKFESQDYK |
| 59 | IAGKKLRDGGPEAVISYLTGKGQAKLKDVK |
| | PPAKAFVIAQSRPFIEWDLVRVSRQIQEKI |
| | FGIPATKGRPKQDGLSETAFNEAVASLEVD |
| | GKSKLNEETRAAFYEVLGLDAPSLHAQAQN |
| | ALIKSAISIREGVLKKVENRNEKNLSKTKR |
| | RKEAGEEATFVEEKAHDERGYLIHPPGVNQ |
| | TIPGYQAVVIKSCPSDFIGLPSGCLAKESA |
| | EALTDYLPHDRMTIPKGQPGYVPEWQHPLL |
| | NRRKNRRRRDWYSASLNKPKATCSKRSGTP |
| | NRKNSRTDQIQSGRFKGAIPVLMRFQDEWV |
| | IIDIRGLLRNARYRKLLKEKSTIPDLLSLF |
| | TGDPSIDMRQGVCTFIYKAGQACSAKMVKT |
| | KNAPEILSELTKSGPVVLVSIDLGQTNPIA |
| | AKVSRVTQLSDGQLSHETLLRELLSNDSSD |
| | GKEIARYRVASDRLRDKLANLAVERLSPEH |
| | KSEILRAKNDTPALCKARVCAALGLNPEMI |
| | AWDKMTPYTEFLATAYLEKGGDRKVATLKP |
| | KNRPEMLRRDIKFKGTEGVRIEVSPEAAEA |
| | YREAQWDLQRTSPEYLRLSTWKQELTKRIL |
| | NQLRHKAAKSSQCEVVVMAFEDLNIKMMHG |
| | NGKWADGGWDAFFIKKRENRWFMQAFHKSL |
| | TELGAHKGVPTIEVTPHRTSITCTKCGHCD |
| | KANRDGERFACQKCGFVAHADLEIATDNIE |
| | RVALTGKPMPKPESERSGDAKKSVGARKAA |
| | FKPEEDAEAAE |
| SEQ ID: | MSKTKELNDYQEALARRLPGVRHQKSVRRA |
| 60 | ARLVYDRQGEDAMVAFLDGKEVDEPYTLQP |
| | PAKCHILAVSRPIEEWPIARVTMAVQEHVY |
| | ALPVHEVEKSRPETTEGSRSAWFKNSGVSN |
| | HGVTHAQTLNAILKNAYNVYNGVIKKVENR |
| | NAKKRDSLAAKNKSRERKGLPHFKADPPEL |
| | ATDEQGYLLQPPSPNSSVYLVQQHLRTPQI |
| | DLPSGYTGPVVDPRSPIPSLIPIDRLAIPP |
| | GQPGYVPLHDREKLTSNKHRRMKLPKSLRA |
| | QGALPVCFRVFDDWAVVDGRGLLRHAQYRR |
| | LAPKNVSIAELLELYTGDPVIDIKRNLMTF |
| | RFAEAVVEVTARKIVEKYHNKYLLKLTEPK |
| | GKPVREIGLVSIDLNVQRLIALAIYRVHQT |
| | GESQLALSPCLHREILPAKGLGDFDKYKSK |
| | FNQLTEEILTAAVQTLTSAQQEEYQRYVEE |
| | SSHEAKADLCLKYSITPHELAWDKMTSSTQ |
| | YISRWLRDHGWNASDFTQITKGRKKVERLW |
| | SDSRWAQELKPKLSNETRRKLEDAKHDLQR |
| | ANPEWQRLAKRKQEYSRHLANTVLSMAREY |
| | TACETVVIAIENLPMKGGFVDGNGSRESGW |
| | DNFFTHKKENRWMIKDIHKALSDLAPNRGV |
| | HVLEVNPQYTSQTCPECGHRDKANRDPIQR |
| | ERFCCTHCGAQRHADLEVATHNIAMVATTG |
| | KSLTGKSLAPQRLQEAAE |
| SEQ ID: | VAFLDGKEVDEPYTLQPPAKCHILAVSRPI |
| 61 | EEWPIARVTMAVQEHVYALPVHEVEKSRPE |
| | TTEGSRSAWFKNSGVSNHGVTHAQTLNAIL |
| | KNAYNVYNGVIKKVENRNAKKRDSLAAKNK |
| | SRERKGLPHFKADPPELATDEQGYLLQPPS |
| | PNSSVYLVQQHLRTPQIDLPSGYTGPVVDP |
| | RSPIPSLIPIDRLAIPPGQPGYVPLHDREK |
| | LTSNKHRRMKLPKSLRAQGALPVCFRVFDD |
| | WAVVDGRGLLRHAQYRRLAPKNVSIAELLE |
| | LYTGDPVIDIKRNLMTFRFAEAVVEVTARK |
| | IVEKYHNKYLLKLTEPKGKPVREIGLVSID |
| | LNVQRLIALAIYRVHQTGESQLALSPCLHR |
| | EILPAKGLGDFDKYKSKFNQLTEEILTAAV |
| | QTLTSAQQEEYQRYVEESSHEAKADLCLKY |
| | SITPHELAWDKMTSSTQYISRWLRDHGWNA |
| | SDFTQITKGRKKVERLWSDSRWAQELKPKL |
| | SNETRRKLEDAKHDLQRANPEWQRLAKRKQ |
| | EYSRHLANTVLSMAREYTACETVVIAIENL |
| | PMKGGFVDGNGSRESGWDNFFTHKKENRWM |
| | IKDIHKALSDLAPNRGVHVLEVNPQYTSQT |
| | CPECGHRDKANRDPIQRERFCCTHCGAQRH |
| | ADLEVATHNIAMVATTGKSLTGKSLAPQRL |
| | Q |

In some instances, effector proteins disclosed herein are engineered proteins. Engineered proteins are not identical to a naturally-occurring protein. Engineered proteins may provide enhanced nuclease or nickase activity as compared to a naturally occurring nuclease or nickase. An engineered protein may comprise a modified form of a wild type counterpart protein. In some instances, effector proteins comprise at least one amino acid change (e.g., deletion, insertion, or substitution) that enhances or reduces the nucleic acid-cleaving activity of the effector protein relative to the wild type counterpart.

In some embodiments, a programmable nuclease may be thermostable. In some instances, known programmable nucleases (e.g., Cas12 nucleases) are relatively thermo-sensitive and only exhibit activity (e.g., cis and/or trans cleavage) sufficient to produce a detectable signal in a diagnostic assay at temperatures less than 40° C., and optimally at about 37° C. A thermostable protein may have enzymatic activity, stability, or folding comparable to those at 37° C. In some instances, the trans cleavage activity (e.g., the maximum trans cleavage rate as measured by fluorescent signal generation) of a programmable nuclease in a trans cleavage assay at 40° C., 45° C., 50° C., 55° C., 60° C., 65° C., 70° C., 75° C., 80° C., or more may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

The programmable nuclease can become activated after binding of (i) a guide nucleic acid that is complexed with the programmable nuclease with (ii) a target nucleic acid, and the activated programmable nuclease can cleave the target nucleic acid, which can result in a trans cleavage activity. Trans cleavage activity can be non-specific cleavage of nearby single-stranded nucleic acids by the activated programmable nuclease, such as trans cleavage of reporters (e.g., detector nucleic acids) with a detection moiety. Once the target nucleic acid or reporter is cleaved by the activated programmable nuclease, the detection moiety can be released or separated from the reporter and can generate a detectable signal. The reporter and/or the detection moiety can be immobilized on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a disease, cancer, or genetic disorder.

The methods and compositions of the present disclosure are compatible with any type of programmable nuclease that is human-engineered or naturally occurring. The programmable nuclease can comprise a nuclease that is capable of being activated when complexed with a guide nucleic acid and a target nucleic acid segment or a portion thereof. A programmable nuclease can become activated when complexed with a guide nucleic acid and a target sequence of a target gene of interest. The programmable nuclease can be activated upon binding of a guide nucleic acid to a target nucleic acid and can exhibit or enable trans cleavage activity once activated. In any instances or embodiments where a CRISPR-based programmable nuclease is described or used, it is recognized herein that any other type of programmable nuclease can be used in addition to or in substitution of such CRISPR-based programmable nuclease.

The methods and compositions of the present disclosure are compatible with a plurality of programmable nucleases, including any of the programmable nucleases described herein. The device can comprise a plurality of programmable nuclease probes comprising the plurality of programmable nucleases and one or more corresponding guide nucleic acids. The plurality of programmable nuclease probes can be the same. Alternatively, the plurality of programmable nuclease probes can be different. For example, the plurality of programmable nuclease probes can comprise different programmable nucleases and/or different guide nucleic acids associated with the programmable nucleases.

As used herein, a programmable nuclease generally refers to any enzyme that can be used to cleave or facilitate cleavage of a nucleic acid. The programmable nuclease can be any enzyme that can be or has been designed, modified, or engineered by human contribution so that the enzyme targets or cleaves the nucleic acid in a sequence-specific manner. Programmable nucleases can include, for example, zinc-finger nucleases (ZFNs), transcription activator-like effector nucleases (TALENs), and/or RNA-guided nucleases such as the bacterial clustered regularly interspaced short palindromic repeat (CRISPR)-Cas (CRISPR-associated) nucleases. Programmable nucleases can also include, for example, PfAgo and/or NgAgo.

ZFNs can cut genetic material in a sequence-specific matter and can be designed, or programmed, to target specific viral targets, A ZFN is composed of two domains; a DNA-binding zinc-finger protein linked to the Fokl nuclease domain. The DNA-binding zinc-finger protein is fused with the non-specific Fokl cleave domain to create ZFNs. The protein will typically dimerize for activity. Two ZFN monomers form an active nuclease; each monomer binds to adjacent half-sites on the target. The sequence specificity of ZFNs is determined by ZFPs, Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers.

Transcription activator-like effector nucleases (TALENs) can cut genetic material in a sequence-specific matter and can be designed, or programmed, to target specific viral targets, TALENs contain the Fokl nuclease domain at their carboxyl termini and a class of DNA binding domains known as transcription activator-like effectors (TALEs), TALENs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target various nucleic acids.

The programmable nuclease can comprise any type of human engineered enzymes. Alternatively, the programmable nuclease can comprise CRISPR enzymes derived from naturally occurring bacterial. A programmable nuclease can be a Cas protein (also referred to, interchangeably, as a Cas nuclease), A crRNA and Cas protein can form a CRISPR enzyme. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. The programmable nuclease can comprise one or more amino acid modifications. The programmable nuclease be a nuclease derived from a CRISPR-Cas system. The programmable nuclease can be a nuclease derived from recombineering.

As described above, trans cleavage activity can be initiated by one or more activated programmable nucleases, including, for instance, trans cleavage of one or more reporters (e.g., reporter nucleic acids) comprising a detection moiety. Once the one or more reporters are cleaved by the activated programmable nuclease, the detection moiety can be released or separated from the reporter and can directly or indirectly generate a detectable signal. The reporter molecules described herein can comprise, in some non-limiting examples, RNA. In some embodiments, the reporter molecules can comprise ssDNA. In some embodiments, a reporter molecule can comprise: RNA, dsDNA, one or more modified nucleotides, and/or any combination thereof. The reporter molecules can comprise at least one nucleic acid and a detectable moiety. In some embodiments, a programmable nuclease probe or a CRISPR probe comprising a Cas enzyme can recognize and detect a target such as dsDNA and, further, can specifically trans-cleave ssDNA reporters. In some embodiments, a programmable nuclease probe or a CRISPR probe comprising a Cas enzyme can recognize and detect a target such as RNA and, further, can specifically trans-cleave RNA reporters. The detection of the target nucleic acid in the sample can indicate the presence of the sequence in the sample and can provide information about the presence or absence of a nucleic acid sequence in a sample, e.g., as a diagnostic for disease.

Cleavage of a protein-nucleic acid can produce a signal. The signal can indicate a presence of the target nucleic acid in the sample, and an absence of the signal can indicate an absence of the target nucleic acid in the sample. In some cases, cleavage of the protein-nucleic acid can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals,

### Sample

The systems, devices, apparatuses, methods, and compositions of the present disclosure may be used to analyze one or more samples to detect a presence or an absence of one or more targets as described elsewhere herein. In some instances, the one or more samples can be taken from a single-cell eukaryotic organism; a plant or a plant cell; an algal cell; a fungal cell; an animal or an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample used for disease testing can comprise at least one target sequence that can bind to a guide nucleic acid of the reagents described herein. In some cases, the target sequence is a portion of a nucleic acid. A nucleic acid can be from a genomic locus, a viral locus, a transcribed mRNA, or a reverse transcribed cDNA. A nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A nucleic acid can be from 10 to 90, from 20 to 80, from 30 to 70, or from 40 to 60 nucleotides in length. A nucleic acid sequence can be from 10 to 95, from 20 to 95, from 30 to 95, from 40 to 95, from 50 to 95, from 60 to 95, from 10 to 75, from 20 to 75, from 30 to 75, from 40 to 75, from 50 to 75, from 5 to 50, from 15 to 50, from 25 to 50, from 35 to 50, or from 45 to 50 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can be reverse complementary to a guide nucleic acid. In some cases, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides of a guide nucleic acid can be reverse complementary to a target nucleic acid.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., SARS-COV-2 (i.e. a virus that causes COVID-19), SARS, MERS, influenza and the like), human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis, Pathogens include viruses, fungi, helminths, protozoa, malarial parasites, Plasmodium parasites, Toxoplasma parasites, and Schistosoma parasites, Helminths include roundworms, heartworms, and phytophagous nematodes, flukes, Acanthocephala, and tapeworms, Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans, Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g. the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g. warts (including genital, anal), herpes (including oral, genital, anal), molluscum contagiosum); hemmorhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid comprises a sequence from a virus or a bacterium or other agents responsible for a disease that can be found in the sample. In some cases, the target nucleic acid is a portion of a nucleic acid from a viral locus, a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis, Pathogens include viruses, fungi, helminths, protozoa, malarial parasites, Plasmodium parasites, Toxoplasma parasites, and Schistosoma parasites, Helminths include roundworms, heartworms, and phytophagous nematodes, flukes, Acanthocephala, and tapeworms, Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii, Fungal pathogens include, but are not limited to Cryptococcus neoformans, Histoplasma capsulatum, Coccidioides immitis, Blastomyces dermatitidis, Chlamydia trachomatis, and Candida albicans, Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some cases, the sample used for cancer testing or cancer risk testing can comprise at least one target sequence or target nucleic acid segment that can bind to a guide nucleic acid of the reagents described herein. The target nucleic acid segment, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes for a cancer biomarker, such as a prostate cancer biomarker or non-small cell lung cancer. In some cases, the assay can be used to detect “hotspots” in target nucleic acids that can be predictive of cancer, such as lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARD1, BLM, BMPR1A, BRCA1. BRCA2, BRIP1, CASR, CDC73, CDH1, CDK4, CDKN1B, CDKN1C, CDKN2A, CEBPA. CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

In some cases, the sample used for genetic disorder testing can comprise at least one target sequence or target nucleic acid segment that can bind to a guide nucleic acid of the reagents described herein. In some embodiments, the genetic disorder is hemophilia, sickle cell anemia, β-thalassemia, Duchene muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6VIB1, ATP7A, ATP7B, ATRX, BBS1, BBS10, BBS12, BBS2, BCKDHA, BCKDHB, BCSIL, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9. FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

In some cases, the sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

In some cases, the sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

In some cases, the sample used for ancestral testing can comprise at least one target nucleic acid segment that can bind to a guide nucleic acid of the reagents described herein. The target nucleic acid segment, in some cases, is a portion of a nucleic acid from a gene associated with a geographic region of origin or ethnic group.

In some embodiments, the sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status. In any of the embodiments described herein, the device can be configured for asymptomatic, pre-symptomatic and/or symptomatic diagnostic applications, irrespective of immunity. In any of the embodiments described herein, the device can be configured to perform one or more serological assays on a sample (e.g., a sample comprising blood).

In some embodiments, the sample can be used to identify a mutation in a target nucleic acid of a plant or of a bacteria, virus, or microbe associated with a plant or soil. The devices and methods of the present disclosure can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation.

The systems, devices, apparatuses, and methods disclosed herein may be used to detect a presence or an absence of one or more targets in one or more samples. The one or more samples can comprise one or more target sequences or nucleic acids for detection of an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop, or an environmental sample can be obtained to test for presence of a disease, virus, pathogen, cancer, genetic disorder, or any mutation or pathogen of interest. A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, any combination thereof. A sample can be an aspirate of a bodily fluid from an animal (e.g., human, animals, livestock, pet, etc.) or plant. A tissue sample can be from any tissue that can be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). A sample can be taken from the environment immediately surrounding a plant, such as hydroponic fluid/water. or soil. A sample from an environment can be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system. In some cases, the sample is contained in no more than about 200 nanoliters (nL). In some cases, the sample is contained in about 200 nL. In some cases, the sample is contained in a volume that is greater than about 200 nL and less than about 20 microliters (μL). In some cases, the sample is contained in no more than 20 μl. In some cases, the sample is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl. In some cases, the sample is contained in from 1 μL to 500 μL, from 10 μL to 500 μL, from 50 μL to 500 μL, from 100 μL to 500 μL, from 200 μL to 500 μL, from 300 μL to 500 μL, from 400 μL to 500 μL, from 1 μL to 200 μL, from 10 μL to 200 μL, from 50 μL to 200 μL, from 100 μL to 200 μL, from 1 μL to 100 μL, from 10 μL to 100 μL, from 50 μL to 100 μL, from 1 μL to 50 μL, from 10 μL to 50 μL, from 1 μL to 20 μL, from 10 μL to 20 μL, or from 1 μL to 10 μL. Sometimes, the sample is contained in more than 500 μl.

In some instances, the sample is taken from a single-cell eukaryotic organism; a plant or a plant cell; an algal cell; a fungal cell; an animal or an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample may comprise nucleic acids from a cell lysate from a eukaryotic cell, a mammalian cell, a human cell, a prokaryotic cell, or a plant cell. In some cases, the sample may comprise nucleic acids expressed from a cell.

The sample used for disease testing can comprise at least one target sequence that can bind to a guide nucleic acid of the reagents described herein. In some cases, the target sequence is a portion of a nucleic acid. A nucleic acid can be from a genomic locus, a transcribed mRNA, or a reverse transcribed cDNA. A nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A nucleic acid can be from 10 to 90, from 20 to 80, from 30 to 70, or from 40 to 60 nucleotides in length. A nucleic acid sequence can be from 10 to 95, from 20 to 95, from 30 to 95, from 40 to 95, from 50 to 95, from 60 to 95, from 10 to 75, from 20 to 75, from 30 to 75, from 40 to 75, from 50 to 75, from 5 to 50, from 15 to 50, from 25 to 50, from 35 to 50, or from 45 to 50 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can be reverse complementary to a guide nucleic acid. In some cases, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides of a guide nucleic acid can be reverse complementary to a target nucleic acid.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., SARS-COV-2 (i.e., a virus that causes COVID-19), SARS, MERS, influenza, Adenovirus, Coronavirus HKUI, Coronavirus NL63, Coronavirus 229E, Coronavirus OC43, Severe Acute Respiratory Syndrome Coronavirus 2 (SARS-COV-2), Human Metapneumovirus (hMPV), Human Rhinovirus/Enterovirus. Influenza A. Influenza A/H1, Influenza A/H3. Influenza A/H1-2009. Influenza B. Influenza C, Parainfluenza Virus 1, Parainfluenza Virus 2, Parainfluenza Virus 3, Parainfluenza Virus 4, Respiratory Syncytial Virus) and respiratory bacteria (e.g. Bordetella parapertussis, Bordetella pertussis, Chlamydia pneumoniae, Mycoplasma pneumoniae). Other viruses include human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis, Pathogens include viruses, fungi, helminths, protozoa, malarial parasites, Plasmodium parasites. Toxoplasma parasites, and Schistosoma parasites, Helminths include roundworms, heartworms, and phytophagous nematodes, flukes, Acanthocephala, and tapeworms, Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii, Fungal pathogens include, but are not limited to Cryptococcus neoformans, Histoplasma capsulatum, Coccidioides immitis, Blastomyces dermatitidis, Chlamydia trachomatis, Chlamydia pneumoniae, Chlamydia psittaci, and Candida albicans, Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g., warts (including genital, anal), herpes (including oral, genital, anal), molluscum contagiosum); hemmorhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Bacillus anthracis, Bortadella pertussis, Burkholderia cepacia, Corynebacterium diphtheriae, Coxiella burnetii, Streptococcus agalactiae, methicillin-resistant Staphylococcus aureus, Legionella longbeachae, Legionella pneumophila, Leptospira interrogans, Moraxella catarrhalis, Streptococcus pyogenes, Escherichia coli, Neisseria gonorrhoeae, Neisseria meningitidis, Neisseria elongate, Neisseria gonorrhoeae, Parechovirus, Pneumococcus, Pneumocystis jirovecii, Cryptococcus neoformans, Histoplasma capsulatum, Haemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid may comprise a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis, Pathogens include viruses, fungi, helminths, protozoa, malarial parasites, Plasmodium parasites, Toxoplasma parasites, and Schistosoma parasites, Helminths include roundworms, heartworms, and phytophagous nematodes, flukes, Acanthocephala, and tapeworms, Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans. Histoplasma capsulatum, Coccidioides immitis, Blastomyces dermatitidis, Chlamydia trachomatis, and Candida albicans, Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Staphylococcus epidermidis, Legionella pneumophila, Streptococcus pyogenes, Streptococcus salivarius, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some instances, the target nucleic acid is a single stranded nucleic acid. Alternatively, or in combination, the target nucleic acid is a double stranded nucleic acid and is prepared into single stranded nucleic acids before or upon contacting the reagents. The target nucleic acid can be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the systems and methods disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample has from 1 to 10,000, from 100 to 8000, from 400 to 6000, from 500 to 5000, from 1000 to 4000, or from 2000 to 3000 target nucleic acids. In some cases, the sample has from 100 to 9500, from 100 to 9000, from 100 to 8500, from 100 to 8000, from 100 to 7500, from 100 to 7000, from 100 to 6500, from 100 to 6000, from 100 to 5500, from 100 to 5000, from 250 to 9500, from 250 to 9000, from 250) to 8500, from 250 to 8000, from 250 to 7500, from 250 to 7000, from 250 to 6500, from 250 to 6000, from 250 to 5500, from 250 to 5000, from 2500 to 9500, from 2500 to 9000, from 2500 to 8500, from 2500 to 8000, from 2500 to 7500, from 2500 to 7000, from 2500 to 6500, from 2500 to 6000, from 2500 to 5500, or from 2500 to 5000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the systems and methods disclosed herein. Some methods described herein can be implemented to detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the sample has from 2 to 50, from 5 to 50, from 10 to 50, from 2 to 25, from 3 to 25, from 4 to 25, from 5 to 25, from 10 to 25, from 2 to 20, from 3 to 20, from 4 to 20, from 5 to 20, from 10 to 20, from 2 to 10, from 3 to 10, from 4 to 10, from 5 to 10, from 6 to 10, from 7 to 10, from 8 to 10, or from 9 to 10 target nucleic acid populations. In some cases, the methods of the present disclosure can be implemented to detect target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

The sample used for cancer testing or cancer risk testing can comprise at least one target sequence or target nucleic acid segment that can bind to a guide nucleic acid of the reagents described herein. The target nucleic acid segment, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes for a cancer biomarker, such as a prostate cancer biomarker or non-small cell lung cancer. In some cases, the assay can be used to detect “hotspots” in target nucleic acids that can be predictive of cancer, such as lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

The sample used for genetic disorder testing can comprise at least one target sequence or target nucleic acid segment that can bind to a guide nucleic acid of the reagents described herein. In some embodiments, the genetic disorder is hemophilia, sickle cell anemia, β-thalassemia, Duchene muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJI1, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MP1, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

The sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

The sample used for ancestral testing can comprise at least one target nucleic acid segment that can bind to a guide nucleic acid of the reagents described herein. The target nucleic acid segment, in some cases, is a portion of a nucleic acid from a gene associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method may comprise obtaining a serum sample from a subject; and identifying a disease status of the subject. Often, the disease status is prostate disease status. In any of the embodiments described herein, the device can be configured for asymptomatic, pre-symptomatic, and/or symptomatic diagnostic applications, irrespective of immunity. In any of the embodiments described herein, the device can be configured to perform one or more serological assays on a sample (e.g., a sample comprising blood).

In some embodiments, the sample can be used to identify a mutation in a target nucleic acid of a plant or of a bacteria, virus, or microbe associated with a plant or soil. The devices and methods of the present disclosure can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation may be a single nucleotide mutation.

### Sample Preparation

In some cases, the sample may be separated into a plurality of droplets, aliquots, or subsamples. One or more targets (e.g., nucleic acids, biomolecules, etc.) may be contained within the plurality of droplets, aliquots, or subsamples.

In some cases, the targets may be amplified before detection occurs. In some cases, the detection devices of the present disclosure may comprise a chamber or subsystem for amplifying the targets. In some cases, the detection devices can be configured to amplify the target sequences or target nucleic acids contained within the plurality of droplets by individually processing each of the plurality of droplets (e.g., by using a thermocycling process or any other suitable amplification process as described in greater detail below). In some cases, the plurality of droplets can undergo separate thermocycling processes. In some cases, the thermocycling processes can occur simultaneously. In other cases, the thermocycling processes can occur at different times for each droplet.

In some cases, the detection devices can be further configured to remix the droplets, aliquots, or subsamples after the target nucleic acids in each of the droplets undergo amplification. The detection device can be configured to provide the remixed sample comprising the droplets, aliquots, or subsamples to a detection chamber of the device. The detection chamber can be configured to direct the remixed droplets, aliquots, or subsamples to a plurality of programmable nuclease probes (described in greater detail below). In some cases, the detection chamber can be configured to direct the remixed droplets, aliquots, or subsamples along one or more fluid flow paths such that the remixed droplets, aliquots, or subsamples are positioned adjacent to and/or in contact with the one or more programmable nuclease probes. In some cases, the detection chamber can be configured to recirculate or recycle the remixed droplets, aliquots, or subsamples such that the remixed droplets, aliquots, or subsamples are repeatedly placed in contact with one or more programmable nuclease probes.

In any of the embodiments described herein, the detection device can comprise a single integrated system that is configured to perform sample collection, sample processing, droplet generation, droplet processing (e.g., amplification of target nucleic acids in droplets), droplet remixing, and/or circulation of the remixed droplets within a detection chamber so that at least a portion of the remixed droplets is placed in contact with one or more programmable nuclease probes coupled to the detection chamber. The detection devices of the present disclosure can be disposable devices configured to perform one or more rapid single reaction or multi-reaction tests to detect a presence and/or an absence of one or more target sequences or target nucleic acids.

In one aspect, the present disclosure provides exemplary methods for programmable nuclease-based detection. The method can comprise collecting a sample. The sample can comprise any type of sample as described herein. The method can comprise preparing the sample. Sample preparation can comprise one or more sample preparation steps. The one or more sample preparation steps can be performed in any suitable order.

In some cases, the one or more sample preparation steps can comprise physical filtration of non-target materials using a macro filter, nucleic acid purification, lysis, heat inactivation, or adding one or more enzymes or reagents to prepare the sample for target detection.

In some cases, the method can comprise generating one or more droplets, aliquots, or subsamples from the sample. The one or more droplets, aliquots, or subsamples can correspond to a volumetric portion of the sample. The sample can be divided into 1, 2, 3, 4, 5, 6, 7, 8, 9, 10, 20, 30, 40, 50, 60, 70, 80, 90, 100, 200, 300, 400, 500, 600, 700, 800, 900, 1000, or more droplets, aliquots, or subsamples. In some embodiments, the sample is not divided into subsamples.

In some cases, the method can comprise amplifying one or more targets within each droplet, aliquot, or subsample. Amplification of the one or more targets within each droplet can be performed in parallel and/or simultaneously for each droplet. Dividing the sample into a plurality of droplets can enhance a speed and/or an efficiency of the amplification process (e.g., a thermocycling process) since the droplets comprise a smaller volume of material than the bulk sample introduced. Amplifying the one or more targets within each individual droplet can also permit effective amplification of various target nucleic acids that cannot be amplified as efficiently in a bulk sample containing the various target nucleic acids if the bulk sample were to undergo a singular amplification process. In some embodiments, amplification is performed on the bulk sample without first dividing the sample into subsamples.

In some cases, the method can further comprise using one or more CRISPR-based or programmable nuclease-based probes (as described elsewhere herein) to detect one or more targets (e.g., target sequences or target nucleic acids) in the sample. In some cases, the sample can be divided into a plurality of droplets, aliquots, or subsamples to facilitate sample preparation and to enhance the detection capabilities of the devices of the present disclosure. In some cases, the sample is not divided into subsamples.

In some embodiments, the sample can be provided manually to a detection device. For example, a swab sample can be dipped into a solution and the sample/solution can be pipetted into the device. In other embodiments, the sample can be provided via an automated syringe. The automated syringe can be configured to control a flow rate at which the sample is provided to the detection device. The automated syringe can be configured to control a volume of the sample that is provided to the detection device over a predetermined period. In some embodiments, the sample can be provided directly to the detection device. For example, a swab sample can be inserted into a sample chamber on the detection device.

The sample can be prepared before one or more targets are detected within the sample. The sample preparation steps described herein can process a crude sample to generate a pure or purer sample. Sample preparation can one or more physical or chemical processes, including, for example, nucleic acid purification, lysis, binding, washing, and/or eluting. In certain instances, sample preparation can comprise the following steps, in any order, including sample collection, nucleic acid purification, heat inactivation, and/or base/acid lysis.

In some embodiments, nucleic acid purification can be performed on the sample, Purification can comprise disrupting a biological matrix of a cell to release nucleic acids, denaturing structural proteins associated with the nucleic acids (nucleoproteins), inactivating nucleases that can degrade the isolated product (RNase and/or DNase), and/or removing contaminants (e.g., proteins, carbohydrates, lipids, biological or environmental elements, unwanted nucleic acids, and/or other cellular debris).

In some embodiments, lysis of a collected sample can be performed. Lysis can be performed using a protease (e.g., a Proteinase K or PK enzyme). In some cases, a solution of reagents can be used to lyse the cells in the sample and release the nucleic acids so that they are accessible to the programmable nuclease. Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. One example protocol may comprise a 4 M guanidinium isothiocyanate, 25 mM sodium citrate.2H20, 0.5% (w/v) sodium lauryl sarcosinate, and 0.1 M β-mercaptoethanol), but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Cell lysis can also be performed by physical, mechanical, thermal or enzymatic means, in addition to chemically-induced cell lysis mentioned previously. In some cases, depending on the type of sample, nanoscale barbs, nanowires, acoustic generators, integrated lasers, integrated heaters, and/or microcapillary probes can be used to perform lysis.

In certain instances, heat inactivation can be performed on the sample. In some embodiments, a processed/lysed sample can undergo heat inactivation to inactivate, in the lysed sample, the proteins used during lysing (e.g. a PK enzyme or a lysing reagent). In some cases, a heating element integrated into the detection device can be used for heat-inactivation. The heating element can be powered by a battery or another source of thermal or electric energy that is integrated with the detection device.

In some cases, a target nucleic acid within the sample can undergo amplification before binding to a guide nucleic acid, for example a crRNA of a CRISPR enzyme. The target nucleic acid within a purified sample can be amplified. In some instances, amplification can be accomplished using loop mediated amplification (LAMP), isothermal recombinase polymerase amplification (RPA), and/or polymerase chain reaction (PCR).

In some instances, digital droplet amplification can used. Droplet digitization or droplet generation can comprise splitting a volume of the sample into multiple droplets, aliquots, or subsamples. The sample can have a volume that ranges from about 10 microliters to about 500 microliters. The plurality of droplets, aliquots, or subsamples can have a volume that ranges from about 0.01 microliters to about 100 microliters. The plurality of droplets, aliquots, or subsamples can have a same or substantially similar volume. In some cases, the plurality of droplets, aliquots, or subsamples can have different volumes. In some cases, the droplets, aliquots, or subsamples can be generated using a physical filter or the one or more movable mechanisms described above. In some cases, each droplet of the sample can undergo one or more sample preparation steps (e.g., nucleic acid purification, lysis, heat inactivation, amplification, etc.) independently and/or in parallel while the droplets are physically constrained or thermally isolated. Such nucleic acid amplification of the sample can improve at least one of a sensitivity, specificity, or accuracy of the detection of the target nucleic acid.

The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification is performed for from 5 to 60, from 10 to 60, from 15 to 60, from 30 to 60, from 45 to 60, from 1 to 45, from 5 to 45, from 10 to 45, from 30 to 45, from 1 to 30, from 5 to 30, from 10 to 30, from 15 to 30, from 1 to 15, from 5 to 15, or from 10 to 15 minutes.

In some embodiments, amplification can comprise thermocycling of the sample. Thermocycling can be carried out in a vessel, for one or more droplets of the sample in parallel, and/or independently in separate locations. This can be accomplished by methods such as (1) by holding droplets stationary in locations where a heating element is in close proximity to the droplet on one of the droplet sides and a heat sink element is in close proximity to the other side of the droplet, or (2) flowing the droplet through zones in a fluid channel where heat flows across it from a heating source to a heat sink. In some cases, one or more resistive heating elements can be used to perform thermocycling. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C., 50° C. 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C. In some cases, the nucleic acid amplification reaction is performed at a temperature of from 20° C. to 45° C., from 25° C. to 40° C., from 30° C. to 40° C., or from 35° C. to 40° C. In some cases, the nucleic acid amplification reaction is performed at a temperature of from 45° C. to 65° C., from 50° C. to 65° C., from 55° C. to 65° C., or from 60° C. to 65° C. In some cases, the nucleic acid amplification reaction can be performed at a temperature that ranges from about 20° C. to 45° C., from 25° C. to 45° C., from 30° C. to 45° C. from 35° C. to 45° C., from 40° C. to 45° C., from 20° C. to 37° C., from 25° C. to 37° C., from 30° C. to 37° C., from 35° C. to 37° C., from 20° C. to 30° C., from 25° C. to 30° C., from 20° C. to 25° C., or from about 22° C. to 25° C. In some cases, the nucleic acid amplification reaction can be performed at a temperature that ranges from about 40° C. to 65° C. from 45° C. to 65° C., from 50° C. to 65° C., from 55° C. to 65° C., from 60° C. to 65° C., from 40)° C. to 60° C., from 45° C. to 60° C., from 50° C. to 60° C., from 55° C. to 60° C., from 40° C. to 55° C., from 45° C. to 55° C., from 50° C. to 55° C., from 40° C. to 50° C., or from about 45° C. to 50° C.)

In some embodiments, thermocycling comprises a plurality of cycles, wherein each cycle comprises denaturation at a first temperature and primer extension by a polymerase at a second temperature that is lower than the first temperature. In some embodiments, each cycle is about or less than about 20 seconds in duration (e.g., about or less than about 15, 14, 13, 12, 11, 10, 9, 8, 7, 6, 5, or 4 seconds in duration). In some embodiments, each cycle is less than 15 seconds in duration. In some embodiments, each cycle is less than 10 seconds in duration. In some embodiments, the plurality of cycles are about 2 seconds to about 20 seconds in duration, about 3 seconds to about 10 seconds in duration, or about 5 seconds in duration. In some embodiments, the cycles are about 4 seconds in duration. In some embodiments, the denaturation step is about 0.5 to about 5 seconds in duration, about 1 to about 3 seconds in duration, or about 2 seconds in duration. In some embodiments, the denaturation is about 1 second in duration. In some embodiments, the first temperature is about 90° C. to about 99° C., about 94° C. to about 98° C., or about 95° C. In some embodiments, the first temperature is about 98° C. In some embodiments, the primer extension step is about 1 to about 15 seconds in duration, about 2 to about 10 seconds in duration, or about 5 seconds in duration. In some embodiments, the primer extension step is about 4 seconds in duration. In some embodiments, the second temperature is about 45° C. to about 75° C. about 50° C. to about 70° C. or about 55° C. to about 65° C. In some embodiments, the second temperature is about 55° C. In some embodiments, each cycle comprises denaturation at the first temperature for about 1 second and primer extension at the second temperature for about 3 seconds. In some embodiments, the plurality of cycles comprises about or at least about 20 cycles (e.g., about or more than about 25, 30, 35, 40, or 45 cycles). In some embodiments, the plurality of cycles comprises about 20 cycles to about 50 cycles, or about 30 cycles to about 45 cycles. In some embodiments, the plurality of cycles comprises about 45 cycles. In some embodiments, the plurality of cycles is preceded by an initial denaturation step at the first temperature that is longer in duration that the durations of the individual denaturation steps in each of the cycles. In some embodiments, the initial denaturation step is about 10 seconds to about 120 seconds in duration, about 15 seconds to about 60 seconds in duration, or about 20 seconds to about 50 seconds in duration. In some embodiments, the initial denaturation step is about 30 seconds in duration. In some embodiments, the total duration of the amplification by thermocycling is about 1 minute to about 20) minutes, about 2 minutes to about 15 minutes, or about 3 minutes to about 10 minutes. In some embodiments, the total duration of the amplification by thermocycling is less than about 10 minutes. In some embodiments, the total duration of the amplification by thermocycling is about 5 minutes.

In some non-limiting embodiments, the target nucleic acid(s) can optionally be amplified before binding to the guide nucleic acid (e.g., crRNA) of the programmable nuclease (e.g., CRISPR enzyme). This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C. 25° C. 30° C. 35° C. 37° C. 40° C. 45° C. 50° C. 55° C. 60° C. or 65° C. The nucleic acid amplification reaction can be performed at a temperature of at least 20° C. 25° C. 30° C. 35° C. 37° C. 40° C. 45° C. 50° C. 55° C. 60° C. or 65° C.

### Nucleic Acids

The nucleic acids described and referred to herein (including, for instance, guide nucleic acids, reporter nucleic acids, and/or target nucleic acids) can comprise a plurality of base pairs. A base pair can be a biological unit comprising two nucleobases bound to each other by hydrogen bonds. Nucleobases can comprise adenine, guanine, cytosine, thymine, and/or uracil. In some cases, the nucleic acids described and referred to herein can comprise different base pairs. In some cases, the nucleic acids described and referred to herein can comprise one or more modified base pairs. The one or more modified base pairs can be produced when one or more base pairs undergo a chemical modification leading to new bases. The one or more modified base pairs can be, for example, Hypoxanthine. Inosine, Xanthine, Xanthosine, 7-Methylguanine, 7-Methylguanosine, 5,6-Dihydrouracil, Dihydrouridine, 5-Methylcytosine, 5-Methylcytidine, 5-hydroxymethylcytosine (5hmC), 5-formylcytosine (5fC), or 5-carboxylcytosine (5caC).

In some instances, the target nucleic acid is a single stranded nucleic acid, Alternatively, or in combination, the target nucleic acid is a double stranded nucleic acid and is prepared into single stranded nucleic acids before or upon contacting the reagents. The target nucleic acid can be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the systems and methods disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample has from 1 to 10,000, from 100 to 8000, from 400 to 6000, from 500 to 5000, from 1000 to 4000, or from 2000 to 3000 target nucleic acids. In some cases, the sample has from 100 to 9500, from 100 to 9000, from 100 to 8500, from 100 to 8000, from 100 to 7500, from 100 to 7000, from 100 to 6500, from 100 to 6000, from 100 to 5500, from 100 to 5000, from 250 to 9500, from 250 to 9000, from 250 to 8500, from 250 to 8000, from 250 to 7500, from 250 to 7000, from 250 to 6500, from 250 to 6000, from 250 to 5500, from 250 to 5000, from 2500 to 9500, from 2500 to 9000, from 2500 to 8500, from 2500 to 8000, from 2500 to 7500, from 2500 to 7000, from 2500 to 6500, from 2500 to 6000, from 2500 to 5500, or from 2500 to 5000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the systems and methods disclosed herein. Some methods described herein can be implemented to detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the sample has from 2 to 50, from 5 to 50, from 10 to 50, from 2 to 25, from 3 to 25, from 4 to 25, from 5 to 25, from 10 to 25, from 2 to 20, from 3 to 20, from 4 to 20, from 5 to 20, from 10 to 20, from 2 to 10, from 3 to 10, from 4 to 10, from 5 to 10, from 6 to 10, from 7 to 10, from 8 to 10, or from 9 to 10 target nucleic acid populations. In some cases, the methods of the present disclosure can be implemented to detect target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

### Programmable Nuclease Probes

In some cases, the systems and methods of the present disclosure may be implemented using one or more programmable nuclease probes. The one or more programmable nuclease probes may be used to detect one or more targets in one or more samples. The one or more targets and the one or more samples may comprise any target or sample described above. In some cases, the one or more programmable nuclease probes may be placed in a detection chamber of a device or apparatus. In some cases, the one or more programmable nuclease probes may be immobilized to a surface of a device or an apparatus. In other cases, the one or more programmable nuclease probes may not or need not be immobilized to a surface of a device or an apparatus.

The programmable nuclease probe can comprise a programmable nuclease and/or a nucleic acid as described elsewhere herein. The nucleic acid may be complexed to the programmable nuclease. The nucleic acid may be a guide nucleic acid. The guide nucleic acid can bind to a target. In some case, to minimize off-target binding (which can slow down detection or inhibit accurate detection), an electro-potential gradient or thermal energy may be provided to one or more regions proximal to the programmable nuclease, to enhance targeting.

In some embodiments, the programmable nuclease probe can comprise a guide nucleic acid that is complexed with or capable of being complexed with a programmable nuclease. The programmable nuclease can comprise any type of programmable nuclease as described herein. In some cases, the programmable nuclease probe may comprise a guide nucleic acid complexed with an enzyme. The enzyme may be, in some instances, a CRISPR enzyme.

The programmable nuclease probe can comprise a programmable nuclease and/or a guide nucleic acid. The guide nucleic acid can bind to a target nucleic acid, as described in greater detail below. In some case, to minimize off-target binding (which can slow down detection or inhibit accurate detection), the device can be configured to generate an electro-potential gradient or to provide heat energy to one or more regions proximal to the programmable nuclease probe, to enhance targeting precision/accuracy.

The guide nucleic acid-enzyme complex may include, in some cases, a reporter. When one or more targets bind to the programmable nuclease probe (e.g., a CRISPR probe), the binding event can trigger a trans-cut that (i) releases the reporter into a detectable region or (ii) changes or modifies (e.g., physically or chemically) the reporter. Detection mechanisms can involve interferometry, surface plasmon resonance, electrochemical detection such as potentiometry, or other detection mechanisms.

In certain instances, the reporter of the programmable nuclease probe can initiate a signal amplification reaction with another molecular species after the complementary binding induced trans-cutting. Such species can be a reactive solid or gel matrix, or other reactive entity to generate an amplified signal during detection. The signal amplification reaction can be physical or chemical in nature. In some embodiments, after a complementary binding induced trans-cut, the released reporter, X, can initiate an interaction and/or a reaction with another entity, Y to produce an amplified or modified signal. Such entities can comprise a molecular species, a solid, a gel, or other entities. The signal amplification interaction can be a physical or chemical reaction. In some embodiments, the interaction involves free-radical, anionic, cationic or coordination polymerization reactions. In other embodiments the cut reporter can trigger aggregation, or agglutination, of molecules, cells, or nanoparticles. In some instances, the cut reporter can interact with a semiconductor material. In some embodiments, the chemical or physical change caused by the interaction is detected by optical detection means such as interferometry, surface plasmon resonance, reflectivity or other. In other embodiments, the chemical or physical change caused by the interaction is detected by potentiometric, amperometric, field effect transistor, or other electronic means.

As discussed above, the programmable nuclease probe can comprise a programmable nuclease probe that comprises a guide nucleic acid complexed with a programmable nuclease. The programmable nuclease can comprise any type of programmable nuclease as described herein. In some cases, the programmable nuclease probe comprises a guide nucleic acid complexed with a CRISPR enzyme. The guide RNA-CRISPR enzyme complex can also include a reporter.

The plurality of programmable nuclease probes as described herein can be arranged in various configurations. For example, the plurality of programmable nuclease probes can be arranged in a lateral configuration. Alternatively, the plurality of programmable nuclease probes can be arranged in a circular configuration such that each programmable nuclease probe is equidistant from a common center point. In some cases, the plurality of programmable nuclease probes can be distributed with a same separation distance or spacing between the programmable nuclease probes. In other cases, a first programmable nuclease probe and a second programmable nuclease probe can be separated by a first separation distance or spacing, and a third programmable nuclease probe and a fourth programmable nuclease probe can be separated by a second separation distance or spacing that is different than the first separation distance or spacing.

### Probe Immobilization

In some embodiments, target nucleic acid amplicons can be detected by immobilized programmable nuclease probes, such as, for example, programmable nuclease guide nucleic acid probes (e.g., a CRISPR probe). Upon a complementary binding event between a target nucleic acid amplicon and a programmable nuclease probe (e.g., an immobilized programmable nuclease/guide nucleic acid complex) a cutting event can occur that releases a reporter or generates a signal that is then detected by a sensor.

In some embodiments, the guide nucleic acid of the programmable nuclease or CRISPR probe can be immobilized adjacent to a bottom surface of the chamber. When a complementary interaction between the probe and the target occurs, the CRISPR enzyme will cut and release a reporter molecule which will then be sensed or detected by a sensor/detector. Since the specific guide RNA of the immobilized programmable nuclease or CRISPR probe can be spatially registered, multiplexed detection can be achieved. In some cases, where one sensor corresponds to one immobilized probe, electrical detection can be used. Other methods of detection can also be used, such as optical imaging, surface plasmon resonance (SPR), and/or interferometric sensing.

In some embodiments, the programmable nuclease probe (e.g., a CRISPR probe) can be immobilized to an immobilization matrix. In some cases, the interior side of the immobilization matrix may be exposed to an inside wall of a circulation chamber of a detection system, device or apparatus. The guide nucleic acid or guide RNA can be exposed to target amplicons inside the circulation chamber. The reporter can be in proximity to or oriented towards an “exterior” side of the immobilization matrix. The exterior side of the immobilization matrix can be in proximity to a detection region. The detection region may correspond to a region from which a detectable signal can originate. The detectable signal may indicate the presence or the absence of one or more targets of interest. The binding event between the guide nucleic acid and a target nucleic acid can trigger a trans-cut that (i) releases the reporter into a detectable region or (ii) physically or chemically changes the reporter. Detection mechanisms for detecting the reporter or any detectable signals generated by the reporter can involve, for instance, interferometry, surface plasmon resonance, electrochemical detection such as potentiometry, or other detection mechanisms.

In some embodiments, the reporter of the programmable nuclease probe can initiate a signal amplification reaction with another molecular species after the complementary binding induced trans-cutting. Such species can be a reactive solid or gel matrix, or other reactive entity to generate an amplified signal during detection. The signal amplification reaction can be physical or chemical in nature. In certain instances, after a complementary binding induced trans-cut, the released reporter, can initiate an interaction and/or a reaction with another entity, Y to produce an amplified or modified signal. Such entities can comprise a molecular species, a solid, a gel, or other entities. The signal amplification interaction can be a physical or chemical reaction. In some embodiments, the interaction involves free-radical, anionic, cationic or coordination polymerization reactions. In other embodiments the cut reporter can trigger aggregation, or agglutination, of molecules, cells or nanoparticles. In some instances, the cut reporter can interact with a semiconductor material. In some embodiments the chemical or physical change caused by the interaction is detected by optical detection means such as interferometry, surface plasmon resonance, reflectivity or other. In other embodiments the chemical or physical change caused by the interaction is detected by potentiometric, amperometric, field effect transistor, or other electronic means.

In some embodiments, the programmable nuclease, guide nucleic acid, reporter, or a combination thereof can be immobilized to a device surface (e.g., by a linkage). In some embodiments, the linkage comprises a covalent bond, a non-covalent bond, an electrostatic bond, an interaction (e.g., a covalent or noncovalent bond) between members of a binding pair (e.g., streptavidin and biotin), an amide bond, or any combination thereof. In embodiments where more than one element is immobilized to the surface (e.g., reporters and guide nucleic acid, programmable nuclease and reporters, or all three), the linkage may be the same or different for each species. For example, the guide nucleic acid may be immobilized to the surface by a single-stranded linker polynucleotide, and the reporters may be immobilized by the interaction between a first member of a binding pair on the reporters and a second member of a binding pair on the surface. In general, the term “binding pair” refers to a first and a second moiety that have a specific binding affinity for each other. In some embodiments, a binding pair has a dissociation constant Kd of less than or equal to about: 10⁻⁸ mol/L, 10⁻⁹ mol/L, 10⁻¹⁰ mol/L, 10⁻¹¹ mol/L, 10⁻¹² mol/L, 10⁻¹³ mol/L, 10⁻¹⁴ mol/L, 10⁻¹⁵ mol/L, or ranges including two of these values as endpoints. Non limiting examples of binding pairs include an antibody or an antigen-binding portion thereof and an antigen (e.g., fluorescein, digoxin, digoxigenin); a biotin (bio) moiety and an avidin (or streptavidin) moiety; a dinitrophenol (DNP) and an anti-DNP antibody: a hapten and an anti hapten; folate and a folate binding protein; vitamin B12 and an intrinsic factor; a carbohydrate and a lectin or carbohydrate receptor; a polysaccharide and a polysaccharide binding moiety; a lectin and a receptor; a ligand and a receptor; a drug and a drug receptor; complementary chemical reactive groups (e.g., sulfhydryl/maleimide. thiol/maleimide, sulfhydryl/haloacetyl derivative, amine/epoxy, amine/isotriocyanate, amine/succinimidyl ester, and amine/sulfonyl halides); an antibody (e.g., IgG) and protein A or protein G; a toxin and a toxin receptor; and an enzyme substrate and an enzyme. In some embodiments, the binding pair comprises biotin and either of avidin or streptavidin.

In some embodiments, the linkage utilizes non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface.

In some embodiments, target nucleic acid amplicons are detected by immobilized programmable nuclease probes, such as, for example, CRISPR CAS guide RNA probes (referred to as CRISPR probe). Upon a complementary binding event between a target nucleic acid amplicon and a programmable nuclease probe (e.g., an immobilized CRISPR CAS/guide RNA complex) a cutting event will occur that release a reporter that is then detected by a sensor.

### Guide Nucleic Acid

In some embodiments, one or more guide nucleic acids can be used to carry out highly efficient, rapid, and accurate reactions for detecting whether a target nucleic acid is present in a sample. The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid binds to the single stranded target nucleic acid comprising a portion of a nucleic acid from a cancer gene or gene associated with a genetic disorder as described herein. The guide nucleic acid is complementary to the target nucleic acid. Often the guide nucleic acid binds specifically to the target nucleic acid. The target nucleic acid can be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid comprises a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid can have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40) nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40) nt, from about 19 nt to about 45 nt, from about 19 nt to about 50 nt, from about 19 nt to about 60 nt, from about 20 nt to about 25 nt, from about 20) nt to about 30 nt, from about 20) nt to about 35 nt, from about 20 nt to about 40) nt, from about 20 nt to about 45 nt, from about 20 nt to about 50 nt, or from about 20 nt to about 60 nt. In some cases, the targeting region of a guide nucleic acid has a length of from about 10 nt to about 60 nt, from about 20 nt to about 50 nt, or from about 30) nt to about 40) nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20) that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid of a strain of an infection or genomic locus of interest. The guide nucleic acid can be selected from a group of guide nucleic acids that have been tiled against the nucleic acid of a strain of HPV 16 or HPV18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances, the tiling of the guide nucleic acids is non-sequential. Often, a method for detecting a target nucleic acid comprises contacting a target nucleic acid to a pool of guide nucleic acids and a programmable nuclease, wherein a guide nucleic acid of the pool of guide nucleic acids has a sequence selected from a group of tiled guide nucleic acid that is reverse complementary to a sequence of a target nucleic acid; and assaying for a signal produce by cleavage of at least some reporters (e.g., detector nucleic acids) of a population of reporters. Pooling of guide nucleic acids can ensure broad spectrum identification, or broad coverage, of a target species within a single reaction. This can be particularly helpful in diseases or indications, like sepsis, that can be caused by multiple organisms.

Guide nucleic acids may be used in conjunction with compositions disclosed herein (e.g., programmable nucleases, reporters, reagents, or any combination thereof) to carry out highly efficient, rapid, and accurate reactions for detecting whether a target (e.g., a nucleic acid) is present in a sample. The guide nucleic acid can bind to a single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), which can confer resistance to a treatment, such as antibiotic treatment. The guide nucleic acid can bind to a single stranded target nucleic acid comprising a portion of a nucleic acid from a cancer gene or gene associated with a genetic disorder as described herein. The guide nucleic acid can be complementary to one or more target nucleic acids. Often the guide nucleic acid binds specifically to the target nucleic acid. The target nucleic acid may be a RNA, DNA, or synthetic nucleic acids. A guide nucleic acid can comprise a sequence that is reverse complementary to the sequence of a target nucleic acid. A guide nucleic acid can be a crRNA. Sometimes, a guide nucleic acid may comprise a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50) nt, from about 12 nt to about 45 nt, from about 12 nt to about 40 nt, from about 12 nt to about 35 nt, from about 12 nt to about 30) nt, from about 12 nt to about 25 nt, from about 12 nt to about 20) nt, from about 12 nt to about 19 nt, from about 19 nt to about 20 nt, from about 19 nt to about 25 nt, from about 19 nt to about 30) nt, from about 19 nt to about 35 nt, from about 19 nt to about 40) nt, from about 19 nt to about 45 nt, from about 19 nt to about 50) nt, from about 19 nt to about 60) nt, from about 20 nt to about 25 nt, from about 20 nt to about 30 nt, from about 20 nt to about 35 nt, from about 20) nt to about 40) nt, from about 20 nt to about 45 nt, from about 20 nt to about 50) nt, or from about 20 nt to about 60 nt. In some cases, the targeting region of a guide nucleic acid has a length of from about 10 nt to about 60 nt, from about 20 nt to about 50 nt, or from about 30) nt to about 40) nt. In some cases, the targeting region of a guide nucleic acid has a length of from 15 nt to 55 nt, from 25 nt to 55 nt, from 35 nt to 55 nt, from 45 nt to 55 nt, from 15 nt to 45 nt, from 25 nt to 45 nt, from 35 nt to 45 nt, from 15 nt to 35 nt, from 25 nt to 35 nt, or from 15 nt to 25 nt. It is understood that the sequence of a polynucleotide need not be 100% complementary to that of its target nucleic acid to be specifically hybridizable or hybridizable, or to bind specifically.

The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20) that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

As discussed herein, a guide nucleic acid may be complexed with a programmable nuclease in order to carry out highly efficient, rapid, and accurate reactions for detecting whether a target nucleic acid is present in a sample. The programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. A signal can be a calorimetric, potentiometric, amperometric, optical (e.g., fluorescent, colorimetric, etc.), or piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a CRISPR-Cas (clustered regularly interspaced short palindromic repeats-CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid comprises a crRNA and a trans-activating crRNA (tracrRNA).

In some embodiments, the programmable nuclease used to detect modified target nucleic acids can comprise CRISPR RNAS (crRNAs), trans-activating crRNAs (tracrRNAs). Cas proteins, and reporters. The programmable nuclease may comprise any of the programmable nucleases described or referenced elsewhere herein.

In another aspect, the present disclosure provides reagents comprising a programmable nuclease capable of being activated when complexed with the guide nucleic acid and the target nucleic acid segment or portion. A programmable nuclease can be capable of being activated when complexed with a guide nucleic acid and the target sequence. The programmable nuclease can be activated upon binding of the guide nucleic acid to its target nucleic acid and degrades non-specifically nucleic acid in its environment. The programmable nuclease has trans cleavage activity once activated. A programmable nuclease can be a Cas protein (also referred to, interchangeably, as a Cas nuclease), A crRNA and Cas protein can form a CRISPR enzyme.

Several programmable nucleases are consistent with the methods and devices of the present disclosure. For example, CRISPR/Cas enzymes are programmable nucleases used in the methods and systems disclosed herein, CRISPR/Cas enzymes can include any of the known Classes and Types of CRISPR/Cas enzymes. Programmable nucleases disclosed herein include Class 1 CRISPR/Cas enzymes, such as the Type I, Type IV, or Type III CRISPR/Cas enzymes. Programmable nucleases disclosed herein also include the Class 2 CRISPR/Cas enzymes, such as the Type II, Type V, and Type VI CRISPR/Cas enzymes. Preferable programmable nucleases included in the several devices disclosed herein (e.g., a microfluidic device such as a pneumatic valve device or a sliding valve device or a lateral flow assay) and methods of use thereof include a Type V or Type VI CRISPR/Cas enzyme.

In some embodiments, the Type V CRISPR/Cas enzyme is a programmable Cas12 nuclease. Type V CRISPR/Cas enzymes (e.g., Cas12 or Cas14) lack an HNH domain, A Cas12 nuclease of the present disclosure cleaves a nucleic acid via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. In some instances, a programmable Cas12 nuclease can be a Cas12a (also referred to as Cpf1) protein, a Cas12b protein, Cas12c protein, Cas12d protein, or a Cas12e protein.

In some embodiments, the programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the Cas12 can be a Cas12 variant (SEQ ID NO: 17), which is a specific protein variant within the Cas12 protein family/classification) In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. Sometimes the programmable nuclease can be an engineered nuclease that is not from a naturally occurring CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp, (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp, (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp, (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HhcCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter and is capable of being activated by a target DNA to initiate trans cleavage of an RNA reporter, such as a Type VI CRISPR/Cas enzyme (e.g., a Cas13 nuclease). For example, Cas13a of the present disclosure can be activated by a target RNA to initiate trans cleavage activity of the Cas13a for the cleavage of an RNA reporter and can be activated by a target DNA to initiate trans cleavage activity of the Cas13a for trans cleavage of an RNA reporter. An RNA reporter can be an RNA-based reporter molecule. In some embodiments, the Cas13a recognizes and detects ssDNA to initiate transcleavage of RNA reporters. Multiple Cas13a isolates can recognize, be activated by, and detect target DNA, including ssDNA, upon hybridization of a guide nucleic acid with the target DNA. For example, Lbu-Cas13a and Lwa-Cas13a can both be activated to transcollaterally cleave RNA reporters by target DNA. Thus, Type VI CRISPR/Cas enzyme (e.g., a Cas13 nuclease, such as Cas13a) can be DNA-activated programmable RNA nucleases, and therefore can be used to detect a target DNA using the methods as described herein, DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 can exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA can not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly taken from organisms or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection.

The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example, target ssDNA detection by Cas13a can be employed in a detection device as disclosed herein.

In any of the embodiments described herein, the programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid, which can initiate trans cleavage activity. In some cases, the trans cut or trans cleavage can cut and/or release a reporter molecule. In other cases, the trans cut or trans cleavage can produce an analog of a target, which can be directly detected. Trans cleavage activity can be non-specific cleavage of nearby nucleic acids by the activated programmable nuclease, such as trans cleavage of reporters comprising a cleavable nucleic acid and a detection moiety. Once the nucleic acid of the reporter is cleaved by the activated programmable nuclease, the detection moiety can be released from the reporter and can generate a signal. The detection moiety can be immobilized on a support medium for detection. The signal can be visualized to assess whether a target nucleic acid is present or absent.

### Reporters

Reporters, which can be referred to interchangeably as reporter molecules, or detector molecules (e.g., detector nucleic acids), can be used in conjunction with the compositions disclosed herein (e.g., programmable nucleases, guide nucleic acids, etc.) to carry out highly efficient, rapid, and accurate reactions for detecting whether a target nucleic acid is present in a sample. The reporter can be suspended in solution or immobilized on a surface. For example, the reporter can be immobilized on the surface of a chamber in a device as disclosed herein. In some cases, the reporter can be immobilized on beads, such as magnetic beads, in a chamber of a device as disclosed herein where they are held in position by a magnet placed below the chamber. The reporter can capable of being cleaved by the activated programmable nuclease, thereby generating a detectable signal. The detectable signal can correspond to a release of one or more elements (X). The release of the one or more elements (X) can initiate a reaction with another element (Y) when the element (Y) is in the presence of the element (X). The reaction between the element (Y) and the element (X) can initiate a chemical chain reaction in a solid phase material. Such a chemical chain reaction can produce one or more physical or chemical changes. In some cases, the physical or chemical changes can be optically detected. In some embodiments, one or more cascade amplification reactions can occur to further amplify the signal before sensing or detection. There can be a single point of attachment between the reporter molecule and the element (X). Cutting the single point of attachment can release a macro molecule (X), which can undergo a series of reactions based on the macro molecule (X) itself. In any of the embodiments described herein, the reporter can comprise a single stranded detector nucleic acid comprising a detection moiety.

The reporters described herein can be, for example, RNA reporters. The RNA reporters can comprise at least one ribonucleic acid and a detectable moiety. In some embodiments, a programmable nuclease probe or a CRISPR probe comprising a Cas enzyme can recognize and detect ssDNA and, further, can specifically trans-cleave RNA reporters. The detection of the target nucleic acid in the sample can indicate the presence of the disease (or disease-causing agent) in the sample and can provide information for taking action to reduce the transmission of the disease to individuals in the disease-affected environment or near the disease-carrying individual.

As described elsewhere herein, cleavage of a reporter (e.g., a protein-nucleic acid) can produce a signal. The signal can indicate a presence of the target nucleic acid in the sample, and an absence of the signal can indicate an absence of the target nucleic acid in the sample. In some cases, cleavage of the protein-nucleic acid can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

In some cases, the reporter may comprise a single-stranded nucleic acid comprising deoxyribonucleotides. In other cases, the reporter may comprise a single-stranded nucleic acid comprising ribonucleotides. The reporter can be a single-stranded nucleic acid comprising at least one deoxyribonucleotide and at least one ribonucleotide. In some cases, the reporter is a single-stranded nucleic acid comprising at least one ribonucleotide residue at an internal position that functions as a cleavage site. In some cases, the reporter comprises at least 2, 3, 4, 5, 6, 7, 8, 9, or 10 ribonucleotide residues at an internal position. In some cases, the reporter comprises from 2 to 10, from 3 to 9, from 4 to 8, or from 5 to 7 ribonucleotide residues at an internal position. In some cases, the reporter comprises from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the reporter has only ribonucleotide residues. In some cases, the reporter has only deoxyribonucleotide residues. In some cases, the reporter comprises nucleotides resistant to cleavage by the programmable nuclease described herein. In some cases, the reporter comprises synthetic nucleotides. In some cases, the reporter comprises at least one ribonucleotide residue and at least one non-ribonucleotide residue. In some cases, the reporter is 5-20, 5-15, 5-10, 7-20, 7-15, or 7-10 nucleotides in length. In some cases, the reporter is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the reporter comprises at least one uracil ribonucleotide. In some cases, the reporter comprises at least two uracil ribonucleotides. Sometimes the reporter has only uracil ribonucleotides. In some cases, the reporter comprises at least one adenine ribonucleotide. In some cases, the reporter comprises at least two adenine ribonucleotides. In some cases, the reporter has only adenine ribonucleotides. In some cases, the reporter comprises at least one cytosine ribonucleotide. In some cases, the reporter comprises at least two cytosine ribonucleotides. In some cases, the reporter comprises at least one guanine ribonucleotide. In some cases, the reporter comprises at least two guanine ribonucleotides. A reporter can comprise only unmodified ribonucleotides, only unmodified deoxyribonucleotides, or a combination thereof. In some cases, the reporter is from 5 to 12 nucleotides in length. In some cases, the reporter is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the reporter is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a reporter nucleic acid can be 5, 8, or 10 nucleotides in length. For cleavage by a programmable nuclease comprising Cas12, a reporter nucleic acid can be 10 nucleotides in length.

The single stranded reporter nucleic acid can comprise a detection moiety capable of generating a first detectable signal. Sometimes the reporter comprises a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the reporter. Sometimes the detection moiety is at the 3′ terminus of the reporter. In some cases, the detection moiety is at the 5′ terminus of the reporter. In some cases, the quenching moiety is at the 3′ terminus of the reporter. In some cases, the single-stranded reporter nucleic acid is at least one population of the single-stranded nucleic acid capable of generating a first detectable signal. In some cases, the single-stranded reporter nucleic acid is a population of the single stranded nucleic acid capable of generating a first detectable signal. Optionally, there can be more than one population of single-stranded reporter nucleic acids. In some cases, there can be 2, 3, 4, 5, 6, 7, 8, 9, 10, 11, 12, 13, 14, 15, 20, 30, 40, 50, or greater than 50, or any number spanned by the range of this list of different populations of single-stranded reporter nucleic acids capable of generating a detectable signal. In some cases, there are from 2 to 50, from 3 to 40, from 4 to 30, from 5 to 20, or from 6 to 10 different populations of single-stranded reporter nucleic acids capable of generating a detectable signal. In some cases there are from 2 to 50, from 5 to 50, from 10 to 50, from 15 to 50, from 20 to 50, from 25 to 50, from 30 to 50, from 35 to 50, from 40 to 50, from 2 to 40, from 5 to 40, from 10 to 40, from 15 to 40, from 20 to 40, from 25 to 40, from 30 to 40, from 35 to 40, from 2 to 30, from 5 to 30, from 10 to 30, from 15 to 30, from 20 to 30, from 25 to 30, from 2 to 20, from 5 to 20, from 10 to 20, from 15 to 20, from 2 to 10, or from 5 to 10 different populations of single-stranded reporter nucleic acids capable of generating a detectable signal.

In some embodiments, the reporter may comprise a single stranded detector nucleic acid comprising a detection moiety. The reporter can be cleaved by an activated programmable nuclease, thereby generating a first detectable signal. In some cases, the reporter is a single-stranded nucleic acid comprising deoxyribonucleotides. In other cases, the reporter is a single-stranded nucleic acid comprising ribonucleotides. The reporter can be a single-stranded nucleic acid comprising at least one deoxyribonucleotide and at least one ribonucleotide. In some cases, the reporter is a single-stranded nucleic acid comprising at least one ribonucleotide residue at an internal position that functions as a cleavage site. In some cases, the reporter may comprise at least 2, 3, 4, 5, 6, 7, 8, 9, or 10 ribonucleotide residues at an internal position. In some cases, the reporter may comprise from 2 to 10, from 3 to 9, from 4 to 8, or from 5 to 7 ribonucleotide residues at an internal position. In some cases, the reporter may comprise from 3 to 10, from 4 to 10, from 5 to 10, from 6 to 10, from 7 to 10, from 8 to 10, from 9 to 10, from 2 to 8, from 3 to 8, from 5 to 8, from 6 to 8, from 7 to 8, from 2 to 5, from 3 to 5, or from 4 to 5 ribonucleotide residues at an internal position. Sometimes the ribonucleotide residues are continuous. Alternatively, the ribonucleotide residues are interspersed in between non-ribonucleotide residues. In some cases, the reporter has only ribonucleotide residues. In some cases, the reporter has only deoxyribonucleotide residues. In some cases, the reporter may comprise nucleotides resistant to cleavage by the programmable nuclease described herein. In some cases, the reporter may comprise synthetic nucleotides. In some cases, the reporter may comprise at least one ribonucleotide residue and at least one non-ribonucleotide residue. In some cases, the reporter is 5-20, 5-15, 5-10, 7-20, 7-15, or 7-10 nucleotides in length. In some cases, the reporter is from 3 to 20, from 4 to 20, from 5 to 20, from 6 to 20, from 7 to 20, from 8 to 20, from 9 to 20, from 10 to 20, from 15 to 20, from 3 to 15, from 4 to 15, from 5 to 15, from 6 to 15, from 7 to 15, from 8 to 15, from 9 to 15, from 10 to 15, from 3 to 10, from 4 to 10, from 5 to 10, from 6 to 10, from 7 to 10, from 8 to 10, from 9 to 10, from 3 to 8, from 4 to 8, from 5 to 8, from 6 to 8, or from 7 to 8 nucleotides in length. In some cases, the reporter may comprise at least one uracil ribonucleotide. In some cases, the reporter may comprise at least two uracil ribonucleotides. Sometimes the reporter has only uracil ribonucleotides. In some cases, the reporter may comprise at least one adenine ribonucleotide. In some cases, the reporter may comprise at least two adenine ribonucleotide. In some cases, the reporter has only adenine ribonucleotides. In some cases, the reporter may comprise at least one cytosine ribonucleotide. In some cases, the reporter may comprise at least two cytosine ribonucleotide. In some cases, the reporter may comprise at least one guanine ribonucleotide. In some cases, the reporter may comprise at least two guanine ribonucleotide. A reporter can comprise only unmodified ribonucleotides, only unmodified deoxyribonucleotides, or a combination thereof. In some cases, the reporter is from 5 to 12 nucleotides in length. In some cases, the reporter is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the reporter is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a reporter can be 5, 8, or 10 nucleotides in length. For cleavage by a programmable nuclease comprising Cas12, a reporter can be 10 nucleotides in length.

### Detectable Signal

As described elsewhere herein, the cleavage of the reporter and/or the release of a detection moiety may generate a detectable signal. The signal can be a colorimetric, potentiometric, amperometric, optical (e.g., fluorescent, colorometric, etc.), or piezo-electric signal. The generation of the detectable signal from the release of the detection moiety can indicate that cleavage by the programmable nuclease has occurred and that the sample contains one or more target nucleic acids. A detection moiety can be any moiety capable of generating a colorimetric, potentiometric, amperometric, optical (e.g., fluorescent, colorometric, etc.), or piezo-electric signal.

In some cases, the reporter can be a protein-nucleic acid that can generate a calorimetric, potentiometric, amperometric, optical (e.g., fluorescent, colorometric, etc.), or piezo-electric signal upon cleavage of the nucleic acid. Often a calorimetric signal is heat produced after cleavage of the reporter. Sometimes, a calorimetric signal is heat absorbed after cleavage of the reporter. A potentiometric signal, for example, is electrical potential produced after cleavage of the reporter. An amperometric signal can be movement of electrons produced after the cleavage of the reporter. Often, the signal is an optical signal, such as a colorometric signal or a fluorescence signal. An optical signal is, for example, a light output produced after the cleavage of the reporter. Sometimes, an optical signal is a change in light absorbance between before and after the cleavage of the reporter. Often, a piezo-electric signal is a change in mass between before and after the cleavage of the reporter.

Detecting the presence or absence of a target nucleic acid of interest can involve measuring a signal emitted from a detection moiety present in a reporter, after cleavage of the reporter by an activated programmable nuclease. The signal can be measured using one or more sensors integrated with the device or operatively coupled to the device. Thus, the detection methods disclosed herein can involve measuring the presence of a target nucleic acid, quantifying how much of the target nucleic acid is present, or, measuring a signal indicating that the target nucleic acid is absent in a sample. In some embodiments, a signal is generated upon cleavage of the reporter by the programmable nuclease. In other embodiments, the signal changes upon cleavage of the reporter by the programmable nuclease. In other embodiments, a signal can be present in the absence of reporter cleavage and disappear upon cleavage of the target nucleic acid by the programmable nuclease. For example, a signal can be produced in a microfluidic device or lateral flow device after contacting a sample with a composition comprising a programmable nuclease. The programmable nuclease may or may not be immobilized as described elsewhere herein.

In some cases, the signal can comprise a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system can detect more than one type of target nucleic acid, wherein the system may comprise more than one type of guide nucleic acid and more than one type of reporter. In some cases, the detectable signal is generated directly by the cleavage event, Alternatively, or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium.

In some cases, the one or more detectable signals generated after cleavage can produce an index of refraction change or one or more electrochemical changes. In some cases, real-time detection of targets can be achieved using fluorescence, electrochemical detection, and/or electrochemiluminescence.

In some cases, the detectable signals can be detected and analyzed in various ways. For example, the detectable signals can be detected using an imaging device. The imaging device can a digital camera, such a digital camera on a mobile device. The mobile device can have a software program or a mobile application that can capture fluorescence, ultraviolet (UV), infrared (IR), or visible wavelength signals. Any suitable detection or measurement device can be used to detect and/or analyze the colorimetric, fluorescence, amperometric, potentiometric, or electrochemical signals described herein. In some embodiments, the colorimetric, fluorescence, amperometric, potentiometric, or another electrochemical sign can be detected using a measurement device connected to a detection chamber of the device (e.g., a fluorescence measurement device, a spectrophotometer, and/or an oscilloscope.

The presently disclosed devices, systems, kits, and methods for detecting the presence of a target nucleic acid in a sample may be used to generate and detect signals indicative of the presence or absence of the target nucleic acid in the sample. The generation of a signal indicative of the presence or absence of the target nucleic acid in the sample can enable highly efficient, rapid, and accurate reactions for detecting whether a target nucleic acid is present in a sample.

As disclosed herein, in some embodiments, detecting the presence or absence of a target nucleic acid of interest involves measuring a signal emitted from a detection moiety present in a reporter, after cleavage of the reporter by an activated programmable nuclease, Alternatively, or in combination, in some embodiments, detecting the presence or absence of a target nucleic acid of interest involves measuring a signal emitted from a conjugate bound to a detection moiety present in a reporter, after cleavage of the reporter by an activated programmable nuclease. The conjugates may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety or another affinity molecule of the cleaved detector molecule as described herein. Thus, the detecting steps disclosed herein involve indirectly (e.g., via a reporter) measuring the presence of a target nucleic acid, quantifying how much of the target nucleic acid is present, or measuring a signal indicating that the target nucleic acid is absent in a sample. In some embodiments, a signal is generated upon cleavage of the reporter by the programmable nuclease. In other embodiments, the signal changes upon cleavage of the reporter by the programmable nuclease. In other embodiments, a signal may be present in the absence of reporter cleavage and disappear upon cleavage of the target nucleic acid by the programmable nuclease. For example, a signal may be produced in a microfluidic device or lateral flow device after contacting a sample with a composition comprising a programmable nuclease.

As described above, the reporter (e.g., a single stranded reporter) can comprise a detection moiety capable of generating a first detectable signal. Sometimes the reporter comprises a protein capable of generating a signal. A signal can be a colorimetric, potentiometric, amperometric, optical (e.g., fluorescent, colorometric, etc.), or piezo-electric signal. The generation of the detectable signal from the release of the detection moiety indicates that cleavage by the programmable nuclease has occurred and that the sample contains the target nucleic acid. A detection moiety can be any moiety capable of generating a colorimetric, potentiometric, amperometric, optical (e.g., fluorescent, colorometric, etc.), or piezo-electric signal. In some cases, the reporter can be a protein-nucleic acid that can generate a calorimetric, potentiometric, amperometric, optical (e.g., fluorescent, colorometric, etc.), or piezo-electric signal upon cleavage of the nucleic acid. Often a calorimetric signal is heat produced after cleavage of the reporters (e.g., detector nucleic acids). Sometimes, a calorimetric signal is heat absorbed after cleavage of the reporters. A potentiometric signal, for example, is electrical potential produced after cleavage of the reporters. An amperometric signal can be movement of electrons produced after the cleavage of reporter. Often, the signal is an optical signal, such as a colorometric signal or a fluorescence signal. An optical signal is, for example, a light output produced after the cleavage of the reporters. Sometimes, an optical signal is a change in light absorbance between before and after the cleavage of reporters. Often, a piezo-electric signal is a change in mass between before and after the cleavage of the reporter.

Detecting the presence or absence of a target nucleic acid of interest can involve measuring a signal emitted from a detection moiety present in a reporter, after cleavage of the reporter by an activated programmable nuclease. The signal can be measured using one or more sensors integrated with the device or operatively coupled to the device. Thus, the detecting steps disclosed herein can involve measuring the presence of a target nucleic acid, quantifying how much of the target nucleic acid is present, or, measuring a signal indicating that the target nucleic acid is absent in a sample. In some embodiments, a signal is generated upon cleavage of the reporter by the programmable nuclease. In other embodiments, the signal changes upon cleavage of the reporter by the programmable nuclease. In other embodiments, a signal can be present in the absence of reporter cleavage and disappear upon cleavage of the target nucleic acid by the programmable nuclease. For example, a signal can be produced in a microfluidic device or lateral flow device after contacting a sample with a composition comprising a programmable nuclease.

In some cases, the signal can comprise a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to a capture molecule in the detection region, where the first detection signal indicates that the sample contained the target nucleic acid. Sometimes the system can detect more than one type of target nucleic acid, wherein the system comprises more than one type of guide nucleic acid and more than one type of reporter. In some cases, the detectable signal is generated directly by the cleavage event, Alternatively, or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium.

In some cases, the one or more detectable signals generated after cleavage can produce an index of refraction change or one or more electrochemical changes. In some cases, real-time detection of the Cas reaction can be achieved using fluorescence, electrochemical detection, and/or electrochemiluminescence.

In some cases, the detectable signals can be detected and analyzed in various ways. For example, the detectable signals can be detected using an imaging device. The imaging device can a digital camera, such a digital camera on a mobile device. The mobile device can have a software program or a mobile application that can capture fluorescence, ultraviolet (UV), infrared (IR), or visible wavelength signals. Any suitable detection or measurement device can be used to detect and/or analyze the colorimetric, fluorescence, amperometric, potentiometric, or electrochemical signals described herein. In some embodiments, the colorimetric, fluorescence, amperometric, potentiometric, or another electrochemical sign can be detected using a measurement device connected to a detection chamber of the device (e.g., a fluorescence measurement device, a spectrophotometer, and/or an oscilloscope).

### Sensors

The detectable signals disclosed herein may be detected or registered using one or more sensors. The one or more sensors can be configured to detect one or more signals that are generated after one or more programmable nucleases of the one or more programmable nuclease probes become activated due to a binding of a guide nucleic acid of the programmable nuclease probes with a target nucleic acid present in the sample. As described elsewhere herein, the activated programmable nuclease can cleave the target nucleic acid, which can result in a trans cleavage activity. Trans cleavage activity can be a non-specific cleavage of nearby single-stranded nucleic acids by the activated programmable nuclease, such as trans cleavage of target nucleic acids or reporter nucleic acids with a detection moiety. Once the target nucleic acids or reporters are cleaved by the activated programmable nucleases, the detection moiety can be released or separated from the reporter, thereby generating one or more detectable signals. The one or more sensors can be configured to register and/or process the one or more detectable signals to confirm a presence and/or an absence of a particular target (e.g., a target nucleic acid,

### Multiplexing

In some embodiments, the systems, device, apparatuses, and methods of the present disclosures may be used to perform or facilitate multiplexing or multiplexed target detection. In certain aspects of this disclosure, multiplexing may refer to parallel sensing of multiple target nucleic acid sequences in one sample by multiple probes.

The present disclosure provides various multiplexing embodiments of a CRISPR-based detection device. In some cases, a capillary flow or mobile sample phase configuration may be used. In other cases, a stationary sample phase configuration may be used.

In some embodiments, a chamber that is in the form of a capillary circuit may be provided. Functionalized programmable nuclease probes, e.g., CRISPR probes, can be disposed on (or immobilized to) the capillary walls, and one or more guide nucleic acids associated with or complexed to the programmable nuclease probes, e.g., CRISPR probes can be exposed to the sample for binding. Upon binding to a complementary target nucleic acid amplicon (or a target nucleic acid sequence), the programmable nuclease probe or CRISPR probe can then cut and release at least a portion of a reporter, which may generate a signal indicating the presence of the particular target nucleic acid amplicon. This process can be repeated in parallel across multiple programmable nuclease probes or CRISPR probes, where each programmable nuclease or CRISPR probe is configured to detect a particular target sequence, nucleic acid amplicon, set of target sequences, or set of target nucleic acid amplicons.

In some aspects, multiplexed detection can also be achieved in a stationary phase, or microarray format. In some embodiments, programmable nuclease probes or CRISPR probes, each designed to detect certain target nucleic acid sequences, are immobilized in known locations. When a sample containing multiple types of target amplicons is exposed to the array of programmable nuclease or CRISPR probes, the specific probe-target pairs will bind and trigger signal events. These signal events can be associated with a particular target nucleic acid amplicon or a set of target nucleic acid amplicons either by its location (e.g., when imaging is used), or by a signal received by a particular sensor (e.g., when various sensors are individually linked to each probe). In some instances, one or more target nucleic acid amplicons can be detected by a programmable nuclease probe. In some instances, the programmable nuclease probe can interact with and/or detect a class of sequences or a class of target nucleic acid amplicons, which can indicate a presence or an absence of a particular organism, disease state, or phenotype present within the sample.

The devices of the present disclosure can be used for detection of one or more target nucleic acids within the sample. In some cases, the detection devices of the present disclosure can comprise one or more pumps, valves, reservoirs, and chambers for sample preparation, optional amplification of a target nucleic acid within the sample, mixing with a programmable nuclease, and detection of a detectable signal arising from cleavage of reporters by a programmable nuclease.

Methods consistent with the present disclosure can include a multiplexing method of assaying for a plurality of target nucleic acids in a sample. A multiplexing method may comprise contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some reporters (e.g., protein-nucleic acids) of a population of reporter molecules (e.g., protein-nucleic acids). In some cases, the signal can indicate a presence of the target nucleic acid in the sample and the absence of the signal can indicate an absence of the target nucleic acid in the sample.

In some embodiments, multiplexing can comprise spatial multiplexing wherein multiple different target nucleic acids are detected at the same time, but the reactions are spatially separated. In some cases, the multiple target nucleic acids are detected using the same programmable nuclease, but different guide nucleic acids. The multiple target nucleic acids sometimes can be detected using the different programmable nucleases. In the case wherein multiple target nucleic acids are detected using different programmable nucleases, the method can involve using a first programmable nuclease, which upon activation (e.g., after binding of a first guide nucleic acid to a first target), cleaves a nucleic acid of a first reporter, and using a second programmable nuclease, which upon activation (e.g., after binding of a second guide nucleic acid to a second target), cleaves a nucleic acid of a second reporter.

Sometimes, multiplexing can be single reaction multiplexing wherein multiple different target acids are detected in a single reaction volume. Often, at least two different programmable nucleases are used in single reaction multiplexing. For example, multiplexing may comprise assaying for a microorganism species using a first programmable nuclease and an antibiotic resistance pattern in a microorganism using a second programmable nuclease. In some cases, multiplexing can be enabled by immobilization of multiple categories of reporters within a fluidic system, to enable detection of multiple target nucleic acids within a single fluidic system. Multiplexing allows for detection of multiple target nucleic acids in one kit or system. In some cases, the multiple target nucleic acids comprise different target nucleic acids to a virus, a bacterium, or a pathogen responsible for one disease. In some cases, the multiple target nucleic acids comprise different target nucleic acids associated with a cancer or genetic disorder. Multiplexing for one disease, cancer, or genetic disorder increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. Multiplexing, thus, allows for multiplexed detection of multiple genomic alleles. Sometimes, multiplexing allows for discrimination between multiple target nucleic acids of different HPV strains, for example, HPV16 and HPV18. In some cases, the multiple target nucleic acids comprise target nucleic acids directed to different cancers or genetic disorders. Often, multiplexing allows for discrimination between multiple target nucleic acids, such as target nucleic acids that comprise different genotypes, for example, for a wild-type genotype and for SNP genotype. Multiplexing for multiple diseases, cancers, or genetic disorders provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

In any of the embodiments described herein, signals from multiplexing can be quantified. For example, a method of quantification for a disease panel may comprise assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of reporters compared to the signal produced in the second aliquot. In this context, a unique target nucleic acid refers to the sequence of a nucleic acid that has an at least one nucleotide difference from the sequences of the other nucleic acids in the plurality. Multiple copies of each target nucleic acid can be present. For example, a unique target nucleic population can comprise multiple copies of the unique target nucleic acid. Often the plurality of unique target nucleic acids is from a plurality of bacterial pathogens in the sample.

In some instances, the multiplexed devices, systems, fluidic devices, kits, and methods can be used to detect at least 2 different target nucleic acids in a single reaction. In some instances, the multiplexed devices, systems, fluidic devices, kits, and methods detect at least 3 different target nucleic acids in a single reaction. In some instances, the multiplexed devices, systems, fluidic devices, kits, and methods detect at least 4 different target nucleic acids in a single reaction. In some instances, the multiplexed devices, systems, fluidic devices, kits, and methods detect at least 5 different target nucleic acids in a single reaction. In some cases, the multiplexed devices, systems, fluidic devices, kits, and methods detect at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit. In some instances, the multiplexed kits detect from 2 to 10, from 3 to 10, from 4 to 10, from 5 to 10, from 6 to 10, from 7 to 10, from 8 to 10, from 9 to 10, from 2 to 9, from 3 to 9, from 4 to 9, from 5 to 9, from 6 to 9, from 7 to 9, from 8 to 9, from 2 to 8, from 3 to 8, from 4 to 8, from 5 to 8, from 6 to 8, from 7 to 8, from 2 to 7, from 3 to 7, from 4 to 7, from 5 to 7, from 6 to 7, from 2 to 6, from 3 to 6, from 4 to 6, from 5 to 6, from 2 to 5, from 3 to 5, from 4 to 5, from 2 to 4, from 3 to 4, or from 2 to 3 different target nucleic acids in a single kit.

In some embodiments, multiplexing can be carried out in a single-pot or “one-pot” reaction, where reverse transcription, amplification, in vitro transcription, or any combination thereof, and detection are carried out in a single volume. Multiplexing can be carried out in a “two-pot reaction”, where reverse transcription, amplification, in vitro transcription, or any combination thereof, are carried out in a first volume and detection is carried out in a second volume.

In some cases, multiplexing can comprise detecting multiple targets with a single probe. Alternatively, multiplexing can comprise detecting multiple targets with multiple probes. The multiple probes can be configured to detect a presence of a particular sequence, target nucleic acid, or a plurality of different target sequences or nucleic acids.

The systems, devices, apparatuses, and methods of the present disclosure can be used to detect one or more target nucleic acids within the sample. The systems, devices, and apparatuses of the present disclosure can comprise, for example, one or more pumps, valves, reservoirs, and chambers for sample preparation, amplification of a target nucleic acid within the sample, mixing with a programmable nuclease, and/or detection of a detectable signal arising from cleavage of reporters by a programmable nuclease.

Methods consistent with the present disclosure include a multiplexing method of assaying for a plurality of target nucleic acids in a sample. A multiplexing method comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Multiplexing can comprise spatial multiplexing wherein multiple different target nucleic acids are detected at the same time, but the reactions are spatially separated. In some cases, the multiple target nucleic acids are detected using the same programmable nuclease, but different guide nucleic acids. The multiple target nucleic acids sometimes are detected using the different programmable nucleases. In the case wherein multiple target nucleic acids are detected using the different programmable nucleases, the method involves using a first programmable nuclease, which upon activation (e.g., after binding of a first guide nucleic acid to a first target), cleaves a nucleic acid of a first reporter and using a second programmable nuclease, which upon activation (e.g., after binding of a second guide nucleic acid to a second target), cleaves a nucleic acid of a second reporter.

Sometimes, multiplexing can be single reaction multiplexing wherein multiple different target acids are detected in a single reaction volume. Often, at least two different programmable nucleases are used in single reaction multiplexing. For example, multiplexing can be enabled by immobilization of multiple categories of reporters within a fluidic system, to enable detection of multiple target nucleic acids within a single fluidic system. Multiplexing allows for detection of multiple target nucleic acids in one kit or system. In some cases, the multiple target nucleic acids comprise different target nucleic acids to a virus, a bacterium, or a pathogen responsible for one disease. In some cases, the multiple target nucleic acids comprise different target nucleic acids associated with a cancer or genetic disorder. Multiplexing for one disease, cancer, or genetic disorder increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. Multiplexing, thus, allows for multiplexed detection of multiple genomic alleles. For example, multiplexing comprises method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different HPV strains, for example, HPV16 and HPV18. In some cases, the multiple target nucleic acids comprise target nucleic acids directed to different cancers or genetic disorders. Often, multiplexing allows for discrimination between multiple target nucleic acids, such as target nucleic acids that comprise different genotypes, for example, for a wild-type genotype and for SNP genotype. Multiplexing for multiple diseases, cancers, or genetic disorders provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel comprises assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of reporters compared to the signal produced in the second aliquot. In this context, a unique target nucleic acid refers to the sequence of a nucleic acid that has an at least one nucleotide difference from the sequences of the other nucleic acids in the plurality. Multiple copies of each target nucleic acid can be present. For example, a unique target nucleic population can comprise multiple copies of the unique target nucleic acid. Often the plurality of unique target nucleic acids are from a plurality of bacterial pathogens in the sample.

In some instances, the multiplexed devices, systems, fluidic devices, kits, and methods detect at least 2 different target nucleic acids in a single reaction. In some instances, the multiplexed devices, systems, fluidic devices, kits, and methods detect at least 3 different target nucleic acids in a single reaction. In some instances, the multiplexed devices, systems, fluidic devices, kits, and methods detect at least 4 different target nucleic acids in a single reaction. In some instances, the multiplexed devices, systems, fluidic devices, kits, and methods detect at least 5 different target nucleic acids in a single reaction. In some cases, the multiplexed devices, systems, fluidic devices, kits, and methods detect at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit. In some instances, the multiplexed kits detect from 2 to 10, from 3 to 10, from 4 to 10, from 5 to 10, from 6 to 10, from 7 to 10, from 8 to 10, from 9 to 10, from 2 to 9, from 3 to 9, from 4 to 9, from 5 to 9, from 6 to 9, from 7 to 9, from 8 to 9, from 2 to 8, from 3 to 8, from 4 to 8, from 5 to 8, from 6 to 8, from 7 to 8, from 2 to 7, from 3 to 7, from 4 to 7, from 5 to 7, from 6 to 7, from 2 to 6, from 3 to 6, from 4 to 6, from 5 to 6, from 2 to 5, from 3 to 5, from 4 to 5, from 2 to 4, from 3 to 4, or from 2 to 3 different target nucleic acids in a single kit. Multiplexing can be carried out in a single-pot or “one-pot” reaction, where reverse transcription, amplification, in vitro transcription, or any combination thereof, and detection are carried out in a single volume. Multiplexing can be carried out in a “two-pot reaction”, where reverse transcription, amplification, in vitro transcription, or any combination thereof, are carried out in a first volume and detection is carried out in a second volume.

In some cases, multiplexing can comprise detecting multiple targets with a single probe. Alternatively, multiplexing can comprise detecting multiple targets with multiple probes. The multiple probes can be configured to detect a presence of a particular sequence, target nucleic acid, or a plurality of different target sequences or nucleic acids.

### Devices

In some non-limiting embodiments, the devices of the present disclosure can be manufactured from a variety of different materials. Exemplary materials that can be used include plastic polymers, such as poly-methacrylate (PMMA), cyclic olefin polymer (COP), cyclic olefin copolymer (COC), polyethylene (PE), high-density polyethylene (HDPE), polypropylene (PP); glass; and silicon. Features of the device (e.g., chambers, channels, etc.) can be manufactured by various processes. For example, the features can be (1) embossed using injection molding, (2) micro-milled or micro-engraved using computer numerical control (CNC) micromachining or non-contact laser drilling (by means of a CO2 laser source); (3) generated using additive manufacturing, and/or (4) generated using one or more photolithographic or stereolithographic methods.

In some embodiments, the device of the present disclosure comprises a sample interface configured to receive a sample that comprises at least one gene of interest. The device can further comprise a channel in fluid communication with the sample interface and a detection chamber. In some cases, the channel comprises one or more movable mechanisms to separate the sample into a plurality of droplets. As used herein, a droplet can refer to a volumetric portion of the sample, a partitioned sub-sample of the sample, and/or an aliquot of the sample. In some cases, the detection chamber is configured to receive and contact the plurality of droplets with at least one programmable nuclease probe disposed on a surface of said detection chamber. The at least one programmable nuclease probe can comprise a guide nucleic acid complexed with a programmable nuclease. In some cases, the programmable nuclease probe comprises a CRISPR/Cas enzyme. In some cases, the guide nucleic acid comprises a guide RNA. In some embodiments, the device comprises a plurality of programmable nuclease probes comprising different guide RNAs.

In some cases, the device can further comprise a plurality of sensors that determine a presence of said at least one gene of interest by detecting a signal produced upon cleavage of a target nucleic acid region in said at least one gene of interest by said at least one programmable nuclease probe. The cleavage of the target nucleic acid region can occur after a complementary binding of said target nucleic acid region to said guide nucleic acid of said at least one programmable nuclease probe.

As described elsewhere herein, the device may comprise one or more movable mechanisms. The one or more movable mechanisms can comprise one or more valves configured to restrict flow through one or more sections of a channel of the device. The one or more movable mechanisms can comprise a plunger or a bristle that is configured to restrict flow through one or more sections of the channel. The one or more movable mechanisms can be operatively coupled to a power source that is integrated with or insertable into the device. The power source can comprise a battery.

In some cases, the device can comprise a physical filter to filter one or more particles from the sample that do not comprise the one or more targets (e.g., a gene of interest). In some cases, the device comprises a sample preparation chamber. The sample preparation chamber can comprise a lysing agent. The sample preparation chamber can comprise a heating unit configured for heat inactivation. The sample preparation chamber can comprise one or more reagents for nucleic acid purification.

In some cases, the channel of the device can comprise one or more heating elements and one or more heat sinks for amplifying the at least one gene of interest or a portion thereof. The one or more heating elements and the one or more heat sinks can be configured to perform one or more thermocycling operations on the plurality of droplets.

In some cases, the device can comprise one or more sensors for detecting signals produced upon cleavage of a target nucleic acid. As described elsewhere herein, the signal produced upon cleavage of a target nucleic acid can be associated with a physical, chemical, or electrochemical change or reaction. The signal can comprise an optical signal, a fluorescent or colorimetric signal, a potentiometric or amperometric signal, and/or a piezo-electric signal. In some cases, the signal is associated with a change in an index of refraction of a solid or gel volume in which the at least one programmable nuclease probe is disposed.

In some embodiments, the device can comprise a sample interface configured to receive a sample that comprises one or more genomic targets of interest. In some cases, the one or more genomic targets of interest comprise a sequence of nucleic acids comprising the target nucleic acid.

In some embodiments, the device can further comprise one or more channels comprising one or more movable mechanisms to separate the sample into partitioned samples. The one or more channels can be in fluid communication with the sample interface and a reaction chamber that is configured to receive and contact the partitioned samples with an enzyme, reagent, or programmable detection agent that is configured to cleave a nucleic acid of said one or more genomic targets of interest.

In some embodiments, the device can further comprise a plurality of sensors for determining a presence of the one or more genomic targets of interest by detecting one or more reporter molecules released by said cleavage of said nucleic acid. The programmable detection agent can be a CRISPR/Cas enzyme. In some cases, the reporter molecule comprises a nucleic acid and a detection moiety. In some cases, the reporter molecule comprises at least one ribonucleotide or at least one deoxyribonucleotide. In some cases, the reporter molecule comprises a DNA nucleic acid or an RNA nucleic acid. The reporter molecule can be immobilized on a surface of the detection chamber (i.e., a movement of the reporter molecule can be physically or chemically constrained).

In some cases, the one or more movable mechanisms can comprise a plurality of valves configured to restrict or modulate flow in a first direction through the one or more channels towards the sample interface. The plurality of valves can be configured to selectively permit flow in a second direction through the one or more channels towards the reaction chamber. A first valve and a second valve of the plurality of valves can be configured to physically, fluidically, or thermally isolate a first portion of the sample from a second portion of the sample when the first valve and the second valve are in a closed state.

In some embodiments, the one or more channels can comprise a plurality of heating elements and a plurality of heat sinks to perform thermocycling on the partitioned samples. A first heating element of the plurality of heating elements and a first heat sink of the plurality of heat sinks can be positioned between a first movable mechanism and a second movable mechanism of the one or more movable mechanisms.

In any of the embodiments described herein, the device can further comprise a telemedicine unit configured to provide one or more detection results to a computing unit that is remote from the device. The computing unit can comprise a mobile device or a computer. The one or more detection results can indicate a presence or an absence of a target nucleic acid of interest in the sample. In some embodiments, the telemedicine unit provides one or more detection results to a computing unit that is remote to the device through a cloud-based connection. In some embodiments, the telemedicine unit is HIPAA compliant. In some embodiments, the telemedicine unit transmits encrypted data. The computing unit can comprise a mobile device or a computer. The one or more detection results can indicate a presence or an absence of a target nucleic acid of interest in the sample.

### Methods

In another aspect, the present disclosure provides a method for target detection. The method can comprise contacting a sample with the device of any of the preceding claims and detecting a presence or an absence of one or more genes of interest in said sample. In some cases, the method can comprise generating one or more detection results indicating the presence or the absence of the one or more genes of interest in the sample. In some cases, the method can comprise transmitting the one or more detection results to a remote computing unit. The remote computing unit can comprise, for example, a mobile device.

In another aspect, the present disclosure provides a method for target detection. The method can comprise providing a sample comprising at least one gene of interest. The method can comprise separating the sample into a plurality of sub-samples using one or more movable mechanisms described herein. The method can comprise receiving the plurality of sub-samples in a detection chamber and contacting the plurality of sub-samples with at least one programmable nuclease probe disposed on a surface of said detection chamber. The at least one programmable nuclease probe can comprise a guide nucleic acid complexed with a programmable nuclease. In some cases, the method can comprise contacting the plurality of sub-samples with a plurality of programmable nuclease probes comprising different guide RNAs. The method can comprise using a plurality of sensors to determine a presence or an absence of said at least one gene of interest by detecting a signal produced upon cleavage of a target nucleic acid region in said at least one gene of interest by said at least one programmable nuclease probe.

In some cases, the method can further comprise amplifying the at least one gene of interest after separating the sample into a plurality of sub-samples. In some cases, the method can further comprise amplifying the at least one gene of interest before mixing the plurality of sub-samples in the detection chamber. Amplifying the at least one gene of interest can comprise using a plurality of heating elements and a plurality of heat sinks to perform thermocycling on the plurality of sub-samples.

In some cases, the method can comprise using a physical filter to filter one or more particles from the sample that do not comprise the one or more target genes of interest. In some cases, the method can comprise lysing the sample before detecting the one or more target genes of interest. In some cases, the method can comprise performing heat inactivation on the sample. In some cases, the method can comprise performing nucleic acid purification on the sample.

In some cases, the detection devices described herein can be configured to implement process control procedures to ensure that the sample preparation, target amplification, and target detection processes are performed accurately and as intended.

### Methods to Immobilize DETECR Assay Components to Surfaces

Programmable nuclease-based diagnostic reactions can be performed in solution where the programmable nuclease-guide nucleic acid complexes (e.g., Cas protein-RNA complexes) can freely bind target molecules and reporter molecules. However, reactions where all components are in solution may limit the designs of programmable nuclease-based diagnostic assays, especially in microfluidic devices. A system where one or more components of the programmable nuclease-based diagnostic reaction could be immobilized on a surface can enable designs where multiple readouts can be accomplished within a single reaction chamber, improve distribution (e.g., transportability) and manufacturing, improve assay time and/or sensitivity, or any combination thereof.

Described herein are various immobilization methods to tether programmable nuclease-based diagnostic reaction components to one or more surfaces of a reaction chamber or other surface (e.g., a surface of a bead or a portion of an immobilization matrix). In certain instances, the presently disclosed systems and methods can involve immobilization of programmable nucleases, reporters, and/or guide nucleic acids. Table 1 presents various examples of guide nucleic acids and reporter immobilization sequences that may be used to enable programmable nuclease-based-based diagnostics and detection of target sequences.

In some embodiments, various programmable nuclease-based diagnostic reaction components can be modified with biotin. In some embodiments, these biotinylated programmable nuclease-based diagnostic reaction components are tested for immobilization on surfaces coated with streptavidin. In some embodiments, the biotin-streptavidin interaction can be used as a model system for other immobilization chemistries,

TABLE 1
presents guide nucleic acid and reporter immobilization sequences.

| Name | Sequence | Description |
|---|---|---|
| R003 | rGrGrCrCrArCrCrCrCrArArArArArUrGrArArGrG | unmodified Cas13 crRNA |
| | rGrGrArCrUrArArArArCrArGrUrGrArUrArArGrU | |
| | rGrGrArArUrGrCrCrArUrG (SEQ ID NO: 63) | |
| mod023 | /5BiotinTEG/rGrGrCrCrArCrCrCrCrArArArArArU | biotin modified Cas13 crRNA |
| | rGrArArGrGrGrGrArCrUrArArArArCrArCrGrArC | |
| | rCrUrArCrUrCrUrCrCrCrArUrArCrUrC (SEQ ID | |
| | NO: 64) | |
| R1763 | rUrArArUrUrUrCrUrArCrUrArArGrUrGrUrArGrA | Unmodified gRNA targeting a |
| | rUrCrCrCrCrCrArGrCrGrCrUrUrCrArGrCrGrUrUr | sequence in SARS-CoV-2 |
| | C (SEQ ID NO: 65) | |
| mod011 | /5Biosg/rUrA rArUrU rUrCrU rArCrUrArArG | R1763 with a 5′ biotin |
| | rUrGrU rArGrArUrCrCrCrCrCrArGrCrGrCrU | |
| | rUrCrA rGrCrG rUrUrC (SEQ ID NO: 66) | |
| mod012 | /5Biosg/T*T*T*T*T*rUrArArUrUrUrCrUrArCrUr | R1763 with 5 |
| | ArArGrUrGrUrArGrArUrCrCrCrCrCrArGrCrGrCr | phosphorothioated nucleotides |
| | UrUrCrArGrCrGrUrUrC (SEQ ID NO: 67) | on 5′ end |
| mod013 | /5Biosg/T*T*T*T*T*T*T*T*T*T*rUrArArUrUrU | R1763 with 10 |
| | rCrUrArCrUrArArGrUrGrUrArGrArUrCrCrCrCrC | phosphorothioated nucleotides |
| | rArGrCrGrCrUrUrCrArGrCrGrUrUrC (SEQ ID | on 5′ end |
| | NO: 68) | |
| mod014 | rUrArArUrUrUrCrUrA/iBiodUK/rCrUrArArGrUrG | R1763 with an internal biotin |
| | rUrArGrArUrCrCrCrCrCrArGrCrGrCrUrUrCrArGr | modification |
| | CrGrUrUrC (SEQ ID NO: 69) | |
| mod015 | rUrArArUrUrUrCrUrArCrUrArArGrUrGrUrArGrA | R1763 with an internal biotin |
| | rUrC/iBiodUK/rCrCrCrCrArGrCrGrCrUrUrCrArGr | modification |
| | CrGrUrUrC (SEQ ID NO: 70) | |
| mod016 | rUrArArUrUrUrC/iBiodUK/rArCrUrArArGrUrGrU | R1763 with an internal biotin |
| | rArGrArUrCrCrCrCrCrArGrCrGrCrUrUrCrArGrCr | modification |
| | GrUrUrC (SEQ ID NO: 71) | |
| mod017 | rUrArArUrUrUrCrUrArCrUrArArGrUrGrUrArGrA | R1763 with an internal biotin |
| | /iBiodUK/rCrCrCrCrCrArGrCrGrCrUrUrCrArGrCr | modification |
| | GrUrUrC (SEQ ID NO: 72) | |
| mod018 | /5BiotinTEG/rUrArArUrUrUrCrUrArCrUrArArGr | R1763 with a 5′ biotin-TEG |
| | UrGrUrArGrArUrCrCrCrCrCrArGrCrGrCrUrUrCr | modification |
| | ArGrCrGrUrUrC (SEQ ID NO: 73) | |
| mod019 | rUrA rArUrU rUrCrU rArCrU rArArGrUrGrU | R1763 with a 3′ biotin |
| | rArGrA rUrCrC rCrCrC rArGrCrGrCrU rUrCrA | modification |
| | rGrCrG rUrUrC/3Bio/ (SEQ ID NO: 74) | |
| mod020 | rUrA rArUrU rUrCrU rArCrU rArArGrUrGrU | R1763 with a 3′ biotin-TEG |
| | rArGrA rUrCrC rCrCrCrArGrCrGrCrU rUrCrA | modification |
| | rGrCrGrUrUrC/3BioTEG/ (SEQ ID NO: 75) | |
| rep072 | /56-FAM/TTATTATT/3Bio/ | FAM reporter with 3′ biotin |
| rep103 | /5Alex488N/TT ATT ATT/3Bio/ | 5′ modified with Alexa488 |
| | | reporter and 3′ modified with |
| | | biotin |
| rep104 | /5Alex488N/TT ATT ATT | 5′ modified with Alexa488 |
| | AT/iBiodT/ATT/3IABKFQ/ (SEQ ID NO: 76) | reporter with internal biotin and |
| | | 3′ modified with a quencher |
| rep 105 | /5BiotinTEG/*T*T*/16-FAMK/*TATTA TTA | 5′ modified with biotin-TEG |
| | TTA TTA TT/3IABKFQ/ (SEQ ID NO: 77) | followed by two |
| | | phosphorothioated nucleotides |
| | | (*) should not be cleavable and |
| | | an internal FAM and 3′ |
| | | modified with a quencher |
| | | *T* could also be some other |
| | | non-cleavable su |
| rep106 | /56-FAM/TT ATT ATT ATT A/3Dig_N/ (SEQ ID | 5′ FAM reporter with a 3′ DIG |
| | NO: 78) | |
| rep 115 | [biotinTEG]TTTTTTTTTTTTTTTTTTTTTTTTTT | biotinTEG modified 5′ and |
| | TTTT[Phycoerythrin] (SEQ ID NO: 79) | Phycoetrythrin modified 3′, |
| | | where 5′ and 3′ linked by T30 |
| rep116 | [FAM]n[branch]TTTTTTTTTTTTTTTTTTTT | 5′ modified with 9 FAMs and |
| | [biotinTEG] (SEQ ID NO: 80) | 3′ modified with biotinTEG, |
| | | where 5′ and 3′ linked by T20 |
| rep117 | [BiotinTEG]*T*T*[internalFAM]*TTTTTTTTTTT | 5′ modified with BiotinTEG |
| | TTTTTTTTT[IABKFQ] (SEQ ID NO: 81) | and 3′ modified with FQ, where |
| | | 5′ and 3′ linked by T20 |
| rep118 | [FAM]TTTTTTTTTTTTTTTTTTTT* | 5′ modified with FAM and 3′ |
| | [internalBiotin]*T[IABKFQ] (SEQ ID NO: 82) | modified with Biotin-FQ, |
| | | where 5′ and 3′ are linked by |
| | | T20 |
| rep119 | [5BiotinTEG]*T*T*[internalCy5]*TTTTTTTTTTT | 5′ modified with BiotinTEG |
| | TTTTTTTTT[RQ] (SEQ ID NO: 83) | and 3′ modified with BHQ-2, |
| | | contains internal Cy5 dye |
| | | linked to quencher by T20 |
| rep121 | /5BiotinTEG//iSp18//iCy5/TTT TTT TTT TTT | 5′ modified with BiotinTEG, an |
| | TTT TTT TT/3IAbRQSp/ (SEQ ID NO: 84) | internal 18 atom spacer, and an |
| | | internal Cy5 followed by T20 |
| | | and a IAbRQSp quencher |
| rep120 | [Cy5]TTTTTTTTTTTTTTTTTTTT*[internalBiotin] | 5′ modified with Cy5 followed |
| | *T[RQ] (SEQ ID NO: 85) | by T20 linker to an internal |
| | | biotin and an RQ quencher. * |
| | | indicates phosphorothioated |
| | | nucleotides |
| rep125 | /5Alex647N//iBiodT/TTT TTT TTT TTT TTT TTT | 5′ modified with Alexa647 |
| | TT/3IAbRQSp/ (SEQ ID NO: 86) | followed by an internal |
| | | biotindT T20 and a 3′ |
| | | IAbRQSp quencher |
| rep126 | /5BiotinTEG/TT/i6- | 5′ biotinTEG modified |
| | FAMK/TTTTTTTTTrUrUrUrUrUTTTTTT | followed by two T, an internal |
| | /3IABKFQ/ (SEQ ID NO: 87) | FAM and, 10T, 5 RNA U, 6T, |
| | | and a quencher. Acts as an |
| | | RNA cleavage reporter. |
| rep110 | /5AmMC6T/TTTTTTTTTTTT/3AlexF488N/ (SEQ | 5′ amino with 6 carbons |
| | ID NO: 88) | followed by T12 and a |
| | | Alexa488 modification on 3′ |
| | | end |
| rep111 | /5AmMC6T//i6- | 5′ amino with 6 carbons |
| | FAMK/TTTTTTTTTTTT/3IABKFQ/ (SEQ ID NO: | followed by internal FAM, T12, |
| | 89) | and a quencher on 3′ |
| rep112 | /5AmMC6T/TT TTT TTT TTT T/36-FAM/ (SEQ | 5′ amino with 6 carbons |
| | ID NO: 90) | followed by T12 and a 3′ FAM |
| | | modification |
| rep122 | /5AmMC12//iSp18//iCy5/TTT TTT TTT TTT TTT | 5′ amino with 12 carbons |
| | TTT TT/3IAbRQSp/ (SEQ ID NO: 91) | followed by 18 atom linker |
| | | followed by Cy5 followed by |
| | | T20 and a 3′ quencher |
| rep123 | /5AmMC12//iCy5/ TTT TTT TTT TTT TTT TTT | 5′ amion with 12 carbons |
| | TT/3IAbRQSp/ (SEQ ID NO: 92) | followed by internal Cy5 and a |
| | | T20 linker to a 3′ quencher |
| rep135 | /5AmMC12//i6- | 5′ amino with 12 carbons |
| | FAMK/TTTTTTTTTTTTTTTTTTT/3IABKFQ/ | followed by internal FAM and |
| | (SEQ ID NO: 93) | a T20 linker to a 3′ quencher |
| rep136 | /5AmMC6//i6- | 5′ amino with 6 carbons |
| | FAMK/TTTTTTTTTTTTTTTTTTT/3IABKFQ/ | followed by internal FAM and |
| | (SEQ ID NO: 94) | a T20 linker to a 3′ quencher |
| mod026 | /5AmMC6/rUrArArUrUrUrCrUrArCrUrArArGrUr | 5′ amino with 6 carbons linked |
| | GrUrArGrArUrCrCrCrCrCrArGrCrGrCrUrUrCrAr | to gRNA for SARS-CoV-2 N- |
| | GrCrGrUrUrC (SEQ ID NO: 95) | gene |
| mod027 | /5AmMC12/rUrArArUrUrUrCrUrArCrUrArArGrU | 5′ amino with 12 carbons |
| | rGrUrArGrArUrCrCrCrCrCrArGrCrGrCrUrUrCrAr | linked to gRNA for SARS- |
| | GrCrGrUrUrC (SEQ ID NO: 96) | CoV-2 N-gene |
| mod028 | /5AmMC6T/rUrArArUrUrUrCrUrArCrUrArArGrU | 5′ amino with 6 carbons on dT |
| | rGrUrArGrArUrCrCrCrCrCrArGrCrGrCrUrUrCrAr | linked to gRNA for SARS- |
| | GrCrGrUrUrC (SEQ ID NO: 97) | CoV-2 N-gene |
| mod029 | rUrArArUrUrUrCrUrArCrUrArArGrUrGrUrArGrA | gRNA for SARS-CoV-2 N- |
| | rUrCrCrCrCrCrArGrCrGrCrUrUrCrArGrCrGrUrUr | gene with 5′ amino with 6 |
| | C/3AmMC6T/ (SEQ ID NO: 98) | carbons on 3′ end |
| mod030 | /5AmMC12/rUrArArUrUrUrCrUrArCrUrArArGrU | 5′ amino with 12 carbons |
| | rGrUrArGrArUrUrUrArCrArUrGrGrCrUrCrUrGrG | linked to gRNA for human |
| | rUrCrCrGrArG (SEQ ID NO: 99) | RNase P POP7 |
| mod031 | /5AmMC12/rUrArArUrUrUrCrUrArCrUrArArGrU | 5′ amino with 12 carbons |
| | rGrUrArGrArUrGrCrCrGrArUrArArUrGrArUrGrU | linked to gRNA for |
| | rArGrGrGrArU (SEQ ID NO: 100) | Mammuthus primigenius |
| | | control sequence |
| mod059 | /5ThioMC6-D/rUrA rArUrU rUrCrU rArCrU | 5′ thiol modification with 6 |
| | rArArGrUrGrU rArGrA rUrCrC rCrCrC rArGrC | carbons linked to gRNA for |
| | rGrCrU rUrCrA rGrCrG rUrUrC (SEQ ID NO: | SARS-CoV-2 N-gene |
| | 101) | |
| rep130 | /5ThioMC6-D/*T*T*/i6- | 5′ modified with BiotinTEG |
| | FAMK/*TTTTTTTTTTTTTTTTTTTT/3IABKFQ/ | and 3′ modified with FQ, where |
| | (SEQ ID NO: 102) | 5′ and 3′ linked by T20; two |
| | | phosphorothioated nucleotides |
| | | between thiol and internal FAM |
| mod024 | /5BiotinTEG/rUrArArUrUrUrCrUrArCrUrArArGr | 5′ modified with BiotinTEG |
| | UrGrUrArGrArUrGrCrCrGrArUrArArUrGrArUrGr | linked to a gRNA that targets |
| | UrArGrGrGrArU (SEQ ID NO: 103) | Mammuthus primigenius |
| | | sequence |
| mod025 | /5BiotinTEG/rUrArArUrUrUrCrUrArCrUrArArGr | 5′ modified with BiotinTEG |
| | UrGrUrArGrArUrUrUrArCrArUrGrGrCrUrCrUrGr | linked to a gRNA that targets |
| | GrUrCrCrGrArG (SEQ ID NO: 104) | human RNase P POP7 |
| mod058 | /5biotinTEG/rUrArArUrUrUrCrUrArCrUrArArGr | 5′ modified with BiotinTEG |
| | UrGrUrArGrArUrCrUrGrCrCrArArUrUrGrCrArGr | linked to a gRNA that targets |
| | GrArArUrGrArU (SEQ ID NO: 105) | Mammuthus primigenius |
| | | sequence |

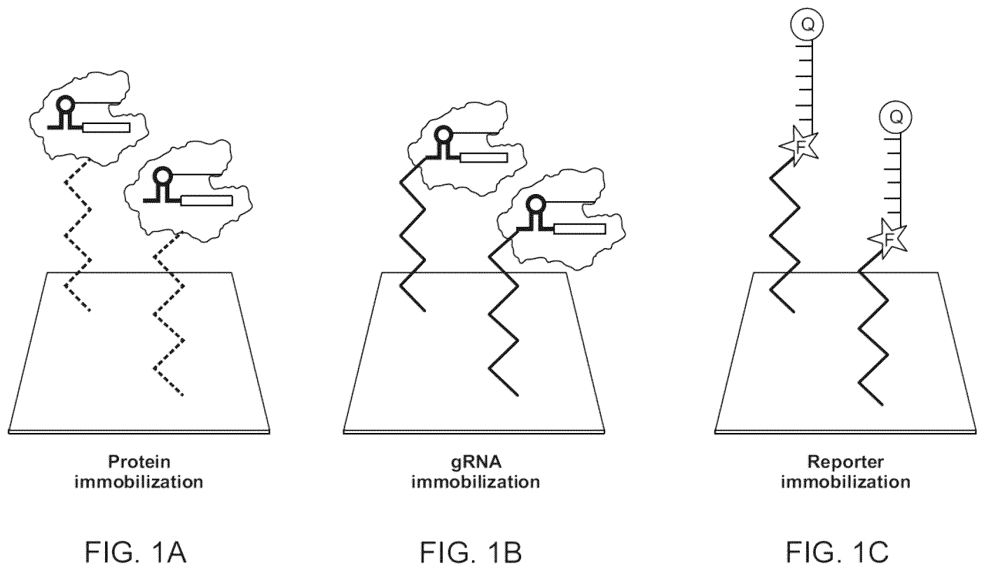

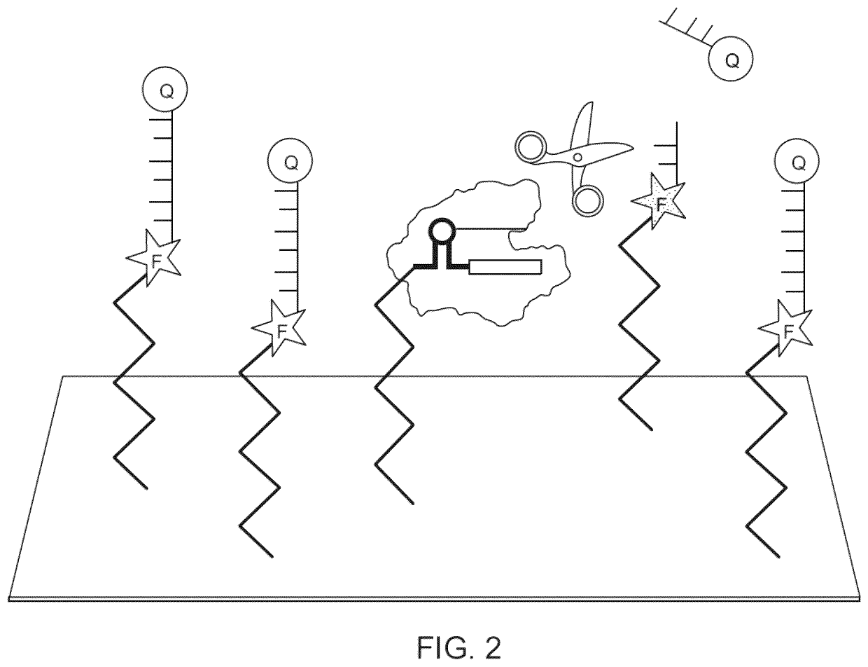

FIGS. 1A-1C illustrate three examples of immobilization strategies for programmable nuclease-based diagnostic assay components. In some embodiments, as seen in FIG. 1A, chemical modifications of amino acid residues in the programmable nuclease enable attachment to a surface. In some embodiments, as seen in FIG. 1B, guide nucleic acids are immobilized by adding various chemical modifications at the 5′ or 3′ end of the guide nucleic acid that are compatible with a selected surface chemistry. In some embodiments, as seen in FIG. 1C fluorescence-quenching (FQ), or other reporter chemistries, are attached to surfaces using similar chemical modifications as those discussed above for guide nucleic acids. In some embodiments, these attached reporters are activated by a programmable nuclease, which leads to either activated molecules that remain attached to the surface or activated molecules that are released into solution.

FIG. 2 provides an illustrative example of immobilization strategies for use with methods and compositions described herein where the RNP complex is immobilized by a guide nucleic acid and cleaves surrounding FQ reporters that are also immobilized to a surface. Here, the quencher is released into solution, leaving a localized fluorescent signal.

In some embodiments, the programmable nuclease, guide nucleic acid, and/or the reporter can be immobilized to a device surface by a linkage or linker. In some embodiments, the linkage comprises a covalent bond, a non-covalent bond, an electrostatic bond, an interaction (e.g., a covalent or noncovalent bond) between members of a binding pair (e.g., streptavidin and biotin), an amide bond, or any combination thereof. In some embodiments, the linkage comprises non-specific absorption. In some embodiments, the programmable nuclease is immobilized to the device surface by the linkage, wherein the linkage is between the programmable nuclease and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface.

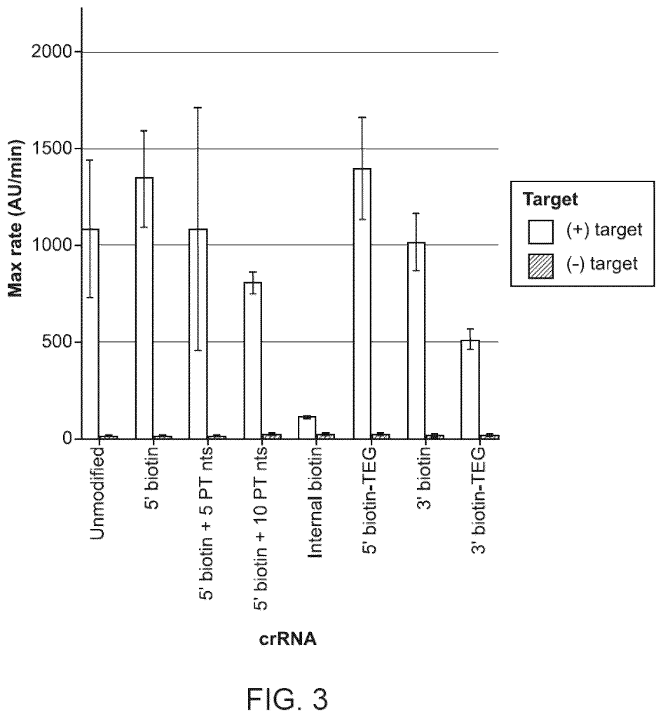

In some embodiments, various chemical modifications to guide nucleic acids are described as shown in FIG. 3. The y-axis shows reaction rate in terms of fluorescence intensity over time and the x-axis presents various modifications of crRNA. In some embodiments, unmodified biotin and variations of biotin modifications are placed at various positions along a Cas12 guide nucleic acid. The modified guide nucleic acids are then complexed with the protein and dsDNA target is added. In some embodiments, higher average fluorescence over the same period of time indicates that modifications are tolerated on the 5′ and 3′ ends of the guide nucleic acid, but not internally in the guide nucleic acid. In some cases, 5′ modified guide nucleic acids appear to be more robust than guide nucleic acids with 3′ modifications.

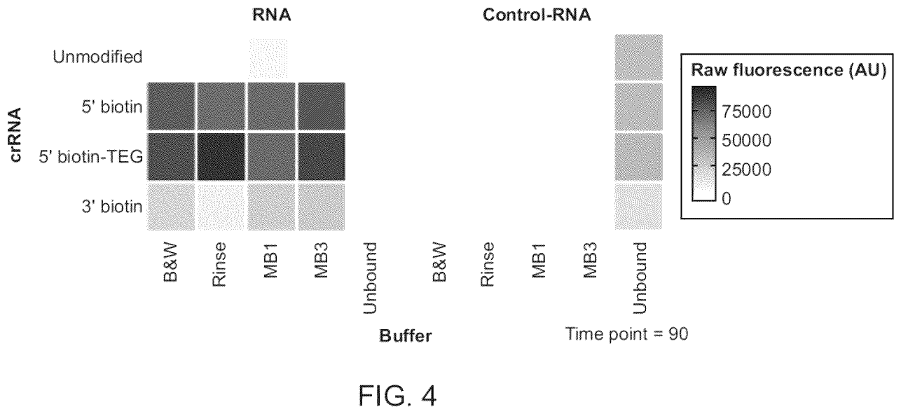

The immobilization of gRNAs to a streptavidin surface is shown in FIG. 4. Two plots are shown, where the left-hand plot depicts gRNA bound to a streptavidin coated surface and the right-hand plot depicts unbound control-gRNA, in solution, mixed with target, reporter and protein solution. In each table, the y-axis depicts, in some embodiments, various modifications of crRNA and the x-axis depicts various buffer conditions that the crRNA is subjected to. The left-hand, experimental plot shows gRNA on 5′ or 3′ side are both functional approaches, but 5″ biotin modified gRNAs show increased signal in comparison to 3′ modified gRNAs. In some embodiments, unmodified gRNAs show no signal when bound to the plate. Conversely, the right-hand control plot depicts an unbound control where, in some embodiments, free gRNA is mixed with target, reporter, and protein solution. In this embodiment, sufficient signal is observed, indicating functionality in unmodified gRNAs.

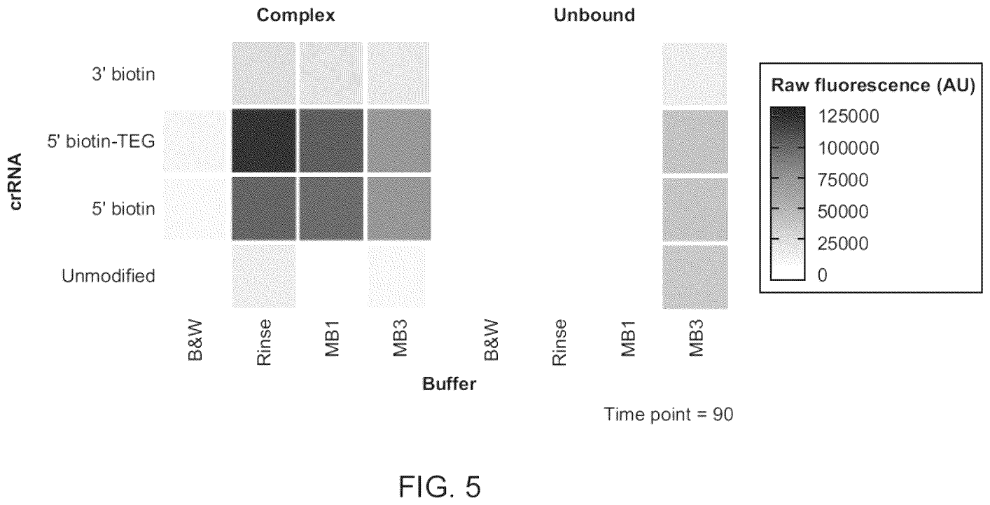

In some embodiments, programmable nucleases (e.g., Cas proteins) are complexed with guide nucleic acids (e.g., gRNA) as described and shown herein, FIG. 5 shows RNP complexes bound by 5′ biotin modified gRNAs exhibit higher signal, indicating functional attachment to the surface of the streptavidin coated plate. Samples exposed to unbound, high salt “B&W” buffer conditions show less fluorescence signal indicating inhibited protein activity or disruption of binding of functional RNP complexes to the surface of the plate. Unmodified gRNAs also exhibit lower fluorescence indicating failed binding to the RNP to the plate surface. Control assays, shown in the right-hand plot, indicate unbound gRNAs are still functional in solution.

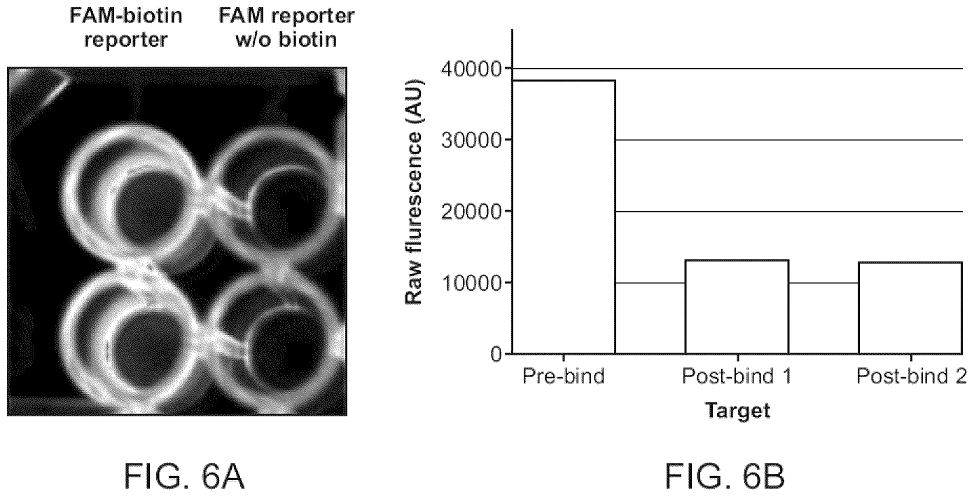

In some embodiments, reporter molecules are immobilized to the surface as shown in FIGS. 6A-6B, FIG. 6A shows a fluorescence image of four wells with streptavidin coated surfaces, where the left-hand column of wells contains FAM-biotin reporter molecules immobilized to a streptavidin coated surface. The right-hand column of wells contains FAM reporter without biotin functionalization. The left-hand column exhibits a higher signal, FIG. 6B shows a comparison of fluorescence intensity of the FAM-biotin pre-binding solution to the solution after incubating on the streptavidin plate. A decrease in signal for both wells containing FAM-biotin is observed.

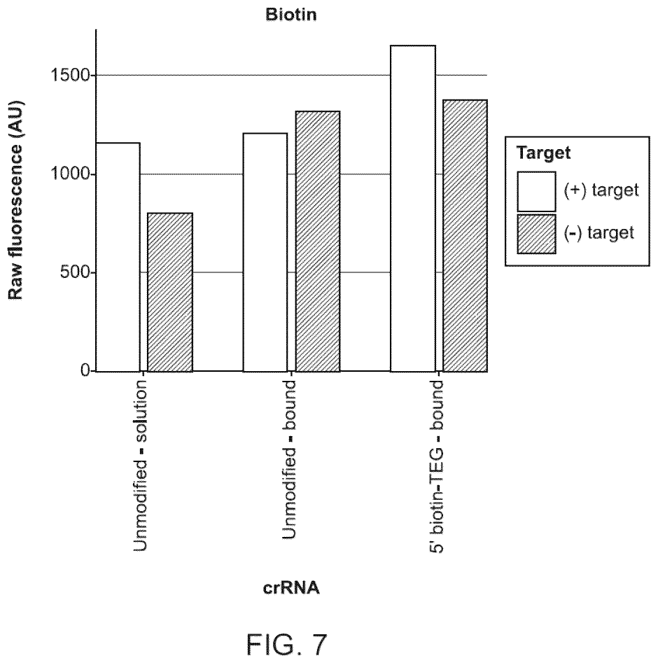

In some embodiments, combined RNP and a reporter system are immobilized for functional testing as shown in FIG. 7. Raw fluorescence (AU) is plotted against three conditions: (1) unmodified crRNA in solution, (2) unmodified crRNA bound to the surface and (3) 5′ biotin-TEG modified crRNA bound to the surface. The combined binding of the reporter and RNP to the plate shows a similar signal to RNP in solution with bound reporter.

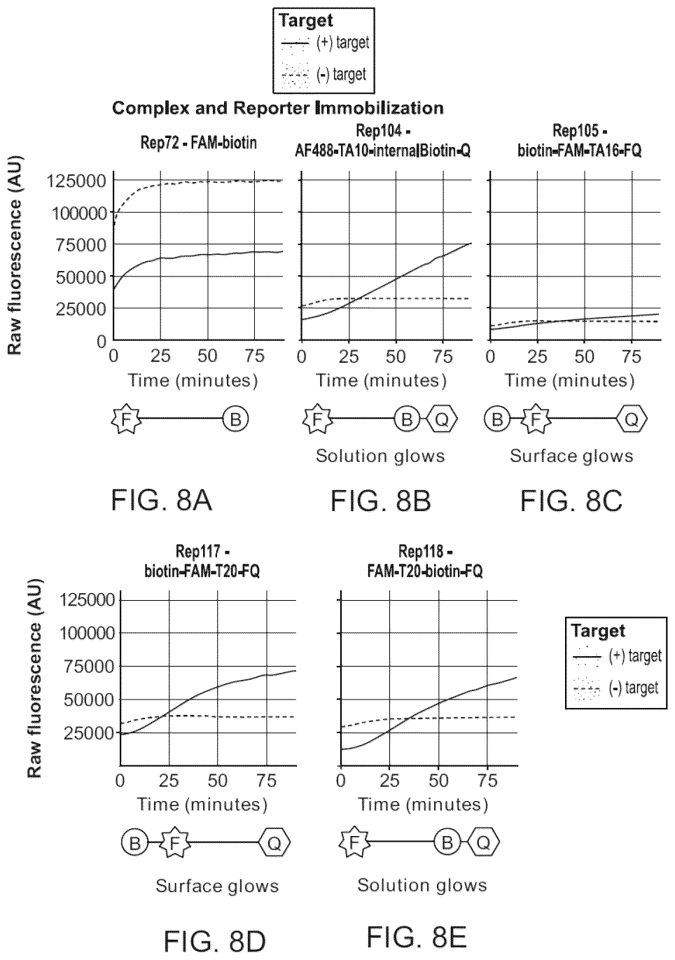

In some embodiments, different reporters are immobilized in combination with Cas complexes on a streptavidin surface for evaluation of the DETECTR assay. FIGS. 8A-8E present results for evaluation of different reporters for immobilization in combination with Cas complex immobilization on a streptavidin surface. In each figure, raw fluorescence is plotted against time in minutes representing kinetic binding curves of the Cas complex for each type of reporter while binding with a positive control (+) and negative control (−) target. FIG. 8A presents the binding results for a FAM-biotin reporter, “rep” composed of the fluorophore FAM and biotin and is listed as rep72. The FIG. 8B plots the raw fluorescence for a reporter composed of the fluorophore AlexaFluor488, “AF488,” and TA10-internalBiotinQ. As predicted, the positive control shows a positive slope indicating increased binding over the course of the reaction. This is due to the release of FAM dye into solution upon binding and transcleavage. In rep104, the cleavage point is between the FAM and the biotin, while the biotin in all reporters tested is the attachment point to the streptavidin surface, FIG. 8C plots the control, target binding kinetic plot for rep105, Rep105 is composed of biotin-FAM-T16-FQ. In this case the streptavidin coated surface emits fluorescence because the region between the FAM dye and the quencher is cleaved upon binding and the quencher is released. FIG. 8D plots the control for rep117, Rep117 is composed of biotin-FAM-T20-FQ. In this embodiment, the reporter is cleaved between the FAM dye and the quencher, thus allowing for release of the quencher in the solution upon binding and transcleavage. This in turn, causes the surface to emit fluorescence. FIG. 8E plots the control for rep118, Rep118 is composed of FAM-T20-biotin-FQ. In this embodiment, the solution emits fluorescence because upon binding the nucleic acid region between the biotin and the FAM is transcleaved, thus releasing the FAM into solution.

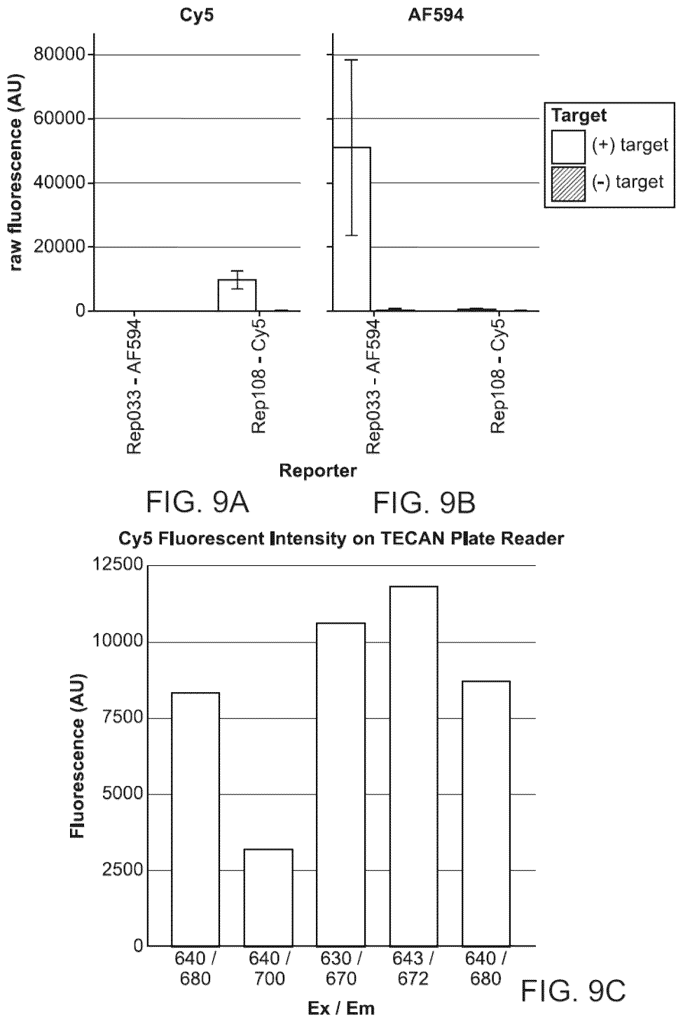

In some embodiments, Cy5 dye may be used as a reporter or a component of a reporter, FIGS. 9A-C present results for the Cy5 reporter (rep108) showing that it is functional for DETECTR but produces a weaker signal (might be gain related), as described herein. FIGS. 9A and 9B plot raw fluorescence versus reporter type for channels configured to read Cy5 dye and/or Alexa Fluor 594 “AF594” dye, respectively. In these plots the average raw fluorescence is shown for each reporter. Reporter, rep033, readout in the “AF594” channel had the most significant fluorescence signal. FIG. 9C plots raw fluorescence for various combinations of excitation and emission wavelengths on a plate reader for Cy5 dye. Under similar assay conditions AF594 exhibits stronger signal than Cy5, but Cy5 is functional. Optimum excitation and emission wavelengths for Cy5 are shown to be about 643 nm and about 672 nm, respectively.

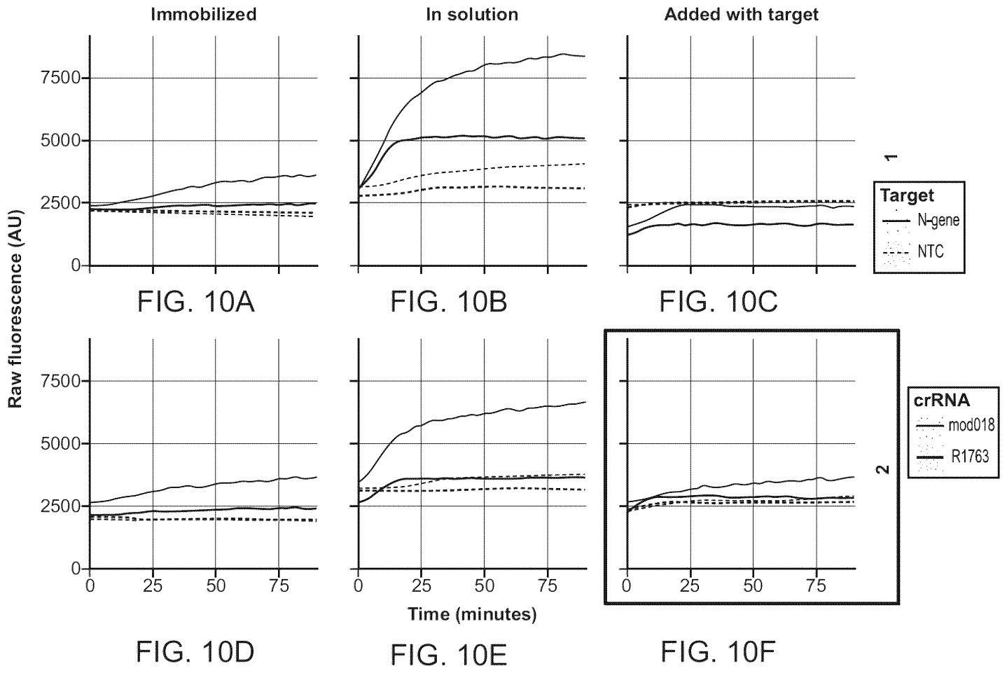

FIGS. 10A-10F present results for optimization of the complex formation step where certain components are immobilized, as described herein. In each figure raw fluorescence is plotted against time in minutes. FIGS. 10A-10C show results for replicate 1. FIGS. 10D-10F show results for replicate 2. In FIGS. 10A and 10D the reporter and guide nucleic acid are immobilized and Cas protein was introduced to form a complex with the immobilized guide nucleic acid before addition of the target. In FIGS. 10B and 10E all components are in solution. In FIGS. 10C and 10F the reporter and guide nucleic acid are immobilized and Cas12 and target are added at the same time.

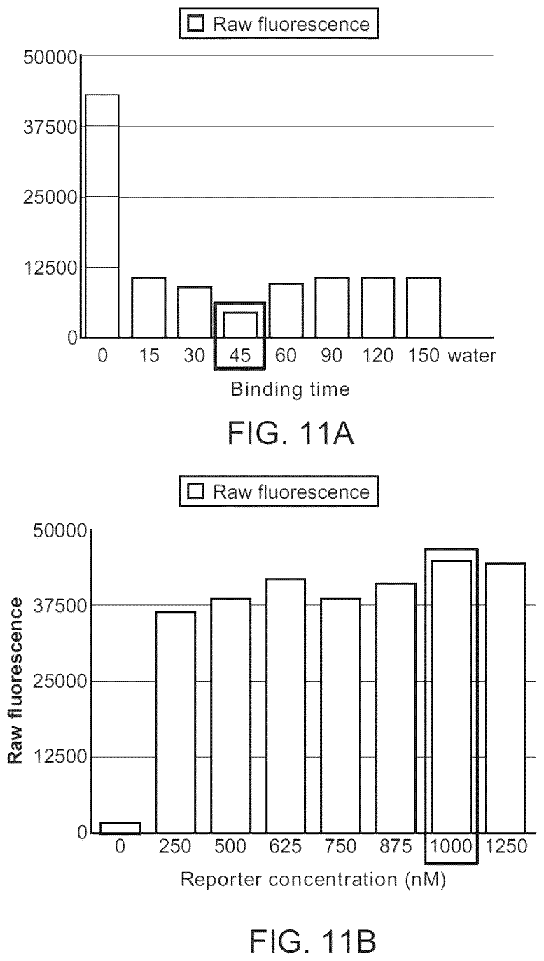

FIGS. 11A-B present results for immobilization optimization involving gRNA/reporter binding time and reporter concentration. FIG. 11A is a measurement of supernatant of the surface reaction over time showing the fluorescence dropping and thus indicating uptake of biotinylated dye reporter by the streptavidin surface. In this embodiment a 15 min binding time was found to be sufficient under the conditions tested. FIG. 11B is a measurement of surface fluorescence after immobilization showing fluorescence increasing and thus indicating reporters binding to the streptavidin surface. In this embodiment a concentration of 250 nM reporters was found to be sufficient under the conditions tested.

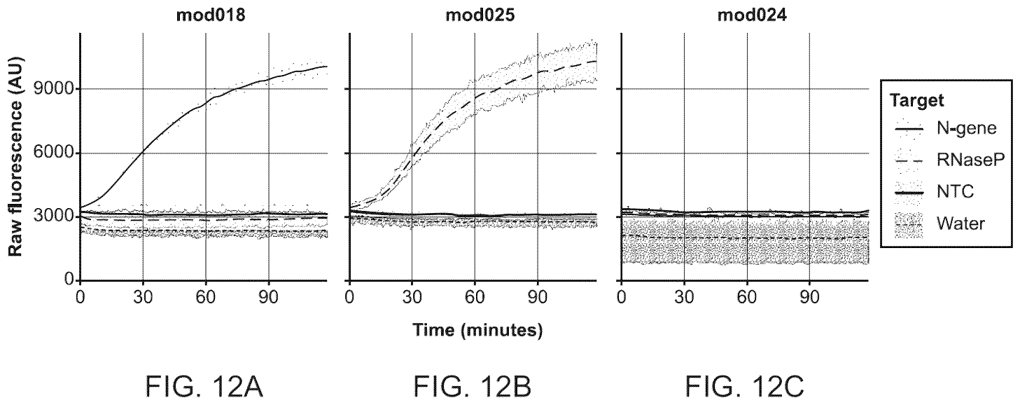

In some embodiments, gRNAs are modified. In some embodiments, the modified gRNAs are modified with linker molecules for immobilization onto a surface. FIGS. 12A-12C present results showing target discrimination of modified gRNAs.

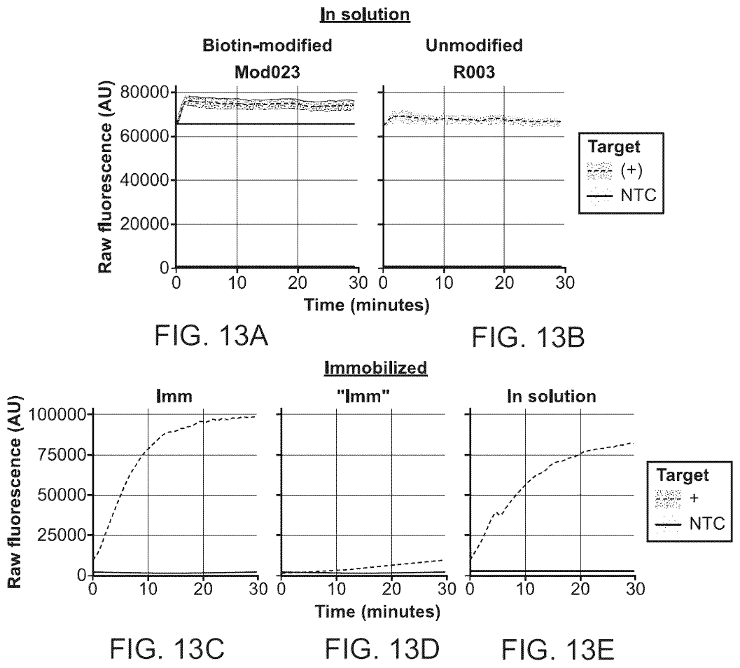

In some embodiments, guide RNAs are modified for surface modification. In some embodiments, reporter molecules are modified for surface immobilization. In these embodiments, an immobilized gRNA, or immobilized reporter or a combination thereof participate in a diagnostic assay including a programmable nuclease. FIGS. 13A-13E present results demonstrating functionality of biotin-modified Cas13a gRNA. In each figure, raw fluorescence is plotted against time in minutes, where the dashed line series represents data for when the target is present, and the thin solid line with low amount of speckle for boundaries series represents when target is not present (no target control or NTC). FIGS. 13A and 13B show results, in solution, for mod023, the biotin modified reporter and R003 the non-biotin-modified reporter, respectively. In some embodiments the biotin-modified gRNA has similar performance to the non-biotin-modified gRNA in solution. FIG. 13C shows results for gRNA that was modified with biotin and immobilized to the surface. FIG. 13D shows results for gRNA that was not modified with biotin but was deposited on the surface in the same manner as in FIG. 13C, FIG. 13E, similar to FIGS. 13A and 13B, shows results for gRNA that was unmodified and in solution. Together these results showed that with biotin modification and surface immobilization functionality was maintained and DETECTR assay performance was not adversely affected.

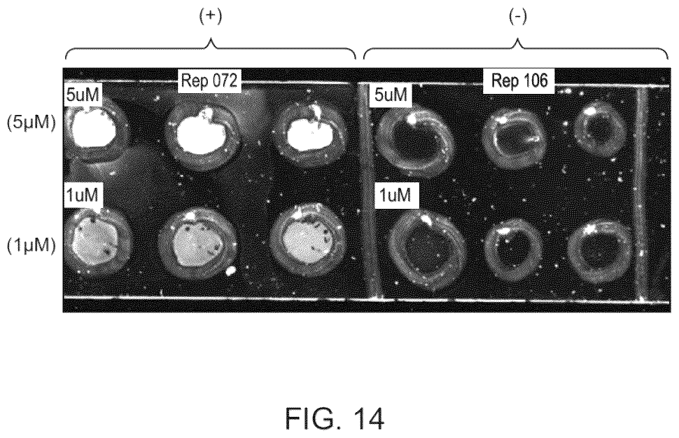

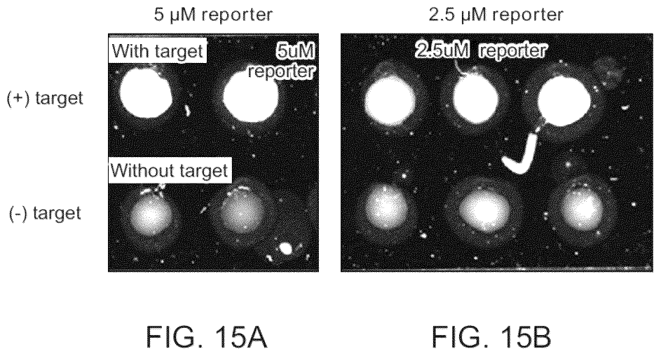

In some embodiments, biomolecules are immobilized to surfaces. In some embodiments, the surfaces were glass. FIG. 14 shows results for the test reporter, Rep072, and the negative control, Rep106. The replicates of Rep072 at 5 μM show the strongest signal and the three replicates of Rep072 at 1 μM concentration show the next strongest signal. The negative control reporter, rep106 shows the same low signal (or none at all) for both 5 μM and 1 μM concentrations. This result shows specific binding of a FAM-biotinylated reporter, rep072, with a 30 minute incubation time at both 5 μM and 1 μM concentrations. FIGS. 15A-15B show similar results with reporters at 5 mM in FIG. 15A and 2.5 mM in FIG. 15B. The top row of FIGS. 15A and 15B shows spots exhibiting bright fluorescence and the bottom row of FIGS. 15A and 15B show spots exhibiting similarly low fluorescence.

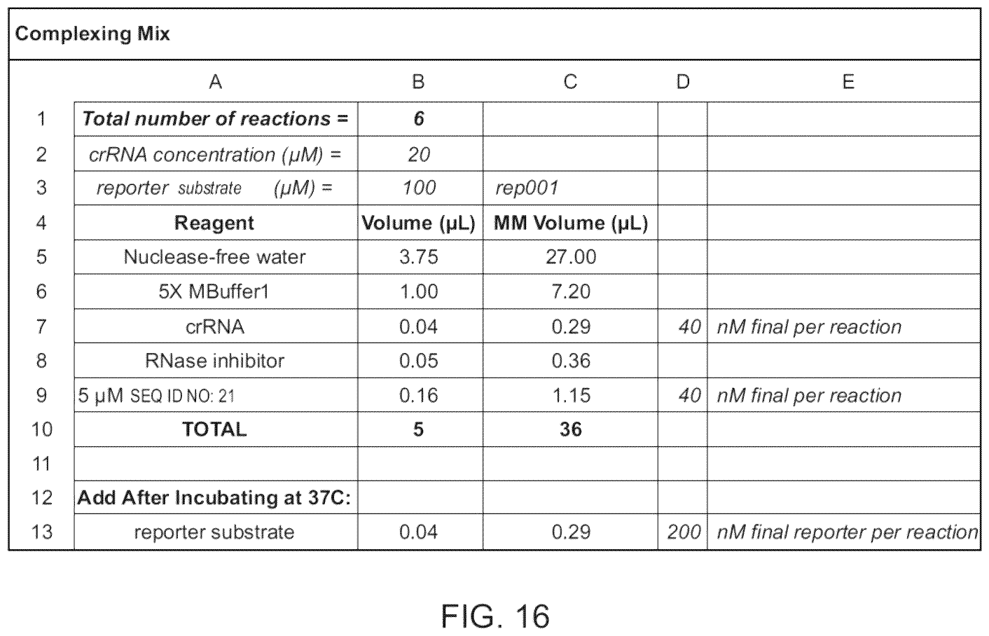

Experimental parameters for the preparation of an embodiment of a complexing mix are shown in FIG. 16. Such a complexing mix may be used to evaluate the function of biotin modified guide nucleic acids for a programmable nuclease (e.g., a Cas13 enzyme), as described in greater detail below.

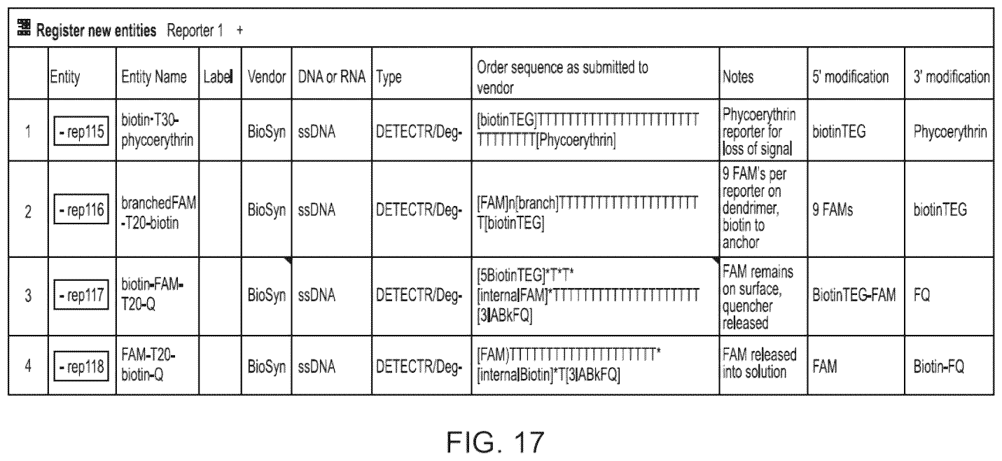

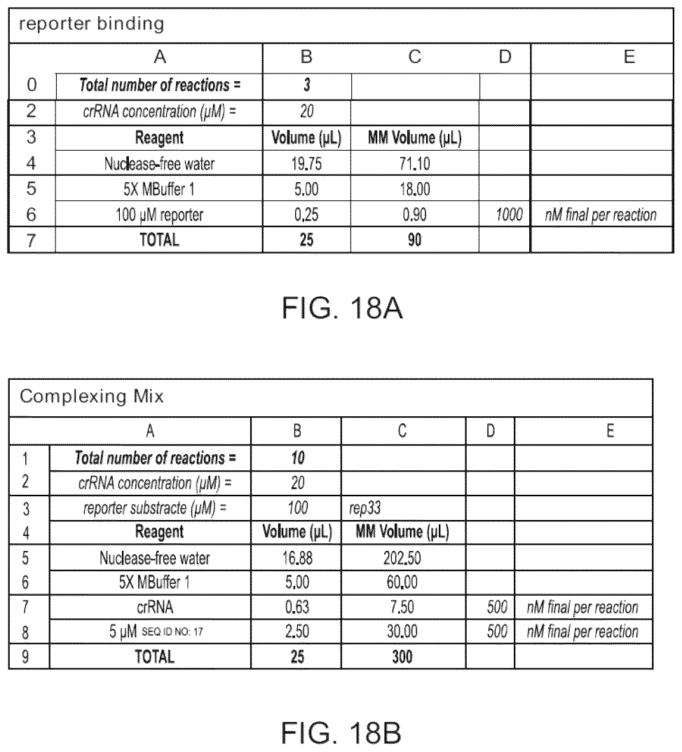

In some embodiments, fluorescent quencher-based reporters are used in the immobilized DETECTR assay. FIG. 17 shows sequence and other details for reporters used in some embodiments. In some embodiments, reporters rep072, rep104, rep105, rep117 and rep118 are used for binding to a reader plate. Reporter binding details and complexing mix parameters are shown in FIGS. 18A and 18B, respectively.

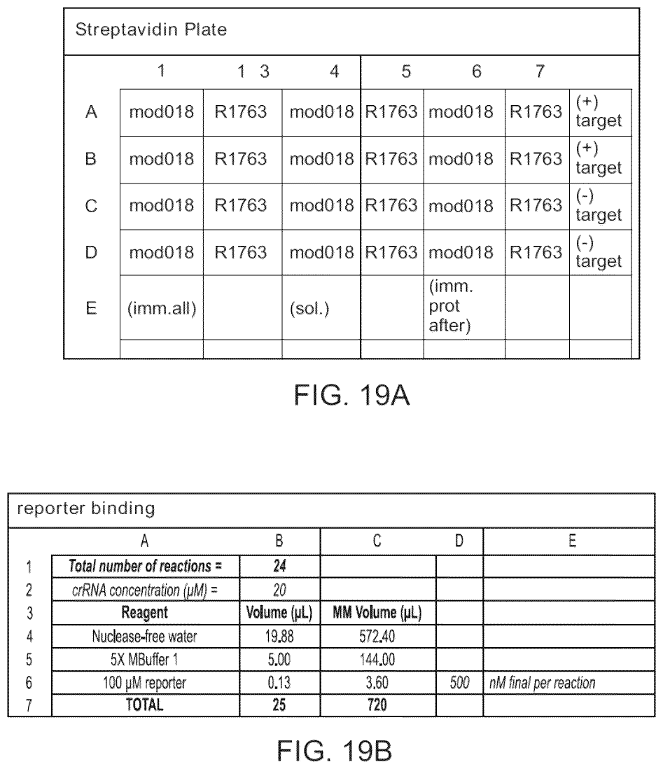

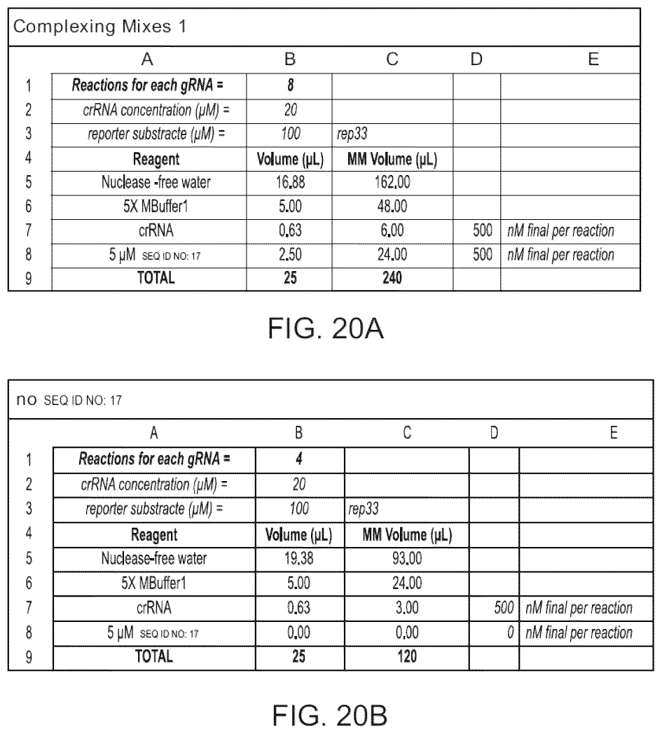

Also described herein are various embodiments where both the guide nucleic acid and reporter are bound to a plate as opposed to the guide nucleic acid, reporter and programmable nuclease. This removes the need to functionalize the surface with a pre-complex of the guide nucleic acid and programmable nuclease, allowing for an easier manufacturing process. Additionally, greater specificity can be achieved by allowing for more stringent washes. An experimental design of this embodiment and exemplary conditions for binding a reporter to a plate in this embodiment are shown in FIGS. 19A and 19B, respectively. Complexing reactions for mod018 (5′ biotin-TEG R1763 SARS-COV-2 N-gene) and R1763 CDC-N2-Wuhan were prepared for a particular embodiment according to the conditions presented in FIG. 20A. Two sets of full complexing mixes according to one non-limiting embodiment are shown in FIG. 20B.

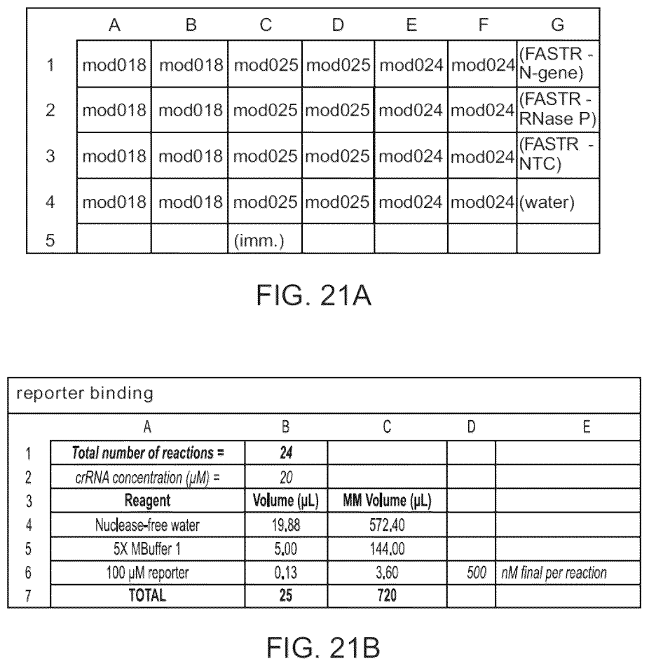

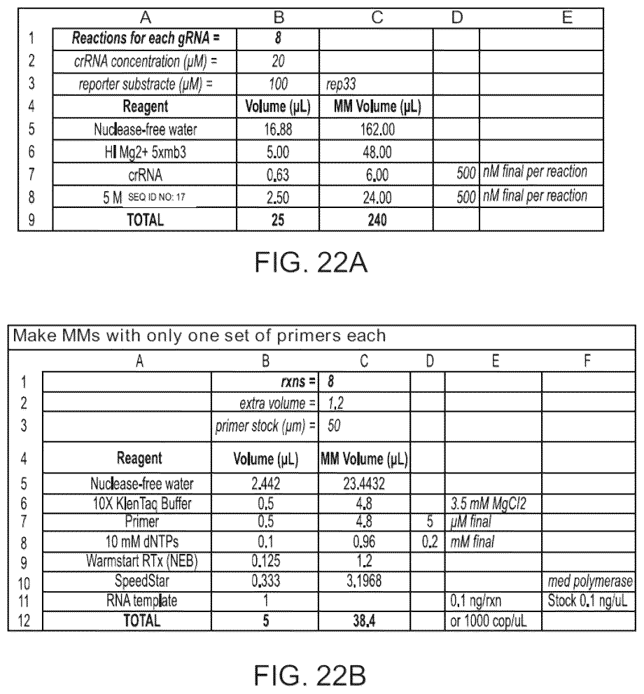

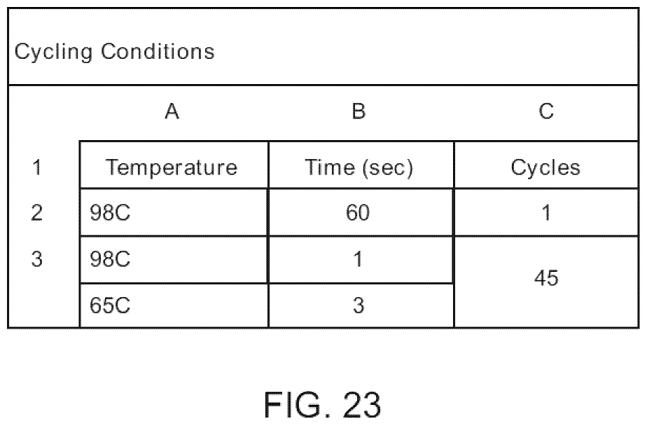

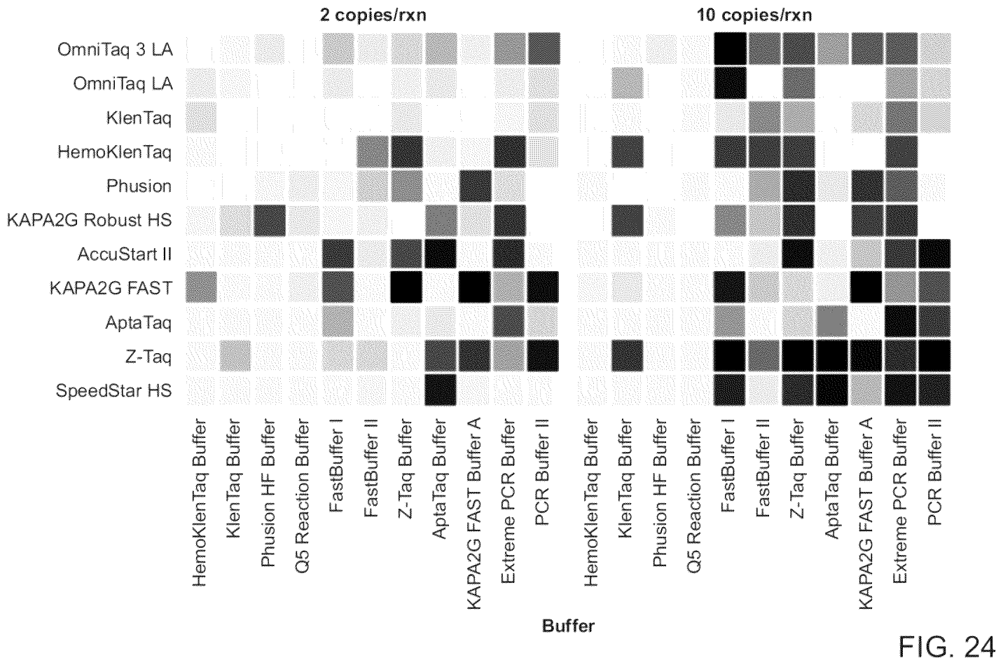

Also described herein are various embodiments that demonstrate target discrimination for immobilized reporters for the DETECTR reaction. An experiment design for such an embodiment is shown in FIG. 21A and reporter binding conditions shown in FIG. 21B. Reaction conditions are shown FIGS. 22A and 22B, PCR conditions are shown in FIG. 23.

### Rapid Thermocycling

In various aspects, the present disclosure provides for a method of detection of SARS-CoV-2 with rapid thermocycling. In some embodiments this method incorporates the optimization of the assay reaction conditions for rapid detection of SARS-COV-2 with rapid thermocycling, herein named as the FASTR assay. In some embodiments, FASTR uses an extreme PCR technique in which the speed of the PCR reaction is decreased to less than 5 minutes by near-instantaneous changes in the reaction temperature. This rapid temperature change may be accomplished by moving the reaction between heat-zones (water baths, heat blocks, etc.) of various temperatures in a thin-walled vessel, instead of cooling or heating the entire instrument for each cycle. Alternatively, the reaction volume can be pumped between two or three heat zones to achieve this rapid thermal change and drive the PCR reaction. In some embodiments, additional speed increases of the PCR reaction can be achieved by increasing the primer, polymerase, and Mg2+ concentrations of the reaction.

In some embodiments, rapid thermocycling and CRISPR diagnostics can be used to detect SARS-COV-2. Results are shown in FIG. 24. For some embodiments, polymerase and buffer combinations were identified that enabled the rapid amplification of SARS-COV-2 using the N2 primers from the CDC assay. The assay of such embodiments was performed at two target concentrations: 2 copies/rxn and 10 copies/rxn. In some embodiments, reaction conditions are as follows: initial denaturation at 98° C. for 30 seconds, followed by 45 cycles consisting of 1 second at 98° C. and 3 seconds at 65° C. Following thermocycling, amplicons were transferred to a Cas12 variant (SEQ ID NO: 17) detection reaction for 30 minutes at 37° C. Best performing enzyme/buffer pairs shown in FIG. 24 were those that gave strong signal in both tested concentrations.

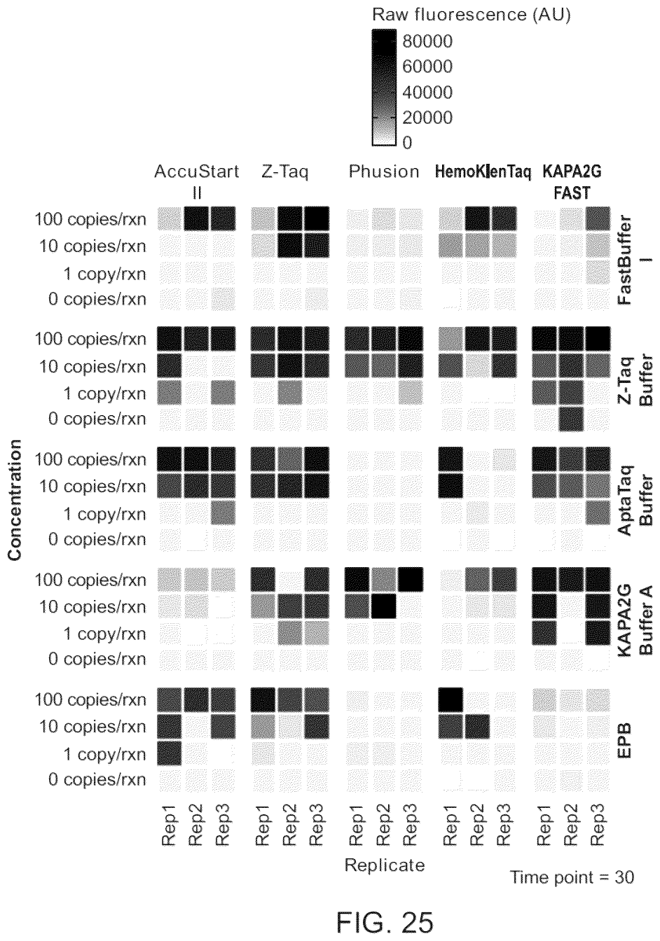

The top enzymes and buffers identified previously at various concentrations and with multiple replicates were tested for the FASTR assay. In some embodiments, the best performing enzymes and buffers as identified in the previously disclosed screening studies were used. Results of such embodiments are shown in FIG. 25. Reaction conditions of such embodiments are as follows: initial denaturation at 98° C. for 30 seconds, followed by 45 cycles consisting of 1 second at 98° C. and 3 seconds at 65° C. Primers used were from the CDC N2 assay for SARS-CoV-2. Following thermocycling, amplicons were transferred to a Cas12 variant (SEQ ID NO: 17) detection reaction for 30 minutes at 37° C. The data present is the signal from the CRISPR reaction. Best performing enzyme/buffer pairs were those that gave strong signal at the lowest tested concentrations and with consistent detection across replicates.

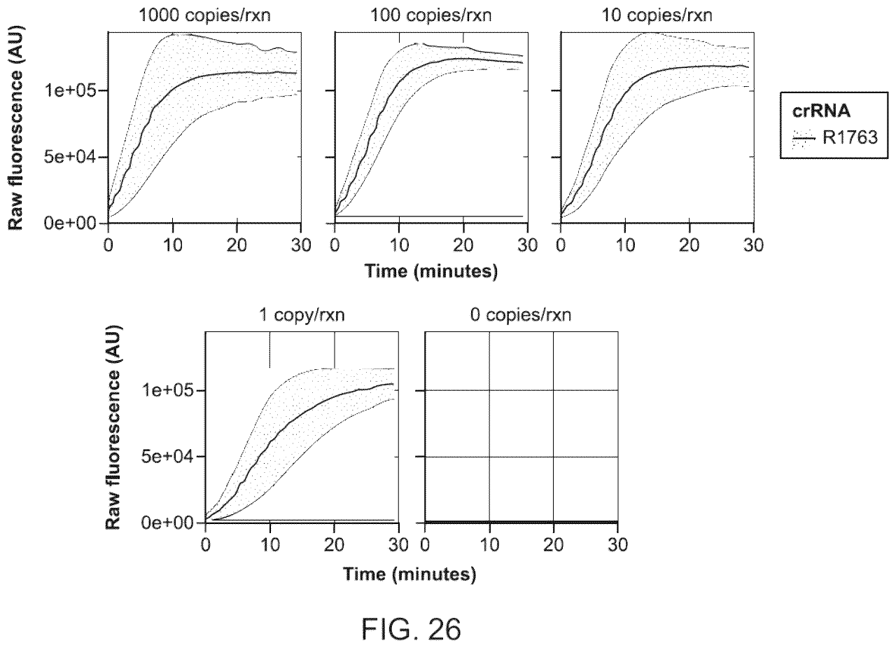

For some embodiments, single copy detection of SARS-COV-2 with FASTR assay has been demonstrated as shown in FIG. 26. In some embodiments, the limit of detection of the FASTR assay was evaluated using solutions composed of 1000 copies of SARS-COV-2 per reaction to 1 copy per reaction. For some embodiments, reaction conditions were as follows: reverse transcription at 55° C. for 60 seconds, initial denaturation at 98° C. for 30 seconds, followed by 45 cycles consisting of 1 second at 98° C. and 3 seconds at 65° C. Primers used were from the CDC N2 assay for SARS-COV-2. Following thermocycling, amplicons were transferred to a Cas12 variant (SEQ ID NO: 17) detection reaction for 30 minutes at 37° C. The data presented in FIG. 26 is the signal from the CRISPR reaction. It was found that the limit of detection of the CRISPR assay was 1 copy of SARS-COV-2 per reaction.

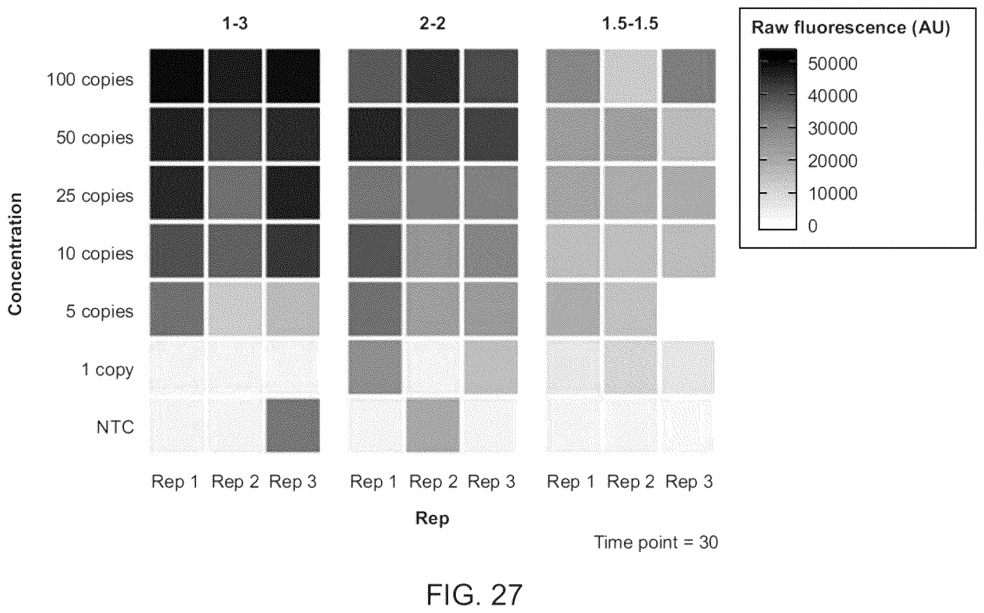

In some embodiments, rapid cycling times were varied to evaluate denaturation and annealing/extension for the FASTR assay. Results for such embodiments are shown in FIG. 27. In some embodiments, reverse transcription was run at 55° C. for 60 seconds and initial denaturation at 98° C. for 30 seconds. In some embodiments, the tested cycling conditions were: 98° C. for 1 second, 65° C. for 3 seconds; 98° C. for 2 seconds, 65° C. for 2 seconds; or 98° C. for 1.5 seconds, 65° C. for 1.5 seconds. In some embodiments, primers used were from the CDC N2 assay for SARS-COV-2. Following thermocycling, amplicons were transferred to a Cas12 variant (SEQ ID NO: 17) detection reaction for 30 minutes at 37° C. The results shown in FIG. 27 indicate that at least about 2 seconds of annealing/extension time at 65° C. is necessary for robust sensitivity.

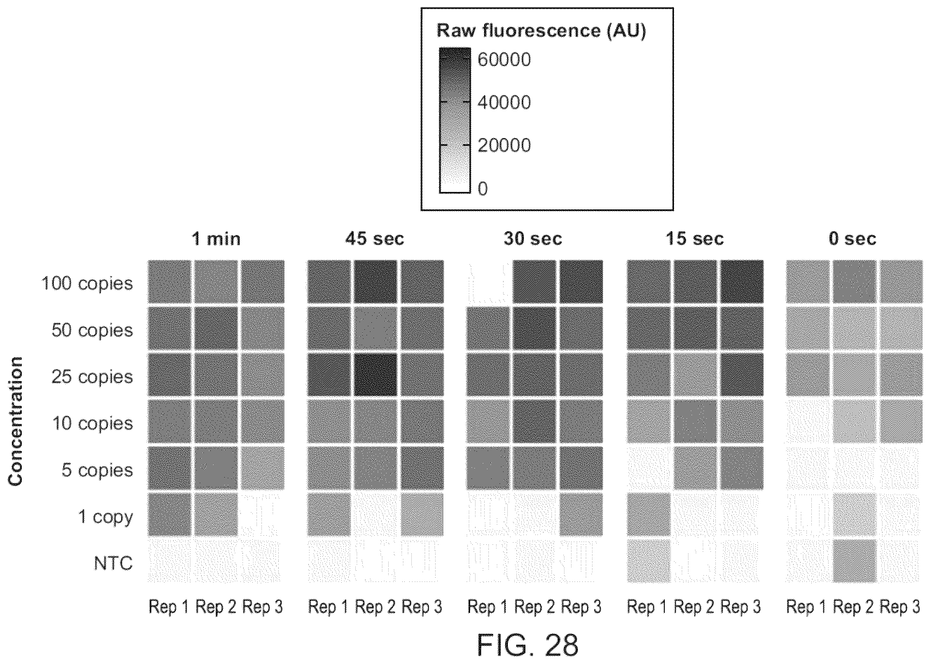

FIG. 28 presents results for Minimizing RT time for FASTR. The performance of the FASTR assay was evaluated, for various embodiments, where the reverse transcription incubation times were varied holding temperature at 55° C. The results shown in FIG. 28 indicate the assay is most robust above 30 seconds of reverse transcription.

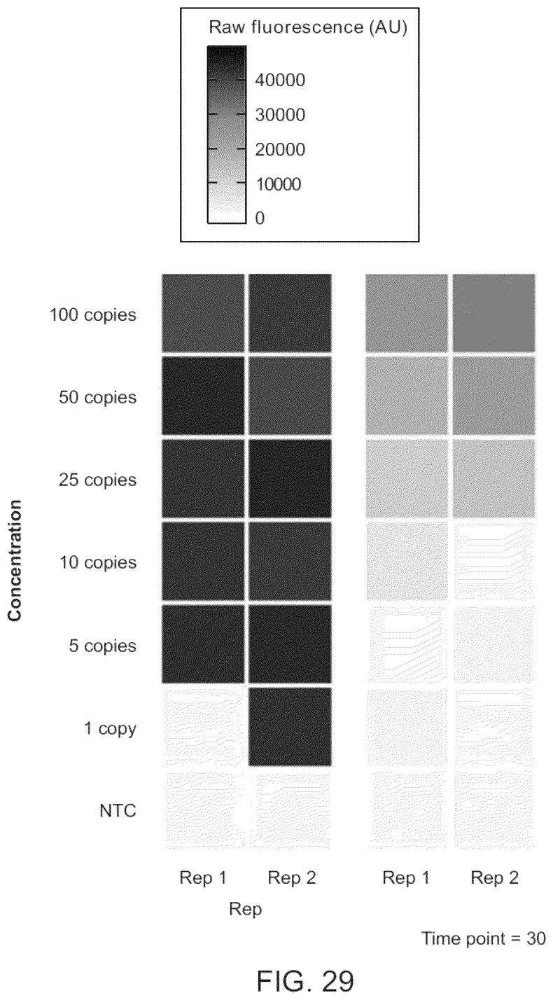

FIG. 29 presents results for Higher pH buffers improve FASTR performance. In some embodiments, the FASTR assay utilizes buffers with pH of either 9.2 or pH 7.8. The FASTR assay was evaluated using these buffer pH values. The results as shown in FIG. 29 indicate that the higher pH buffer produced superior results in terms of amplicon yield and sensitivity.

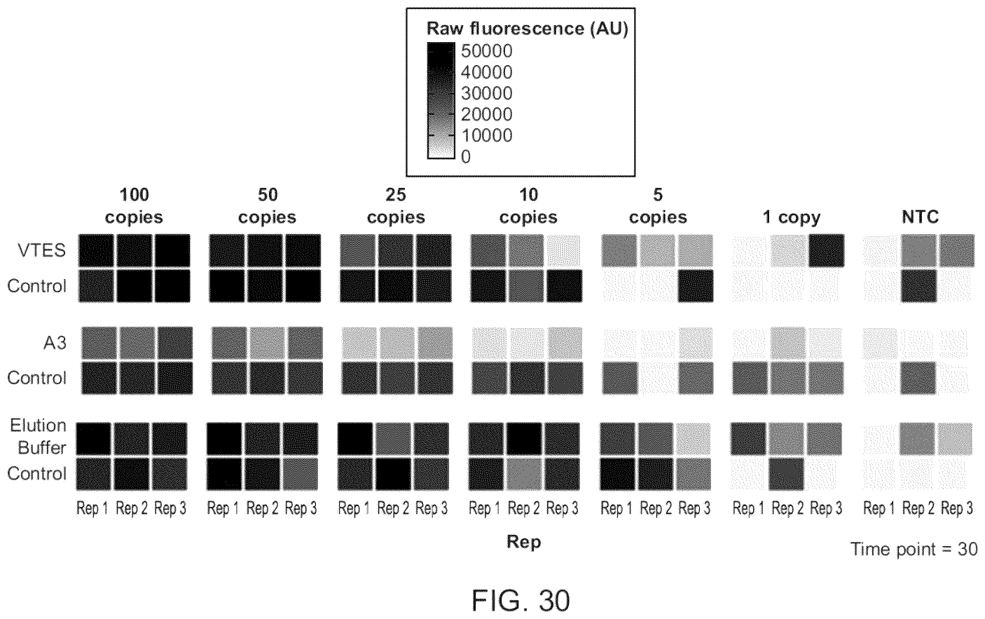

For some embodiments, the FASTR assay compatibility with crude lysis buffers was investigated. Results are shown in FIG. 30 where there are three row groups, each consisting of two sub rows representing a buffer and control from top to bottom respectively. The buffers, VTES, A3 and Elution buffer are plotted against a control, from top to bottom, respectively. In FIG. 30 there are also 7 subgroups showing the number of copies decreasing from left to right. For certain embodiments, the performance of the FASTR assay when combined with various crude lysis buffers was evaluated, where crude lysis buffers VTE5, A3, and the Elution Buffer from the ChargeSwitch kit (Thermo) were tested. For certain embodiments, the FASTR assay performed best for VTE5, but performed slightly less robustly in the A3 buffer and the Elution Buffer from the ChargeSwitch kit, and performed similarly to the control reactions (water).

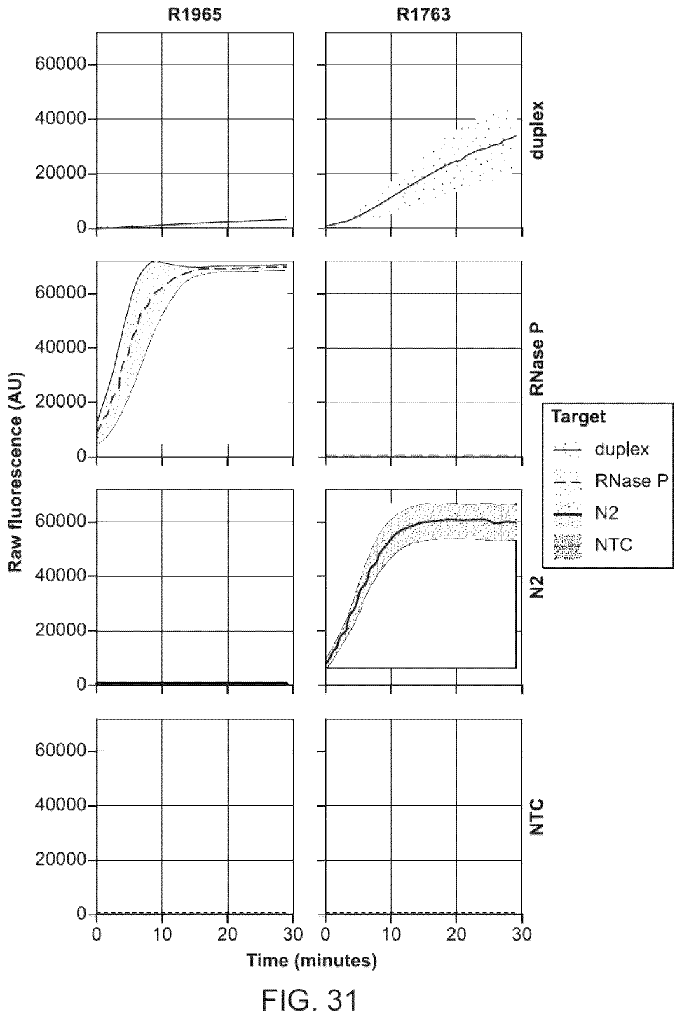

For some embodiments non-optimized multiplexing of FASTR was demonstrated as shown in FIG. 31. In FIG. 31, raw fluorescence is plotted in the y-axis and time is plotted in the x-axis for each sub-plot. Each column illustrates a particular guide nucleic acid sequence: R1965 and R1763, respectively. Each row represents duplex, RNase P, N2 and the no target control, from top to bottom respectively. For some embodiments, initial testing of multiplexed FASTR for SARS-COV-2 and RNase P POP7 (endogenous control) showed that while the single-plex assays generated a robust signal in DETECTR, the duplex assay tended to generate a weak signal for SARS-COV-2 (R1763) and almost no signal for RNase P (R1965). In some embodiments, reaction conditions were as follows: reverse transcription at 55° C. for 60 seconds, initial denaturation at 98° C. for 30 seconds, followed by 45 cycles consisting of 1 second at 98° C. and 3 seconds at 65° C. In some embodiments, primers from the CDC N2 assay for SARS-COV-2, and M3637/M3638 were used.

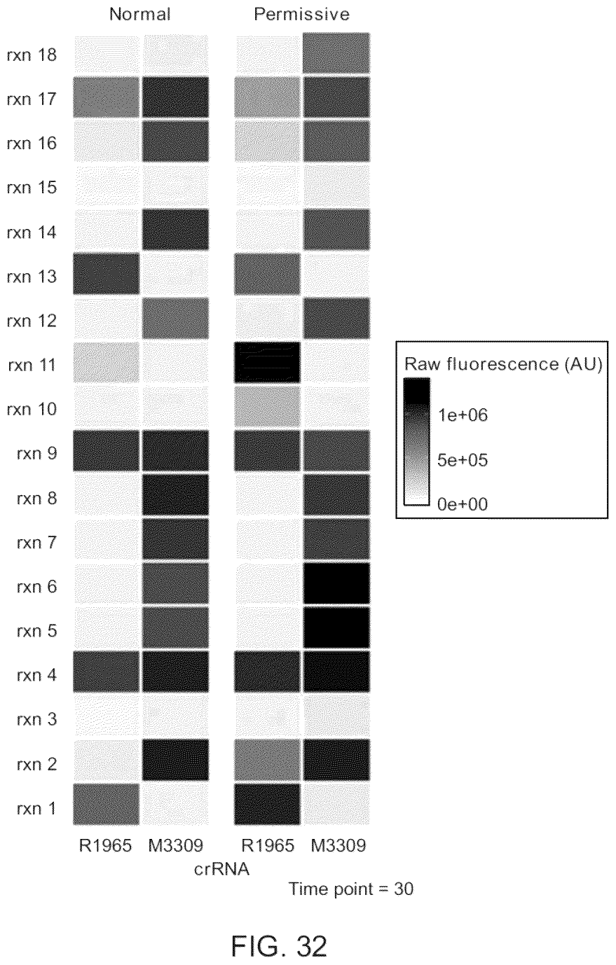

In various aspects described herein, FASTR can be used for multiplexed detection, as shown in FIG. 32. The components of a FASTR reaction, such as primer concentration, dNTP concentration, presence/absence of DMSO, and other factors, can impact the performance of a multiplex FASTR reaction. FIG. 32 shows 18 different experimental conditions for a multiplex FASTR reaction targeting human RNase P POP7 and SARS-COV-2. In FIG. 32 each row of the y-axis represents experimental runs 1-18 and each column represents the detection signal from a particular crRNA at a time point of 30 minutes in the reaction. The shading represents the value of the raw fluorescence. In some embodiments, the multiplexed FASTR assay for SARS-COV-2 and RNase P, comprise a set of SARS-COV-2 primers (M3257/M3258). A series of experiments of such embodiments was performed with varied reaction conditions containing different combinations of buffers, primer concentrations, dNTPs, and DMSO. Results identified two reaction conditions that performed robustly for the multiplex reaction. In one of these embodiments, reaction 4, conditions consisted of: 1× FastBuffer 2; 1 μM RNase P primers; 0.5 uM CoV primers; 0.2 mM dNTPs; and 2% DMSO. In another embodiment, reaction 9, conditions consisted of: 1× Klentaq1 buffer; 1 μM RNase P primers; 0.5 μM CoV primers; 0.4 mM dNTPs; and 0% DMSO. In some embodiments, normal reaction conditions consisted of reverse transcription at 55° C. for 60 seconds, initial denaturation at 98° C. for 30 seconds, followed by 45 cycles consisting of 1 second at 98° C. and 3 seconds at 65° C. In various aspects, permissive reaction conditions consisted of reverse transcription at 55° C. for 60 seconds, initial denaturation at 98° C. for 30 seconds, followed by 45 cycles consisting of 3 seconds at 98° C. and 5 seconds at 65° C.

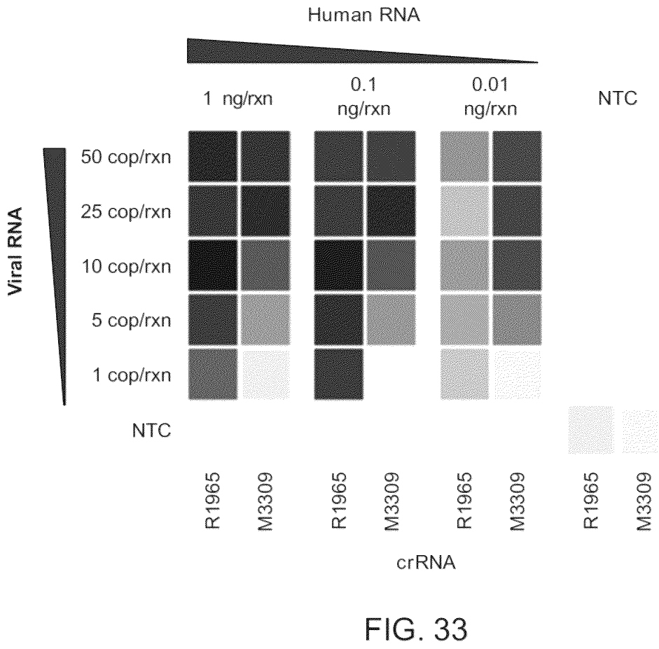

In some embodiments, the FASTR assay enables multiplexed detection. Results of a limit of detection (LOD) study of such embodiments are shown in FIG. 33. In FIG. 33, the x-axis shows the number of copies per reaction for viral RNA and the y-axis of each subplot identifies the particular crRNA. Each subplot shows nanograms of human RNA per reaction, decreasing in concentration from left to right. The 4th subplot contains no human RNA, labeled as the no target control (NTC). For some embodiments, an optimized multiplexed FASTR assay was ran at various concentrations of human RNA and viral RNA. In some embodiments, results indicated that the assay performed at a range of human RNA concentrations, while maintaining a sensitivity of ˜5 copies per reaction. In certain aspects, results shown are from DETECTR reactions using either R1965 to detect the human RNase P, or R3185 (labeled M3309) to detect SARS-COV-2. In various aspects, reaction conditions are as follows: reverse transcription at 55° C. for 60) seconds, initial denaturation at 98° C. for 30 seconds, followed by 45 cycles consisting of 1 second at 98° C. and 3 seconds at 65° C. In some embodiments, primers used were M3257/M3258 (SARS-COV-2) and M3637/M3638 (RNase P).

Table 2B lists various exemplary primers and guide nucleic acids that may be used compatibly with the systems and methods disclosed herein. The key primers and guide nucleic acids may have any of the sequences listed in Table 2B below.

### EXAMPLES

The following examples are given for the purpose of illustrating various embodiments of the invention and are not meant to limit the present invention in any fashion. The present examples, along with the methods described herein, are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Evaluating Function of Biotin Modified gRNAs for Cas13

The purpose of this experiment was to evaluate biotinylated gRNA functionality with Cas13a both in solution and immobilized on a surface. In this experiment three replicate runs of a biotin-modified gRNA (mod023) and three replicate runs of a non-biotin modified gRNA (R0003) were carried out. Three replicate “no target control,” or NTC runs were carried out for both the mod023 reporter and R0003 control. The procedure was carried out as follows: (1) gRNAs were diluted to 20 μM.

(2) Cas13 variant (SEQ ID NO: 21) complexing reactions using gRNAs were prepared. The testing and control complexes were diluted to 100 pM final concentrations. Complexing reactions were carried out in two conditions with 3 replicates each resulting in 6 reactions per gRNA. FIG. 16 schematically illustrates complexing mix details.

(3) Sample was incubated for 30 min at 37° C.

(4) Reporter substrate was added.

(5) Reaction was kept on ice until the next step.

(6) 13 μL of 1× MBuffer 1 was added in wells on 384-well plates.

(7) 5 μL of complexing reaction was added.

(8) In a post-amp hood, 2 μL of 1 nM target (respective) was transferred to and kept in a 384 well plate on ice.

(9) Sealed plate with optically clear seal.

(10) Spun down for 30 sec at 2000 rcf.

(11) Read on plate reader with extended gain settings for 30 min at 37° C.

FIGS. 13A and 13B show results, in solution, for mod023, the biotin modified reporter, and R003 the non-biotin-modified reporter, respectively. In some embodiments, the biotin-modified guide nucleic acid has similar performance to the non-biotin-modified guide nucleic acid in solution. FIG. 13C shows results for gRNA that was modified with biotin and immobilized to the surface. FIG. 13D shows results for gRNA that was not modified with biotin but was deposited on the surface in the same manner as FIG. 13C, FIG. 13E, similar to FIGS. 13A and 13B, shows results for gRNA that was unmodified and in solution. Together these results showed that with biotin modification and surface immobilization, functionality was maintained and DETECTR assay performance was not adversely affected.

### Example 2: Optimizing Reporter Incubation Time on Streptavidin Slides

The objective of this experiment was to verify if a 30 min incubation time was sufficient to produce a strong immobilization signal. Two concentrations were run. The procedure used is as follows:

1. Dilutions of rep072 or rep106 at 1uM and 5 μM in 1× Wash Buffer were prepared, 1× Wash Buffer is composed of 25 mM Tris, 150 mM NaCl; pH 7.2; 0.1% BSA, and 0.05% Tween®-20 Detergent.

2. The wells of a fresh streptavidin slide were marked as shown in FIGS. 14, 15A, and 15B using a hydrophobic pen.

3. Three μL of each dilution were spotted on the streptavidin coated slide (in triplicate).

4. The slide was incubated for 30 min at room temperature and covered from light.

5. Three μL of the dilution were removed from each spot.

6. Five μL of 1× wash buffer was added to each spot and mixed up and down 3 times. This wash step was repeated three times.

7. 50 μL of 1× wash buffer was added and incubated for 5 min.

8. The slide was then flicked over a kim wipe to remove all the solution, then dabbed gently on all four sides against a kim wipe to clean up the edges.

9. The slides were then imaged on GelDoc (SYBR Blue setting, autoexposure with adjusted gain settings).

FIG. 14 shows results for the test reporter, Rep072, and the negative control, Rep106. The replicates of Rep072 at 5 μM show the strongest signal and the three replicates of Rep072 at 1 μM concentration show the next strongest signal. The negative control reporter, rep106 shows the same low signal (or none at all) for both 5 μM and 1 μM concentrations. This result shows specific binding of a FAM-biotinylated reporter with a 30 minute incubation time at both 5 μM and 1 μM concentrations. FIGS. 15A-15B show similar results with reporters at 5 mM in FIG. 15A and 2.5 mM in FIG. 15B. The top row of FIGS. 15A and 15B shows spots exhibiting bright fluorescence and the bottom row of FIGS. 15A and 15B show spots exhibiting similarly low fluorescence.

### Example 3: Quencher-Based Reporter Testing for Immobilization

Fluorescent quencher-based reporters were tested in an immobilized DETECTR assay. Streptavidin functionalized plates and biotin labeled reporters were used. FIG. 17 shows sequence and other details for reporters used in this experiment. The following procedure was used:

1. Stocks of the reporters rep072, rep104, rep105, rep117, and rep118 were prepared for binding to the reader plate. Reporter binding details can be seen in FIG. 18A.

2. Complexing reactions were then prepared using the mod018 sequence that is 5′ modified with biotin TEG. See FIG. 17 for more details on sequence mod018. Complexing mix details can be seen in FIG. 18B.

3. Complexing reactions were incubated for 30 min at 37° C.

4. Grid of dilutions of RNP and reporter were prepared with (50:50 ratio) with enough material for 2 reactions each.

5. Wells of a 96-well streptavidin coated plate were pre-rinsed with 100 μL of 1× MBuffer1, twice.

6. 25 μL of complex and 25 μL reporter mix were then added.

7. Sealed plate with foil seal.

8. Binding was then carried out at 25° C. for 30 minutes with intermittent shaking (1000 rpm 15 see every 2 min on Thermomixer).

9. Plates were then spun down briefly.

10. Supernatant was removed.

11. Washed once with 100 μL 1× MBuffer-1, 1× MBuffer-1 is composed of 20 mM Imidazole 7.5, 25 mM KCl, 5 mM MgCl2, 10 μg/mL BSA, 0.01% Igepal Ca-630, and 5% Glycerol.

12. Washed once with 100 μL 1× MBuffer-3, 1× MBuffer-3 is composed of 20 mM HEPES pH 7.5, 2 mM KOAc, 5 mM MgOAc, 1% Glycerol, and 0.00016% Triton-X 100.

13. Added 50 μl of 1× MBuffer3 to each well.

14. Added 5 μL of target/no-target in 1× MBuffer3 a. target volume=5 μL per reaction (GF577 PCR product 1:10).

15. Sealed plate with foil seal.

16. Incubated at 37° C. for 90 minutes with intermittent shaking in plate reader measuring FAM intensity.

17. Spun down briefly.

18. Transferred 20 μL of supernatant to wells of 384-well plate and measured FAM fluorescent intensity (single-read).

Results are illustrated in FIGS. 8A-8E. FIG. 8A presents the binding results for a FAM-biotin reporter, “rep” composed of the fluorophore FAM and biotin and is listed as rep72. FIG. 8B plots the raw fluorescence for a reporter composed of the fluorophore AlexaFluor488, “AF488,” and TA10-internalBiotinQ. As predicted, the positive control shown in FIG. 8A shows a positive slope indicating increased binding of Cas enzyme and target (and subsequent reporter cleavage) over the course of the reaction. This is due to the release of FAM dye into solution upon binding and transcleavage as seen in FIG. 8B. In rep104, the cleavage point is between the FAM and the biotin, while the biotin in all reporters is the attachment point to the streptavidin surface. FIG. 8C plots the control, target binding kinetic plot for rep105, Rep105 is composed of biotin-FAM-T16-FQ. In this case the streptavidin coated surface emits fluorescence because the region between the FAM dye and the quencher is cleaved upon binding and the quencher is released. FIG. 8D plots the control for rep117, Rep117 is composed of biotin-FAM-T20-FQ. In this embodiment, the reporter is cleaved between the FAM dye and the quencher, thus allowing for release of the quencher in the solution upon binding and transcleavage. This in turn, causes the surface to emit fluorescence. FIG. 8E plots the control for rep118, Rep118 is composed of FAM-T20-biotin-FQ. In this embodiment, the solution emits fluorescence because upon binding the nucleic acid region between the biotin and the FAM is transcleaved, thus releasing the FAM into solution.

### Example 4: Immobilization Optimization-Complex Formation Step

The objective of this experiment was to determine whether binding both the gRNA and reporter to a plate allows the DETECTR assay to be as effective as binding the CAS protein-gRNA complex and reporter. This removes the need to functionalize the surface with the pre-complex of guide nucleic acid and programmable nuclease, allowing for an easier manufacturing process. Additionally, greater specificity can be achieved by allowing for more stringent washes. The following procedure was used.

1. The experiment was designed as shown in FIG. 19A.

2. A stock solution of reporter rep117 was bound to the plate according to the conditions presented in FIG. 19B.

3. Complexing reactions for mod018 (5′ biotin-TEG R1763 SARS-COV-2 N-gene) and R1763 CDC-N2-Wuhan were then prepared according to the conditions presented in FIG. 20A.

4. Two sets of full complexing mix were made for each and two mixes without Cas12 variant (SEQ ID NO: 17) according to FIG. 20B.

5. Incubated complexing reactions for 30 min at 37° C.

6. Pre-rinsed wells of 96-well streptavidin coated plate with 50 μL of 1× MBuffer1, twice.

7. Added 25 μL reporter to each well.

8. Added 25 μL of complex to A1-D2, 25 μL 1× MB1 to A3-D4, and 25 μL cRNA mix A5-D6.

9. Sealed plate with foil seal.

10. Ran binding reaction at 25C for 30 minutes with intermittent shaking, 1000 rpm 15 sec every 2 min on Thermomixer.

11. Spun streptavidin plate down briefly.

12. Removed supernatant.

13. Washed twice with 100 μL 1× MBuffer-1.

14. Washed once with 100 μL 1× MBuffer-3.

15. Added 50 μl of 1× MBuffer3 to wells A1-D2.

16. Added 25 μL 1×MB3 and 25 μL of complex to “in-solution” wells A3-D4.

17. Added 47.5 μL 1×MB3 and 2.5 μL Cas12 variant (SEQ ID NO: 17) (50 μL MM) to each “prot after” well A5-D6.

18. Added 5 μL of 1:10 diluted purified LAMP product to (+) target wells.

19. Sealed plate with optically clear seal.

20. Read on plate reader—FAM, 37° C., 90 min.

The results of this experiment (see FIGS. 10A-10F) show that is it possible to add CAS protein with the target and still achieve complexing and signal. FIGS. 10A-10C illustrate results for a first replicate of tests. FIGS. 10D-10F illustrate results for a second replicate of tests, FIGS. 10A and 10D show results where both a biotinylated reporter and a complex of biotinylated RNA and CAS protein were immobilized. Here activity buffer and target were then added. FIGS. 10B and 10E illustrate results where the biotinylated reporter is immobilized and all other reaction components including guide nucleic acids and programmable nucleases are introduced in solution. FIGS. 10C and 10F illustrate results where the biotinylated reporter and biotinylated gRNA are immobilized and then buffer, CAS protein and target are added. In these results it is observed that complexation of programmable nucleases and guide nucleic acids and a reporter signal emitted upon binding can be detected when only guide nucleic acid and reporter are immobilized as shown in FIG. 10F.

### Example 5: Demonstration of Immobilized Target Discrimination

The purpose of this experiment was to demonstrate target discrimination for systems comprising immobilized reporters for the DETECTR reaction. The experiment design used in this experiment is shown in FIG. 21A. The following procedure was used.

1. Experiment planned as shown in FIG. 21A. The experiment included 3 gRNAs including mod018, mod025, and mod024. Two targets and two controls were used. The two targets were N-gene and RNaseP. The two controls were: (1) no target with all other reaction components and (2) water.

2. Stock solution of reporter rep117, later bound to plate, was prepared as shown in FIG. 21B.

3. Complexing reactions were prepared for the three gRNAs: mod018, mod024 and mod025 with reporters:
(1) biotin-TEG R1763 SARS-COV-2 N-gene (mod018),
(2) 5′ biotin-TEG R777 Mammuthus (mod024),
(3) 5′ biotin-TEG R1965 RNase P (mod025), respectively.

The reaction conditions are shown FIG. 22A.

4. Pre-rinsed wells of 96-well streptavidin coated plate with 50 μL of 1× MBuffer1, twice.

5. Added 25 μL reporter to each well.

6. Added 25 μL complexing mix to wells.

7. Sealed plate with foil seal.

8. Ran binding reaction at 25° C. for 30 minutes with intermittent shaking (1000 rpm 15 sec every 2 min on Thermomixer).

9. Ran FASTR protocol as follows:
a. Primers used:
I. SARS-COV-2: M2062 CDC N2-FWD/M2063 CDC N2-REV.
II. RNase P: POP7 8F/6R.
See FIG. 22B for reaction conditions,
a. Pipette 4 μL of master mix into wells of MBS 96-well plate.
b. Added 1uL twist RNA dilution.
c. 1000 copies/uL; 7.8 uL of 6400c/uL in 42.2 uL H2O.
d. Sealed plate with foil seal at 165° C. for 1.5 seconds.
e. Ran the following PCR protocol on the MBS NEXTGENPCR thermocycler according to conditions shown in FIG. 23.
f. Removed plate from thermocycler.
g. Spun down at 2000 rpm for 30 sec.
h. Kept on ice until ready to use.

10. Spun streptavidin plate down briefly.

11. Removed supernatant.

12. Washed twice with 100 μL 1× MBuffer-1.

13. Washed once with 100 μL 1× MBuffer-3.

14. Added 50 μL 1×MB3 15 mM Mg2+.

15. Added 4 μL of target from FASTR to target wells.

16. Sealed plate with optically clear seal.

17. Read on plate reader-FAM, 37° C., 90 min.

Results are shown in FIGS. 12A-12C. FIG. 12A presents results for reporter mod018 showing specificity for the N-gene target. FIG. 12B presents results for reporter mod025 showing specificity for the RNaseP target. FIG. 12C presents results for mod024 showing no signal as predicted since no target was present.

### Example 6: Detection of SARS-COV-2 with Rapid Thermocycling

This example describes the steps taken for the optimization of assay reaction conditions for rapid detection of SARS-COV-2 with rapid thermocycling, herein referred to as the FASTR assay, FASTR uses an extreme PCR technique in which the speed of the PCR reaction is decreased to less than 5 minutes by near-instantaneous changes in the reaction temperature. This rapid temperature change may be accomplished by moving the reaction between two or more heat-zones (water baths, heat blocks, etc.) of various temperatures in a thin-walled vessel, instead of cooling or heating the entire instrument for each cycle. Alternatively, the reaction volume can be pumped between two or three heat zones to achieve this rapid thermal change and drive the PCR reaction. Additional speed increases of the PCR reaction can be achieved by increasing the primer, polymerase, and/or Mg2+ concentrations of the reaction.

FIG. 24 depicts the results from the polymerase and buffer combinations that enabled the rapid amplification of SARS-COV-2 using primers directed to the N-gene of SARS-COV-2 (primer sequences presented in Table 2B, “CDC N2 assay for SARS-COV-2”). The assay was performed at two target concentrations: 2 copies/reaction (rxn) and 10 copies/reaction (rxn). Reaction (rxn) conditions are as follows: initial denaturation at 98° C. for 30 seconds, followed by 45 cycles consisting of 1 second at 98° C. and 3 seconds at 65° C. Following thermocycling, target amplicons were transferred to a Cas12 variant (SEQ ID NO: 17) detection reaction for 30 minutes at 37° C. The data presented in FIG. 24 is the signal from the CRISPR reaction. Best performing enzyme/buffer pairs were those that gave strong signal in both tested concentrations.

The top enzymes and buffers identified in FIG. 24 were tested at various concentrations and with multiple replicates as shown in FIG. 25 to further optimize the reaction conditions for the FASTR assay. Reaction conditions were as follows: initial denaturation at 98° C. for 30 seconds, followed by 45 cycles consisting of 1 second at 98° C. and 3 seconds at 65° C. Primers used were from the CDC N2 assay for SARS-COV-2 (primer sequences presented in Table 2B). Following thermocycling, amplicon was transferred to a Cas12 variant (SEQ ID NO: 17) detection reaction for 30 minutes at 37° C. The data presented in FIG. 25 is the signal from the CRISPR reaction. Best performing enzyme/buffer pairs were those that gave strong signal at the lowest tested concentrations and with detection across replicates.

To further evaluate the performance of the FASTR assay, the limit of detection of the assay was evaluated from 1000 copies/reaction to 1 copy/reaction. Reaction conditions were as follows: reverse transcription at 55° C. for 60 seconds, initial denaturation at 98° C. for 30 seconds, followed by 45 cycles consisting of 1 second at 98° C. and 3 seconds at 65° C. Primers used were from the CDC N2 assay for SARS-COV-2 (sequences presented in Table 2B). Following thermocycling, amplicon was transferred to a Cas12 variant (SEQ ID NO: 17) detection reaction for 30 minutes at 37° C. The data presented in FIG. 26 is the signal from the CRISPR reaction. The assay performed well at 1 copy/reaction and was able to detect SARS-COV-2 at a single copy level. It was found that the limit of detection of the CRISPR assay was 1 copy of SARS-CoV-2 per reaction.

The effect of variations in rapid cycling times for denaturation and annealing/extension in FASTR assay was also evaluated. To determine the best cycling conditions for the FASTR assay, the performance of the assay was evaluated with varied cycling conditions. For all reactions, reverse transcription was performed at 55° C. for 60 seconds and initial denaturation at 98° C. for 30 seconds. The tested cycling conditions were: 98° C. for 1 second, 65° C. for 3 seconds; 98° C. for 2 seconds, 65° C. for 2 seconds; or 98° C. for 1.5 seconds, 65° C. for 1.5 seconds. Primers used were from the CDC N2 assay for SARS-COV-2 (sequences presented in Table 2B). Following thermocycling, amplicon was transferred to a Cas12 variant (SEQ ID NO: 17) detection reaction for 30 minutes at 37° C. The results shown in FIG. 27 indicate that >2 seconds of annealing/extension time at 65° C. are necessary for robust sensitivity.

In order to minimize the reverse transcription (RT) time for FASTR, the performance of the FASTR assay was evaluated with various reverse transcription incubation times at 55° C. to determine the minimal reverse transcription conditions for the FASTR assay. The results of this assay optimization in FIG. 28 indicate the assay is most robust above 30 seconds of reverse transcription.

In order to test the effect of pH of the reaction buffer on the FASTR assay performance, the performance of the FASTR assay with buffers with pH of either 9.2 or pH 7.8 was evaluated. The results, as shown in FIG. 29 indicate that the higher pH buffer produced superior results in terms of amplicon yield and sensitivity.

In order to test the compatibility of the FASTR assay with crude lysis buffers, the performance of the FASTR assay when combined with various crude lysis buffers was evaluated, including Crude lysis buffers VTE5, A3, and the Elution Buffer from the ChargeSwitch kit (Thermo). In FIG. 30 there are also 7 subgroups showing the number of copies decreasing from left to right. As seen in FIG. 30, the FASTR assay performed the best in the VTE5 lysis buffer, but performed slightly less robustly in the A3 buffer. The Elution Buffer from the ChargeSwitch kit performed similarly to the control reactions (water).

As shown in FIG. 31, initial non-optimized testing of multiplexed FASTR for SARS-CoV-2 and RNase P POP7 (endogenous control) showed that while the single-plex assays generated a robust signal in DETECTR, the duplex assay tended to generate a weak signal for SARS-COV-2 (R1763) and almost no signal for RNase P (R1965). Reaction conditions were as follows: reverse transcription at 55° C. for 60 seconds, initial denaturation at 98° C. for 30 seconds, followed by 45 cycles consisting of 1 second at 98° C. and 3 seconds at 65° C. Primers used were from the CDC N2 assay for SARS-COV-2, and M3637/M3638 as shown in Table 2B.

Considering the results of the non-optimized multiplexed FASTR assay in FIG. 31, in order to optimize multiplex FASTR for SARS-COV-2 and RNase P, a new set of SARS-COV-2 primers (M3257/M3258) were designed (sequences presented in Table 2B). A series of experiments with varied reaction conditions containing different combinations of buffers, primer concentrations, dNTPs, and DMSO were then performed. The results of this experiment, as shown in FIG. 32, identified two reaction conditions that performed robustly for the multiplex reaction (depicted by arrows at Reaction 4 and Reaction 9). In Reaction 4, the following conditions were used: 1× FastBuffer 2, 1 μM RNase P primers, 0.5 μM CoV primers, 0.2 mM dNTPs, 2% DMSO. In Reaction 9, the following conditions were used: 1× Klentaq1 buffer, 1 μM RNase P primers, 0.5 μM CoV primers, 0.4 mM dNTPs, 0% DMSO. Under the normal reaction conditions, reverse transcription was performed at 55° C. for 60 seconds, initial denaturation at 98° C. for 30 seconds, followed by 45 cycles consisting of 1 second at 98° C. and 3 seconds at 65° C. Under the permissive reaction conditions, reverse transcription was performed at 55° C. for 60 seconds, initial denaturation at 98° C. for 30 seconds, followed by 45 cycles consisting of 3 seconds at 98° C. and 5 seconds at 65° C.

Once these conditions were optimized, the optimized multiplexed FASTR assay were evaluated at various concentrations of human RNA and viral RNA to evaluate the limit of detection of multiplex FASTR reaction. The results as shown in FIG. 32 indicate that the assay performs at a range of human RNA concentrations, while maintaining a sensitivity of ˜5 copies/reaction. Results shown in FIG. 33 are from DETECTR reactions using either primer R1965 to detect the human RNase P, or primer R3185 (labeled M3309) to detect SARS-COV-2. The primer sequences of R1965 and R3185 are presented in Table 2B. The reaction conditions tested were as follows: reverse transcription at 55° C. for 60 seconds, initial denaturation at 98° C. for 30 seconds, followed by 45 cycles consisting of 1 second at 98° C. and 3 seconds at 65° C. Primers used were M3257/M3258 (SARS-COV-2) and M3637/M3638 (RNase P) (presented in Table 2B).

TABLE 2B
Primers and guide nucleic acids used for optimization of reaction (rxn)
conditions for testing of SARS-CoV2 using rapid thermocycling

| Name | Sequence | Purpose | Note |
|---|---|---|---|
| M2062 | TTACAAACATTGG | PCR primer | CDC N2 assay for SARS- |
| | CCGCAAA (SEQ ID | | CoV-2 |
| | NO: 106) | | |
| M2063 | GCGCGACATTCCG | PCR primer | CDC N2 assay for SARS- |
| | AAGAA (SEQ ID | | CoV-2 |
| | NO: 107) | | |
| R1763 | UAAUUUCUACUAA | Cas 12 gRNA | Compatible with M2062/ |
| | GUGUAGAUCCCCC | | M2063 |
| | AGCGCUUCAGCGU | | |
| | UC (SEQ ID NO: | | |
| | 108) | | |
| M3637 | CCTCCGTGATATG | PCR primer | Human RNase P POP7 |
| | GCTCTTC (SEQ ID | | |
| | NO: 109) | | |
| M3638 | AGAGTCCTTTGGG | PCR primer | Human RNase P POP7 |
| | CTTCC (SEQ ID NO: | | |
| | 110) | | |
| R1965 | UAAUUUCUACUAA | Cas12 gRNA | Compatible with M3637/ |
| | GUGUAGAUUUAC | | M3638 |
| | AUGGCUCUGGUCC | | |
| | GAG (SEQ ID NO: | | |
| | 111) | | |
| M3257 | AGGTGCCTGGAAT | PCR primer | SARS-CoV-2, orf1ab |
| | ATTGGTGAACAG | | |
| | (SEQ ID NO: 112) | | |
| M3258 | TCAAGAGTGCGGG | PCR primer | SARS-CoV-2, orf1ab |
| | AGAAAATTGATCG | | |
| | (SEQ ID NO: 113) | | |
| R3185 | UAAUUUCUACUAA | Cas12 gRNA | Compatible with M3257, |
| | GUGUAGAUCAUCA | | M3258 |
| | GAGGCUGCUCGUG | | |
| | UU (SEQ ID NO: 114) | | |

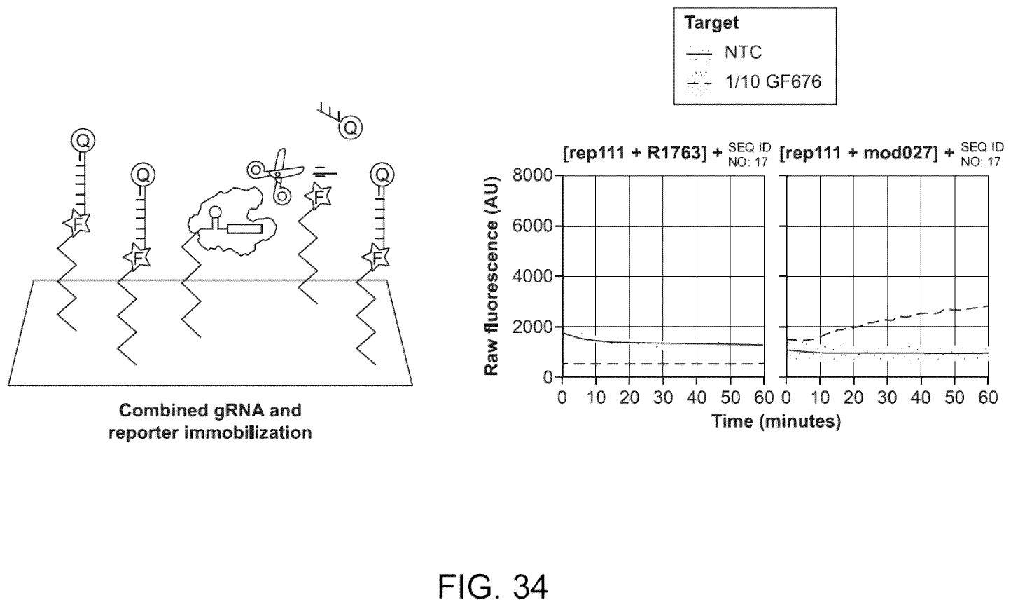

In some embodiments, N-Hydroxysuccinimide (NHS)-Amine chemistry can be used for immobilization of DETECTR components. FIG. 34 presents a schematic of combined guide nucleic acid (e.g., gRNA) and reporter immobilization and results for such embodiments. In this embodiment, a functional DETECTR reaction was immobilized to a solid substrate (NHS plate) using primary amine modified reporters and guide nucleic acids (e.g., gRNAs). In the example shown, a modified reporter (rep111) was bound to the surface in combination with either an unmodified crRNA (R1763) or a modified crRNA (mod027). After incubating these nucleic acids on the surface, the surface was washed 3 times, and then a programmable nuclease (e.g., a Cas12 variant (SEQ ID NO: 17)) was added with the target dsDNA. The immobilized DETECTR reaction was then incubated in a plate reader at 37 degrees Celsius for 60 minutes with continuous monitoring of the fluorescence.

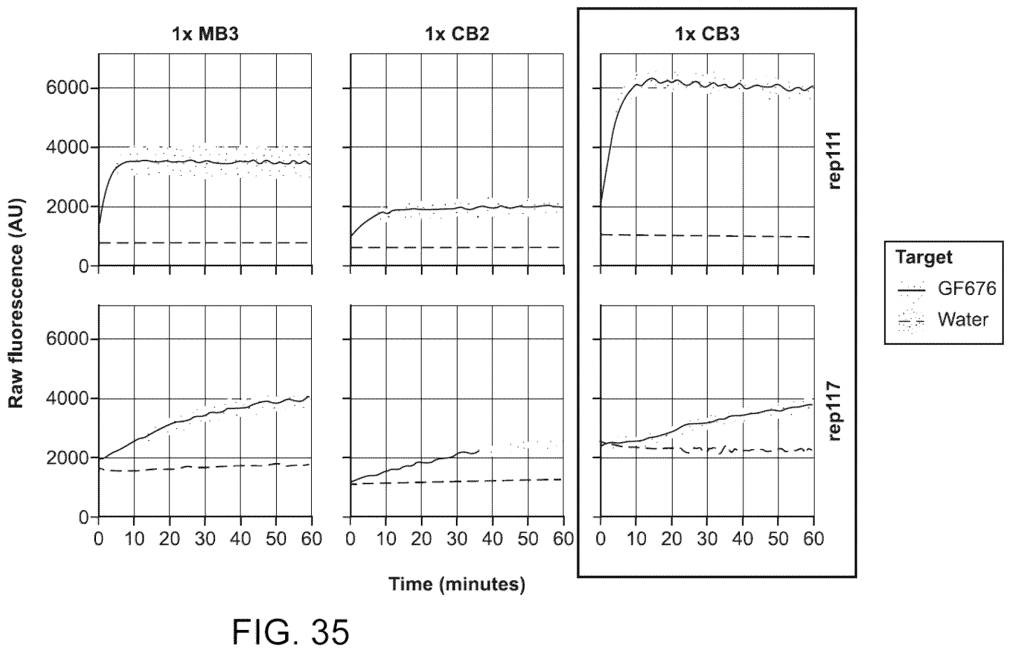

FIG. 35 presents results for various embodiments involving the use of an optimizing conjugation buffer to reduce non-specific binding of DETECTR reagents. For some embodiments, 1× Conjugation Buffer 3 (CB3) was selected as the buffer to perform binding studies. It was found that CB3 improved the binding of the amine-modified reporter (rep111) and reduced the binding of a biotinylated reporter (rep117) which should not bind to NHS covalently. In some embodiments, the wash buffer used was 1× MB3. In some embodiments, 1× MB3: 20 mM HEPES, pH 7.2, 2 mM KOAc, 5 mM MgAc, 1% Glycerol, 0.0016% Triiton X-100 was used. In some embodiments, 1× CB2: 20 mM HEPES, pH 8.0, 2 mM KOAc, 5 mM MgAc, 1% Glycerol, 0.0016% Triiton X-100 was used. In this embodiment, 1×CB3: 100 mM HEPES, pH 8.0, 10 mM KOAc, 25 mM MgAc was used.

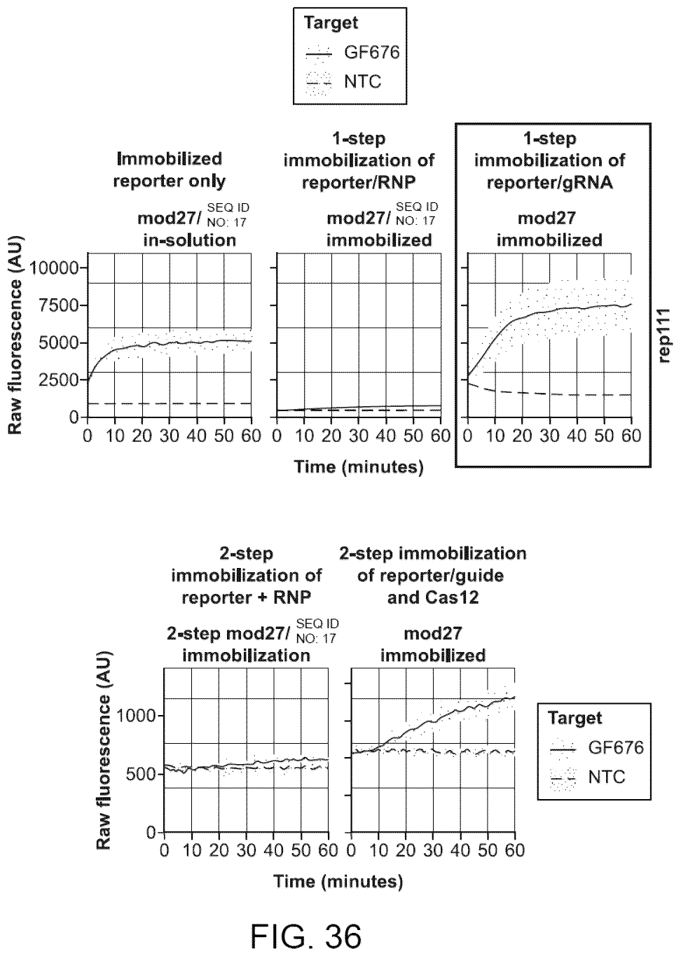

In some embodiments, different combinations of reporters, guide nucleic acids, and/or programmable nucleases (including, for example, Cas12 variant (SEQ ID NO: 17)) may be immobilized. FIG. 36 presents results of such embodiments, involving the optimization of the assay. For such embodiments, it was found that immobilizing guide nucleic acids and/or reporters first followed by the addition of a programmable nuclease (e.g., Cas12 variant (SEQ ID NO: 17)) and a target at the same time gave sufficient signal. As used herein, a sufficient signal may correspond to any signal that is observable or detectable using any of the sensors described herein. Alternatively or in addition, the sufficient signal may correspond to any signal that is observable or detectable by the human eye without needing or requiring the use of any sensors.

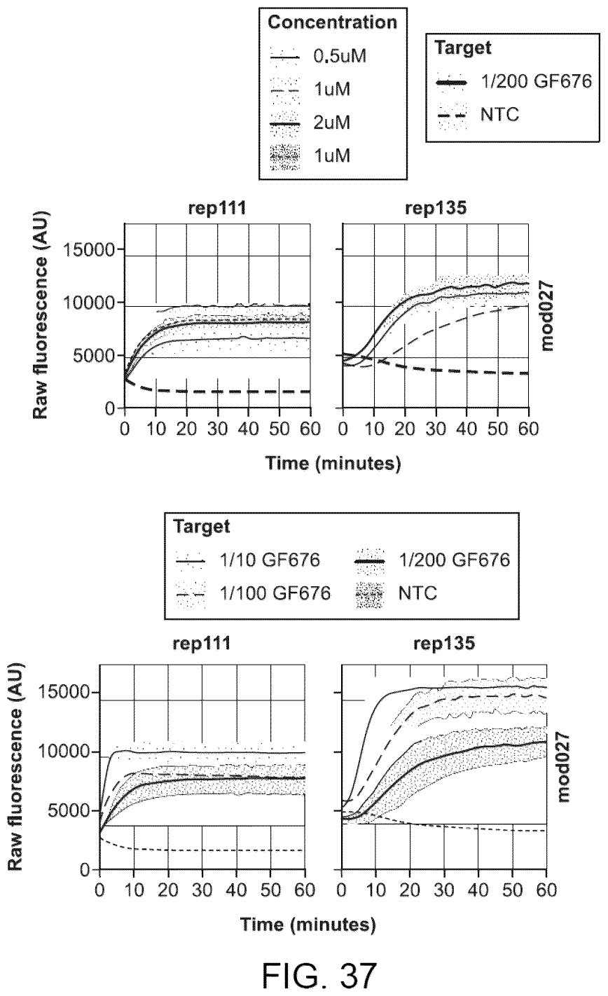

The results for optimizing guide nucleic acid and/or target concentrations to improve signal-to-noise ratios for immobilized DETECTR assays are shown in FIG. 37. In some embodiments, guide nucleic acid concentrations are increased while keeping reporter concentrations constant at 0.5 μM, as seen on the left of FIG. 37. In such embodiments, the signal is not substantially changed. In some embodiments, as seen on the right of FIG. 37, increasing target concentrations 2-fold helped improve the overall signal with rep135. Additionally, for such embodiments, rep135 gave a better signal strength compared to rep111. The sequences for the two reporters are: rep111: 5AmMC6T//16-FAMK/TTTTTTTTTTTT/3IABKFQ/(SEQ ID NO: 115) and rep135: 5AmMC12/i16-FAMK/TTTTTTTTTTTTTTTTTTT/3IABKFQ/(SEQ ID NO: 116).

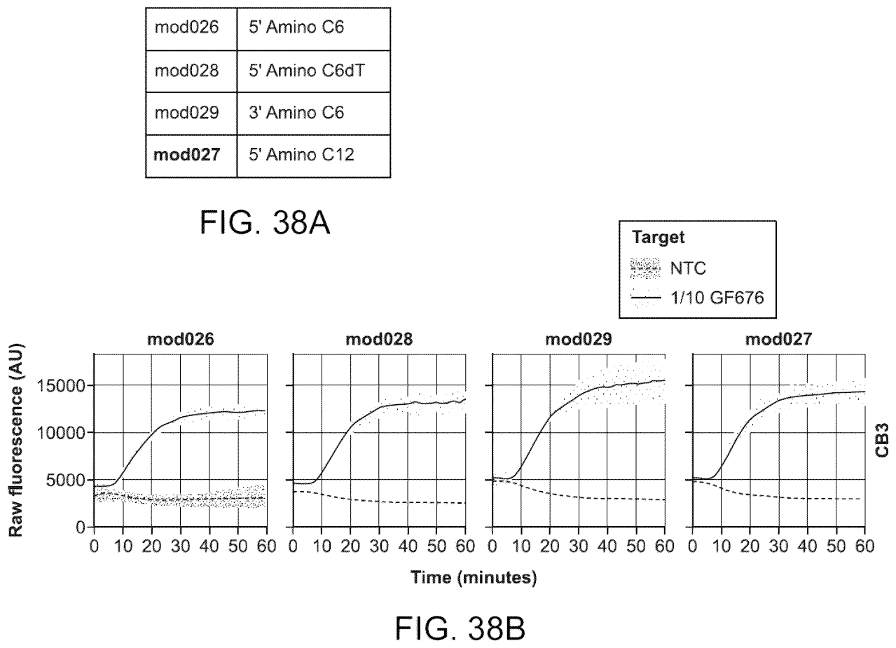

In some embodiments, one or more amino modifications are used for DETECTR immobilization, as shown in FIG. 38A. FIG. 38B presents results for such embodiments. The results are shown as plots of raw fluorescence (AU) as a function of time (minutes). Each of the four subplots represents different amino modifications. The dashed line traces represent the no target control (NTC) and the solid line traces represent a 1:10 dilution of target-GF676.

Methods of Making Polymer Matrices with Immobilized Reporters

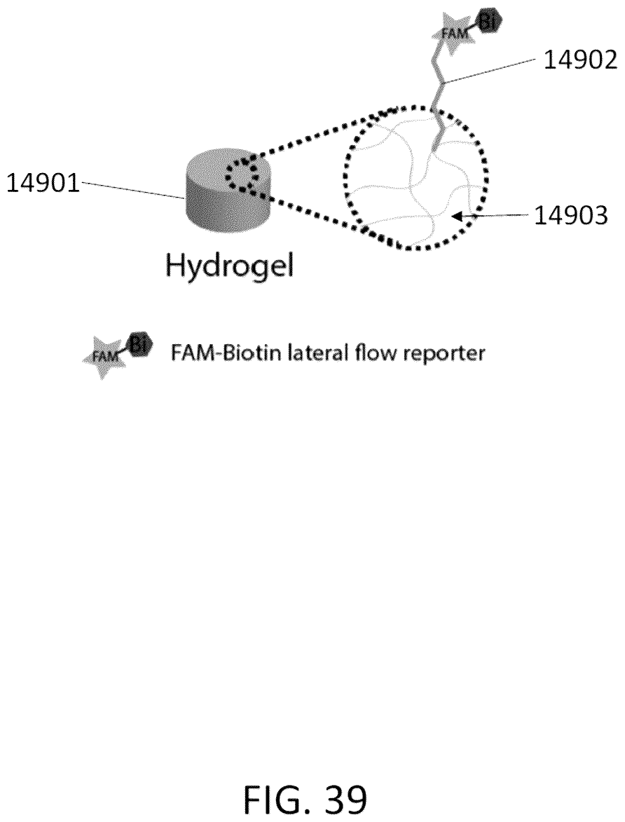

FIG. 39 shows an exemplary polymer immobilization matrix (14901) comprising a plurality of immobilized DETECTR reaction components. The DETECTR reaction components may comprise one or more reporters, one or more programmable nucleases, and/or one or more guide nucleic acids. In some embodiments, the polymer matrix may comprise a hydrogel. In the exemplary embodiment shown in FIG. 39, a plurality of reporters (14902) may be immobilized to or within a hydrogel (14901) matrix (e.g., via polymerization or co-polymerization).

In one aspect, the present disclosure provides methods of immobilizing a reporter and/or other DETECTR reaction components. In some embodiments, the methods of immobilizing the reporter (14902) and/or the other DETECTR reaction components may comprise (a) providing a polymerizable composition comprising: (i) a plurality of oligomers, (ii) a plurality of polymerizable (e.g., functionalized) oligomers, (iii) a set of polymerizable (e.g., functionalized) reporters (and/or other DETECTR reaction components), and (iv) a set of polymerization initiators. In some embodiments, the methods may further comprise (b) initiating the polymerization reaction by providing an initiation stimulus.

In some cases, co-polymerization of the reporter into or onto the hydrogel may result in a higher density of reporter/unit volume or reporter/unit area than other immobilization methods utilizing surface immobilization (e.g., immobilization onto beads). Co-polymerization of the reporter into or onto the hydrogel may result in fewer undesired releases of the reporter (e.g., during an assay, a measurement, or on the shelf), and thus may cause less background noise than other immobilization strategies (e.g., conjugation to a pre-formed hydrogel, bead, etc.). In at least some instances, this may be due to better incorporation of reporters into the hydrogel as a co-polymer and fewer “free” reporter molecules retained on the hydrogel via non-covalent interactions or non-specific binding interactions.

In some embodiments, the plurality of oligomers and/or the plurality of polymerizable oligomers may comprise an irregular or non-uniform mixture. The irregularity of the mixture of polymerizable oligomers and unfunctionalized oligomers may allow pores to form within the hydrogel (i.e., the unfunctionalized oligomers may act as a porogen). For example, the irregular mixture of oligomers may result in phase separation during polymerization that allows for the generation of pores of sufficient size for programmable nucleases to diffuse into the hydrogel and access internal reporter molecules. The relative percentages and/or molecular weights of the oligomers may be varied to optimize the pore size of the hydrogel. For example, pore size may be tailored to increase the diffusion coefficient of the programmable nucleases.

In some embodiments, the functional groups attached to the reporters may be selected to preferentially incorporate the reporters into the hydrogel matrix via covalent binding at the functional group versus other locations along the nucleic acid of the reporter. In some embodiments, the functional groups attached to the reporters may be selected to favorably transfer free radicals from the functionalized ends of polymerizable oligomers to the functional group on the end of the reporter (e.g., 5′ end), thereby forming a covalent bond and immobilizing the reporter rather than destroying other parts of the reporter molecules.

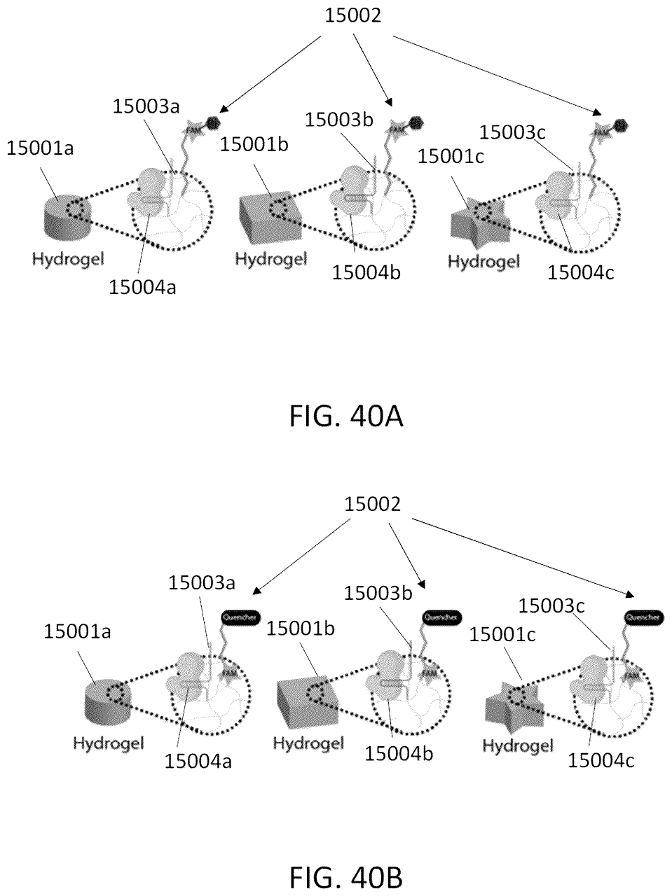

In some embodiments, the polymerizable composition may further comprise one or more polymerizable nucleic acids. In some embodiments, the polymerizable nucleic acids may comprise guide nucleic acids (e.g., guide nucleic acids 15003a, 15003b, or 15003c shown in FIGS. 40A-40B). In some embodiments, the polymerizable nucleic acids may comprise linkers or tether nucleic acids. In some embodiments, the polymerizable nucleic acids may be configured to bind to one or more programmable nuclease (e.g., programmable nuclease 15004a, 15004b, or 15004c shown in FIGS. 40A-40B). In some embodiments, the one or more programmable nucleases may be immobilized in, on, or to the polymer matrix.

In some embodiments, the oligomers may form a polymer matrix comprising a hydrogel. In some embodiments, the oligomers may comprise poly(ethylene glycol) (PEG), poly(siloxane), poly(hydroxyethyl acrylate, poly(acrylic acid), poly(vinyl alcohol), poly(butyl acrylate), poly(2-ethylhexyl acrylate), poly(methyl acrylate), poly(ethyl acrylate), poly(acrylonitrile), poly(methyl methacrylate), poly(acrylamide), poly(TMPTA methacrylate), chitosan, alginate, or the like, or any combination thereof. The oligomers may comprise any oligomer or mix of oligomers capable of forming a hydrogel.

In some embodiments, the oligomers may comprise polar monomers, nonpolar monomers, protic monomers, aprotic monomers, solvophobic monomers, solvophillic monomers, or any combination thereof.

In some embodiments, the oligomers may comprise a linear topology, branched topology, star topology, dendritic topology, hyperbranched topology, bottlebrush topology, ring topology, catenated topology, or any combination thereof. In some embodiments, the oligomers may comprise 3-armed topology, 4-armed topology, 5-armed topology, 6-armed topology, 7-armed topology, 8-armed topology, 9-armed topology, or 10-armed topology.

In some embodiments, the oligomers may comprise at least about 2 monomers, at least about 3 monomers, at least about 4 monomers, at least about 5 monomers, at least about 6 monomers, at least about 7 monomers, at least about 8 monomers, at least about 9 monomers, at least about 10 monomers, at least about 20) monomers, at least about 30 monomers, at least about 40) monomers, at least about 50) monomers, at least about 60 monomers, at least about 70) monomers, at least about 80) monomers, at least about 90 monomers, at least about 100) monomers, at least about 200 monomers, at least about 300 monomers, at least about 400) monomers, at least about 500) monomers, at least about 600) monomers, at least about 700) monomers, at least about 800) monomers, at least about 900 monomers, at least about 1000) monomers, at least about 2000) monomers, at least about 3000 monomers, at least about 4000) monomers, at least about 5000) monomers, at least about 6000 monomers, at least about 7000) monomers, at least about 8000 monomers, at least about 9000 monomers, at least about 10000 monomers, at least about 20000 monomers, at least about 30000 monomers, at least about 40000) monomers, at least about 50000 monomers, at least about 60000 monomers, at least about 70000 monomers, at least about 80000 monomers, at least about 90000 monomers, or at least about 100000 monomers.

In some embodiments, the oligomers may comprise a homopolymer, a copolymer, a random copolymer, a block copolymer, an alternative copolymer, a copolymer with regular repeating units, or any combination thereof.

In some embodiments, the oligomers may comprise 1 type of monomer, 2 types of monomers, 3 types of monomers, 4 types of monomers, 5 types of monomers, 6 types of monomers, 7 types of monomers, 8 types of monomers, 9 types of monomers, or 10 types of monomers.

The polymerizable oligomers may comprise any of the oligomers described herein. In some embodiments, the polymerizable oligomers may comprise one or more functional groups. In some embodiments, the functional group may comprise an acrylate group, N-hydroxysuccinimide ester group, thiol group, carboxyl group, azide group, alkyne group, an alkene group, or any combination thereof. A variety of functional groups may be used to functionalize oligomers into polymerizable oligomers depending on the desired properties of the polymerizable oligomers.

In some embodiments, the polymerizable oligomers may form a polymer matrix comprising a hydrogel. In some embodiments, the polymerizable oligomers may comprise PEG, poly(siloxane), poly(hydroxyethyl acrylate, poly(acrylic acid), poly(vinyl alcohol), or any combination thereof. The set of polymerizable oligomers may comprise any polymer capable of forming a hydrogel. In some embodiments, the hydrogel may comprise a circular cross-sectional shape, a rectangular cross-sectional shape, a star cross-sectional shape, a dollop shape, an amorphous shape, or any shape of interest, or any combination thereof (e.g., as shown in FIGS. 40A-40B).

In some embodiments, the set of polymerizable oligomers may comprise, for example, polar monomers, nonpolar monomers, protic monomers, aprotic monomers, solvophobic monomers, or solvophillic monomers.

In some embodiments, the set of polymerizable oligomers may comprise a linear topology, branched topology, star topology, dendritic topology, hyperbranched topology, bottlebrush topology, ring topology, catenated topology, or any combination thereof. In some embodiments, the set of polymerizable oligomers may comprise 3-armed topology, 4-armed topology, 5-armed topology, 6-armed topology, 7-armed topology, 8-armed topology, 9-armed topology, or 10-armed topology.

In some embodiments, the set of polymerizable oligomers may comprise at least about 2 monomers, at least about 3 monomers, at least about 4 monomers, at least about 5 monomers, at least about 6 monomers, at least about 7 monomers, at least about 8 monomers, at least about 9) monomers, at least about 10 monomers, at least about 20 monomers, at least about 30 monomers, at least about 40) monomers, at least about 50) monomers, at least about 60) monomers, at least about 70) monomers, at least about 80) monomers, at least about 90) monomers, at least about 100 monomers, at least about 200 monomers, at least about 300) monomers, at least about 400 monomers, at least about 500) monomers, at least about 600 monomers, at least about 700) monomers, at least about 800 monomers, at least about 900 monomers, at least about 1000 monomers, at least about 2000 monomers, at least about 3000 monomers, at least about 4000 monomers, at least about 5000 monomers, at least about 6000 monomers, at least about 7000 monomers, at least about 8000 monomers, at least about 9000 monomers, at least about 10000 monomers, at least about 20000 monomers, at least about 30000 monomers, at least about 40000 monomers, at least about 50000 monomers, at least about 60000 monomers, at least about 70000 monomers, at least about 80000 monomers, at least about 90000 monomers, or at least about 100000 monomers. As used herein, “about” may mean plus or minus 1 monomer, plus or minus 10 monomers, plus or minus 100 monomers, plus or minus 1000 monomers, plus or minus 10000 monomers, or plus or minus 100000 monomers.

In some embodiments, the set of polymerizable oligomers may comprise a homopolymer, a copolymer, a random copolymer, a block copolymer, an alternative copolymer, a copolymer with regular repeating units, or any combination thereof.

In some embodiments, the set of polymerizable oligomers may comprise 1 type of monomer, 2 types of monomers, 3 types of monomers, 4 types of monomers, 5 types of monomers, 6 types of monomers, 7 types of monomers, 8 types of monomers, 9 types of monomers, or 10 types of monomers.

In some embodiments, the polymerizable composition may comprise a mix of unfunctionalized or unmodified oligomers and polymerizable oligomers as described herein. In some embodiments, the unfunctionalized or unmodified oligomers may act as porogens to generate pores within the polymer matrix.

The polymerizable reporters may comprise any of the reporters described herein. In some embodiments, the set of polymerizable reporters may comprise one or more functional groups. In some embodiments, the functional groups may comprise a single stranded nucleic acid, a double stranded nucleic acid, an acrydite group, a 5′ thiol modifier, a 3′ thiol modifier, an amine group, a I-Linker™ group, methacryl group, or any combination thereof. In any of the embodiments described herein, a variety of functional groups may be used with the set of polymerizable reporters depending on the desired properties of the polymerizable reporters.

In some embodiments, a set of initiators may be used to initiate or facilitate any one or more of the polymerization reactions described above. In some embodiments, the set of initiators may comprise one or more photoinitiators or thermal initiators. In some embodiments, the set of initiators may comprise cationic initiators, anionic initiators, or radical initiators. In some embodiments, the set of initiators may comprise AIBN, AMBN, ADVN, ACVA, dimethyl 2,2-azo-bis(2methylpropionate), AAPH, 2,2-azobis[2-(2-imidazolin-2-yl)-propane] dihydrochloride, TBHP, cumene hydroperoxide, di-tert-butyl peroxide, dicumyl peroxide, BPO, dicyandamide, cyclohexyl tosylate, diphenyl(methyl)sulfonium tetrafluoroborate, benzyl(4-hydroxyphenyl)-methylsulfonium hexafluoroantimonate, (4-hydroxyphenyl)methyl-(2-methylbenzyl)sulfonium hexafluoroantimonate, camphorquinone, acetophenone, 3-acetophenol, 4-acetophenol, benzophenone, 2-methylbenzophenone, 3-methylbenzophenone, 3-hydroxy benzophenone, 3,4-dimethylbenzophenone, 4-hydroxy benzophenone, 4-benzoylbenzoic acid, 2-benzoylbenzoic acid, methyl 2-benzoylbenzoate, 4,4′-dihydroxy benzophenone, 4-(dimethylamino)-benzophenone, 4,4′-bis(dimethylamino)-benzophenone, 4,4′-bis(diethylamino)-benzophenone, 4,4-dichlorobenzophenone, 4-(p-tolylthio)benzophenone, 4-phenylbenzophenone, 1,4-dibenzoylbenzene, benzil, 4,4-dimethylbenzil, p-anisil, 2-benzoyl-2-propanol, 2-hydroxy-4-(2-hydroxyethoxy)-2-methylpropiophenone, 1-benzoy Ichclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio)-2-morpholinopropiophenone, 2-benzyl-2-(dimethylamino)-4′-morpholinobutyrophenone, 2-isonitrosopropiophenone, anthraquinone, 2-ethylantraquinone, sodium anthraquinone-2-sulfonate monohydrate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2 bis(2-chlorophenyl)-4,4′,5,5′-tetraphenyl-1,2-biimidazole, diphenyl(2,4,6-trimethyl-benzoyl)phosphine oxide, phenylbis(2,4,6-trimethyl-benzoyl)phosphine oxide, lithium phenyl(2,4,6-trimethylbenzoyl)phosphinate, diphenyliodonium trifluoromethanesulfonate, diphenyliodonium hexafluorophosphate, diphenyliodonium hexafluoroarsenate, bis(4-tert-butylphenyl)-iodonium triflate, bis(4-tert-butylphenyl)iodonium hexafluorophosphate, 4-isopropyl-4′-methyl-diphenyliodonium tetrakis(pentafluorophenyl)borate, [4-[(2-hydroxytetradecyl)-oxy]phenyl]phenyliodonium hexafluoroantimonate, bis[4-(tert-butyl)phenyl]-iodonium tetra(nonafluoro-tert-butoxy)aluminate, cyclopropyldiphenylsulfonium tetrafluoroborate, triphenylsulfonium bromide, triphenylsulfonium tetrafluoroborate, tri-p-tolylsulfonium triflate, tri-p-tolylsulfonium hexafluorophosphate, 4-nitrobenzenediazonium tetrafluoroborate, 2-(4-methoxyphenyl)-4,6-bis(trichloromethyl)-1,3,5-triazine, 2-(1,3-benzodioxol-5-yl)-4,6-bis(trichloromethyl)-1,3,5-triazine, 2-(4-methoxystyryl)-4,6-bis(trichloromethyl)-1,3,5-triazine, 2-(3,4-dimethoxystyryl)-4,6-bis(trichloromethyl)-1,3,5-triazine, 2-[2-(Furan-2-yl)vinyl]-4,6-bis(trichloromethyl)-1,3,5-triazine, 2-[2-(5-methylfuran-2-yl)vinyl]-4,6-bis(trichloromethyl)-1,3,5-triazine, 2-(9)-oxoxanthen-2-yl)proprionic acid 1,5,7-triazabicyclo[4.4.0]dec-5-ene salt, 2-(9-oxoxanthen-2-yl)proprionic acid 1,5-diazabicyclo[4.3.0]non-5-ene salt, 2-(9-oxoxanthen-2-yl)proprionic acid 1,8-diazabicyclo[5.4.0]-undec-7-ene salt, acetophenone O-benzoyloxime, 2-nitrobenzyl cyclohexylcarbamate, 1,2-bis(4-methoxyphenyl)-2-oxoethyl cyclohexylcarbamate, tert-amyl peroxy benzoate, 4,4-azobis(4-cyanovaleric acid), 1,1′-azobis(cyclohexanecarbonitrile), 2,2′-azobisisobutyronitrile, benzoyl peroxide, 2,2-bi(tert-butylperoxy)butane, 1,1-bis(tert-butylperoxy)cyclohexane, 2,5-bis(tert-butylperoxy)-2,5-dimethylhexane, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butyl peroxy benzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, 2-Hydroxy-2-methylpropiophenone, or any combination thereof. In any of the embodiments described herein, a variety of initiators may be used depending on the desired reaction conditions and chemistries.

In some embodiments, the initiation stimulus may comprise heat or thermal energy. In other embodiments, the initiation stimulus may comprise light (e.g., UV light). In some embodiments, the initiation stimulus may comprise UV light transmitted through a mask (e.g., a photomask). In some embodiments, a mask may be used to shape the initiation stimulus deposition on the polymerizable components (e.g., oligomers, etc.) and thereby shape the resulting polymer matrix. In some embodiments, the mask may comprise a circular shape, an oval shape, an elliptical shape, a rectangular shape, a star shape, a dollop shape, an amorphous shape, a polygonal shape comprising three or more sides, and/or any shape of interest, or any combination thereof.

Hydrogel Compositions with Immobilized Reporters

FIG. 39 and FIGS. 40A-40B show examples of hydrogels comprising immobilized reporters. In some aspects, provided herein are compositions comprising a hydrogel (14901) comprising (a) a network of covalently bound oligomers (14903) and (b) immobilized reporters (14902) covalently bound to said network (14903).

FIG. 39 shows an exemplary hydrogel (14901) comprising a plurality of reporters (14902) co-polymerized with a plurality of oligomers (modified and unmodified) to form a network or matrix (14903). FIGS. 40A-40B show exemplary multiplexing schemes utilizing hydrogel-immobilized reporters which may be implemented in any of the devices or methods described herein. Multiplexing can be distinguished through spatial multiplexing based on (i) the location of hydrogels functionalized with each guide nucleic acid and/or (ii) the shape of the hydrogels, by using different shapes of hydrogels for each guide nucleic acid.

In some embodiments, the composition may comprise a hydrogel (15001) comprising (a) a polymer network comprising covalently bound oligomers co-polymerized with reporters (15002) to covalently bind and immobilize the reporters to said network, and (b) immobilized programmable nuclease complexes covalently bound to said network (e.g., via co-polymerization or after reporter-immobilized polymer formation). In some embodiments, the programmable nuclease complexes may comprise a programmable nuclease (15004) and a guide nucleic acid (15003). In some embodiments, the guide nucleic acid (15003) and/or the programmable nuclease (15004) may be immobilized on, to, or in the hydrogel as described elsewhere herein (e.g., during or after formation of the hydrogel).

In some embodiments, the network of covalently bound oligomers may comprise a network formed by polymerizing one or more PEG species. In some embodiments, the network of covalently bound oligomers may comprise a network formed by polymerizing PEG comprising acrylate functional groups. In some embodiments, the acrylate functional groups may comprise PEG end groups. In some embodiments, the network may be formed by polymerizing PEG comprising acrylate functional groups with unmodified PEG. The molecular weight of the acrylate-modified PEG (e.g., PEG-diacrylate) and the unmodified PEG may be the same or similar. Alternatively, the molecular weight of the acrylate-modified PEG (e.g., PEG-diacrylate) and the unmodified PEG may be different.

In some embodiments, the network of covalently bound oligomers may comprise a network that can be formed by polymerizing one or more PEG species. In some cases, each PEG species may comprise a linear topology, branched topology, star topology, dendritic topology, hyperbranched topology, bottlebrush topology, ring topology, catenated topology, or any combination thereof. In some embodiments, the network of covalently bound oligomers may comprise a network formed from polymerizing one or more PEG species comprising a 3-armed topology, a 4-armed topology, a 5-armed topology, a 6-armed topology, a 7-armed topology, a 8-armed topology, a 9-armed topology, or a 10-armed topology.

In some embodiments, the immobilized reporter may comprise a reporter molecule covalently bound to a linker molecule. In some cases, the linker molecule may be covalently bound to the hydrogel (e.g., via co-polymerization with the oligomers as described herein). In some embodiments, the linker molecule may comprise a single stranded nucleic acid, a double stranded nucleic acid, an acrydite group, a 5′ thiol modifier, a 3′ thiol modifier, an amine group, a I-Linker™ group, or any combination thereof. In any of the embodiments described herein, a variety of linker molecules may be used to immobilize the reporter.

In some cases, the immobilized guide nucleic acid may comprise a guide nucleic acid covalently bound to a linker molecule. In some cases, the linker molecule may be covalently bound to the hydrogel. In some embodiments, the linker molecule may comprise a single stranded nucleic acid, a double stranded nucleic acid, an acrydite group, a 5′ thiol modifier, a 3′ thiol modifier, an amine group, a I-Linker™ group, or any combination thereof. In any of the embodiments described herein, a variety of linker molecules may be used to immobile the guide nucleic acid.

In some cases, the immobilized programmable nuclease may comprise a programmable nuclease covalently bound to a linker molecule. The linker molecule may be covalently bound to the hydrogel. In some embodiments, the linker molecule may comprise a single stranded nucleic acid, a double stranded nucleic acid, an acrydite group, a 5′ thiol modifier, a 3′ thiol modifier, an amine group, a I-Linker™ group, or any combination thereof. In any of the embodiments described herein, a variety of linker molecules may be used to immobilize the programmable nuclease.

Methods of Using Hydrogels with Immobilized Reporters

Any of the methods described herein may utilize hydrogels (14901) with immobilized reporters (14902) for target detection assays. In some embodiments, the hydrogel (14901) may comprise (a) a network of covalently bound oligomers (14903) and (b) immobilized reporters (14902) covalently bound to the network (14903) as shown in FIG. 39. A solution comprising target nucleic acid molecules and programmable nuclease complexes may be applied to the hydrogel (e.g., by pipetting or flowing over the hydrogel). The immobilized reporters (14902) may comprise a nucleic acid having a sequence cleavable by the programmable complex when the programmable nuclease complex is activated by binding of its associated guide nucleic acid to a target nucleic acid molecule as described herein. When activated, the programmable nuclease complex may trans-cleave the cleavable nucleic acid of the reporter molecule and generate a detectable signal as described herein. For example, the reporter may comprise a detection moiety which may be released upon cleavage of the reporter as described herein. The detection moiety may comprise FAM-biotin which may be captured by one or more capture molecules coupled to a substrate (e.g., a lateral flow assay strip) at a detection location as described herein. Detection of the detectable signal generated at the detection location by the detection moiety may indicate the presence or absence of the target nucleic acid in the sample as described herein.

Any of the multiplexing methods described herein may utilize hydrogels (15001a, 15001b, 15001c, etc.) with immobilized reporters (15002) for multiplexed target detection assays. In some embodiments, each hydrogel (15001a, 150001b, 15001c, etc.) may comprise (a) a polymer network of covalently bound oligomers co-polymerized with reporters (15002) to covalently bind and immobilize the reporters to said network, and (b) one or more immobilized programmable nuclease complexes covalently bound to said network as shown in FIGS. 40A-40B. Each of the programmable nuclease complexes may comprise a programmable nuclease (15004a, 15004b, 15004c, etc.) and a guide nucleic acid (15003a, 15003b, 15003c, etc.). In some embodiments, the guide nucleic acid (15003) and/or the programmable nuclease (15004) may be immobilized to or in the hydrogel as described herein (e.g., during or after formation of the hydrogel). In some embodiments, multiplexing for a plurality of different targets may be facilitated by providing a plurality of different and/or spatially separated hydrogels comprising a plurality of different DETECTR reaction components. In some embodiments, each hydrogel may comprise a different programmable nuclease as described herein, Alternatively, or in combination, each hydrogel may comprise a different guide nucleic acid configured to bind to a different target nucleic acid sequence as described herein, Alternatively, or in combination, each hydrogel may comprise a different reporter as described herein, Alternatively, or in combination, each hydrogel may comprise a different shape and may be deposited on a substrate at different detection locations. For example, as shown in FIGS. 40A-40B, a first hydrogel (15001a) may comprise a first programmable nuclease (15004a), a first guide nucleic acid (15003a) configured to bind a first target nucleic acid, and a first reporter (15002). A second hydrogel (15001b) may comprise a second programmable nuclease (15004b), a second guide nucleic acid (15003b) configured to bind a second target nucleic acid, and a second reporter (15002). A third hydrogel (15001c) may comprise a third programmable nuclease (15004c), a third guide nucleic acid (15003c) configured to bind a third target nucleic acid, and a third reporter (15002). The programmable nucleases (15004a, 15004b, 15004c) may be the same programmable nuclease or different programmable nuclease. The guide nucleic acids (15003a, 15003b, 15003c) may be different guide nucleic acids configured to recognize different target nucleic acids. The reporters (15002) may be the same reporter or different reporters. A solution comprising one or more target nucleic acid molecules may be applied to the hydrogels (15001a, 15002b, 15003c), e.g., by pipetting or flowing over the hydrogels. The immobilized reporters (15002) may comprise a nucleic acid with a sequence cleavable by the programmable nuclease complexes (15004a, 15004b, 15004c) when the programmable nuclease complexes are activated by binding of their respective guide nucleic acids (15003a, 15003b, 15003c) to their respective target nucleic acid molecules as described herein. When activated, the programmable nuclease complexes may trans-cleave the cleavable nucleic acid of the reporter molecule and generate a detectable signal at the detection location as described herein. For example, the reporter may comprise a detection moiety which may be released upon cleavage of the reporter as described herein. The detection moiety may comprise FAM-biotin as shown in FIG. 40A which may be captured by one or more capture molecules coupled to a substrate (e.g., a lateral flow assay strip) at a detection location as described herein. Alternatively, the detection moiety may comprise a quencher moiety which may be released from the hydrogel upon cleavage of the reporter, thereby allowing a fluorescent moiety on another end of the reporter to fluoresce at the detection location comprising the hydrogel as shown in FIG. 40B. Detection of the detectable signal generated at the detection locations by the detection moiety may indicate the presence or absence of the target nucleic acid in the sample as described herein. Each hydrogel (15001a, 15001b, 15001c) may have a different shape and detection of a target nucleic acid may comprise detecting a particular fluorescent shape corresponding to the hydrogel shape at the detection location.

Devices Comprising Hydrogels with Immobilized Reporters

Any of the systems or devices described herein may comprise one or more hydrogels with immobilized reporters. In some embodiments, the systems and devices described herein may comprise a plurality of hydrogels each comprising reporter molecules (e.g., in order to facilitate multiplexing and/or to improve or enhance signals). In some embodiments, a first hydrogel may comprise a shape different from a shape of a second hydrogel. In some embodiments, the first hydrogel may comprise a plurality of first reporter molecules that are different than a plurality of second reporter molecules of the second hydrogel. In some embodiments, the reporters can be the same in the first and second hydrogels. In some embodiments, the first hydrogel may comprise a circular shape, a square shape, a star shape, or any other shape distinguishable from a shape of the second hydrogel. In some embodiments, the plurality of first reporter molecules may each comprise a sequence cleavable by a programmable nuclease complex comprising a first programmable nuclease and a first guide nucleic acid. In some embodiments, the plurality of second reporter molecules may each comprise a sequence not cleavable by the first programmable nuclease complex.

Any of the systems or devices described herein may comprise a plurality of hydrogels each comprising one or more reporter molecules. For example, a first hydrogel may comprise a plurality of first reporter molecules that are different than a plurality of second reporter molecules of a second hydrogel. In some embodiments, the plurality of first reporter molecules may each comprise a first fluorescent moiety. The first fluorescent moiety can be different than the fluorescent moieties of each of the plurality of second reporter molecules. In some embodiments, the plurality of first reporter molecules may each comprise a sequence cleavable by a first programmable nuclease complex comprising a first programmable nuclease and a first guide nucleic acid. In some embodiments, the plurality of second reporter molecules may each comprise a sequence cleavable by a second programmable nuclease complex comprising a second programmable nuclease and a second guide nucleic acid.

Any of the systems or devices described herein may comprise at least about 2 hydrogels, at least about 3 hydrogels, at least about 4 hydrogels, at least about 5 hydrogels, at least about 6 hydrogels, at least about 7 hydrogels, at least about 8 hydrogels, at least about 9 hydrogels, at least about 10 hydrogels, at least about 20 hydrogels, at least about 30 hydrogels, at least about 40 hydrogels, at least about 50 hydrogels, at least about 60 hydrogels, at least about 70 hydrogels, at least about 80 hydrogels, at least about 90 hydrogels, at least about 100 hydrogels, at least about 200 hydrogels, at least about 300 hydrogels, at least about 400 hydrogels, at least about 500 hydrogels, at least about 600 hydrogels, at least about 700 hydrogels, at least about 800 hydrogels, at least about 900 hydrogels, or at least about 1000 hydrogels.

Any of the systems or devices described herein may comprise one or more compartments, chambers, channels, or locations comprising the one or more hydrogels. In some embodiments, two or more of the compartments may be in fluid communication, optical communication, and/or thermal communication with one another, or any combination thereof. In some embodiments, two or more compartments may be arranged in a sequence or in series. In some embodiments, two or more compartments may be arranged in parallel. In some embodiments, two or more compartments may be arranged in sequence (i.e., in series), in parallel, or both. In some embodiments, the one or more compartments may comprise a well. In some embodiments, the one or more compartments may comprise a flow strip. In some embodiments, the one or more compartments may comprise a heating element.

In some embodiments, the devices of the present disclosure may comprise a handheld device. In some embodiments, the device may be a point-of-need device. In some embodiments, the device may comprise any one of the device configurations described herein. In some embodiments, the device may comprise one or more parts, components, or features of any one of the device configurations described herein.

### Amplifying Signals Using Positive Feedback Systems

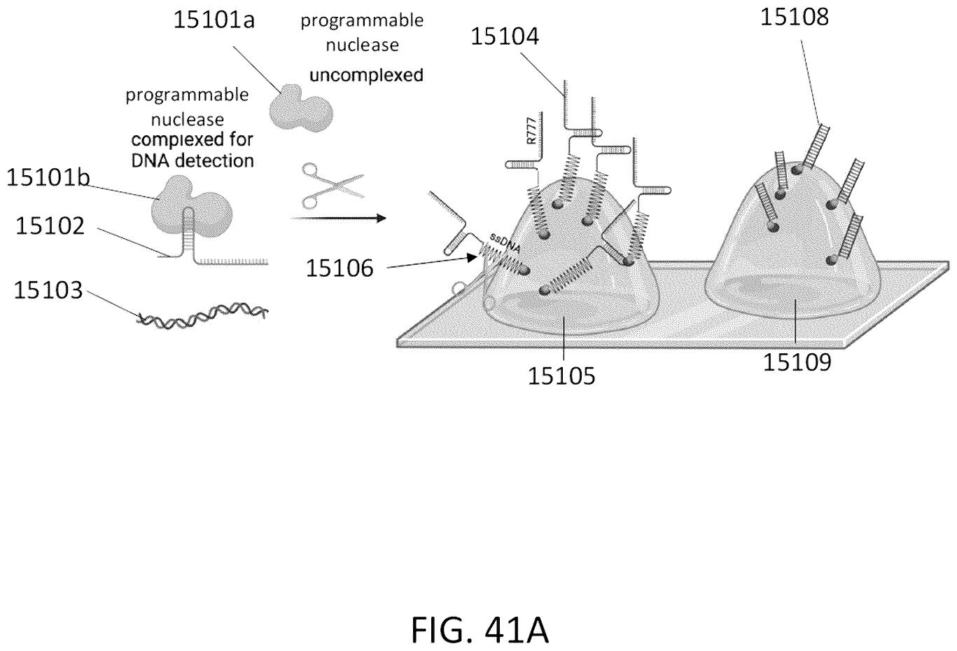

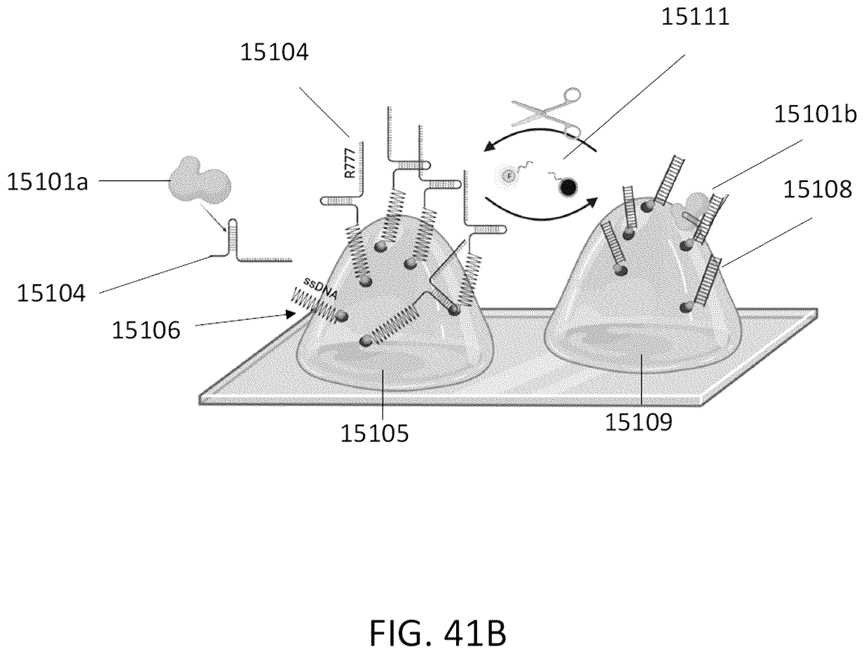

Any of the methods described herein may comprise amplifying a detection signal using a positive feedback system. FIGS. 41A-41B illustrate an exemplary positive feedback system for signal amplification. In some embodiments, a method for signal amplification may comprise binding a first nuclease, e.g., a first programmable nuclease (15101a) bound to a first guide nucleic acid (15102) with a first target nucleic acid (15103) to generate a first activated programmable nuclease complex (15101b), as shown in FIG. 41A. The first target nucleic acid (15103) may be present in a sample. Activation of the first programmable nuclease may result in release of one or more secondary target-specific guide nucleic acids (15104) from a first location (15105). The secondary target-specific guide nucleic acids (15104) may each comprise a nucleic acid tether (15106) capable of being cleaved by the first activated programmable nuclease complex. The secondary target-specific guide nucleic acids may be released by trans-cleaving the nucleic acid tethers (15106) via the first activated programmable nuclease complex. The secondary target-specific guide nucleic acids (15104) may then bind to an uncomplexed second programmable nuclease (15101a) present at the first location (15105), as shown in FIG. 41B. The second programmable nuclease (15101a) may then bind a second target nucleic acid (15108) at a second location (15109) to generate a second activated programmable nuclease complex (15101b). The second activated programmable nuclease complex (15101b) may then cleave the second target nucleic acid (15108) or remain immobilized at the second location (15109). One or more reporters may be present (e.g., free-floating, immobilized to a substrate at a third location, etc.) which may be cleaved by the first and/or second activated programmable nuclease complexes as described herein, Alternatively, or in combination, one or more of the secondary target-specific guide nucleic acid (15104), the tether (15106), and/or the second target nucleic acid (15108) may comprise a detection moiety which may provide a detectable signal upon cleavage of the nucleic acid species to which it is bound. In some embodiments, the detection moiety may comprise a quencher-fluorophore pair as described herein.

In some instances, a single first target nucleic acid can lead to the release of a plurality of secondary target-specific guide nucleic acids and the generation of the plurality of second activated programmable nuclease complexes as described herein. Then each second activated programmable nuclease complex can lead to the generation of another plurality of secondary target-specific guide nucleic acids and another plurality of second activated programmable nuclease complexes, and so on. The second activated programmable nuclease complexes can generate additional signals beyond that of the first activated programmable nuclease complex alone. Thus, the detection of the single first target nucleic acid can activate a positive feedback loop for amplifying its signal.

In some aspects, provided herein are compositions for amplifying a detection signal using a positive feedback system. In some embodiments, the composition may comprise: (a) a first set of programmable nucleases (15101) each comprising a first guide nucleic acid (15102), wherein each programmable nuclease in the first set of programmable nucleases is configured to bind with a first target nucleic acid (15103) and then trans-cleave a plurality of nucleic acids comprising a first sequence; (b) a plurality of secondary target-specific guide nucleic acids (15104) each comprising a nucleic acid tether (15106) comprising the first sequence; (c) a second set of programmable nucleases (15101) each configured to bind with a secondary target-specific guide nucleic acid which is configured to bind with a second target nucleic acid and then cleave a plurality of nucleic acids comprising a second sequence; and a plurality of second target nucleic acids (15108) each comprising the second sequence.

In some embodiments, the first programmable nuclease (15101) may be free in solution, as illustrated in FIGS. 41A-41B. In some embodiments, the first programmable nuclease (15101) may be immobilized to a substrate (e.g., 15105, 15109, etc.). In some embodiments, the second programmable nuclease (15101) may be free in solution. In some embodiments, the second programmable nuclease (15101) may be immobilized to a substrate (e.g., 15105, 15109, etc.). In some embodiments, any one or more components of the composition may be immobilized on a substrate (e.g., 15105, 15109).

In some embodiments, the substrate (15105, 15109) may comprise one or more hydrogels, as illustrated in FIGS. 41A-41B. In some embodiments, a first hydrogel (15105) may comprise a secondary target-specific guide nucleic acid (15104) immobilized by a single-stranded nucleic acid (15106). In some embodiments, a second hydrogel (15109) may comprise an immobilized second target nucleic acid (15108). In some embodiments, the substrate (15105, 15109) may comprise a reporter-incorporated hydrogel as described herein. In some embodiments, the substrate (15105, 15109) may be in the form of a bead. In some embodiments, the substrate (15105, 15109) may be a glass or glass-like material. In some embodiments, the substrate (15105, 15109) may be a polymeric material.

In some embodiments, the secondary target-specific guide nucleic acids (15104) may be immobilized to a substrate (15105, 15109). In some embodiments, the secondary target-specific guide nucleic acids (15104) may be immobilized with a single stranded nucleic acid tether (15106). In some embodiments, the secondary target-specific guide nucleic acids (15104) may be free in solution. In some embodiments, the secondary target-specific guide nucleic acids (15104) may comprise one or more reporters. In some embodiments, the secondary target-specific guide nucleic acids (15104) may comprise one or more detection moieties (15111).

In some embodiments, the second target nucleic acids (15108) may be immobilized on a substrate (15105, 15109). In some embodiments, the second target nucleic acids (15108) may be free in solution. In some embodiments, the second target nucleic acids (15108) may comprise one or more reporters. In some embodiments, the second target nucleic acids (15108) may comprise one or more detection moieties (15111).

The programmable nucleases may comprise any of the programmable nucleases described herein. In some embodiments, the nuclease may comprise an endonuclease. In some embodiments, the nuclease may comprise a Cas9 enzyme. In some embodiments, the nuclease may comprise a mutant Cas9 enzyme. In some embodiments, the nuclease may comprise an engineered Cas9 enzyme. In some embodiments, the nuclease may comprise a Cas12 enzyme. In some embodiments, the nuclease may comprise a Cas13 enzyme. In some embodiments, the nuclease may comprise a Cas14 enzyme. In some embodiments, the nuclease may comprise a CasPhi enzyme.

In some embodiments, the first and second programmable nucleases may be the same. In some embodiments, the first and second programmable nucleases may be different. In some embodiments, the programmable nucleases may be configured to carry out cis cleavage. In some embodiments, the programmable nucleases may be configured to carry out trans cleavage.

In some embodiments, cleaving by a programmable nuclease activates a reporter. In some embodiments, cleaving by a programmable nuclease activates (e.g., releases, unquenches, etc.) a detection moiety. In some embodiments, cleaving a nucleic acid tether activates a reporter. In some embodiments, cleaving a second target nucleic acid activates a reporter.

### Devices for Amplifying Signals Using Positive Feedback Systems

Any of the devices described herein may be configured for amplifying a detection signal using a positive feedback system. In some embodiments, the device may comprise one or more compartments configured to: (a) bind a first nuclease (15101) with a first guide nucleic acid (15102) and a first target nucleic acid (15103) to generate a first complex; (b) release one or more second guide nucleic acids (15104) each comprising a nucleic acid tether (15106) by cleaving the nucleic acid tether(s) (15106) with the first complex; (c) bind the second guide nucleic acids (15104) each with a second nuclease (15101) and a second target nucleic acid (15108) to generate a plurality of second complexes; and (d) cleave a plurality of reporters with the first and second complexes as described herein. Additional second complexes may be formed by further cleavage by the first and second complexes as described herein.

In some embodiments, the device may comprise one or more compartments comprising: (a) a first set of nucleases (15101) each comprising a first guide nucleic acid (15102), wherein each nuclease in the first set of nucleases is configured to bind with a first target nucleic acid (15103) and then cleave a plurality of nucleic acids comprising a first sequence; (b) a plurality of secondary target-specific guide nucleic acids (15104) each comprising a nucleic acid tether (15106) comprising the first sequence; (c) a second set of nucleases (15101) each configured to bind with the secondary target-specific guide nucleic acid (15104) and a second target nucleic acid (15108) and then cleave a plurality of nucleic acids; and (d) a plurality of second target nucleic acids (15108).

In some embodiments, the one or more compartments may be in fluid communication, optical communication, and/or thermal communication with one another, or any combination thereof. In some embodiments, the one or more compartments may be arranged in a sequence (e.g., in series). In some embodiments, one or more compartments may be arranged in parallel. In some embodiments, the one or more compartments may be arranged in sequence, in parallel, or both. In some embodiments, the one or more compartments may comprise a well. In some embodiments, the one or more compartments may comprise a flow strip. In some embodiments, the one or more compartments may comprise a heating element.

In some embodiments, the device may comprise a handheld device. In some embodiments, the device may comprise a point-of-need device. In some embodiments, the device may comprise any one of the device configurations described in this disclosure. In some embodiments, the device may comprise one or more parts, components, or features of any one of the device configurations described herein.

### Example: DETECTR-based OnePot and HotPot reactions using reporter immobilization within hydrogels

Experiments were carried out to synthesize hydrogels containing immobilized reporters co-polymerized with a mixture of oligomers as described in FIG. 39 and FIGS. 40A-40B and to determine their applicability for OnePot and HotPot DETECTR assays. FIG. 39 illustrates the hydrogel structure with a covalently incorporated reporter that was generated via co-polymerization with the reporter.

The reporter was covalently incorporated into PEG hydrogels during polymerization, A 2:1 ratio mixture of unfunctionalized PEG (MW=600 monomers) and PEG-diacrylate (MW=700) monomers) were mixed together with a photoinitiator (2-Hydroxy-2-methylpropiophenone (Darocur 1173)) and 100 μM of Acrydite-modified Reporter 172 (/5 Acryd/TTT TTT TTT TTT TTT TTT TT/16-FAMK//3Bio/) (SEQ ID NO: 117). The mixture was exposed to UV light (365 nm, 200 ms) under a photomask. The mask was configured to polymerize the mix into circular cross-sectional rods of hydrogel 400 μm in diameter. Excess material was washed off the hydrogels after polymerization. The acrydite group on the 5′ end of the reporter was covalently reacted with the acrylate groups of PEG-diacrylate oligomers during co-polymerization in order to incorporate the reporter into or onto the hydrogel.

OnePot (using a Cas12 programmable nuclease, SEQ ID NO: 17) and HotPot (using a Cas14a.1 programmable nuclease, SEQ ID NO: 3) DETECTR reactions were run as described herein by applying the programmable nuclease complexes and target nucleic acids to a tube containing the hydrogels. Six hydrogels/reaction were added for Cas12 (SEQ ID NO: 17) OnePot DETECTR and ten hydrogels/reaction were added for Cas14a.1 (SEQ ID NO: 3) HotPot DETECTR assays, DETECTR reactions were run for 60 minutes at 37° C. with mixing for the Cas12 (SEQ ID NO: 17) OnePot assays and 60 minutes at 55° C. with mixing for Cas14a.1 (SEQ ID NO: 3) HotPot assays. Duplicate reactions were run for each of a target nucleic acid (e.g., a target RNA) and the no target control (NTC) for both Cas12 (SEQ ID NO: 17) OnePot and Cas14a.1 (SEQ ID NO: 3) HotPot assays.

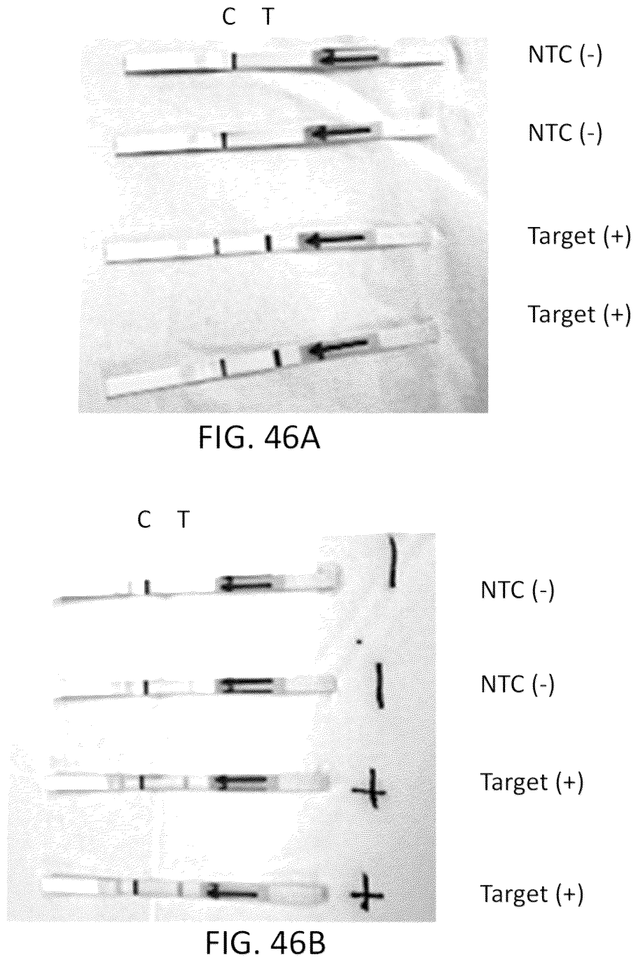

The tubes were then spun down and a supernatant was applied to lateral flow strips. The sample pad of lateral flow strip contained anti-FITC conjugate particles (colloidal gold). If the target was present, the supernatant contained cleaved FAM-biotin-labeled reporter molecules which bound to an anti-biotin (e.g., streptavidin) target line on the lateral flow strip. The anti-FITC conjugate particles bound the FAM moiety on the reporter molecules and a target band appeared on lateral flow strips at the anti-biotin target line. If the target was not present (as in NTC DETECTR reactions), the supernatant did not contain any FAM-biotin-labeled molecules and nothing bound to the anti-biotin target line. The lateral flow assay strip also contained an anti-IgG flow control line, downstream of the anti-biotin target line, which bound to the anti-FITC moiety of the conjugate particles to confirm that the lateral flow assay functioned properly. FIG. 46A shows the results of the Cas12 (SEQ ID NO: 17) OnePot DETECTR assays, FIG. 46B shows the results of the Cas14a.1 (SEQ ID NO: 3) HotPot DETECTR assays. Strong signals were seen in both positive sample replicates while minimal background noise appeared in NTC replicate strips at the target line.

Example: DETECTR-Based OnePot and HotPot Reactions Using Guide Nucleic Acid and Reporter Immobilization within Hydrogels

The following example demonstrates a method of making and using a hydrogel comprising immobilized guide nucleic acids and reporters. Guide nucleic acids are covalently incorporated into PEG hydrogels during polymerization, A 2:1 ratio mixture of unfunctionalized PEG (MW=600 monomers) and PEG-diacrylate (MW=700 monomers) are mixed together with a photoinitiator (2-Hydroxy-2-methylpropiophenone (Darocur 1173)), 100 μM of Acrydite-modified Reporter 172 (/5Acryd/TTT TTT TTT TTT TTT TTT TT/16-FAMK//3Bio/)(SEQ ID NO: 117), and 100 μM of Acrydite-modified guide nucleic acids (e.g., R1763 with acrydite modification: /5Acryd/UAA UUU CUA CUA AGU GUA GAU CCC CCA GCG CUU CAG CGU UC(SEQ ID NO: 118), or R1965 with acrydite modification: /5Acryd/rUrArAr UrUrUr CrUrAr CrUrAr ArGrUr GrUrAr GrArUr UrUrAr CrArUr GrGrCr UrCrUr GrGrUr CrCrGr Ar G)(SEQ ID NO: 119). The mixture is exposed to UV light (365 nm, 200 ms) under a photomask, The mask is configured to polymerize the mix into circular cross-sectional rods of hydrogel 400 μm in diameter. Excess material is washed off hydrogels after polymerization. The acrydite group on the 5′ end of the reporters and the acrydite group on the 5′ end of the guide nucleic acids are covalently reacted with the acrylate groups of PEG-diacrylate oligomers during co-polymerization in order to incorporate the reporters and the guide nucleic acids into the hydrogel.

OnePot (using a Cas12 programmable nuclease, SEQ ID NO: 17) and HotPot (using a Cas14a.1 programmable nuclease, SEQ ID NO: 3) DETECTR reactions are run as described herein by applying the programmable nucleases and target nucleic acids to a tube containing the hydrogels. Six hydrogels/reaction are added for the Cas12 (SEQ ID NO: 17) OnePot DETECTR assays and ten hydrogels/reaction for the Cas14a.1 (SEQ ID NO: 3) HotPot DETECTR assays, DETECTR reactions are run for 60 minutes at 37° C. with mixing for the Cas12 (SEQ ID NO: 17) OnePot assay and 60 minutes at 55° C. with mixing for the Cas14a.1 (SEQ ID NO: 3) HotPot assay. Duplicate reactions are run for each of a target nucleic acid (e.g., a target RNA) and the no target control (NTC) for both the Cas12 (SEQ ID NO: 17) OnePot and Cas14a.1 (SEQ ID NO: 3) HotPot assays.

The tubes are then spun down and a supernatant is applied to lateral flow strips. The sample pad of lateral flow strip contains anti-FITC conjugate particles (colloidal gold). If a target is present, the supernatant contains cleaved FAM-biotin-labeled reporter molecules which bind to an anti-biotin (e.g., streptavidin) target line on the lateral flow strip. The anti-FITC conjugate particles bind the FAM moiety on the reporter molecules and a target band appeared on lateral flow strips at the anti-biotin target line. If the target is not present, the supernatant does not contain any FAM-biotin-labeled molecules and nothing binds to the anti-biotin target line. The lateral flow assay strip also contains an anti-IgG flow control line, downstream of the anti-biotin target line, which binds to the anti-FITC moiety of the conjugate particles to confirm that the lateral flow assay functions properly.

Example: DETECTR-Based OnePot and HotPot Reactions Using Programmable Nuclease, Guide Nucleic Acid, and Reporter Immobilization within Hydrogels

The following example demonstrates a method of making and using a hydrogel comprising immobilized programmable nucleases, guide nucleic acids, and reporter. Guide nucleic acids are covalently incorporated into PEG hydrogels during polymerization, A 2:1 ratio mixture of unfunctionalized PEG (MW=600 monomers) and PEG-diacrylate (MW=700 monomers) are mixed together with a photoinitiator (2-Hydroxy-2-methylpropiophenone (Darocur 1173)), 100 μM of programmable nuclease (e.g., Cas12 variant SEQ ID NO: 17 or Cas14a.1 SEQ ID NO: 3), 100 μM of Acrydite-modified Reporter 172 (/5Acryd/TTT TTT TTT TTTTTT TTT TT/16-FAMK//3Bio/)(SEQ ID NO: 117), and 100 UM of Acrydite-modified guide nucleic acids (e.g., R1763 with acrydite modification: /5 Acryd/UAA UUU CUA CUA AGU GUA GAU CCC CCA GCG CUU CAG CGU UC, (SEQ ID NO: 118) or R1965 with acrydite modification: /5Acryd/rUrArAr UrUrUr CrUrAr CrUrAr ArGrUr GrUrAr GrArUr UrUrAr CrArUr GrGrCr UrCrUr GrGrUr CrCrGr Ar G (SEQ ID NO: 119)). The mixture is exposed to UV light (365 nm, 200 ms) under a photomask. The mask is configured to polymerize the mix into circular cross-sectional rods of hydrogel 400 μm in diameter. Excess material is washed off the hydrogels after polymerization. The acrydite group on the 5′ end of the reporters and the acrydite group on the 5′ end of the guide nucleic acids are covalently reacted with the acrylate groups of PEG-diacrylate oligomers during co-polymerization in order to incorporate the reporters and the guide nucleic acids into the hydrogel. The programmable nucleases are immobilized by complexing with guide nucleic acids.

OnePot and HotPot DETECTR reactions are run as described herein by applying target nucleic acids to a tube containing the hydrogels. Six hydrogels/reaction are added for the OnePot DETECTR assays and ten hydrogels/reaction are added for the HotPot DETECTR assays. The DETECTR reactions are run for 60 minutes at 37° C. with mixing for the OnePot assays and 60 minutes at 55° C. with mixing for the HotPot assays. Duplicate reactions are run for each of a target nucleic acid (e.g., a target RNA) and the no target control (NTC) for both the OnePot and HotPot assays.

The tubes are then spun down and the supernatant is applied to lateral flow strips. The sample pad of lateral flow strip contains anti-FITC conjugate particles (colloidal gold). If a target is present, the supernatant contains cleaved FAM-biotin-labeled reporter molecules which bind to an anti-biotin (e.g., streptavidin) target line on the lateral flow strip. The anti-FITC conjugate particles bind the FAM moiety on the reporter molecules and a target band appeared on lateral flow strips at the anti-biotin target line. If the target is not present, the supernatant does not contain any FAM-biotin-labeled molecules and nothing binds to the anti-biotin target line. The lateral flow assay strip also contains an anti-IgG flow control line, downstream of the anti-biotin target line, which binds to the anti-FITC moiety of the conjugate particles to confirm that the lateral flow assay functions properly,

### Example: Positive Feedback Loop System for Amplifying Signals

The following example demonstrates a positive feedback loop system for amplifying the signal for each target nucleic acid molecule in a sample as described in FIGS. 41A-41B.

A mixture comprising one or more programmable nucleases (15101), primary target-specific guide nucleic acids (15102), and primary target nucleic acids (15103) is flowed over a well. The well comprises two types of hydrogels. The first type of hydrogel (15105) contains secondary target-specific guide nucleic acids (15104) that are each immobilized by a single-stranded DNA molecule (15106) onto or into the first hydrogel (15105). The second type of hydrogel (15109) contains double stranded secondary target nucleic acids (15108) immobilized onto or into the second hydrogel (15109). The first programmable nuclease is complexed to the primary target-specific guide nucleic acid (15102). The second programmable nuclease is not bound to a guide nucleic acid.

The first programmable nucleases complexed with the primary target guide nucleic acids (15102) binds to the primary target nucleic acids (15103) to create activated programmable nuclease complexes that are configured to trans-cleave nearby single-stranded nucleic acid species. The species include reporters (15111), e.g., free-floating in solution or immobilized to a substrate or a hydrogel e.g., adjacent the single-stranded DNA molecules (15106) and/or secondary target nucleic acids, and the single-stranded DNA molecules (15106) immobilizing the secondary target-specific guide nucleic acids (15104) on or in the first hydrogel (15105). Cleavage of the single-stranded DNA linkers (15104) releases the secondary target-specific guide nucleic acids (15104) from the first hydrogel (15105). This leads to a free-floating population of secondary target-specific guide nucleic acids (15104) in solution. The free-floating secondary target-specific guide nucleic acids (15104) complex with the second programmable nucleases. The second programmable nucleases are then able to bind to the secondary target nucleic acids (15108) immobilized on the second hydrogel (15109) to form activated programmable nuclease complexes that trans-cleave nearby single-stranded nucleic acid species, including reporters (15111) and the single-stranded DNA molecules (15106) on the first hydrogel (15105). This in turn leads to additional cleavage of reporters (15111) and secondary target-specific guide nucleic acids (15104) in solution, which in turn yields more activated programmable nuclease complexes that release further secondary target-specific guide nucleic acids (15104) into the solution, and so on, thereby amplifying the initial signal from the primary target nucleic acids.

In any of the embodiments described herein, the immobilized nucleic acids (guide nucleic acids, secondary target nucleic acids) can comprise fluorescent moieties and quencher moieties. Cleavage of the immobilized nucleic acids separates the quencher moieties from the fluorescent moieties, thereby allowing the fluorescent moieties to produce a detectable fluorescent signal. The fluorescent moieties can remain bound to the hydrogels (15105, 15109) after cleavage, which can result in an increase in fluorescence at the hydrogel locations. These amplified signals can then be detected by an optical instrument or any one of the sensors described herein.

### Example: Assay Testing Using Bead Immobilized Reporters Following HotPot Protocol

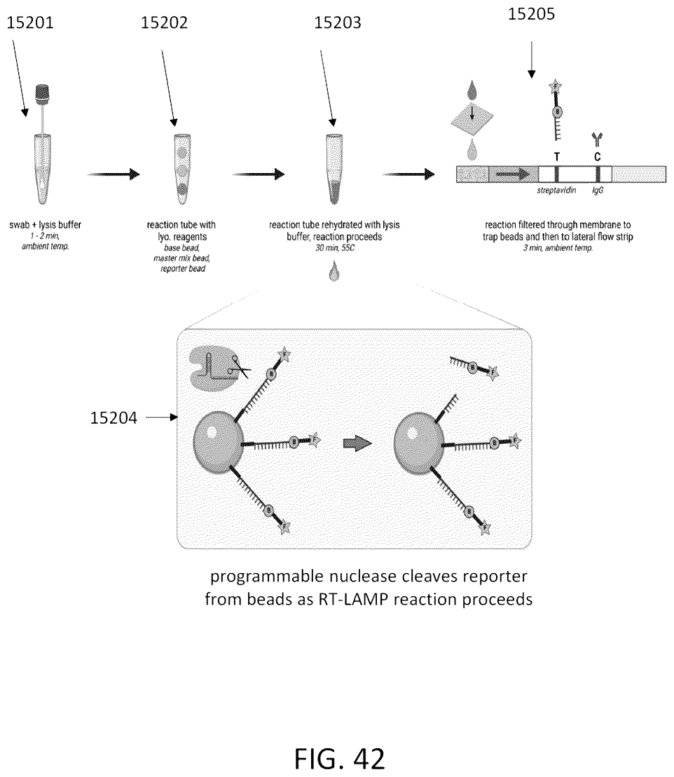

FIG. 42 illustrates the manual HotPot experimental protocol used to test bead-immobilized reporter cleavage. A sample containing target nucleic acids was added to a tube containing a lysis buffer (15201). After lysing for 1-2 minutes at ambient temperature, the solution was transferred to a reaction tube (15202) containing lyophilized reagents (i.e., the base bead, the master mix bead, and the reporter bead). Contents of the reaction tube were rehydrated and reconstituted with lysis buffer, and the HotPot reaction was started and maintained at 55° C. for 30 minutes (15203). During the reaction, programmable nucleases in the solution cleaved reporter molecules from the beads at the same time as an RT-LAMP reaction proceeded to amplify the target nucleic acids (15204). The reaction medium was then filtered through a membrane to trap the beads, and a first portion of the filtered product was used to measure fluorescence thereof on a fluorescence reader. A second portion of the filtered product was applied to a sample pad of a lateral flow strip. The lateral flow strip included a target capture area (T) comprising streptavidin and a control area (C) comprising IgG. The lateral flow strip assay was allowed to run for 3 minutes at ambient temperature (15205) before pictures were taken of the resulting bands at the target capture area T and the control area C.

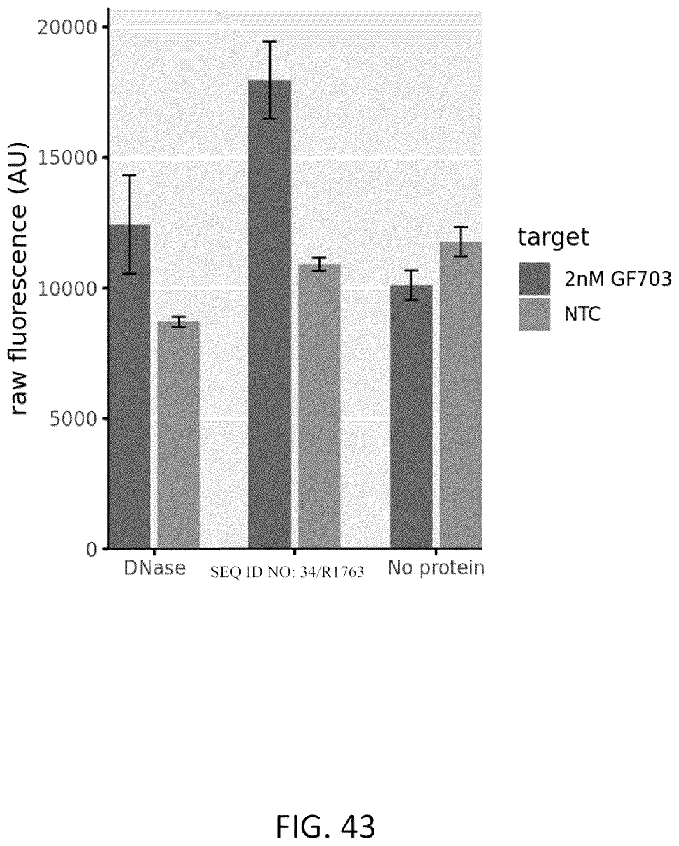

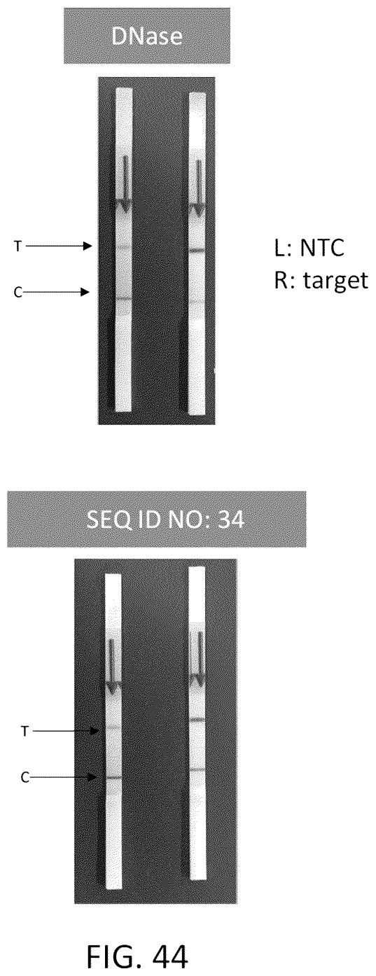

FIG. 43 shows fluorescence DETECTR results with reporters immobilized onto glass beads. Experiments with both DNase and CasM.21526 (SEQ ID NO: 34)/R1763 showed larger fluorescence signal in the presence of target nucleic acids (2 nM, GF703) compared to the no target control experiments (NTC), thus the HotPot DETECTR reaction successfully cleaved the immobilized reporters from the glass beads. Experiments with CasM.21526 (SEQ ID NO: 34) were carried out at 55° C. with H2.B buffer. Experiments with DNase were carried out at 37° C. with 1× Turbo DNase buffer. FIG. 44 shows photographs of the lateral flow strips to which the DNase and CasM.21526 (SEQ ID NO: 34) samples from FIG. 43 were applied.

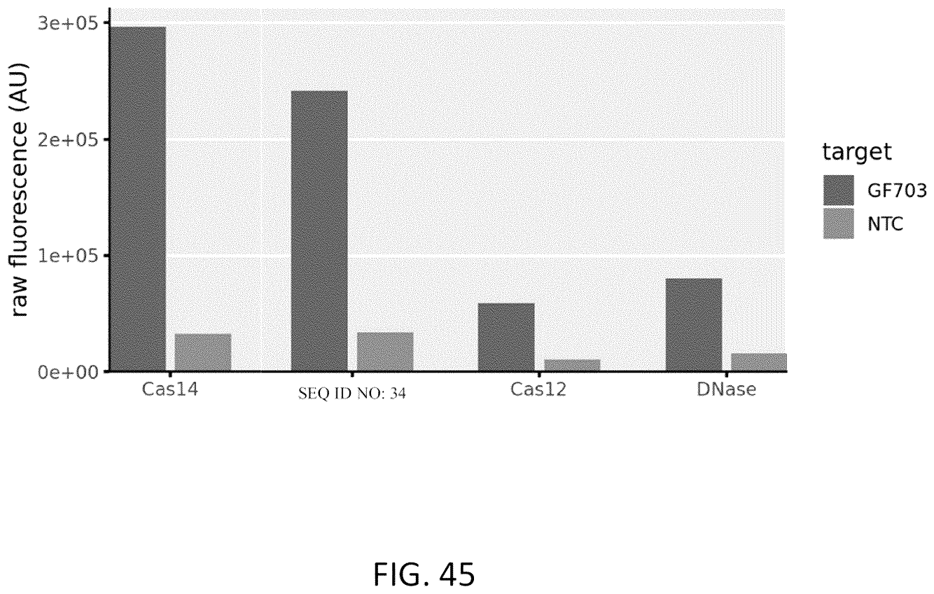

FIG. 45 shows results with maleimide-coated magnetic beads immobilized with thiol-FAM reporter. Experiments with each protein (a Cas14 variant (SEQ ID NO: 3), CasM.21526 (SEQ ID NO: 34), and a Cas12 variant (SEQ ID NO: 17)) resulted in larger signals with target nucleic acids (GF703) compared to the no target control experiments (NTC), thus the HotPot DETECTR reaction successfully cleaved the immobilized reporters from the maleimide-coated magnetic beads.

### Immobilization

In any of the embodiments described herein, the programmable nuclease, guide nucleic acid, and/or the reporter can be immobilized to a device surface by a linkage. In some embodiments, the linkage may comprise a covalent bond, a non-covalent bond, an electrostatic bond, an interaction (e.g., a covalent or noncovalent bond) between members of a binding pair (e.g., streptavidin and biotin), an amide bond, or any combination thereof. In some embodiments, the linkage may comprise non-specific absorption. In some embodiments, the programmable nuclease may be immobilized to the device surface by the linkage, wherein the linkage is between the programmable nuclease and the surface. In some embodiments, the reporter may be immobilized to the device surface by the linkage, wherein the linkage is between the reporter and the surface. In some embodiments, the guide nucleic acid may be immobilized to the surface by the linkage, wherein the linkage is between the 5′ end of the guide nucleic acid and the surface. In some embodiments, the guide nucleic acid may be immobilized to the surface by the linkage, wherein the linkage is between the 3′ end of the guide nucleic acid and the surface. In some embodiments, each guide nucleic acid of the plurality of guide nucleic acids may be complementary, or partially complementary to a different segment of the target nucleic acid. In some embodiments, the samples described and referred to herein may comprise one or more target nucleic acid(s), amplification reagents, amplified targets, and/or detection moieties.

### Lateral Flow Assay Devices

In some embodiments, the device may comprise one or more lateral flow assay strips in a detection region disposed downstream of the amplification region. Each lateral flow assay strip may comprise one or more detection regions or spots, where each detection region or spot contains a different type of capture molecule. In some embodiments, each lateral flow assay strip contains a different type of capture molecule. In some embodiments, each capture molecule type specifically binds to a particular label type of a reporter. In some embodiments, a first lateral flow assay strip contains a first capture molecule. In some embodiments, a first DETECTR region or surface location (e.g., within a reaction chamber or heating region) contains the immobilized programmable nuclease (e.g., Cas-complex) including the guide nucleic acid (e.g., sgRNA) specific to the first target nucleic acid sequence. In some embodiments, the first DETECTR region or surface location additionally contains a first immobilized reporter which is labeled with a first detection moiety (e.g., FITC). In some embodiments, upon binding of the first target nucleic acid sequence, the first immobilized reporter is cleaved and released into solution. In some embodiments, the first detection moiety is released into solution and the remainder of the first reporter remains immobilized on the surface, Alternatively, or in combination. In some embodiments, a second lateral flow assay strip contains a second capture molecule. In some embodiments, a second DETECTR region or surface location (e.g., within a reaction chamber or heating region) contains the immobilized programmable nuclease (e.g., Cas complex) including the guide nucleic acid (e.g., sgRNA) specific to the second target nucleic acid sequence. In some embodiments, the second DETECTR region or surface location additionally contains a second immobilized reporter which is labeled with a second detection moiety. Therefore, in some embodiments, upon binding of the second target nucleic acid sequence, the second immobilized reporter is cleaved and released into solution. In some embodiments, the second detection moiety is released into solution and the remainder of the second reporter remains immobilized on the surface.

In some embodiments, the solution containing the first and second cleaved reporters is transferred from the amplification region to the lateral flow region comprising the first lateral flow assay strip and the second lateral flow assay strip. In some embodiments, a chase buffer or diluent is introduced into a diluent input and negative pressure is applied to the negative pressure port to contact the solution containing the first and second cleaved reporters to the lateral flow assay strips of the lateral flow region, where the reporters are bound to conjugate molecules e.g., anti-biotin-AuNPs. In some embodiments, the AuNP-reporter conjugates having the first reporter labeled with the first detection moiety may selectively bind to a first detection region or spot containing the first capture molecule on the first lateral flow assay strip, thus indicating the presence of the first target nucleic acid sequence in the sample. In some embodiments, the AuNP-reporter conjugates having the second reporter labeled with the second detection moiety may selectively bind to a second detection region or spot containing the second capture molecule on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the device may comprise one or more lateral flow assay strips in a detection region disposed downstream of the amplification region. Each lateral flow assay strip contains one or more detection regions or spots, where each detection region or spot contains a different type of capture molecule. In some embodiments, each lateral flow assay strip may contain a different type of capture molecule. In some embodiments, each capture molecule type specifically binds to a particular label type of a reporter. In some embodiments, a first lateral flow assay strip contains a first capture molecule. In some embodiments, a first surface (e.g., bead) contains the immobilized programmable nuclease (e.g., Cas-complex) including the guide nucleic acid (e.g., sgRNA) specific to the first target nucleic acid sequence. In some embodiments, the first surface additionally contains a first immobilized reporter which is labeled with a first detection moiety. In some embodiments, upon binding of the first target nucleic acid sequence, the first immobilized reporter is cleaved and released into solution as described herein, Alternatively, or in combination, in some embodiments, a second lateral flow assay strip contains a second capture molecule. In some embodiments, a second surface (e.g., bead) contains the second immobilized programmable nuclease including the guide nucleic acid specific to the second target nucleic acid sequence. In some embodiments, the second surface additionally contains a second immobilized reporter which is labeled with a second detection moiety. Therefore, in some embodiments, upon binding of the second target nucleic acid sequence, the second immobilized reporter is cleaved and released into solution.

In some embodiments, the solution containing the first and second cleaved reporters is transferred from the amplification region to the lateral flow region comprising the first lateral flow assay strip and the second lateral flow assay strip. In some embodiments, a chase buffer or diluent is introduced into a diluent input or reservoir and negative pressure is applied to the negative pressure port to contact lateral flow region. A pressure valve may be disposed between the amplification region and the lateral flow region in order to regulate flow of the sample solution from the amplification region to the lateral flow region before amplification has occurred. Actuation of the pressure valve enables the solution containing the first and second cleaved reporters to contact the lateral flow assay strips of the lateral flow region, where the reporters are bound to conjugate molecules, e.g., anti-biotin-AuNPs. In some embodiments, the AuNP-reporter conjugates having the first reporter labeled with the first detection moiety can selectively bind to a first detection region or spot containing a first capture molecule on the first lateral flow assay strip, thus indicating the presence of the first target nucleic acid sequence in the sample. In some embodiments, the AuNP-reporter conjugates having the second reporter labeled with the second detection moiety may selectively bind to a second detection region or spot containing a second capture molecule on the second lateral flow assay strip, thus indicating the presence of the second target nucleic acid sequence in the sample. In this manner, for some embodiments, parallel detection of two or more target nucleic acid sequences present in a multiplexed sample is enabled.

In some embodiments, the device may include multiple lateral flow strips. Each reaction chamber may be configured to interface with a detection region comprising a lateral flow strip. In some embodiments, the detection region may be in fluid communication with the reaction chamber. In some embodiments, the detection region may be contacted to the reaction chamber after amplification and the programmable nuclease-based reactions have been performed in the reaction chamber. In some embodiments, the lateral flow strips may be configured to be inserted into the reaction chambers. In some embodiments, the lateral flow strips may be configured to be inserted into the reaction chambers at the same time or at different times.

In some embodiments, each reaction chamber may comprise one or more guide nucleic acids (e.g., sgRNAs) immobilized to a surface (e.g., a glass bead disposed within a reaction chamber). In some embodiments, the guide nucleic acids are part of a programmable nuclease (e.g., Cas-complex) probe immobilized to a surface. In some embodiments, a guide nucleic acid is designed to specifically bind to a target nucleic acid in the sample. In some embodiments, there are different guide nucleic acids corresponding to different reaction chambers, where each different guide nucleic acid is complementary for a different target nucleic acid sequence that may or may not be present in the sample. In some embodiments, in addition to the programmable nuclease probes containing guide nucleic acids, each reaction chamber may contain or may be functionalized with one or more reporters having distinct functional groups as described herein. In some embodiments, the reporters may be in close enough proximity to be cleaved by the programmable nuclease probes. In some embodiments, reporters are cleaved and a portion thereof (e.g., a detection moiety) may be released into the solution upon binding between a particular guide nucleic acid and the target nucleic acid to which the guide nucleic acid is designed to specifically bind. In some embodiments, reporters are functionalized with a detection moiety (e.g., a label).

In some embodiments, the device may comprise one or more lateral flow assay strips in a detection region disposed downstream of the reaction chamber. In some embodiments, the device may comprise one or more lateral flow assay strips in a detection region which may be brought into fluid communication with the reaction chamber. Each lateral flow assay strip may contain one or more detection regions or spots, where each detection region or spot contains a different type of capture molecule. In some embodiments, each lateral flow assay strip contains a different type of capture molecule. In some embodiments, each capture molecule type specifically binds to a particular label type of a reporter. In some embodiments, a first lateral flow assay strip contains a first capture molecule. In some embodiments, a first DETECTR region or surface location (e.g., within a first reaction chamber) contains the immobilized programmable nuclease (e.g., Cas-complex) including the guide nucleic acid (e.g., sgRNA) specific to the first target nucleic acid sequence. In some embodiments, the first DETECTR region or surface location additionally contains a first immobilized reporter which is labeled with a first detection moiety (e.g., FITC). In some embodiments, upon binding of the first target nucleic acid sequence, the first immobilized reporter is cleaved and released into solution. In some embodiments, the first detection moiety is released into solution and the remainder of the first reporter remains immobilized on the surface, Alternatively, or in combination. In some embodiments, a second lateral flow assay strip contains a second capture molecule. In some embodiments, a second DETECTR region or surface location (e.g., within a second reaction chamber) may contain the immobilized programmable nuclease (e.g., Cas complex) including the guide nucleic acid (e.g., sgRNA) specific to the second target nucleic acid sequence. In some embodiments, the second DETECTR region or surface location additionally contains a second immobilized reporter which is labeled with a second detection moiety. Therefore, in some embodiments, upon binding of the second target nucleic acid sequence, the second immobilized reporter is cleaved and released into solution. In some embodiments, the second detection moiety is released into solution and the remainder of the second reporter remains immobilized on the surface.

In some embodiments, the solutions containing the first or second cleaved reporters can be transferred from their respective reaction chambers to a first lateral flow assay strip and a second lateral flow assay strip, respectively. In some embodiments, a chase buffer or diluent is introduced into a diluent input and negative pressure is applied to the negative pressure port to contact the solutions containing the first or second cleaved reporters to their respective lateral flow assay strips. The reporters may be bound to conjugate molecules e.g., anti-biotin-AuNPs. In some embodiments, the AuNP-reporter conjugates having the first reporter labeled with the first detection moiety may selectively bind to a first detection region or spot containing the first capture molecule on the first lateral flow assay strip, thus indicating the presence of the first target nucleic acid sequence in the sample. In some embodiments, the AuNP-reporter conjugates having the second reporter labeled with the second detection moiety may selectively bind to a second detection region or spot containing the second capture molecule on the second lateral flow assay strip, thus indicating the presence of the second target nucleic acid sequence in the sample. In this manner, parallel detection of two or more target nucleic acid sequences present in a multiplexed sample is enabled.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein can be employed. It is intended that the following claims define the scope of the invention and that methods and structures within the scope of these claims and their equivalents be covered thereby.

1. A system for detecting any of a plurality of different target nucleic acids in a sample, the system comprising:
(a) a plurality of different non-naturally occurring guide nucleic acids, wherein each of the different non-naturally occurring guide nucleic acids is immobilized to a surface at a known location identified with the particular non-naturally occurring guide nucleic acid;
(b) a plurality of reporters immobilized to the surface in proximity to each of the different non-naturally occurring guide nucleic acids at each of the known locations;
wherein each of the different non-naturally occurring guide nucleic acids comprises a sequence that hybridizes to a segment of one of the plurality of different target nucleic acids or an amplicon thereof;
wherein each of the non-naturally occurring guide nucleic acids is effective to form a complex with a programmable nuclease that is activated upon binding the corresponding target nucleic acid or amplicon thereof at the known location; and
wherein formation of the activated complex is effective to induce detectable trans cleavage of the reporters at the respective known location.

2. The system of claim 1, wherein the plurality of different non-naturally occurring guide nucleic acids are each immobilized to the surface by a linkage.

3. The system of claim 2, wherein the linkage comprises a covalent bond, a non-covalent bond, an electrostatic bond, a bond between members of a binding pair, an amide bond, or any combination thereof.

4. The system of claim 2, wherein the linkage comprises a chain of at least 6 carbons, or at least 12 carbons.

5. The system of claim 2, wherein the linkage comprises a linker polynucleotide.

6. (canceled)

7. The system of claim 5, wherein the linker polynucleotide is double-stranded.

8. The system of claim 5, wherein the linker polynucleotide comprises double-stranded DNA or single-stranded DNA.

9. The system of claim 8, wherein the double-stranded DNA linker polynucleotide is about 60 to about 80 base pairs in length.

10. The system of claim 5, wherein the linker polynucleotide is a cleavage substrate for the activated complex.

11. The system of claim 1, wherein the reporters and the non-naturally occurring guide nucleic acids are immobilized at separate discrete positions within each of the known locations.

12. The system of claim 1, wherein a) each of the reporters comprises a fluorescent label and a quencher, and wherein cleavage of the reporters is effective to produce a detectable loss of the quencher from the respective known location, or b) each of the reporters comprises a detection moiety, and wherein cleavage of the reporters is effective to produce a detectable loss of the detection moiety from the respective known location.

13.-14. (canceled)

15. The system of claim 1, further comprising programmable nucleases immobilized at the known locations by a linkage, wherein the plurality of different non-naturally occurring guide nucleic acids are immobilized to the surface by being releasably bound by the programmable nucleases.

16. The system of claim 1, further comprising programmable nucleases bound to the non-naturally occurring guide nucleic acids.

17.-27. (canceled)

28. The system of claim 1, wherein the surface is a surface of a fluidic chamber or a bead.

29. The system of claim 1, wherein the surface comprises a polymer matrix.

30.-34. (canceled)

35. A method of assaying for a plurality of different target nucleic acids in a sample, the method comprising:
(a) contacting a system of claim 1 with the sample;
(b) detecting at one or more of the known locations a change in signal resulting from cleavage of the reporters;
wherein the known location at which the change in signal is detected identifies the target nucleic acid in the sample.

36. The method of claim 35, wherein the sample comprises products of a nucleic acid amplification reaction or products of a reverse transcription reaction.

37. (canceled)

38. A method of assaying for a plurality of different target nucleic acids in a sample, the method comprising:
(a) contacting a surface with the sample, wherein the surface comprises:
(i) a plurality of different non-naturally occurring guide nucleic acids, wherein each of the different non-naturally occurring guide nucleic acids is immobilized to the surface at a known location identified with the particular non-naturally occurring guide nucleic acid; and
(ii) a plurality of reporters immobilized to the surface in proximity to each of the different non-naturally occurring guide nucleic acids at each of the known locations.
(b) forming activated complexes at one or more of the known locations, wherein the activated complexes comprise (i) one of the different non-naturally occurring guide nucleic acids, (ii) a programmable nuclease, and (iii) one of the different target nucleic acids or an amplicon thereof;
(c) cleaving the reporters with the activated complexes at the one or more known locations by trans cleavage; and
(d) detecting a change in a signal at the one or more known locations comprising the activated complexes, wherein the change in signal is a product of the trans cleavage, and wherein the known location at which the change in signal is detected identifies the target nucleic acid in the sample.

39. The method of claim 38, wherein the step of cleaving the reporters comprises incubation at a temperature of about 37° C. to about 70° C.

40.-70. (canceled)

71. The method of claim 1, further comprising performing a nucleic acid amplification reaction targeting the plurality of different target nucleic acids, wherein the nucleic acid amplification reaction is: (i) performed on an initial sample to prepare the sample prior to step (a); or (ii) performed after step (a) and before or concurrently with step (b).

72.-75. (canceled)

76. A method of assaying for one or more target nucleic acids in a sample, the method comprising:
(a) amplifying the one or more target nucleic acids to produce DNA amplicons of the one or more target nucleic acids, wherein the amplifying comprises:
(i) a plurality of cycles, wherein each cycle comprises denaturation at a first temperature and primer extension by a polymerase at a second temperature that is lower than the first temperature;
(ii) each cycle is less than 15 seconds in duration; and
(iii) the plurality of cycles comprises at least 20 cycles;
(b) forming a complex comprising one of the DNA amplicons, a programmable nuclease, and a non-naturally occurring guide nucleic acid that hybridizes to a segment of the DNA amplicon, thereby activating the programmable nuclease;
(c) cleaving reporters with the activated programmable nuclease; and
(d) detecting a change in a signal, wherein the change in the signal is produced by cleavage of the reporters.

77.-89. (canceled)
